(19) United States
(12) Patent Application Publication
(10) Pub. No.: US 2020/0219587 A1
(43) Pub. Date: Jul. 9, 2020

(54) SYSTEMS AND METHODS FOR USING FRAGMENT LENGTHS AS A PREDICTOR OF CANCER

(71) Applicant: GRAIL, Inc., Menlo Park, CA (US)

(72) Inventor: Earl Hubbell, Palo Alto, CA (US)

(21) Appl. No.: 16/723,369

(22) Filed: Dec. 20, 2019

Related U.S. Application Data
(60) Provisional application No. 62/827,682, filed on Apr. 1, 2019.
(60) Provisional application No. 62/784,332, filed on Dec. 21, 2018.

(51) Int. Cl. G16B 20/20; G16B 30/00; G16B 10/00; G16H 10/40; G16H 70/60; G16H 50/70

(52) U.S. Cl. CPC G16B 20/20; G16B 30/00; G16H 50/70; G16H 10/40; G16H 70/60; G16B 10/00

(57) ABSTRACT

Systems and methods are provided for determining relevant medical information about a cancer based on the distribution of fragment lengths of cell-free DNA sequenced from a biological fluid sample. In certain embodiments, the systems and methods are useful for segmenting a cancer genome, phasing alleles in a cancer genome, detecting the loss of heterozygosity in a cancer genome, assigning an origin of a variant allele, validating a sequencing mapping, and validating use of an allele in a cancer classifier.

### CROSS REFERENCE TO RELATED APPLICATIONS

This application claims priority to U.S. Provisional Patent Application No. 62/784,332, filed Dec. 21, 2018, and U.S. Provisional Patent Application No. 62/827,682, filed Apr. 1, 2019, the contents of which are hereby incorporated by reference in their entireties for all purposes.

### TECHNICAL FIELD

The present disclosure relates generally to using cell-free DNA fragment length distributions to classify subjects for a cancer condition.

### BACKGROUND

The increasing knowledge of the molecular pathogenesis of cancer and the rapid development of next generation sequencing techniques are advancing the study of early molecular alterations involved in cancer development in body fluids. Specific genetic and epigenetic alterations associated with such cancer development are found in cell-free DNA (cfDNA) in plasma, serum, and urine. Such alterations could potentially be used as diagnostic biomarkers for several types of cancers. See Salvi et al., 2016, “Cell-free DNA as a diagnostic marker for cancer: current insights,” Onco Targets Ther. 9:6549-6559.

Cancer represents a prominent worldwide public health problem. The United States alone in 2015 had a total of 1,658,370 cases reported. See, Siegel et al., 2015, “Cancer statistics,” CA Cancer J Clin. 65(1):5-29. Screening programs and early diagnosis have an important impact in improving disease-free survival and reducing mortality in cancer patients. As noninvasive approaches for early diagnosis foster patient compliance, they can be included in screening programs.

Noninvasive serum-based biomarkers used in clinical practice include carcinoma antigen 125 (CA 125), carcinoembryonic antigen, carbohydrate antigen 19-9 (CA19-9), and prostate-specific antigen (PSA) for the detection of ovarian, colon, and prostate cancers, respectively. See, Terry et al., 2016, “A prospective evaluation of early detection biomarkers for ovarian cancer in the European EPIC cohort,” Clin Cancer Res. 2016 Apr. 8; Epub and Zhang et al., “Tumor markers CA19-9, CA242 and CEA in the diagnosis of pancreatic cancer: a meta-analysis,” Int J Clin Exp Med. 2015; 8(7):11683-11691.

These biomarkers generally have low specificity (high number of false-positive results). Thus, new noninvasive biomarkers are actively being sought. The increasing knowledge of the molecular pathogenesis of cancer and the rapid development of new molecular techniques such as next generation nucleic acid sequencing techniques is promoting the study of early molecular alterations in body fluids.

Cell-free DNA (cfDNA) can be found in serum, plasma, urine, and other body fluids (Chan et al., “Clinical Sciences Reviews Committee of the Association of Clinical Biochemists Cell-free nucleic acids in plasma, serum and urine: a new tool in molecular diagnosis,” Ann Clin Biochem. 2003; 40(Pt 2):122-130) representing a “liquid biopsy,” which is a circulating picture of a specific disease. See, De Mattos-Arruda and Caldas, 2016, “Cell-free circulating tumour DNA as a liquid biopsy in breast cancer,” Mol Oncol. 2016; 10(3):464-474.

The existence of cfDNA was demonstrated by Mandel and Metais (Mandel and Metais), “P. Les acides nucleiques du plasma sanguin chez l' homme [The nucleic acids in blood plasma in humans],” C R Seances Soc Biol Fil. 1948; 142(3-4):241-243). cfDNA originates from necrotic or apoptotic cells, and it is generally released by all types of cells. Stroun et al. showed that specific cancer alterations could be found in the cfDNA of patients. See, Stroun et al., “Neoplastic characteristics of the DNA found in the plasma of cancer patients,” Oncology. 1989; 46(5):318-322). A number of following papers confirmed that cfDNA contains specific tumor-related alterations, such as mutations, methylation, and copy number variations (CNVs), thus confirming the existence of circulating tumor DNA (ctDNA). See, Goessl et al., “Fluorescent methylation-specific polymerase chain reaction for DNA-based detection of prostate cancer in bodily fluids,” Cancer Res. 2000; 60(21):5941-5945 and Frenel et al., 2015, “Serial next-generation sequencing of circulating cell-free DNA evaluating tumor clone response to molecularly targeted drug administration. Clin Cancer Res. 21(20):4586-4596.

cfDNA in plasma or serum is well characterized, while urine cfDNA (ucfDNA) has been traditionally less characterized. However, recent studies demonstrated that ucfDNA could also be a promising source of biomarkers. See, Casadio et al., 2013, “Urine cell-free DNA integrity as a marker for early bladder cancer diagnosis: preliminary data,” Urol Oncol. 2013; 31(8):1744-1750.

In blood, apoptosis is a frequent event that determines the amount of cfDNA. In cancer patients, however, the amount of cfDNA seems to be also influenced by necrosis. See Hao et al., “Circulating cell-free DNA in serum as a biomarker for diagnosis and prognostic prediction of colorectal cancer,” Br J Cancer. 2014; 111(8):1482-1489 and Zonta et al., “Assessment of DNA integrity, applications for cancer research,” Adv Clin Chem. 2015; 70:197-246. Since apoptosis seems to be the main release mechanism, circulating cfDNA has a size distribution that reveals an enrichment in short fragments of about 167 bp, (see, Heitzer et al., 2015, “Circulating tumor DNA as a liquid biopsy for cancer,” Clin Chem. 61(1):112-123 and Lo et al., 2010, “Maternal plasma DNA sequencing reveals the genome-wide genetic and mutational profile of the fetus,” Sci Transl Med. 2(61):61ra91) corresponding to nucleosomes generated by apoptotic cells.

The amount of circulating cfDNA in serum and plasma seems to be significantly higher in patients with tumors than in healthy controls, especially in those with advanced-stage tumors than in early-stage tumors. See, Sozzi et al., 2003 “Quantification of free circulating DNA as a diagnostic marker in lung cancer,” J Clin Oncol. 21(21):3902-3908, Kim et al., 2014, “Circulating cell-free DNA as a promising biomarker in patients with gastric cancer: diagnostic validity and significant reduction of cfDNA after surgical resection,” Ann Surg Treat Res. 2014; 86(3):136-142; and Shao et al. 2015 “Quantitative analysis of cell-free DNA in ovarian cancer,” Oncol Lett. 2015; 10(6):3478-3482). The variability of the amount of circulating cfDNA is higher in cancer patients than in healthy individuals, (Heitzer et al., 2013, “Establishment of tumor-specific copy number alterations from plasma DNA of patients with cancer,” Int J Cancer. 133(2):346-356) and the amount of circulating cfDNA is influenced by several physiological and pathological conditions, including proinflammatory diseases. See, Raptis and Menard, 1980, “Quantitation and characterization of plasma DNA in normals and patients with systemic lupus erythematosus,” J Clin Invest. 66(6):1391-1399, and Shapiro et al., 1983, “Determination of circulating DNA levels in patients with benign or malignant gastrointestinal disease,” Cancer. 51(11):2116-2120.

Studies on transplanted tissue or single cancers have indicated that the fragment lengths of plasma-derived cfDNA reflect their respective source. Specifically, non-hematopoietically-derived cfDNA molecules are shorter than those that are hematopoietically-derived (Zheng et al., 2012, Clin Chem., 58(3), pp. 549-58), and circulating tumor DNA (ctDNA) is shorter than normal cfDNA (Jiang et al., 2015, Proc Natl Acad Sci U.S.A., 112(11), pp. E1317-25); Underhill H R et al., 2016, PLoS Genet., 12(7), e1006162). This has fueled research on the detection of tumor-derived mutations in cfDNA, commonly via whole-genome sequencing or PCR-based methods (Adalsteinsson et al., 2017, Nat Commun. 8(1), p. 1324; Przybyl et al., 2018, Clin Cancer Res. 24(11), pp. 2688-99). The results of such studies, however, are often clouded by interfering (non-tumor-specific) somatic and clonal-hematopoiesis (CH)-derived mutations (Liu et al., 2018 Ann Oncol., doi: 10.1093/annonc/mdy513. [Epub ahead of print]; Hu et al., 2018, Clin Cancer Res. 24(18), pp. 4437-43). Given that CH increases with age (Genovese et al., 2014, N Engl J Med. 371(26), pp. 2477-87; Coombs et al., 2017, Cell Stem Cell 21(3), pp. 374-82; Jaiswal et al., 2014, N Engl J Med. 371(26), pp. 2488-98), and given the prevalence of cancer in the general population (SEER), most individuals in a cancer screening population will have no tumor-derived alleles and mostly alleles from CH.

Conventional cancer diagnostics, performed by identifying the presence or absence of one or more well-characterized genomic and/or epigenetic markers indicative of a particular cancer status, facilitates personalized medicine. However, the genomes of each cancer are unique and much more complex than can be measured using a small number of well-characterized alleles that may or may not be biologically relevant to the individual cancer. Moreover, conventional cancer diagnostics rely on the identification of these alleles in biopsied samples of the cancer from the subject. This requirement for biopsy samples is costly and causes delay in providing diagnostic information to the doctor.

### SUMMARY

Accordingly, improved methods for identifying variant cancer alleles in a subject are needed. Specifically, there is a need for increased understanding about the nature of cfDNA variants derived from different sources, to improve the detection of non-metastatic tumors. The present disclosure addressed the shortcomings identified in the background by providing methods for quick and accurate identification of variant alleles arising from cancer in a subject. These methodologies are based, in part, on the development of various models of cell-free DNA fragment-length distributions that are capable of differentiating between different possible origins of variant alleles detected in cell-free DNA, as described below. Additionally, in some aspects, the present disclosure provides methods for characterizing a cancer genome in a subject through the detection of shifts in cell-free DNA fragment-length distributions in a biological fluid sample. Further, in some aspects, the disclosure provides methods that assist in the validation of sequence alignments between cell-free DNA fragment sequences and a reference genome. Finally, in some aspects, the disclosure provides methods for validating the use of genetic, epigenetic, and/or epigenomic data from a particular allele in a cancer classifier.

One aspect of the present disclosure provides a method for segmenting all or a portion of a reference genome for a species of a subject. A dataset is obtained that includes nucleic acid fragment sequences in electronic form from cell-free DNA in a first biological sample from the subject. Each respective nucleic acid fragment sequence in the nucleic acid fragment sequences represents all or a portion of a respective cell-free DNA molecule in a population of cell-free DNA molecules in the biological sample, the respective nucleic acid fragment sequence encompassing a corresponding locus in a plurality of loci, where each locus in the plurality of loci is represented by at least two different alleles within the population of cell-free DNA molecules. For each respective allele represented at each locus in the plurality of loci, a size-distribution metric is assigned based on a characteristic of the distribution of the fragment lengths of the cell-free DNA molecules in the population of cell-free DNA molecules that encompass the allele, thereby generating a set of size-distribution metrics. For each respective allele represented at each locus in the plurality of loci, one or both of: (1) a read-depth metric based on a frequency of nucleic acid fragment sequences, in the plurality of nucleic acid fragment sequences, associated with the respective allele, thereby obtaining a set of read-depth metrics, and (2) an allele-frequency metric based on (i) a frequency of occurrence of the respective allele of the respective locus across the plurality of nucleic acid fragment sequences and (ii) a frequency of occurrence of a second allele of the respective locus across the plurality of nucleic acid fragment sequences is assigned, thereby obtaining a set of allele-frequency metrics. The set of size-distribution metrics and one or both of the set of (1) read-depth metrics and (2) allele-frequency metrics is used to segment all or a portion of the reference genome for the species of the subject.

One aspect of the present disclosure provides a method for phasing alleles present on a matching pair of chromosomes in a cancerous tissue of a subject that is a member of a species. A dataset is obtained that includes nucleic acid fragment sequences in electronic form from a first biological sample of the subject. Each respective nucleic acid fragment sequence in the plurality of nucleic acid fragment sequences represents all or a portion of a respective cell-free DNA molecule in a population of cell-free DNA molecules in the first biological sample, the respective nucleic acid fragment sequence encompassing a corresponding locus in a plurality of loci, where each locus in the plurality of loci is represented by at least two different alleles within the population of cell-free DNA molecules. For each respective allele represented at each locus in the plurality of loci, a size-distribution metric is assigned based on a characteristic of a distribution of the fragment lengths of the cell-free DNA molecules in the population of cell-free DNA molecules that encompass the respective allele, thereby generating a set of size-distribution metrics. A first locus in the plurality of loci is identified, the first locus represented by both (i) a first allele having a first size-distribution metric and (ii) a second allele having a second size-distribution metric, where a threshold probability or likelihood exists that the copy number of the first allele is different than the copy number of the second allele in a subpopulation of cells within the cancerous tissue of the subject as determined by a parametric or non-parametric based classifier that evaluates one or more properties of the cell-free DNA molecules in the sample that encompass the first locus. The one or more properties includes the first size-distribution metric and the second size-distribution metric. For a second locus in the plurality of loci located proximate to the first locus on a reference genome for the species of the subject, the second locus represented by both (iii) a third allele having a third size-distribution metric and (iv) a fourth allele having a fourth size-distribution metric, it is determined whether a threshold probability exists that the copy number of the third allele is different than the copy number of the fourth allele in the sub-population of cells as determined by a parametric or non-parametric based classifier that evaluates one or more properties of the cell-free DNA molecules in the sample that encompass the second locus. The one or more properties includes the third size-distribution metric and the fourth size-distribution metric. When the threshold probability or likelihood exists that the copy number of the third allele is different than the copy number of the fourth allele in the sub-population of cells, it is determined whether it is more likely that the copy number of the first allele is more similar to the copy number of the third allele or the copy number of the fourth allele in the subpopulation of cancer cells. When it is more likely that the copy number of the first allele is more similar to the copy number of the third allele in the subpopulation of cancer cells, the first allele and the third allele are assigned to a first chromosome in a matching pair of chromosomes and the second allele and the fourth allele are assigned to a second chromosome in the matching pair of chromosomes that is different than the first chromosome. When it is more likely that the copy number of the first allele is more similar to the copy number of the fourth allele in the sub-population, the first allele and the fourth allele are assigned to a first chromosome in a matching pair of chromosomes and the second allele and the third allele are assigned to a second chromosome in the matching pair of chromosomes that is different than the first chromosome. Accordingly, the allele sequences at the first and second loci present on a matching pair of chromosomes in the cancerous tissue are phased.

One aspect of the present disclosure provides a method for detecting a loss in heterozygosity at a genomic locus in a cancerous tissue of a subject. A dataset is obtained that includes a plurality of nucleic acid fragment sequences in electronic form from a first biological sample of the subject. Each respective nucleic acid fragment sequence in the plurality of nucleic acid fragment sequences represents all or a portion of a respective cell-free DNA molecule, in a population of cell-free DNA molecules in the first biological sample, the respective nucleic acid fragment sequence encompassing a corresponding locus in a plurality of loci, where each locus in the plurality of loci is represented by at least two different germline alleles. For each respective germline allele represented at each locus in the plurality of loci, a size-distribution metric is assigned based on a characteristic of the distribution of the fragment lengths of the cell-free DNA molecules in the population of cell-free DNA molecules that encompass the respective germline allele, thereby generating a set of size-distribution metrics. An indicia that a loss of heterozygosity has occurred at a respective locus in the plurality of locus is determined using a parametric or non-parametric based classifier that evaluates one or more properties of the cell-free DNA molecules in the population of cell-free DNA molecules that encompass the respective locus. The one or more properties include the size-distribution metrics for the corresponding at least two different germline alleles of the respective locus in the set of size-distribution metrics.

One aspect of the present disclosure provides a method for determining the cellular origin of variant alleles present in a biological sample. A dataset is obtained that includes a first plurality of nucleic acid fragment sequences in electronic form from a first biological sample from a subject. Each respective nucleic acid fragment sequence in the first plurality of nucleic acid fragment sequences represents all or a portion of a respective cell-free DNA molecule in a population of cell-free DNA molecules in the first biological sample, the respective nucleic acid fragment sequence encompassing a corresponding locus, in a plurality of loci, represented by at least a reference allele and a variant allele within the population of cell-free DNA molecules. For each respective allele represented at each locus in the plurality of loci, a size-distribution metric is assigned based on a characteristic of the distribution of the fragment lengths of the cell-free DNA molecules in the population of cell-free DNA molecules that encompass the respective allele, thereby generating a set of size-distribution metrics. Each respective variant allele of a respective locus in the plurality of loci is assigned to either to a first category of alleles originating from non-cancerous cells or to a second category of alleles originating from cancer cells using a parametric or non-parametric based classifier that evaluates one or more properties of the cell-free DNA molecules in the sample that encompass the respective locus. The one or more properties include the size-distribution metric for the variant allele of the respective locus.

One aspect of the present disclosure provides a method for identifying and canceling an incorrect mapping of a nucleic acid fragment sequence to a position within a reference genome. A dataset is obtained that includes a plurality of nucleic acid fragment sequences in electronic form from a first biological sample from a subject, where each respective nucleic acid fragment sequence in the plurality of nucleic acid fragment sequences represents all or a portion of a respective cell-free DNA molecule in a population of cell-free DNA molecules in the first biological sample, the respective nucleic acid fragment sequence encompassing a corresponding locus, in a plurality of loci, represented by at least two different alleles within the population of cell-free DNA molecules. Each respective nucleic acid fragment sequence in the plurality of nucleic acid fragment sequences is mapped to a position within a reference genome for the species of the subject, the position within the reference genome encompassing a putative locus in the plurality of loci encompassed by the population of cell-free DNA molecules, based on sequence identity shared between the respective nucleic acid fragment sequence and the nucleic acid sequence at the position within the reference genome. For each respective allele of each respective locus in the plurality of loci, a size-distribution metric is assigned based on characteristic of the distribution of the fragment lengths of the cell-free DNA molecules that are both (i) represented by a respective nucleic acid fragment sequence in the plurality of nucleic acid fragment sequences that encompass the respective allele and (ii) mapped to a same corresponding position within the reference genome, thereby obtaining a set of size-distribution metrics. A confidence metric is determined for the mapping of respective nucleic acid fragment sequences encompassing an allele of a respective locus to a corresponding position within the reference genome encompassing a putative allele by using a parametric or non-parametric based classifier that evaluates one or more properties of the cell-free DNA molecules that are both (i) represented by a respective nucleic acid fragment sequence that encompasses the respective allele and (ii) mapped to the corresponding position within the reference genome. The one or more properties include the size-distribution metric for the respective allele. When the confidence metric fails to satisfy a threshold measure of confidence, canceling the mapping of the respective nucleic acid fragment sequences to the corresponding position within the reference genome.

One aspect of the present disclosure provides a method for validating the use of genotypic data from a particular genomic locus in a subject classifier for classifying a cancer condition for a species. A subject classifier that uses data from the particular genomic locus to classify the cancer condition for a query subject of the species is obtained. For each respective validation subject in a plurality of validation subjects of the species, the following is obtained: (i) a cancer condition and (ii) a validation genotypic data construct that includes one or more genotypic characteristics, thereby obtaining a set of cancer conditions and a correlated set of validation genotypic data constructs. Each genotypic data construct in the set of genotypic data constructs is obtained from a respective first plurality of nucleic acid fragment sequences in electronic form from a corresponding first biological sample from a respective validation subject in the plurality of validation subjects. Each respective nucleic acid fragment sequence in the respective first plurality of nucleic acid fragment sequences represents all or a portion of a respective cell-free DNA molecule in a population of cell-free DNA molecules in the corresponding biological sample, the respective nucleic acid fragment sequence encompassing a corresponding locus, in a plurality of loci, represented by at least two different alleles within the population of cell-free DNA molecules. The one or more genotypic characteristics in the validation genotypic data construct include a size-distribution metric corresponding to a characteristic of the distribution of the fragment lengths of the cell-free DNA molecules that encompass a respective allele of the particular genomic locus. A confidence metric is determined for use of genotypic data from the particular genomic locus in the subject classifier by using a parametric or non-parametric based test classifier that evaluates the size distribution metric for the respective allele in each respective validation genotype data construct and each correlated cancer status in the set of cancer conditions.

Other embodiments are directed to systems, portable consumer devices, and computer readable media associated with methods described herein.

As disclosed herein, any embodiment disclosed herein when applicable can be applied to any aspect.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIGS. 1A and 1B collectively illustrate a block diagram of an example computing device in accordance with some embodiments of the present disclosure.

FIG. 2 illustrates the distribution of cell-free DNA fragment lengths determined for nucleic acid fragment sequences encompassing either a reference (204) or variant (202) allele at a locus, where the variant allele arose from a cancerous cell of the subject.

FIG. 3 illustrates the frequency of white blood cell-matched variant alleles in white blood cells (gdna) plotted against the frequency of the variant alleles in total cell-free DNA (cfdna).

FIG. 4 illustrates the distribution of cell-free DNA fragment lengths determined for nucleic acid fragment sequences encompassing either a reference (402) or variant (404) allele at a locus, where the variant allele arose from clonal hematopoiesis in the subject.

FIG. 5 illustrates the distribution of cell-free DNA fragment lengths determined for nucleic acid fragment sequences encompassing either a reference (502) or germline variant (504) allele at 785 loci known to have allele variation in the germline of a subject.

FIG. 6 illustrates allele frequency measured in nucleic acid fragment sequences from white blood cells (open circles) and total cell free DNA (closed circles) for loci across the genome of a metastatic cancer patient.

FIG. 7 illustrates allele frequency, from loci across the genome of a metastatic cancer patient, measured in nucleic acid fragment sequences from white blood cells of the patient as a function of the allele frequency of the same alleles measured in nucleic acid fragment sequences from total cell free DNA from the same patient.

FIG. 8 illustrates the distribution of cell-free DNA fragment lengths determined for nucleic acid fragment sequences encompassing either a reference (804) or germline variant (802) allele at locus 116382034 of a metastatic cancer patient.

FIG. 9 illustrates the distribution of cell-free DNA fragment lengths determined for nucleic acid fragment sequences encompassing either a reference (902) or germline variant (904) allele at locus 12011772 of a metastatic cancer patient.

FIG. 10 illustrates median fragment length of cell-free DNA fragments determined for nucleic acid fragment sequences encompassing either a reference (closed circles) or variant (open circles) allele for loci across the genome of a metastatic cancer patient.

FIG. 11 illustrates median fragment length (y-axis) of cell-free DNA fragments as a function of allele frequency (x-axis) for loci across the genome of a metastatic cancer patient.

FIG. 12 illustrates allele frequency, as phased by fragment length, measured in nucleic acid fragment sequences from white blood cells (open circles) and total cell free DNA (closed circles) for loci across the genome of a metastatic cancer patient.

FIG. 13 illustrates chromosome copy number determined by segmenting, across the genome of a metastatic cancer patient.

FIG. 14A illustrates the distribution of cell-free DNA fragment lengths determined for nucleic acid fragment sequences encompassing either a reference (1404) or variant (1402) allele at a locus, where the variant allele arose from a cancerous cell of the subject.

FIG. 14B illustrates the distribution of cell-free DNA fragment lengths determined for nucleic acid fragment sequences encompassing either a reference (1406) or variant (1408) allele at a locus, where the variant allele arose from clonal hematopoiesis in the subject.

FIG. 14C illustrates the distribution of cell-free DNA fragment lengths determined for nucleic acid fragment sequences encompassing either a reference (1410) or variant (1412) allele at a locus, where the variant allele is in the germline of the subject.

FIG. 14D illustrates the distribution of cell-free DNA fragment lengths determined for nucleic acid fragment sequences encompassing either a reference (1416) or variant (1414) allele at a locus, where the origin of the variant allele is unknown.

FIG. 15 illustrates the distribution of cell-free DNA fragment lengths determined for nucleic acid fragment sequences encompassing either a reference (1504) or variant (1502) allele at a locus, where the origin of the variant allele is unknown.

FIG. 16 illustrates likelihoods that the origin of variant alleles detected in nucleic acid fragment sequences of cell-free DNA from a metastatic cancer patient is a cancerous cell in the subject, based on an EM mixture model trained against the distribution of fragment lengths of cell-free DNA encompassing a locus having a variant allele that is known to have arisen from a cancer cell in the subject.

FIG. 17A illustrates the distribution of cell-free DNA fragment lengths determined for nucleic acid fragment sequences encompassing either a reference (1704) or variant (1702) allele at a locus, where the variant allele arose from a cancerous cell of the subject.

FIG. 17B illustrates the distribution of cell-free DNA fragment lengths determined for nucleic acid fragment sequences encompassing either a reference (1706) or variant (1708) allele at a locus, where the variant allele arose from clonal hematopoiesis in the subject.

FIG. 17C illustrates the distribution of cell-free DNA fragment lengths determined for nucleic acid fragment sequences encompassing either a reference (1712) or variant (1710) allele at a locus, where the variant allele is in the germline of the subject.

FIG. 17D illustrates the distribution of cell-free DNA fragment lengths determined for nucleic acid fragment sequences encompassing either a reference (1716) or variant (1714) allele at a locus, where the origin of the variant allele is unknown.

FIG. 18 illustrates likelihoods that the origin of variant alleles detected in nucleic acid fragment sequences of cell-free DNA from a metastatic cancer patient is a cancerous cell in the subject, based on an EM mixture model trained against the distribution of fragment lengths of cell-free DNA encompassing a locus having a variant allele that is known to have arisen from a cancer cell in the subject.

FIG. 19A illustrates the distribution of cell-free DNA fragment lengths determined for nucleic acid fragment sequences encompassing loci encompassing a variant allele matched to a variant allele from a cancerous cell of the subject.

FIG. 19B illustrates the distribution of cell-free DNA fragment lengths determined for nucleic acid fragment sequences encompassing either a reference (1902) or variant (1904) allele at a locus, where the variant allele arose from clonal hematopoiesis in the subject.

FIG. 19C illustrates the distribution of cell-free DNA fragment lengths determined for nucleic acid fragment sequences encompassing either a reference (1908) or variant (1906) allele at a locus, where the variant allele is in the germline of the subject.

FIG. 19D illustrates the distribution of cell-free DNA fragment lengths determined for nucleic acid fragment sequences encompassing either a reference (1912) or variant (1910) allele at a locus, where the origin of the variant allele is unknown.

FIG. 20A illustrates the distribution of cell-free DNA fragment lengths determined for nucleic acid fragment sequences encompassing either a reference (2004) or variant (2002) allele at a locus, where the variant allele arose from a cancerous cell of the subject.

FIG. 20B illustrates the distribution of cell-free DNA fragment lengths determined for nucleic acid fragment sequences encompassing either a reference (2006) or variant (2008) allele at a locus, where the variant allele arose from clonal hematopoiesis in the subject.

FIG. 20C illustrates the distribution of cell-free DNA fragment lengths determined for nucleic acid fragment sequences encompassing either a reference (2010) or variant (2012) allele at a locus, where the variant allele is in the germline of the subject.

FIG. 20D illustrates the distribution of cell-free DNA fragment lengths determined for nucleic acid fragment sequences encompassing either a reference (2016) or variant (2014) allele at a locus, where the origin of the variant allele is unknown.

FIG. 21 illustrates likelihoods that the origin of variant alleles detected in nucleic acid fragment sequences of cell-free DNA from a metastatic cancer patient is a cancerous cell in the subject, based on an EM mixture model trained against the distribution of fragment lengths of cell-free DNA encompassing a locus having a variant allele that is known to have arisen from a cancer cell in the subject.

FIG. 22A illustrates likelihoods that the origin of individual white blood cell-matched variant alleles detected in nucleic acid fragment sequences of cell-free DNA from a metastatic cancer patient is a cancerous cell in the subject, based on an EM mixture model trained against the distribution of fragment lengths of cell-free DNA encompassing a locus having a variant allele that is known to have arisen from a cancer cell in the subject.

FIG. 22B illustrates likelihoods that the origin of individual biopsy-matched variant alleles detected in nucleic acid fragment sequences of cell-free DNA from a metastatic cancer patient is a cancerous cell in the subject, based on an EM mixture model trained against the distribution of fragment lengths of cell-free DNA encompassing a locus having a variant allele that is known to have arisen from a cancer cell in the subject.

FIG. 22C illustrates likelihoods that the origin of individual variant alleles that were not matched to a biopsy, white blood cells, or the germline detected in nucleic acid fragment sequences of cell-free DNA from a metastatic cancer patient is a cancerous cell in the subject, based on an EM mixture model trained against the distribution of fragment lengths of cell-free DNA encompassing a locus having a variant allele that is known to have arisen from a cancer cell in the subject.

FIG. 23A illustrates the distribution of cell-free DNA fragment lengths determined for nucleic acid fragment sequences encompassing either a reference (2304) or variant (2302) allele at a locus, where the variant allele arose from a cancerous cell of the subject.

FIG. 23B illustrates the distribution of cell-free DNA fragment lengths determined for nucleic acid fragment sequences encompassing either a reference (2306) or variant (2308) allele at a locus, where the variant allele arose from clonal hematopoiesis in the subject.

FIG. 23C illustrates the distribution of cell-free DNA fragment lengths determined for nucleic acid fragment sequences encompassing either a reference (2310) or variant (2312) allele at a locus, where the variant allele is in the germline of the subject.

FIG. 23D illustrates the distribution of cell-free DNA fragment lengths determined for nucleic acid fragment sequences encompassing either a reference (2316) or variant (2314) allele at a locus, where the origin of the variant allele is unknown.

FIG. 24A illustrates likelihoods that the origin of individual variant alleles that were not matched to a biopsy, white blood cells, or the germline detected in nucleic acid fragment sequences of cell-free DNA from an early lung cancer patient is a cancerous cell in the subject, based on an EM mixture model trained against the distribution of fragment lengths of cell-free DNA encompassing a locus having a variant allele that is known to have arisen from a cancer cell in the subject.

FIG. 24B illustrates likelihoods that the origin of individual white blood cell-matched variant alleles detected in nucleic acid fragment sequences of cell-free DNA from a metastatic cancer patient is a cancerous cell in the subject, based on an EM mixture model trained against the distribution of fragment lengths of cell-free DNA encompassing a locus having a variant allele that is known to have arisen from a cancer cell in the subject.

FIG. 25A illustrates the distribution of cell-free DNA fragment lengths determined for nucleic acid fragment sequences encompassing either a reference (2504) or variant (2502) allele at a locus, where the variant allele arose from a cancerous cell of the subject.

FIG. 25B illustrates the distribution of cell-free DNA fragment lengths determined for nucleic acid fragment sequences encompassing either a reference (2506) or variant (2508) allele at a locus, where the variant allele arose from clonal hematopoiesis in the subject.

FIG. 25C illustrates the distribution of cell-free DNA fragment lengths determined for nucleic acid fragment sequences encompassing either a reference (2510) or variant (2512) allele at a locus, where the variant allele is in the germline of the subject.

FIG. 25D illustrates the distribution of cell-free DNA fragment lengths determined for nucleic acid fragment sequences encompassing either a reference (2516) or variant (2514) allele at a locus, where the origin of the variant allele is unknown.

FIG. 26 illustrates likelihoods that the origin of variant alleles detected in nucleic acid fragment sequences of cell-free DNA from an early lung cell patient is a cancerous cell in the subject, based on an EM mixture model trained against the distribution of fragment lengths of cell-free DNA encompassing a locus having a variant allele that is known to have arisen from a cancer cell in the subject.

FIG. 27A illustrates the distribution of cell-free DNA fragment lengths determined to be nucleic acid fragment sequences encompassing loci encompassing a variant allele originating from a cancerous cell of the subject.

FIG. 27B illustrates the distribution of cell-free DNA fragment lengths determined for nucleic acid fragment sequences encompassing either a reference (2704) or variant (2702) allele at a locus, where the variant allele arose from clonal hematopoiesis in the subject.

FIG. 27C illustrates the distribution of cell-free DNA fragment lengths determined for nucleic acid fragment sequences encompassing either a reference (2708) or variant (2706) allele at a locus, where the variant allele is in the germline of the subject.

FIG. 27D illustrates the distribution of cell-free DNA fragment lengths determined for nucleic acid fragment sequences encompassing either a reference (2712) or variant (2710) allele at a locus, where the origin of the variant allele is unknown.

FIG. 28A illustrates the distribution of cell-free DNA fragment lengths determined for nucleic acid fragment sequences encompassing either a reference (2804) or variant (2802) allele at a locus, where the variant allele arose from a cancerous cell of the subject.

FIG. 28B illustrates the distribution of cell-free DNA fragment lengths determined for nucleic acid fragment sequences encompassing either a reference (2806) or variant (2808) allele at a locus, where the variant allele arose from clonal hematopoiesis in the subject.

FIG. 28C illustrates the distribution of cell-free DNA fragment lengths determined for nucleic acid fragment sequences encompassing either a reference (2810) or variant (2812) allele at a locus, where the variant allele is in the germline of the subject.

FIG. 28D illustrates the distribution of cell-free DNA fragment lengths determined for nucleic acid fragment sequences encompassing either a reference (2816) or variant (2814) allele at a locus, where the origin of the variant allele is unknown.

FIG. 29 illustrates likelihoods that the origin of variant alleles detected in nucleic acid fragment sequences of cell-free DNA from a patient with hypermutation metastatic cancer is a cancerous cell in the subject, based on an EM mixture model trained against the distribution of fragment lengths of cell-free DNA encompassing a locus having a variant allele that is known to have arisen from a cancer cell in the subject.

FIG. 30A illustrates the distribution of cell-free DNA fragments lengths for nucleic acid fragment sequences that map to locus 236649 and putatively encompass either a reference (3004) or variant (3002) allele.

FIG. 30B illustrates the distribution of cell-free DNA fragments lengths for nucleic acid fragment sequences that map to locus 236653 and putatively encompass either a reference (3008) or variant (3006) allele.

FIG. 30C illustrates the distribution of cell-free DNA fragments lengths for nucleic acid fragment sequences that putatively map to locus 236678 and putatively encompass either a reference (3012) or variant (3010) allele.

FIGS. 31A, 31B, 31C, and 31D each illustrate distribution of cell-free DNA fragments lengths for nucleic acid fragment sequences that map to the incorrect locus and putatively encompass either a reference (3102, 3106, and 3110) or variant allele (3104, 3108, 3112, and 3114).

FIG. 32 illustrates the diagnostic use of fragment length for verifying variant calling algorithms, with respect to mutations identified in the TP53 gene.

FIG. 33 illustrates the diagnostic use of fragment length for verifying variant calling algorithms, with respect to mutations identified in the PIK3CA gene.

FIG. 34 illustrates the diagnostic use of fragment length for verifying variant calling algorithms, with respect to mutations identified in the EGFR gene.

FIG. 35 illustrates the diagnostic use of fragment length for verifying variant calling algorithms, with respect to mutations identified in the TET2 gene.

FIG. 36 is a graphical representation of the process for obtaining nucleic acid fragment sequences in accordance with some embodiments of the present disclosure.

FIGS. 37A, 37B, 37C, and 37D collectively provide a flow chart of processes and features for identifying segmenting all or a portion of a reference genome, in which optional steps are depicted by dashed boxes, in accordance with various embodiments of the present disclosure.

FIGS. 38A, 38B, 38C, 38D, 38E, 38F, and 38G collectively provide a flow chart of processes and features for phasing alleles present on a matching pair of chromosomes in a cancerous tissue, in which optional steps are depicted by dashed boxes, in accordance with various embodiments of the present disclosure.

FIGS. 39A, 39B, 39C, 39D, and 39E collectively provide a flow chart of processes and features for detecting a loss in heterozygosity at a genomic locus in a cancerous tissue, in which optional steps are depicted by dashed boxes, in accordance with various embodiments of the present disclosure.

FIGS. 40A, 40B, 40C, 40D, 40E, and 40F collectively provide a flow chart of processes and features for determining the cellular origin of variant alleles present in a biological sample, in which optional steps are depicted by dashed boxes, in accordance with various embodiments of the present disclosure.

FIGS. 41A, 41B, 41C, 41D, and 41E collectively provide a flow chart of processes and features for identifying and canceling an incorrect mapping of a nucleic acid fragment sequence to a position within a reference genome, in which optional steps are depicted by dashed boxes, in accordance with various embodiments of the present disclosure.

FIGS. 42A, 42B, 42C, 42D, and 42E collectively provide a flow chart of processes and features for validating the use of genotypic data from a particular genomic locus in a subject classifier for classifying a cancer condition for a species, in which optional steps are depicted by dashed boxes, in accordance with various embodiments of the present disclosure.

FIG. 43A illustrates the distribution of cell-free DNA fragment lengths determined for nucleic acid fragment sequences encompassing either a reference (4304) or variant (4302) allele at a locus, where the variant allele arose from a cancerous cell of the subject.

FIG. 43B illustrates the distribution of cell-free DNA fragment lengths determined for nucleic acid fragment sequences encompassing either a reference (4306) or variant (4308) allele at a locus, where the variant allele arose from clonal hematopoiesis in the subject.

FIG. 43C illustrates the distribution of cell-free DNA fragment lengths determined for nucleic acid fragment sequences encompassing either a reference (4312) or variant (4310) allele at a locus, where the variant allele is in the germline of the subject.

FIG. 43D illustrates the distribution of cell-free DNA fragment lengths determined for nucleic acid fragment sequences encompassing either a reference (4316) or variant (4314) allele at a locus, where the origin of the variant allele is unknown.

FIG. 44 illustrates a plot of the underlying fragment length distributions for a global background length distribution obtained from the germline variants (4402), a shifted distribution of fragment lengths based on a typical shift (e.g., seen in cell-free DNA fragments from cancer cells) of about 11 bases (4404), the observed distribution from the alternate alleles in biopsy matched fragments (4406), and a blend of the two distributions, for use when few alternate alleles are available (4408).

FIGS. 45A and 45B illustrates likelihoods that the origin of variant alleles detected in nucleic acid fragment sequences of cell-free DNA from a cancer patient is a cancerous cell in the subject, based on an EM mixture model trained against a distribution of fragment lengths of cell-free DNA encompassing a locus having a variant allele that arose from a non-cancerous origin.

FIG. 46 illustrates a flowchart of a method for preparing a nucleic acid sample for sequencing in accordance with some embodiments of the present disclosure.

FIGS. 47A and 47B illustrate plasma cfDNA allele frequencies (posterior mean) as determined by targeted panel sequencing for each variant source (posterior mean is always positive allowing for log-scale plotting), as described in Example 15. The source of each allele is shown in FIG. 47B (4708: WBC-matched (WM); 4706: tumor biopsy-matched (TBM); 4702: ambiguous (AMB); 4704: non-matched (NM)). Each dot represents a single SNV.

FIG. 48 illustrates the observed fragment length distributions of variant alleles by variant category, as described in Example 15.

FIGS. 49A, 49B, 49C, 49D, 49E, and 49F illustrate examples of classification within two individual samples (Subject A=FIG. 49A-49C; Subject B=FIG. 49D-49F), as described in Example 15.

FIGS. 50A and 50B illustrate plots of predictive statistics for distinguishing tumor- versus WBC-derived variants, as described in Example 15.

Like reference numerals refer to corresponding parts throughout the several views of the drawings.

### DETAILED DESCRIPTION

The present disclosure provides systems and methods useful for classifying a subject for a cancer condition based on analysis of the distribution of cell-free DNA fragment lengths in biological fluids. Advantageously, as described herein, Applicants have developed various methodologies that facilitate analysis of cell-free DNA, which is useful for classifying subjects for a cancer condition. These methodologies leverage information about the biology of the subject, and specifically information about the various genomes of the subject (e.g., the subject's cancer genome(s), germline genome, and/or hematopoietic genome(s)), that can be obtained from the relative distributions of cell-free DNA fragment lengths in biological fluids of the subject.

Applicants have developed various models based on observations that the length distributions of cell-free DNA fragments that originate from cancer cells are shifted by a number of nucleotides (e.g., around 5 to 25 nucleotides, such as around 10 nucleotides) relative to the length distributions of cell-free DNA fragments that originate from non-cancerous cells, e.g., non-cancerous germline tissues and hematopoietic cell lineages (e.g., white blood cells). Because the population of cell-free DNA fragments in bodily fluids is a mixture of fragments originating from germline cells, hematopoietic cell lineages (e.g., white blood cells), and cancer cells (e.g., when the subject is afflicted with cancer), the global distribution of cell-free DNA fragment lengths varies along with the biology of the subject. Applicants have also leveraged the discovery that cell-free DNA fragment length distributions are also influenced by copy number aberrations to develop methods for phasing and mapping out chromosomal copy number aberrations in a cancer genome based on analysis of cell-free DNA fragment lengths.

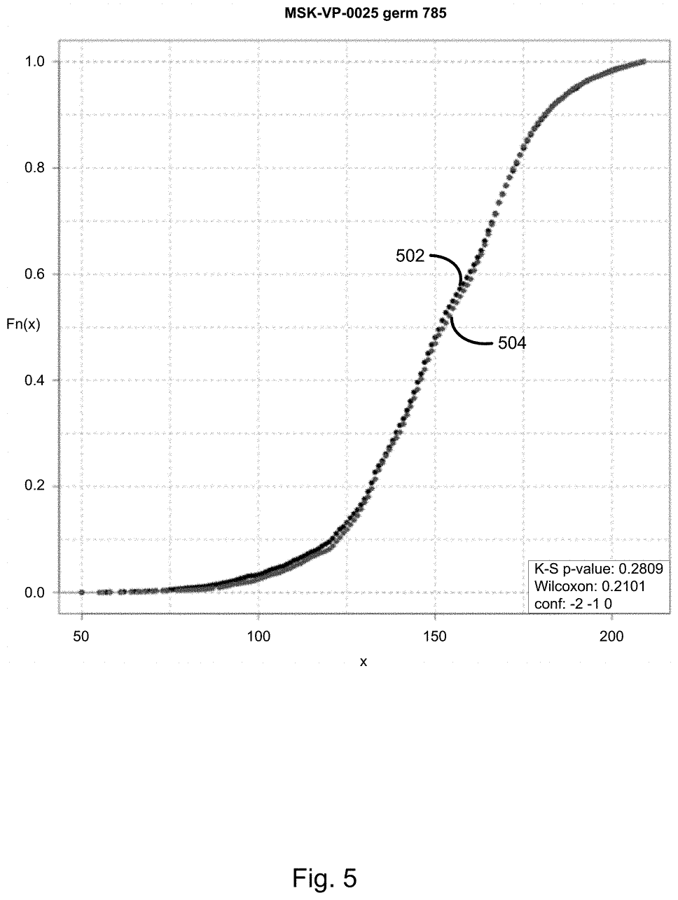

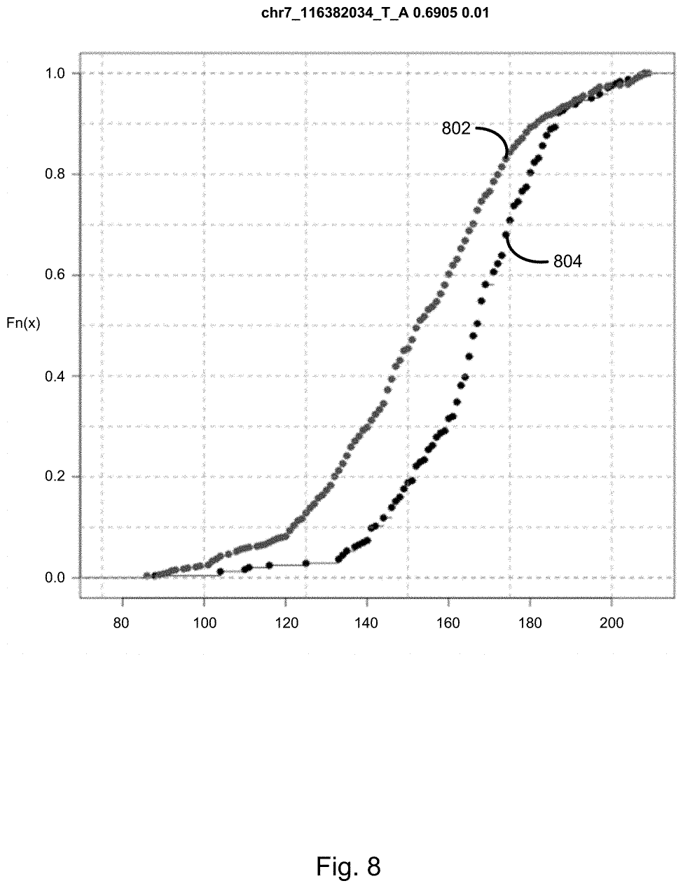

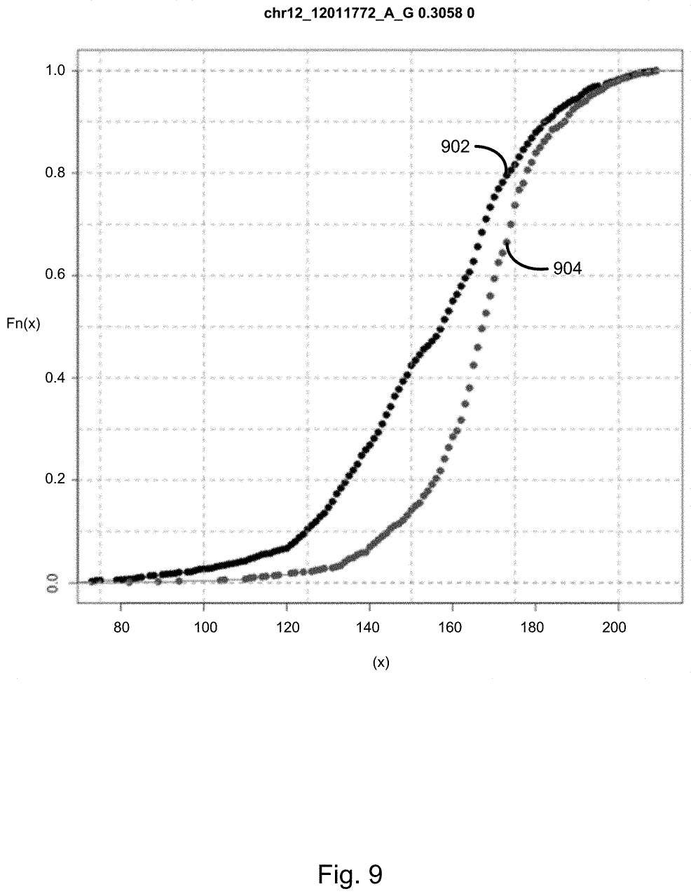

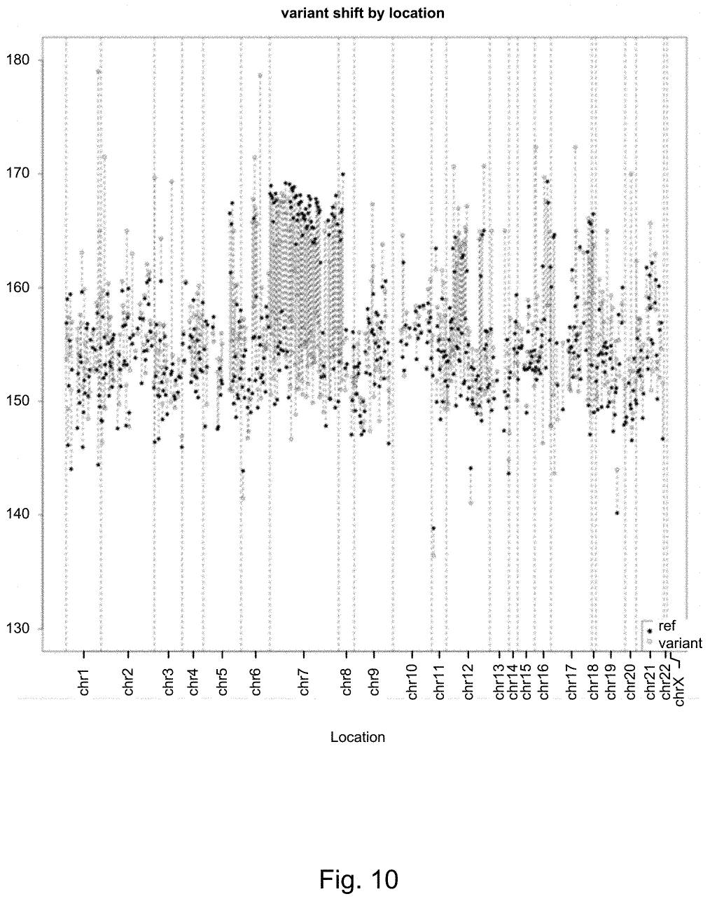

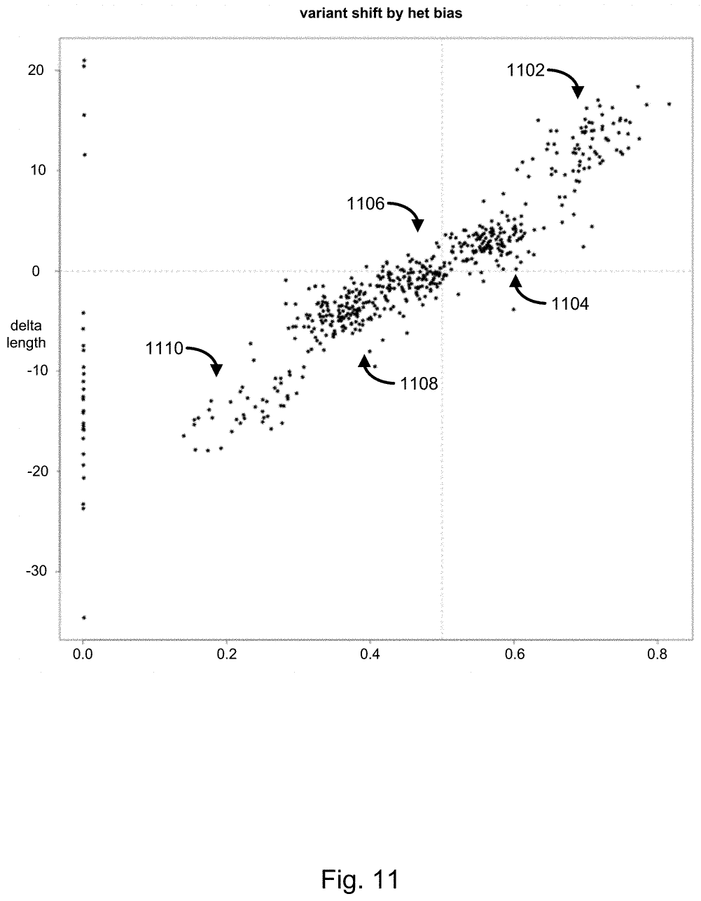

For example, in on aspect, the disclosure provides methods for mapping chromosomal copy number aberrations in the genome of a cancer based, at least in part, on the identification of shifts in the distribution of fragment lengths of cell-free DNA molecules encompassing a locus represented by a germline variant allele. These shifts are representative of the loss or gain of an allele at the locus in the cancer. For example, as described in Example 3, when the fragment length distribution of all loci represented by a variant germline allele are plotted in aggregate, no difference in the mean fragment length is observed between cell-free DNA fragments encompassing a variant allele or a reference allele (see, FIG. 5). However, when the fragment length distribution of individual loci is plotted, significant shifts in the distribution of cell-free DNA fragments are seen where there is loss or gain of either the reference allele (see, FIG. 8) or the germline variant allele (see, FIG. 9). These shifts can be mapped across the genome (see, FIG. 10), indicating positions at which chromosomal copy number aberrations have occurred. Further, when coupled with conventional metrics, e.g., allele-frequency metrics and/or read-depth metrics for individual alleles, clear groupings of loci having similar chromosomal copy number aberrations can be observed (see, FIG. 11).

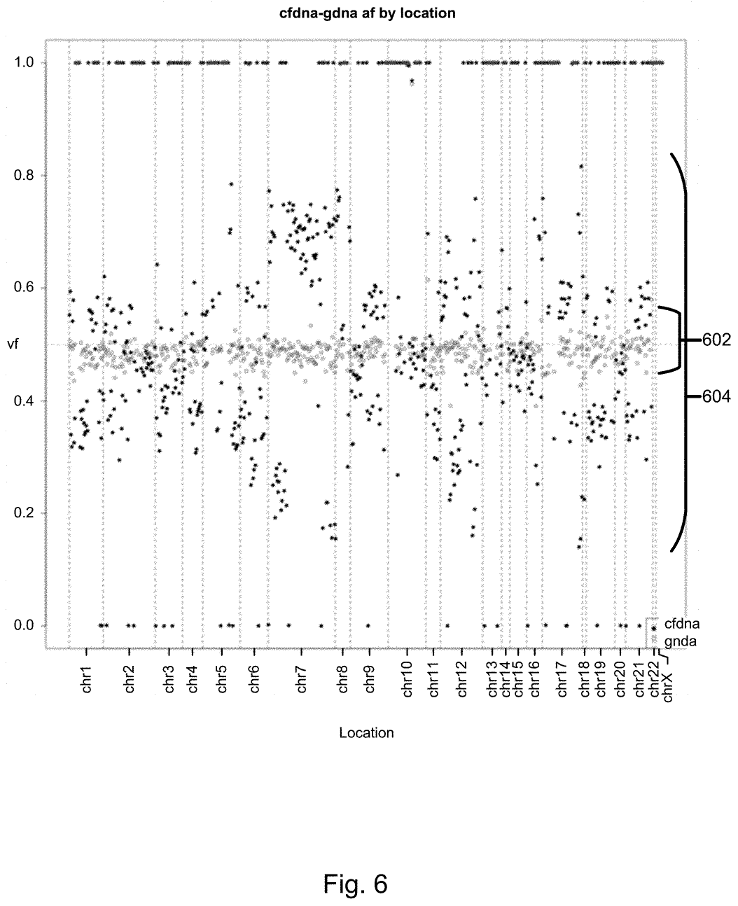

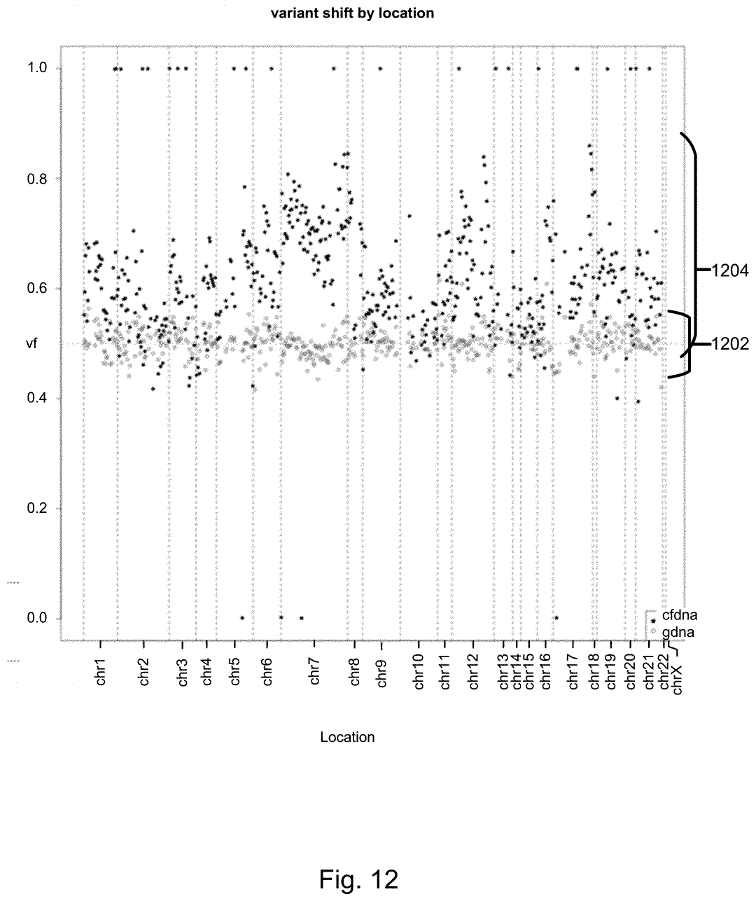

In another aspect, the disclosure provides methods for phasing alleles on individual chromosomes within the cancer genome based, at least in part, on the identification of shifts in the distribution of fragment lengths of cell-free DNA molecules encompassing a locus represented by a germline variant allele. As described above, these shifts are representative of the loss or gain of an allele at the locus in the cancer. Thus, when larger regions of a chromosome, or entire chromosomes themselves, are subject to a copy number aberration, alleles that are located on the same chromosome, e.g., either the maternal chromosome or the paternal chromosome, should be encompassed by cell-free DNA fragments that display the same characteristic shifts in fragment lengths, relative to the other allele represented on the other chromosome. For example, when the allele frequencies of germline variant alleles are plotted as a function of genome position, a distribution of allele frequencies, from about 0.2 to about 0.8, are seen throughout the genome, representative of various losses and gains of allele copy numbers on either the chromosome harboring the variant allele or on the opposite chromosome (see, FIG. 6). However, when cell-free DNA fragment length distribution shifts are used to phase the allele frequencies, that is used to define whether it is the variant allele frequency or the reference allele frequency that is plotted across the genome, the resulting plot is phased to show only the alleles that are in excess in the cancer cells (see, FIG. 12), or vice versa. Thus, the identity of alleles that are present on the same chromosome together can be identified.

In another aspect, the disclosure provides methods for detecting and/or mapping loss of heterozygosity at a segment of a cancer genome (e.g., within a particular chromosome) based, at least in part, on the identification of shifts in the distribution of fragment lengths of cell-free DNA molecules encompassing loci located within the segment of the genome. As described above, shifts in the fragment length distribution of cell-free DNA encompassing a locus associated with a germline variant allele are representative of the loss or gain of that allele at the locus in the cancer. Thus, the detection of characteristic shifts in the length distribution of cell-free DNA encompassing a locus represented by a germline variant allele indicate loss of either the reference allele (see, FIG. 8) or the germline variant allele (see, FIG. 9), at the locus in the cancer genome.

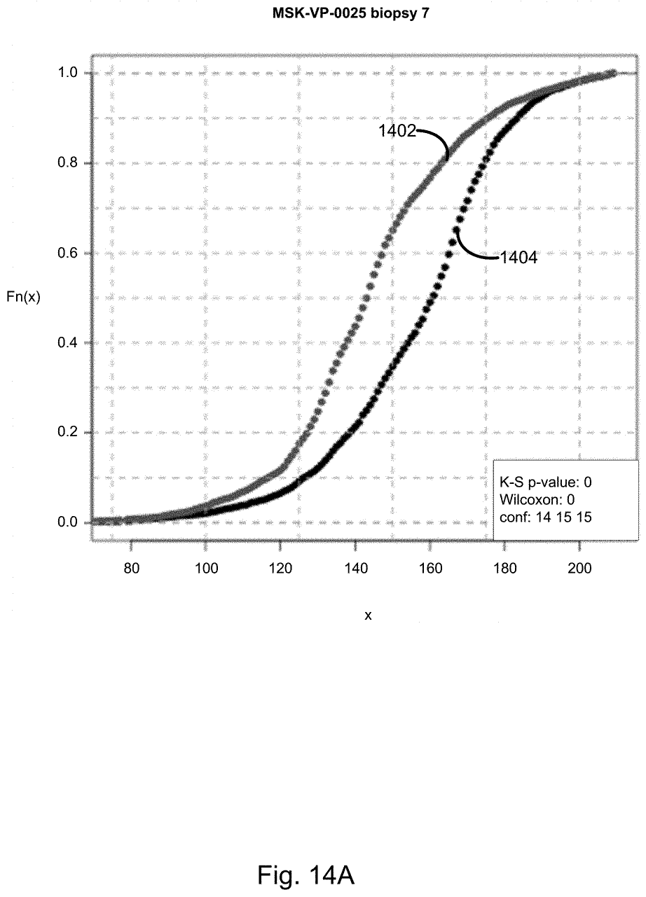

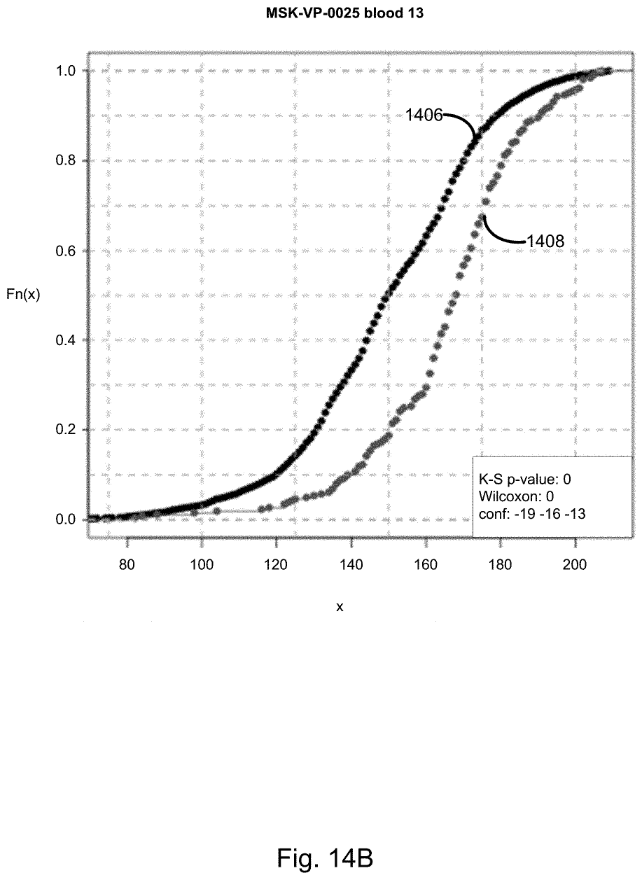

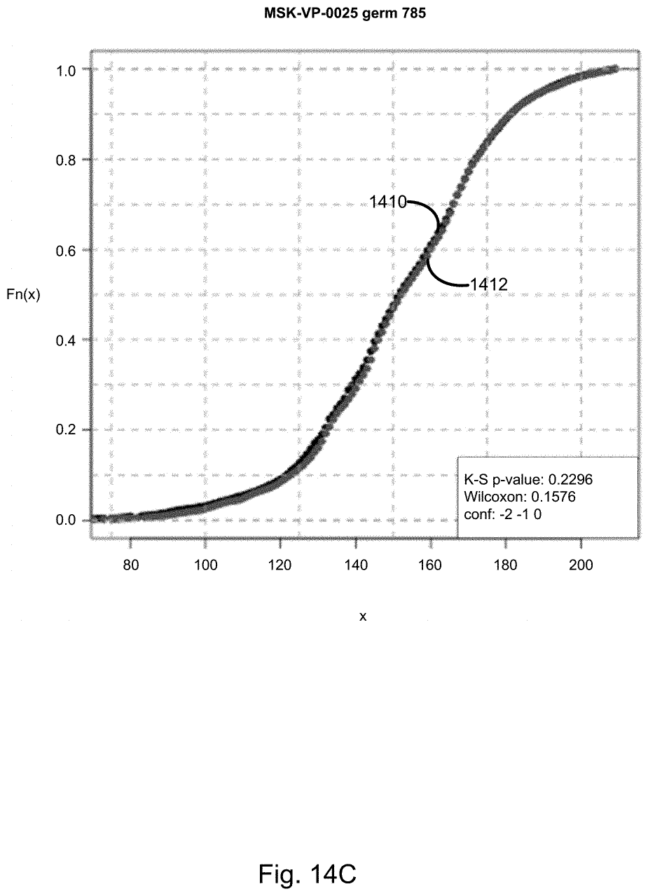

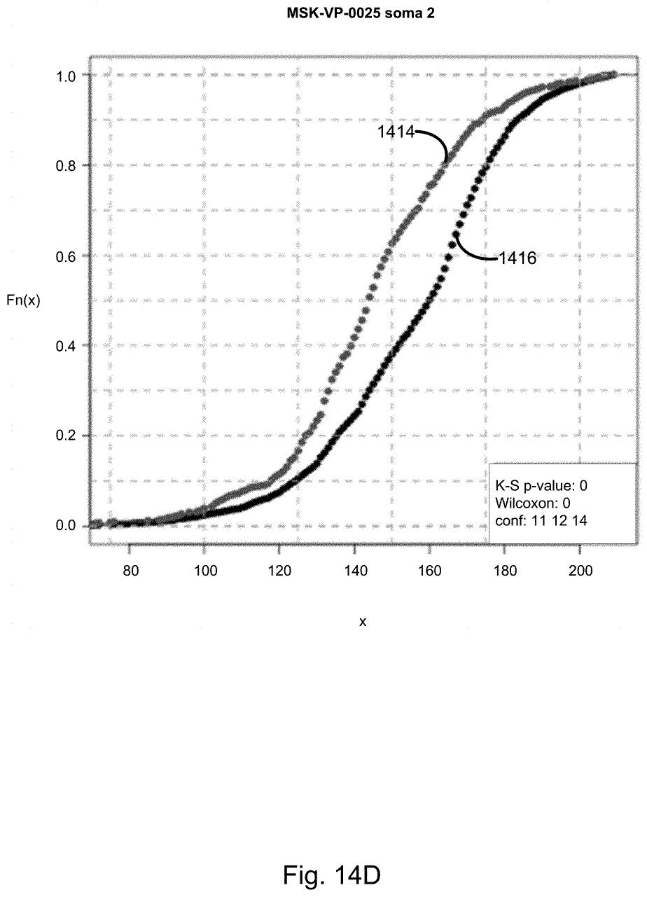

In another aspect, the disclosure provides methods for determining the origin of a variant allele detected in cell-free DNA fragments. As described above, the identification of novel variant alleles in a cancer genome allows for tailored treatment of the particular cancer in a subject. While it was known that variant cancer alleles could be detected in cell-free DNA fragments, the majority of variant alleles found in cell-free DNA fragments originate from other sources. For example, as described in Example 4, targeted, capture-based DNA sequencing of cell-free DNA in a blood sample from a subject confirmed to have metastatic prostate cancer let to the identification of 807 single nucleotide variants. Of these, 798 variants were confirmed to originate from either clonal hematopoiesis (13; see, FIG. 14B) or the germline (785; see, FIG. 14C). Thus, only 9 of the 807 variants detected arose from the cancer and, thus, are putatively relevant to the biology of the individual cancer.

Conventionally, determining which variants detected in a cell-free DNA sample are novel to the cancer is a burdensome and time-consuming process, e.g., requiring sequencing of a biopsy-matched sample from the subject. Moreover, where the subject has not yet been diagnosed with cancer, conventional methods would require two visits to the physician in order to even obtain the material required for such an analysis: a first visit in which tests can be performed to diagnose the subject with cancer, and a second visit in which a biopsy can be taken to provide the material required for the analysis. Advantageously, Applicants have developed methods that facilitate cancer variant allele identification from a single biological sample (e.g., a blood sample), e.g., which could subsequently be used to diagnose the cancer.

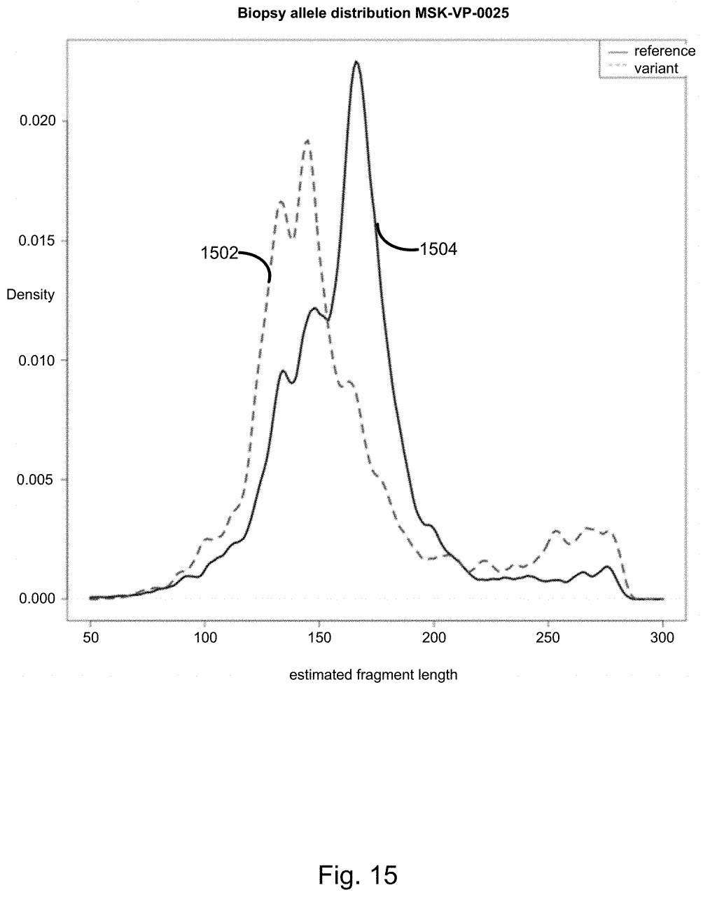

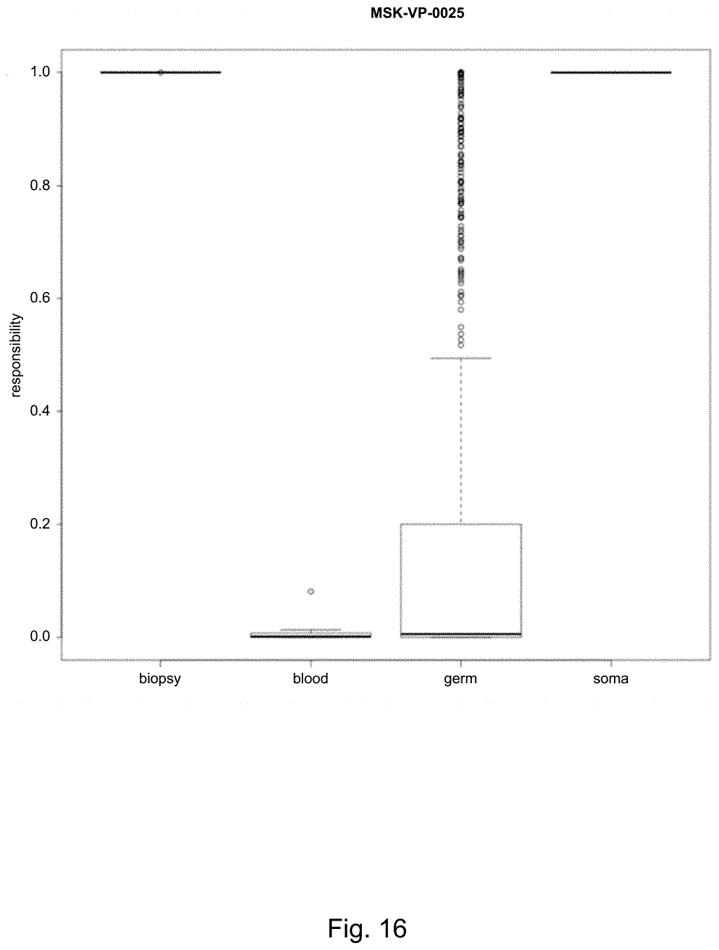

These methods, as described herein, leverage the different distributions of cell-free DNA fragment lengths of cell-free DNA fragments encompassing a locus represented in the population by a novel cancer variant allele (e.g., see, FIG. 14A), a clonal hematopoiesis variant allele (e.g., see, FIG. 14B), and a germline variant allele (FIG. 14C). For example, as demonstrated in FIG. 16, two variant alleles were detected in the blood of the same metastatic cancer patient, that were not matched to variants sequenced in any of a matching tumor biopsy, a red-blood cell sample, or a non-cancerous tissue sample from the subject (see, FIG. 14D). However, a mixed model of cell-free DNA fragments lengths (see, FIG. 15) was used to train an expectation maximization (EM) algorithm, which then assigned a high responsibility (e.g., probability) that the unmatched ‘novel somatic’ variant, in fact, did originate from cancer cells (see, FIG. 16) and, thus, are relevant to the biology of the cancer in the subject. Advantageously, these methods (i) simplify and speed up the identification of variant alleles originating from a cancer, e.g., by allowing identification from a single blood sample from the subject, and (ii) facilitate identification of alleles that would not otherwise be matched to sequencing of biopsy-matched samples from the subject (e.g., such as the two novel somatic variant alleles identified as highly likely to be cancer derived in Example 4).

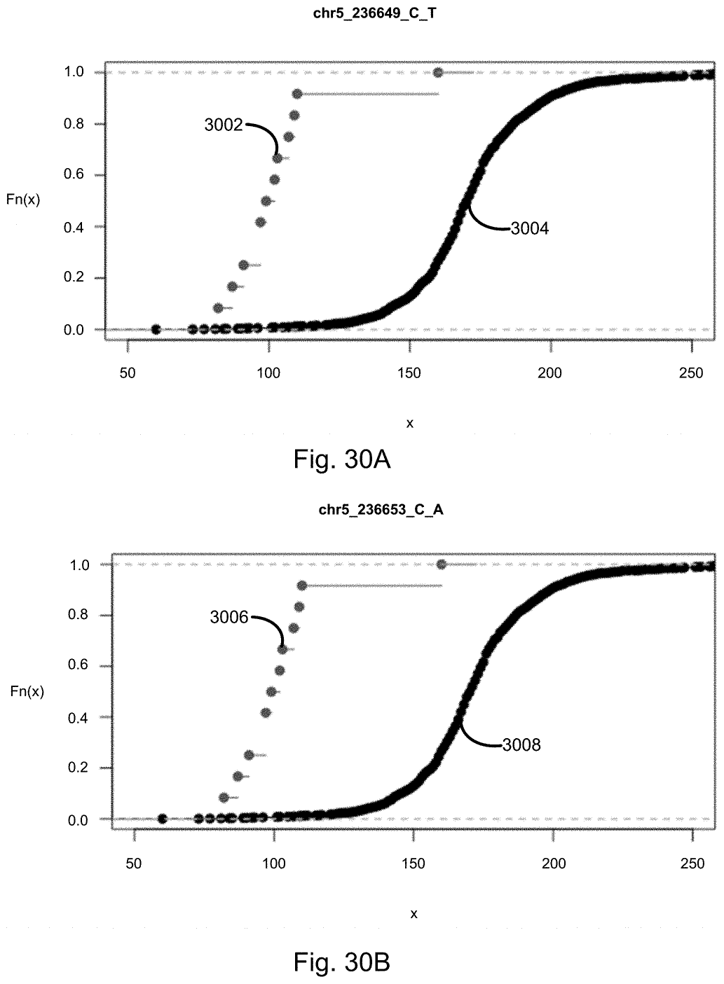

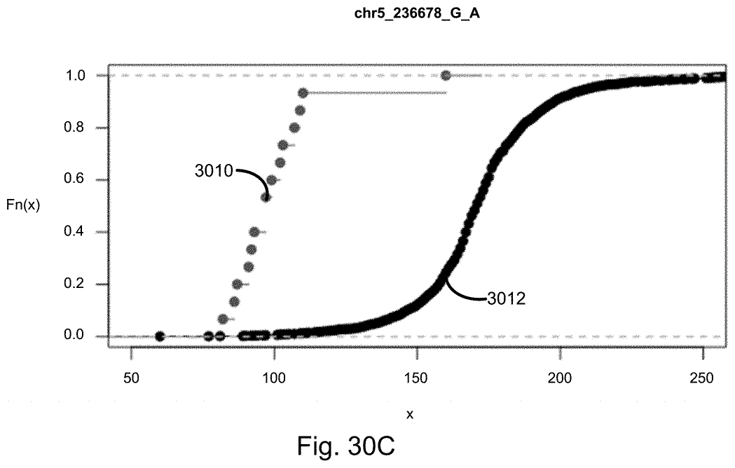

In another aspect, the disclosure provides methods for identifying misalignment of sequencing data of cell-free DNA fragments. The alignment of sequencing data from cell-free DNA fragments to positions within a reference genome is not trivial, as one of the purposes of the sequencing is to identify the presence of variant allele sequences which, by definition, diverge from the sequence of the reference genome. Thus, the sequence alignment methodologies must allow for the alignment of sequences that do not perfectly match to the reference genome in order to properly identify the sequenced genomic loci. As described in Example 12, however, this also results in misalignments of sequencing data. However, the use of distribution patterns of cell-free DNA fragments mapped to a particular position in the reference genome can be used to identify mis-mappings based on the identification of substantially non-ideal fragment-length distributions, because the information contained within the distribution is not tied to the sequences of the fragments themselves. For example, as shown in FIGS. 30A-30C, short fragments containing putative variant alleles were mapped to chromosome 5 in a cancer patient, as the best alignment to the reference genome. However, inspection of the fragment distribution at the loci represented by the putative variant alleles revealed an abnormal distribution of fragment lengths, in which almost no fragments longer than 100 nucleotides were mapped to the loci. In fact, the fragments encompassing the same putative variant alleles mapped to a different position in the reference genome. Accordingly, Applicants developed a method for screening the alignment of cell-free DNA fragment sequences to a reference genome, in which the distribution of fragment lengths of the nucleic acid fragment sequences encompassing the locus are compared to one or more expected fragment length distributions, and alignments corresponding to fragment length distributions that significantly deviate from the one or more fragment length distributions are canceled.

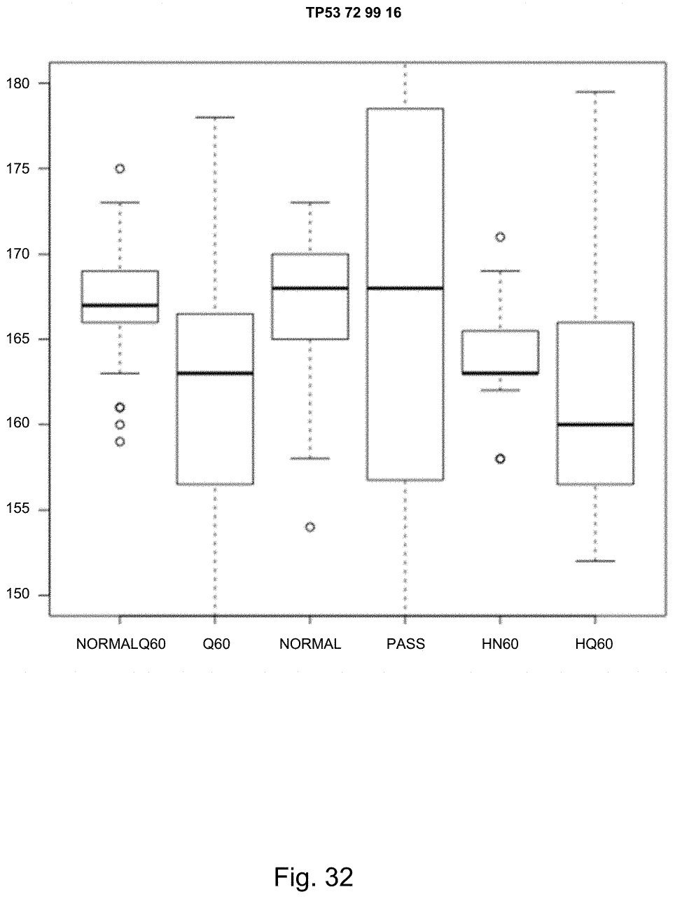

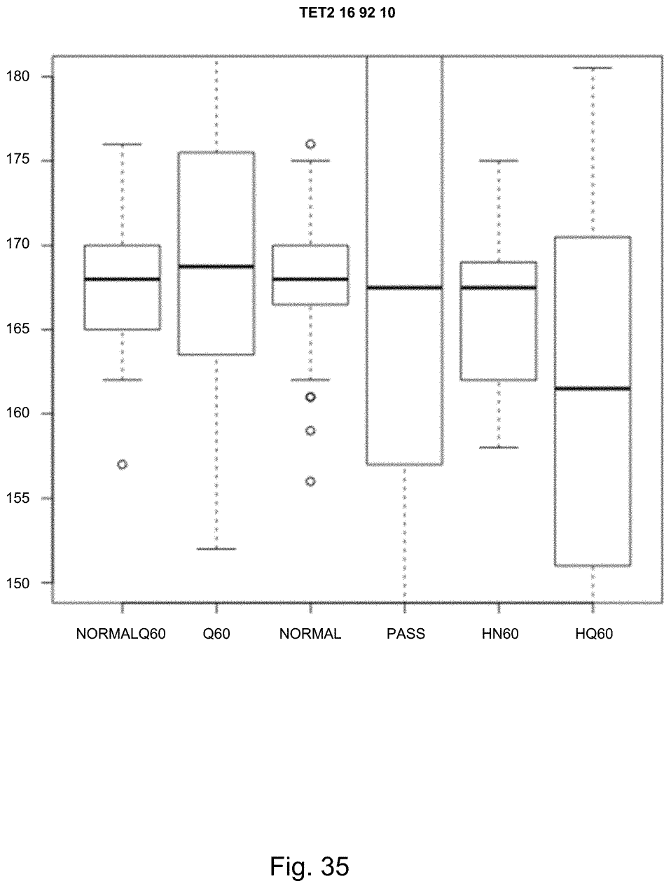

In another aspect, the disclosure provides methods for validating the use of genomic and/or epigenetic information from a particular allele in a cancer classifier. For example, as described in Example 13, fragment length can be used to evaluate the performance of a classifier with respect to a particular allele. As shown in FIGS. 32, 33, and 34, analysis of the lengths of cell-free DNA fragments encompassing a loci associated with a variant allele identified as informative, e.g., as originating from a cancer, suggests that the Q60 noise model filter, but not the PASS bioinformatics model, enriches for variant alleles that are relevant to cancer biology in the subjects. As shown in FIG. 35, however, this analysis suggests that even the Q60 noise model filter fails to enrich for informative variants within the TET2 gene, which is associated with high rates of mutagenesis in clonal hematopoiesis. Accordingly, Applicants developed methods for validating the use of a particular cancer classifier and/or information relating to a particular allele in a cancer classifier.

### Definitions

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any stage (e.g., a man, a women or a child).

As used herein, the phrase “healthy” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used herein, the term “biological fluid sample,” “biological sample,” “patient sample,” or “sample” refers to any sample taken from a subject, which can reflect a biological state associated with the subject, and that includes cell free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. In some embodiments, the biological sample consists of blood, whole blood, plasma, serum, urine, cerebrospinal fluid, fecal, saliva, sweat, tears, pleural fluid, pericardial fluid, or peritoneal fluid of the subject. In such embodiments, the biological sample is limited to blood, whole blood, plasma, serum, urine, cerebrospinal fluid, fecal, saliva, sweat, tears, pleural fluid, pericardial fluid, or peritoneal fluid of the subject and does not contain other components (e.g., solid tissues, etc.) of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A biological sample can be obtained from a subject invasively (e.g., surgical means) or non-invasively (e.g., a blood draw, a swab, or collection of a discharged sample).

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map nucleic acid fragment sequences obtained from sequencing a sample from the subject. A reference genome can refer to a haploid or diploid genome to which nucleic acid fragment sequences from the biological sample and a constitutional sample can be aligned and compared. An example of constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. A nucleic acid may be prepared using a nucleic acid obtained from a subject as a template.

As used herein, the term “cell-free nucleic acids” refers to nucleic acid molecules that can be found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids originate from one or more healthy cells and/or from one or more cancer cells Cell-free nucleic acids are used interchangeably as circulating nucleic acids. Examples of the cell-free nucleic acids include but are not limited to RNA, mitochondrial DNA, or genomic DNA. As used herein, the terms “cell free nucleic acid,” “cell free DNA,” and “cfDNA” are used interchangeably. As used herein, the term “circulating tumor DNA” or “ctDNA” refers to nucleic acid fragments that originate from tumor cells or other types of cancer cells, which may be released into a fluid from an individual's body (e.g., bloodstream) as result of biological processes such as apoptosis or necrosis of dying cells or actively released by viable tumor cells.

As used herein, the term “locus” refers to a position (e.g., a site) within a genome, i.e., on a particular chromosome. In some embodiments, a locus refers to a single nucleotide position within a genome, i.e., on a particular chromosome. In some embodiments, a locus refers to a small group of nucleotide positions within a genome, e.g., as defined by a mutation (e.g., substitution, insertion, or deletion) of consecutive nucleotides within a cancer genome. Because normal mammalian cells have diploid genomes, a normal mammalian genome (e.g., a human genome) will generally have two copies of every locus in the genome, or at least two copies of every locus located on the autosomal chromosomes, i.e., one copy on the maternal autosomal chromosome and one copy on the paternal autosomal chromosome.

As used herein, the term “allele” refers to a particular sequence of one or more nucleotides at a chromosomal locus.

As used herein, the term “reference allele” refers to the sequence of one or more nucleotides at a chromosomal locus that is either the predominant allele represented at that chromosomal locus within the population of the species (e.g., the “wild-type” sequence), or an allele that is predefined within a reference genome for the species.

As used herein, the term “variant allele” refers to a sequence of one or more nucleotides at a chromosomal locus that is either not the predominant allele represented at that chromosomal locus within the population of the species (e.g., not the “wild-type” sequence), or not an allele that is predefined within a reference genome for the species.

As used herein, the term “single nucleotide variant” or “SNV” refers to a substitution of one nucleotide to a different nucleotide at a position (e.g., site) of a nucleotide sequence, e.g., a nucleic acid fragment sequence from an individual. A substitution from a first nucleobase X to a second nucleobase Y may be denoted as “X>Y.” For example, a cytosine to thymine SNV may be denoted as “C>T.”

As used herein, the term “mutation,” refers to a detectable change in the genetic material of one or more cells. In a particular example, one or more mutations can be found in, and can identify, cancer cells (e.g., driver and passenger mutations). A mutation can be transmitted from apparent cell to a daughter cell. A person having skill in the art will appreciate that a genetic mutation (e.g., a driver mutation) in a parent cell can induce additional, different mutations (e.g., passenger mutations) in a daughter cell. A mutation generally occurs in a nucleic acid. In a particular example, a mutation can be a detectable change in one or more deoxyribonucleic acids or fragments thereof. A mutation generally refers to nucleotides that is added, deleted, substituted for, inverted, or transposed to a new position in a nucleic acid. A mutation can be a spontaneous mutation or an experimentally induced mutation. A mutation in the sequence of a particular tissue is an example of a “tissue-specific allele.” For example, a tumor can have a mutation that results in an allele at a locus that does not occur in normal cells. Another example of a “tissue-specific allele” is a fetal-specific allele that occurs in the fetal tissue, but not the maternal tissue.

As used herein, the terms “size profile” and “size distribution” can relate to the sizes of DNA fragments in a biological sample. A size profile can be a histogram that provides a distribution of an amount of DNA fragments at a variety of sizes. Various statistical parameters (also referred to as size parameters or just parameter) can distinguish one size profile to another. One parameter can be the percentage of DNA fragment of a particular size or range of sizes relative to all DNA fragments or relative to DNA fragments of another size or range.

As used herein, the terms “somatic cells” and “germline cells” refer interchangeably to non-cancerous cells within a subject.

As used herein, the term “hematopoietic cells” refers to cells produced through hematopoiesis. Particularly relevant to the present disclosure are hematopoietic white blood cells, which contribute cell-free DNA fragments encompassing variant alleles that are created by clonal hematopoiesis, but which do not appear to be relevant to at least

As used herein the term “cancer” or “tumor” refers to an abnormal mass of tissue in which the growth of the mass surpasses and is not coordinated with the growth of normal tissue. A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

As used herein, the Circulating Cell-free Genome Atlas or “CCGA” is defined as an observational clinical study that prospectively collects blood and tissue from newly diagnosed cancer patients as well as blood only from subjects who do not have a cancer diagnosis. The purpose of the study is to develop a pan-cancer classifier that distinguishes cancer from non-cancer and identifies tissue of origin.

As used herein, the term “level of cancer” refers to whether cancer exists (e.g., presence or absence), a stage of a cancer, a size of tumor, presence or absence of metastasis, an estimated tumor fraction concentration, a total tumor mutational burden value, the total tumor burden of the body, and/or other measure of a severity of a cancer (e.g., recurrence of cancer). The level of cancer can be a number or other indicia, such as symbols, alphabet letters, and colors. The level can be zero. The level of cancer can also include premalignant or precancerous conditions (states) associated with mutations or a number of mutations. The level of cancer can be used in various ways. For example, screening can check if cancer is present in someone who is not known previously to have cancer. Assessment can investigate someone who has been diagnosed with cancer to monitor the progress of cancer over time, study the effectiveness of therapies or to determine the prognosis. In one embodiment, the prognosis can be expressed as the chance of a subject dying of cancer, or the chance of the cancer progressing after a specific duration or time, or the chance of cancer metastasizing. Detection can comprise ‘screening’ or can comprise checking if someone, with suggestive features of cancer (e.g., symptoms or other positive tests), has cancer. A “level of pathology” can refer to level of pathology associated with a pathogen, where the level can be as described above for cancer. When the cancer is associated with a pathogen, a level of cancer can be a type of a level of pathology.

As used herein, the term “read segment” or “read” refers to any nucleotide sequences including sequence reads obtained from an individual and/or nucleotide sequences derived from the initial sequence read from a sample obtained from an individual. For example, a read segment can refer to an aligned sequence read, a collapsed sequence read, or a stitched read. Furthermore, a read segment can refer to an individual nucleotide base, such as a single nucleotide variant.

As used herein, the term “size-distribution metric” refers to a single value, or a set of values, that are characteristic of the distribution of cell-free DNA nucleic acid fragment sequences from a biological sample that encompass a particular allele. Subjects that have a single allele at a particular genomic locus will likewise have a single cell-free DNA fragment size distribution for the particular locus. Subjects that have two alleles at a particular genomic locus (e.g., a reference allele and a variant allele, regardless of the type of cell the variant allele originates from), however, will have two cell-free DNA fragment size distribution for the particular locus, from which two size-distribution metrics can be determined, e.g., one for the reference allele and one for the variant allele. In some embodiments, a size-distribution metric for an allele refers to a vector containing the lengths of each cell-free DNA fragment that was sequenced from a biological sample encompassing the allele. In some embodiments, a size-distribution metric refers to a single value that is representative of the distribution, e.g., a central tendency of length across the distribution, such as an arithmetic mean, weighted mean, midrange, midhinge, trimean, Winsorized mean, median, or mode of the distribution.

As used herein, the term “vector” is an enumerated list of elements, such as an array of elements, where each element has an assigned meaning. As such, the term “vector” as used in the present disclosure is interchangeable with the term “tensor.” As an example, if a vector comprises the bin counts for 10,000 bins, there exists a predetermined element in the vector for each one of the 10,000 bins. For ease of presentation, in some instances a vector may be described as being one-dimensional. However, the present disclosure is not so limited. A vector of any dimension may be used in the present disclosure provided that a description of what each element in the vector represents is defined (e.g., that element 1 represents bin count of bin 1 of a plurality of bins, etc.).

The terms “sequencing depth,” “coverage” and “coverage rate” are used interchangeably herein to refer to the number of times a locus is covered by a consensus sequence read corresponding to a unique nucleic acid target molecule (“nucleic acid fragment”) aligned to the locus; e.g., the sequencing depth is equal to the number of unique nucleic acid target fragments (excluding PCR sequencing duplicates) covering the locus. The locus can be as small as a nucleotide, or as large as a chromosome arm, or as large as an entire genome. Sequencing depth can be expressed as “YX”, e.g., 50×, 100×, etc., where “Y” refers to the number of times a locus is covered with a sequence corresponding to a nucleic acid target; e.g., the number of times independent sequence information is obtained covering the particular locus. In some embodiments, the sequencing depth corresponds to the number of genomes that have been sequenced. Sequencing depth can also be applied to multiple loci, or the whole genome, in which case Y can refer to the mean or average number of times a loci or a haploid genome, or a whole genome, respectively, is sequenced. When a mean depth is quoted, the actual depth for different loci included in the dataset can span over a range of values. Ultra-deep sequencing can refer to at least 100× in sequencing depth at a locus.

As used herein, the term “read-depth metric” refers to a value that is characteristic of the total number of read segments from a biological sample that encompass a particular allele. In some embodiments, the read-depth metric refers to a value that is characteristic of the collapsed fragment coverage for a particular allele in a biological sample.

As used herein, the term “allele frequency” refers to the frequency at which a particular allele is represented at a particular genomic locus in the cell-free DNA of a biological sample, e.g., relative to the total occurrence of the loci in the biological sample. In some embodiments, allele frequency is calculated by dividing the read-depth of the allele in the biological sample by the read depth of the loci in the biological sample.

As used herein, the term “allele-frequency metric” refers to a value that is characteristic of the allele frequency for a particular allele in the biological sample.

As used herein, the terms “sequencing,” “sequence determination,” and the like refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “nucleic acid fragment sequence” refers to all or a portion of a polynucleotide sequence of at least three consecutive nucleotides. In the context of sequencing cell-free nucleic acid fragments found in a biological sample, the term “nucleic acid fragment sequence” refers to the sequence of a cell-free nucleic acid molecule (e.g., a cell-free DNA fragment) that is found in the biological sample or a representation thereof (e.g., an electronic representation of the sequence). Similarly, in the context of sequencing a locus within a larger polynucleotide, e.g., genomic DNA, the term “nucleic acid fragment sequence” refers to the sequence of the locus or a representation thereof. In such contexts, sequencing data (e.g., raw or corrected sequence reads from whole genome sequencing, targeted sequencing, etc.) from a unique nucleic acid fragment (e.g., a cell-free nucleic acid, genomic fragment, or a locus within a larger polynucleotide that is defined by a pair of PCR primers) are used to determine a nucleic acid fragment sequence. Such sequence reads, which in fact may be obtained from sequencing of PCR duplicates of the original nucleic acid fragment, therefore “represent” or “support” the nucleic acid fragment sequence. There may be a plurality of sequence reads that each represent or support a particular nucleic acid fragment in a biological sample (e.g., PCR duplicates), however, there will only be one nucleic acid fragment sequence for the particular nucleic acid fragment. In some embodiments, duplicate sequence reads generated for the original nucleic acid fragment are combined or removed (e.g., collapsed into a single sequence, e.g., the nucleic acid fragment sequence). Accordingly, when determining metrics relating to a population of nucleic acid fragments, in a sample, that each encompass a particular locus (e.g., an abundance value for the locus or a metric based on a characteristic of the distribution of the fragment lengths), the nucleic acid fragment sequences for the population of nucleic acid fragments, rather than the supporting sequence reads (e.g., which may be generated from PCR duplicates of the nucleic acid fragments in the population, should be used to determine the metric. This is because, in such embodiments, only one copy of the sequence is used to represent the original (e.g., unique) nucleic acid fragment (e.g., unique cell-free nucleic acid molecule). It is noted that the nucleic acid fragment sequences for a population of nucleic acid fragments may include several identical sequences, each of which represents a different original nucleic acid fragment, rather than duplicates of the same original nucleic acid fragment. In some embodiments, a cell-free nucleic acid is considered a nucleic acid fragments.

As used herein the term “sequencing breadth” refers to what fraction of a particular reference genome (e.g., human reference genome) or part of the genome has been analyzed. The denominator of the fraction can be a repeat-masked genome, and thus 100% can correspond to all of the reference genome minus the masked parts. A repeat-masked genome can refer to a genome in which sequence repeats are masked (e.g., nucleic acid fragment sequences are aligned to unmasked portions of the genome). Any parts of a genome can be masked, and thus one can focus on any particular part of a reference genome. Broad sequencing can refer to sequencing and analyzing at least 0.1% of the genome.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes.

Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art can be used to detect any of the properties of nucleic acids mentioned herein. Properties of a nucleic acids can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid fragments). An assay or method can have a particular sensitivity and/or specificity, and their relative usefulness as a diagnostic tool can be measured using ROC-AUC statistics.

The term “classification” can refer to any number(s) or other characters(s) that are associated with a particular property of a sample. For example, a “+” symbol (or the word “positive”) can signify that a sample is classified as having deletions or amplifications. In another example, the term “classification” can refer to an amount of tumor tissue in the subject and/or sample, a size of the tumor in the subject and/or sample, a stage of the tumor in the subject, a tumor load in the subject and/or sample, and presence of tumor metastasis in the subject. The classification can be binary (e.g., positive or negative) or have more levels of classification (e.g., a scale from 1 to 10 or 0 to 1). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

As used herein, the term “true positive” (TP) refers to a subject having a condition. “True positive” can refer to a subject that has a tumor, a cancer, a precancerous condition (e.g., a precancerous lesion), a localized or a metastasized cancer, or a non-malignant disease. “True positive” can refer to a subject having a condition, and is identified as having the condition by an assay or method of the present disclosure.

As used herein, the term “true negative” (TN) refers to a subject that does not have a condition or does not have a detectable condition. True negative can refer to a subject that does not have a disease or a detectable disease, such as a tumor, a cancer, a precancerous condition (e.g., a precancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity can characterize the ability of a method to correctly identify one or more markers indicative of cancer.

As used herein, the term “false positive” (FP) refers to a subject that does not have a condition. False positive can refer to a subject that does not have a tumor, a cancer, a precancerous condition (e.g., a precancerous lesion), a localized or a metastasized cancer, a non-malignant disease, or is otherwise healthy. The term false positive can refer to a subject that does not have a condition, but is identified as having the condition by an assay or method of the present disclosure.

As used herein, the term “false negative” (FN) refers to a subject that has a condition. False negative can refer to a subject that has a tumor, a cancer, a precancerous condition (e.g., a precancerous lesion), a localized or a metastasized cancer, or a non-malignant disease. The term false negative can refer to a subject that has a condition, but is identified as not having the condition by an assay or method of the present disclosure.

As used herein, the “negative predictive value” or “NPV” can be calculated by TN/(TN+FN) or the true negative fraction of all negative test results. Negative predictive value can be inherently impacted by the prevalence of a condition in a population and pre-test probability of the population intended to be tested. The term “positive predictive value” or “PPV” can be calculated by TP/(TP+FP) or the true positive fraction of all positive test results. PPV can be inherently impacted by the prevalence of a condition in a population and pre-test probability of the population intended to be tested. See, e.g., O'Marcaigh and Jacobson, 1993, “Estimating The Predictive Value of a Diagnostic Test, How to Prevent Misleading or Confusing Results,” Clin. Ped. 32(8): 485-491, which is entirely incorporated herein by reference.

As used herein, the term “relative abundance” can refer to a ratio of a first amount of nucleic acid fragments having a particular characteristic (e.g., a specified length, ending at one or more specified coordinates/ending positions, or aligning to a particular region of the genome) to a second amount nucleic acid fragments having a particular characteristic (e.g., a specified length, ending at one or more specified coordinates/ending positions, or aligning to a particular region of the genome). In one example, relative abundance may refer to a ratio of the number of DNA fragments ending at a first set of genomic positions to the number of DNA fragments ending at a second set of genomic positions. In some aspects, a “relative abundance” can be a type of separation value that relates an amount (one value) of cell-free DNA molecules ending within one window of genomic position to an amount (other value) of cell-free DNA molecules ending within another window of genomic positions. The two windows can overlap, but can be of different sizes. In other implementations, the two windows cannot overlap. Further, the windows can be of a width of one nucleotide, and therefore be equivalent to one genomic position.

As used herein the term “untrained classifier” refers to a classifier that has not been trained on a target dataset. For instance, consider the case of a target dataset that is a value training set discussed in further detail below. The value training set is applied as collective input to an untrained classifier, in conjunction with the cancer class of each respective reference subject represented by the value training set, to train the untrained classifier on cancer class thereby obtaining a trained classifier. The target dataset may represent raw or normalized measurements from subjects represented by the target dataset, principal components derived from such raw or normalized measurements, regression coefficients derived from the raw or normalized measurements (or the principal components of the raw or normalized measurements), or any other form of data from subjects with known disease class that is used to train classifiers in the art. In general, a target dataset is the dataset that is used to directly train an untrained classifier. However, it will be appreciated that the term “untrained classifier” does not exclude the possibility that transfer learning techniques are used in such training of the untrained classifier. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In the case where transfer learning is used, the untrained classifier described above is provided with additional data over and beyond that of the disease class labeled target dataset. That is, in non-limiting examples of transfer learning embodiments, the untrained classifier receives (i) the disease class labeled target training dataset (e.g., the value training set with each respective reference subject represented by the value training set labeled by cancer class) and (ii) additional data. Typically, this additional data is in the form of coefficients (e.g. regression coefficients) that were learned from another, auxiliary training dataset. More specifically, in some embodiments, the target training dataset is in the form of a first two-dimensional matrix, with one axis representing patients, and the other axis representing some property of respective patients, such as bin counts across all or a portion of the genome of respective patients in the target training set. Application of pattern classification techniques to the auxiliary training dataset yields a second two-dimensional matrix, where one axis is the learned coefficients and the other axis is the property of respective patients in the auxiliary training dataset, such as bin counts across all or a portion of respective patients in the first auxiliary training dataset. Matrix multiplication of the first and second matrices by their common dimension (e.g. bin counts) yields a third matrix of auxiliary data that can be applied, in addition to the first matrix to the untrained classifier. One reason it might be useful to train the untrained classifier using this additional information from an auxiliary training dataset is a paucity of subjects in one or more categories in the target dataset (e.g., the value training set). This is a particular issue for many healthcare datasets, where there may not be a large number of patients who have a particular disease or who are at a particular stage of a given disease. Making use of as much of the available data as possible can increase the accuracy of classifications and thus improve patient results. Thus, in the case where an auxiliary training dataset is used to train an untrained classifier beyond just the target training dataset (e.g. value training set), the auxiliary training dataset is subjected to classification techniques (e.g., principal component analysis followed by logistic regression) to learn coefficients (e.g., regression coefficients) that discriminate disease class based on the auxiliary training dataset. Such coefficients can be multiplied against a first instance of the target training dataset (e.g., the value training set) and inputted into the untrained classifier in conjunction with the target training dataset (e.g., the value training set) as collective input, in conjunction with the disease class (e.g. cancer class) of each respective reference subject in the target training dataset. As one of skill in the art will appreciate, such transfer learning can be applied with or without any form of dimension reduction technique on the auxiliary training dataset or the target training dataset. For instance, the auxiliary training dataset (from which coefficients are learned and used as input to the untrained classifier in addition to the target training dataset) can be subjected to a dimension reduction technique prior to regression (or other form of label based classification) to learn the coefficients that are applied to the target training dataset. Alternatively, no dimension reduction other than regression or some other form of pattern classification is used in some embodiments to learn such coefficients from the auxiliary training dataset prior to applying the coefficients to an instance of the target training dataset (e.g., through matrix multiplication where one matrix is the coefficients learned from the auxiliary training dataset and the second matrix is an instance of the target training dataset). Moreover, in some embodiments, rather than applying the coefficients learned from the auxiliary training dataset to the target training dataset, such coefficients are applied (e.g., by matrix multiplication based on a common axis of bin counts) to the bin count data that was collected from the first plurality of reference subjects that was used as a basis for forming the value training set as disclosed herein. Moreover, while a description of a single auxiliary training dataset has been disclosed, it will be appreciated that there is no limit on the number of auxiliary training datasets that may be used to complement the target training dataset in training the untrained classifier in the present disclosure. For instance, in some embodiments, two or more auxiliary training datasets, three or more auxiliary training datasets, four or more auxiliary training datasets or five or more auxiliary training datasets are used to complement the target training dataset through transfer learning, where each such auxiliary dataset is different than the target training dataset. Any manner of transfer learning may be used in such embodiments. For instance, consider the case where there is a first auxiliary training dataset and a second auxiliary training dataset in addition to the target training dataset (where, as before the target training dataset is any dataset that is directly used to train the untrained classifier). The coefficients learned from the first auxiliary training dataset (by application of a classifier such as regression to the first auxiliary training dataset) may be applied to the second auxiliary training dataset using transfer learning techniques (e.g., the above described two-dimensional matrix multiplication), which in turn may result in a trained intermediate classifier whose coefficients are then applied to the target training dataset and this, in conjunction with the target training dataset itself, is applied to the untrained classifier. Alternatively, a first set of coefficients learned from the first auxiliary training dataset (by application of a classifier such as regression to the first auxiliary training dataset) and a second set of coefficients learned from the second auxiliary training dataset (by application of a classifier such as regression to the second auxiliary training dataset) may each independently be applied to a separate instance of the target training dataset (e.g., by separate independent matrix multiplications) and both such applications of the coefficients to separate instances of the target training dataset in conjunction with the target training dataset itself (or some reduced form of the target training dataset such as principal components learned from the target training set) may then be applied to the untrained classifier in order to train the untrained classifier. In either example, knowledge regarding disease (e.g., cancer) classification derived from the first and second auxiliary training datasets is used, in conjunction with the disease labeled target training dataset (e.g., the value training dataset), to train the untrained classifier.

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Example System Embodiments.

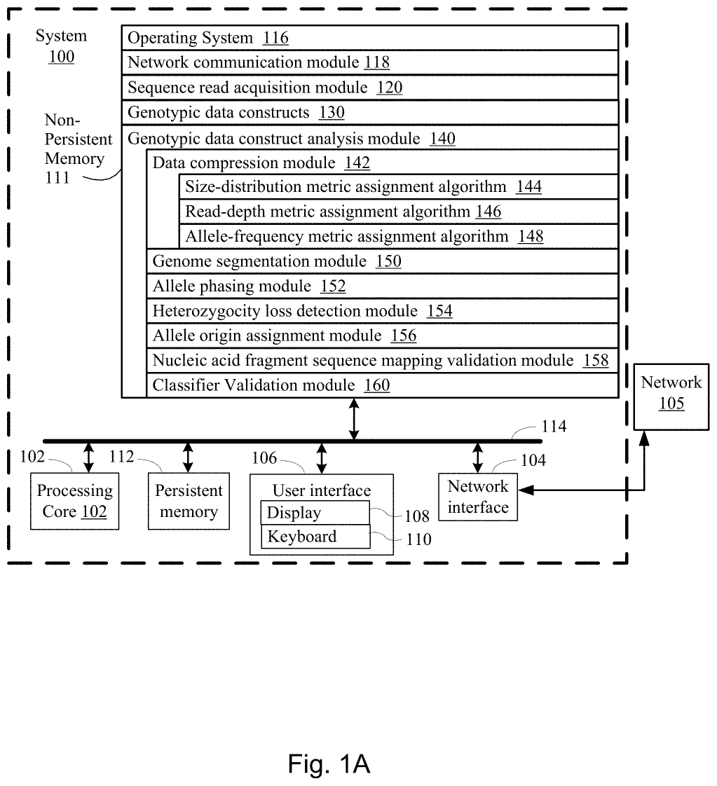

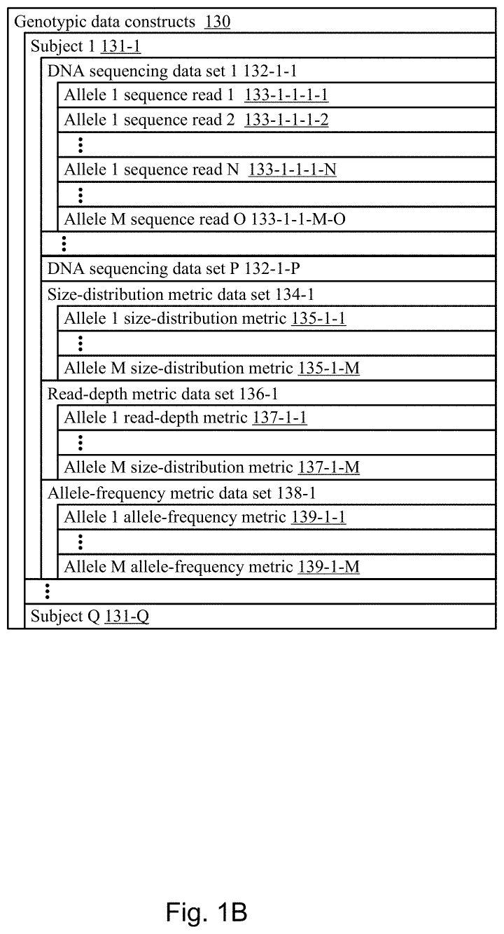

Details of an example system are described in relation to FIGS. 1A and 1B. FIG. 1A is a block diagram illustrating a system 100 for using size-distribution metrics of nucleosomal-derived, cell-free DNA fragments for the classification of cancer in a subject, in accordance with some implementations. Device 100, in some implementations, includes one or more processing units CPU(s) 102 (also referred to as processors or processing cores), one or more network interfaces 104, a user interface 106, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices and/or a communication network 105;
an optional sequence read acquisition module 120 for sequencing nucleic acids from a biological sample from a subject;
genotypic data construct data store 130 including genotypic data from one or more subject 131, where the genotypic data includes one or more of a DNA sequencing data set 132 that includes a plurality of sequences reads 133 for each of a plurality of cell-free DNA fragments encompassing a plurality of alleles, a size-distribution metric data set 134 that includes a size distribution metric 135 for each of a plurality of alleles that are encompassed by a plurality of fragments, a read-depth metric data set 136 that includes a read-depth metric 137 for each of a plurality of alleles that are encompassed by a plurality of cell-free DNA fragments, and an allele-frequency metric data set 138 that includes an allele-frequency metric 139 for each of a plurality of alleles that are encompassed by a plurality of fragments; and
a genotypic data construct analysis module 140 for analyzing genotypic data constructs (e.g., stored in genotypic data construct data store 130) in order to classify a cancer status of a subject, where genotypic data construct analysis module includes:
   an optional data compression module 142 that uses one or more of a size-distribution metric assignment algorithm 144, a read-depth metric assignment algorithm 146, and an allele-frequency metric assignment algorithm 148, to compress a DNA sequencing data set 132 into one or more of a size-distribution metric data set 134, a read-depth metric data set 136, and an allele-frequency metric data set 138, and
   one or more of a genome segmentation module 150 for segmenting the genome of a subject in accordance with embodiments of method 3700, an allele phasing module 152 for phasing alleles within the genome of a subject in accordance with embodiments of method 3800, a heterozygosity loss detecting module 154 for detecting loss of heterozygosity within the genome of a subject in accordance with embodiments of method 3900, an allele origin assignment module 156 for assigning the origin of variant alleles detected in a cell-free DNA sample from a subject in accordance with embodiments of method 4000, a nucleic acid fragment sequence mapping validation module 158 for validating the mapping of nucleic acid fragment sequences derived from cell-free DNA fragments in a sample from a subject to a position within a reference genome for the species of the subject in accordance with embodiments of method 4100, and a classification validation module 160 for validating the use of information from one or more alleles in a cancer classifier in accordance with embodiments of method 4100.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “system 100,” the figure is intended more as functional description of the various features which may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112.

While a system in accordance with the present disclosure has been disclosed with reference to FIG. 1, methods in accordance with the present disclosure are now detailed. It will be appreciated that any of the disclosed methods can make use of any of the assays or algorithms disclosed in U.S. patent application Ser. No. 15/793,830, filed Oct. 25, 2017, U.S. patent application Ser. No. 16/352,602, entitled “Anomalous Fragment Detection and Classification,” filed Mar. 13, 2019, U.S. Provisional Patent Application No. 62/847,223, entitled “Model-Based Featurization and Classification,” filed May 13, 2019, United States Patent Publication No. US 2019/0287652, and/or International Patent Publication No. PCT/US17/58099, having an International Filing Date of Oct. 24, 2017, each of which is hereby incorporated by reference, in order to determine a cancer condition in a test subject or a likelihood that the subject has the cancer condition. For instance, any of the disclosed methods can work in conjunction with any of the disclosed methods or algorithms disclosed in the patent applications and publications described above. Similarly, any of the disclosed methods can work in conjunction with any of the disclosed methods or algorithms in U.S. Patent Application Publication No. 2010/0112590 or U.S. Pat. No. 8,741,811, the disclosures of which are incorporated herein by reference, in their entireties, for all purposes, and specifically for methods of genome segmentation. Similarly, any of the disclosed methods can work in conjunction with any of the disclosed methods or algorithms for allele phasing, detecting heterozygosity, and/or allele/fragment origin assignment disclosed in U.S. Pat. No. 8,741,811.

Example Classification Models.

In some aspects, the disclosed methods can work in conjunction with cancer classification models. For example, a machine learning or deep learning model (e.g., a disease classifier) can be used to determine a disease state based on values of one or more features determined from one or more cell-free DNA molecules or nucleic acid fragment sequences (derived from one or more cfDNA molecules). In various embodiments, the output of the machine learning or deep learning model is a predictive score or probability of a disease state (e.g., a predictive cancer score). Therefore, the machine learning or deep learning model generates a disease state classification based on the predictive score or probability.

In some embodiments, the machine-learned model includes a logistic regression classifier. In other embodiments, the machine learning or deep learning model can be one of a decision tree, an ensemble (e.g., bagging, boosting, random forest), gradient boosting machine, linear regression, Naïve Bayes, or a neural network. The disease state model includes learned weights for the features that are adjusted during training. The term “weights” is used generically here to represent the learned quantity associated with any given feature of a model, regardless of which particular machine learning technique is used. In some embodiments, a cancer indicator score is determined by inputting values for features derived from one or more DNA sequences (or DNA fragment sequences thereof) into a machine learning or deep learning model.

During training, training data is processed to generate values for features that are used to train the weights of the disease state model. As an example, training data can include cfDNA data, cancer gDNA, and/or WBC gDNA data obtained from training samples, as well as an output label. For example, the output label can be an indication as to whether the individual is known to have a specific disease (e.g., known to have cancer) or known to be healthy (i.e., devoid of a disease). In other embodiments, the model can be used to determine a disease type, or tissue of origin (e.g., cancer tissue of origin), or an indication of a severity of the disease (e.g., cancer stage) and generate an output label therefor. Depending on the particular embodiment, the disease state model receives the values for one or more of the features determine from a DNA assay used for detection and quantification of a cfDNA molecule or sequence derived therefrom, and computational analyses relevant to the model to be trained. In one embodiment, the one or more features comprise a quantity of one or more cfDNA molecules or nucleic acid fragment sequences derived therefrom. Depending on the differences between the scores output by the model-in-training and the output labels of the training data, the weights of the predictive cancer model are optimized to enable the disease state model to make more accurate predictions. In various embodiments, a disease state model may be a non-parametric model (e.g., k-nearest neighbors) and therefore, the predictive cancer model can be trained to make more accurately make predictions without having to optimize parameters.

Example Method Embodiments.

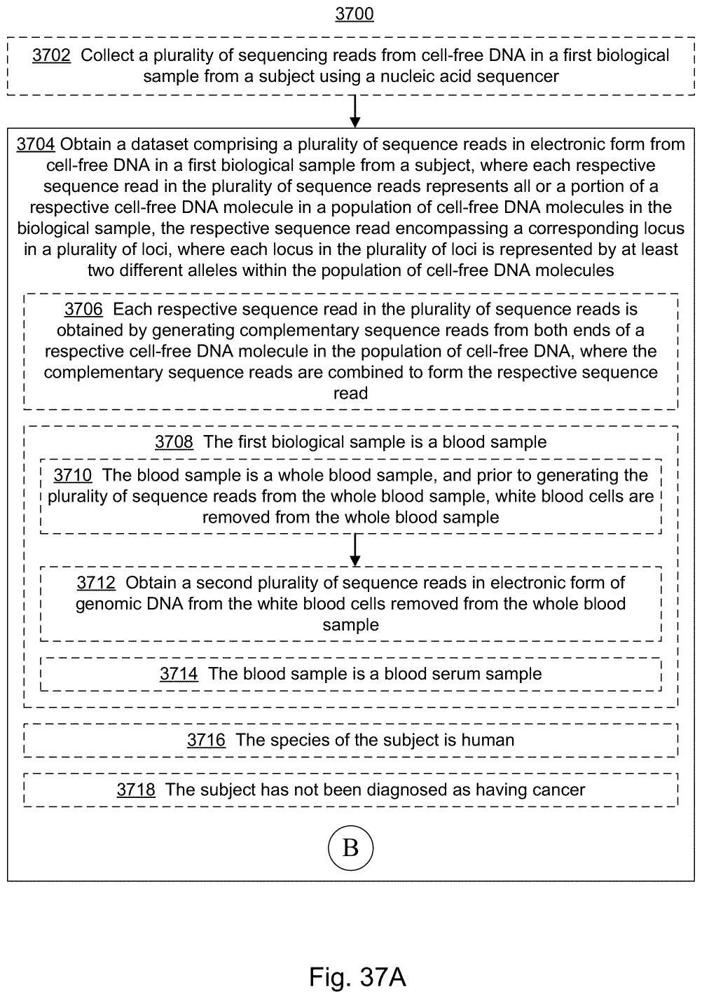

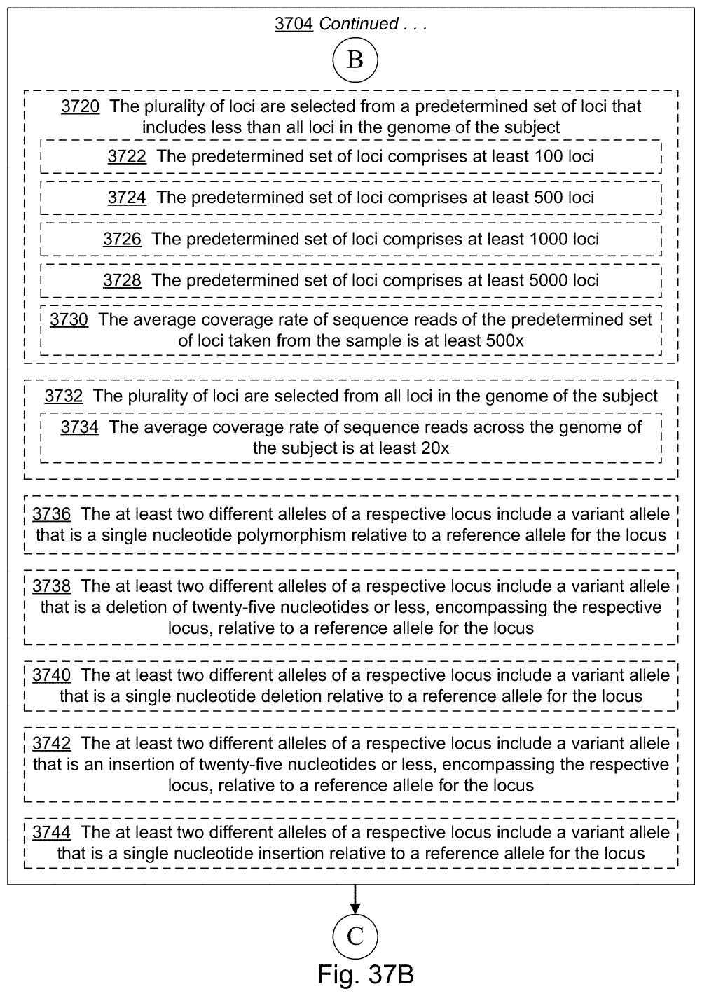

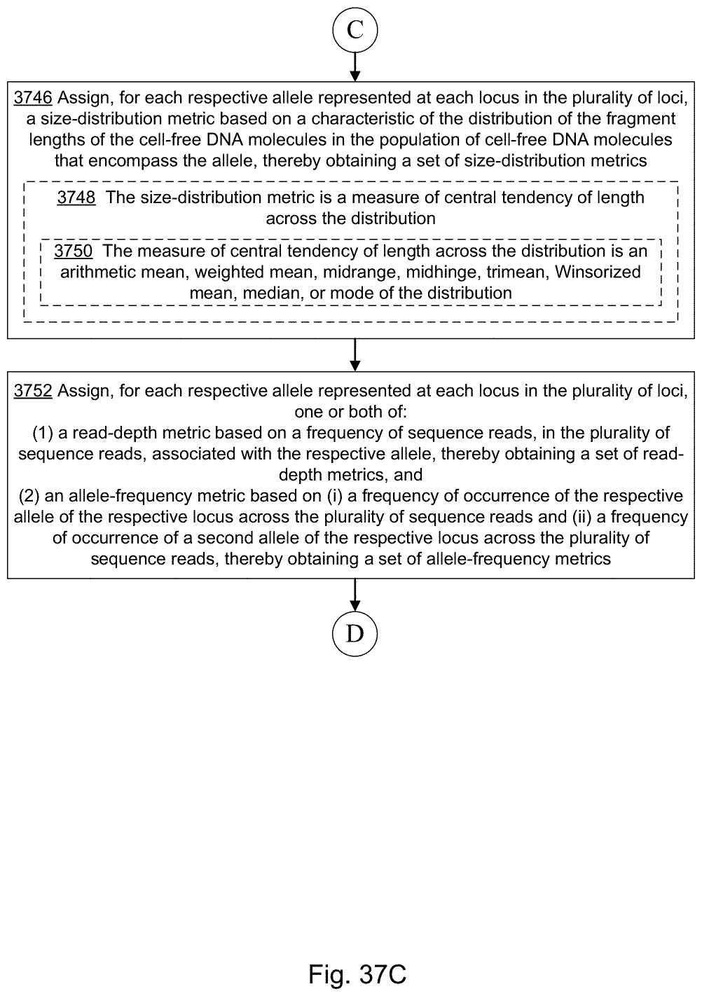

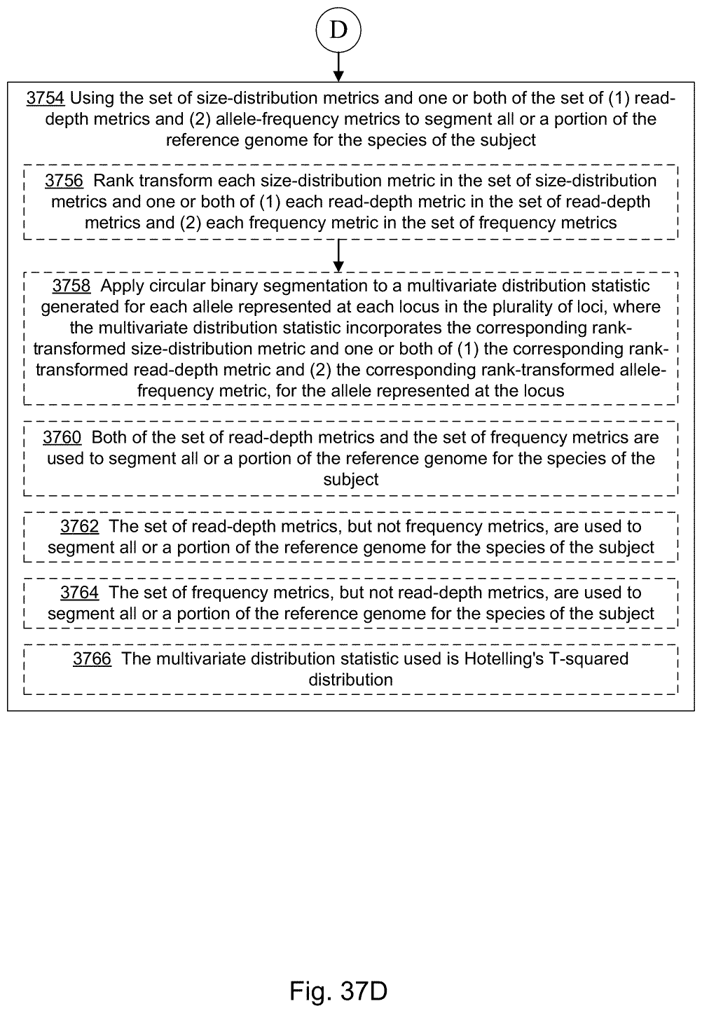

Now that details of a system 100 for using cell-free DNA fragment lengths in cancer detection and diagnostics has been disclosed, details regarding the processes and features of the system, in accordance with various embodiments of the present disclosure, are disclosed with reference to FIGS. 37 through 42. In some embodiments, such processes and features of the system are carried out by the various fragment-length utilization modules, e.g., data compression module 142, genome segmentation module 150, allele phasing module 152, heterozygosity loss detection module 154, allele assignment module 156, nucleic acid fragment sequence mapping validation module 158, and classifier validation module 160, as illustrated in FIG. 1).

The embodiments described below relate to analyses performed using nucleic acid fragment sequences of cell-free DNA fragments obtained from a biological sample, e.g., a blood sample. Generally, these embodiments are independent and, thus, not reliant upon any particular sequencing methodologies. However, in some embodiments, the methods described below include one or more steps of generating the nucleic acid fragment sequences used for the analysis, and/or specify certain sequencing parameters that are advantageous for the particular type of analysis being performed.

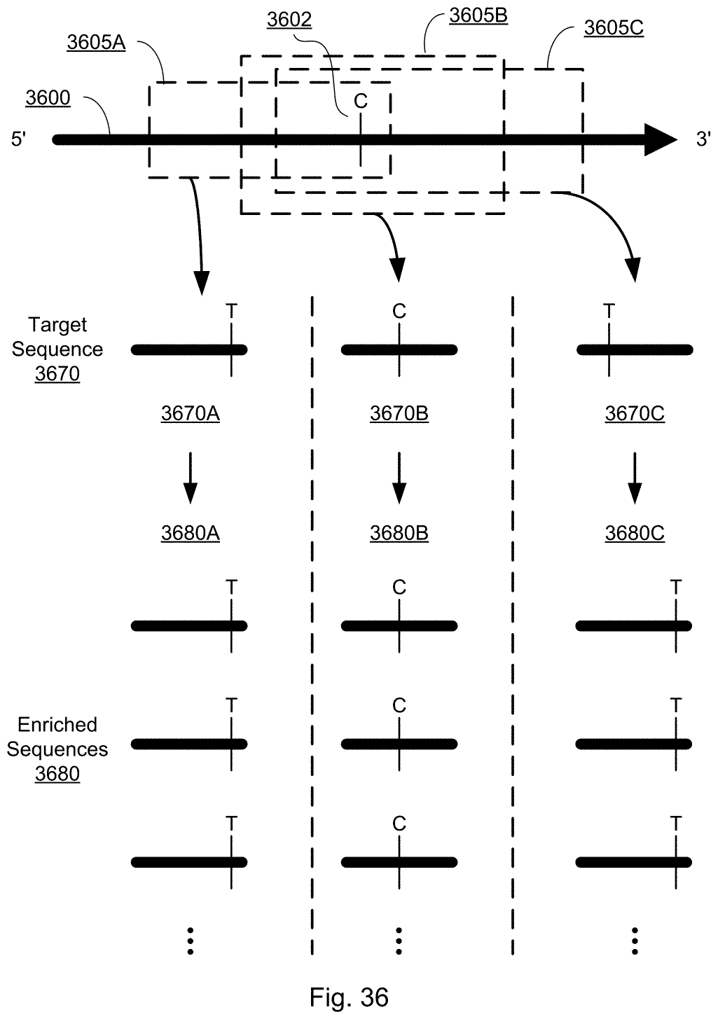

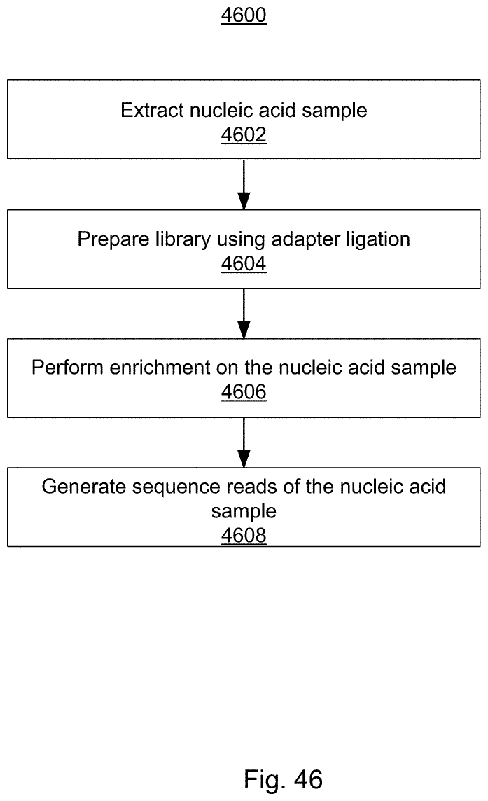

Methods for sequencing are well known in the art and include, without limitations, next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators. Described below, with reference to FIGS. 46 and 36, is an example of a method used for generating sequencing data from cell-free DNA fragments that is useful in the methods of analyzing fragment-length distributions described herein.

FIG. 46 is flowchart of a method 4600 for preparing a nucleic acid sample for sequencing according to one embodiment. The method 4600 includes, but is not limited to, the following steps. For example, any step of the method 4600 may comprise a quantitation sub-step for quality control or other laboratory assay procedures known to one skilled in the art.

In block 4602, a nucleic acid sample (DNA or RNA) is extracted from a subject. The sample may be any subset of the human genome, including the whole genome. The sample may be extracted from a subject known to have or suspected of having cancer. The sample may include blood, plasma, serum, urine, fecal, saliva, other types of bodily fluids, or any combination thereof. In some embodiments, methods for drawing a blood sample (e.g., syringe or finger prick) may be less invasive than procedures for obtaining a tissue biopsy, which may require surgery. The extracted sample may comprise cfDNA and/or ctDNA. For healthy individuals, the human body may naturally clear out cfDNA and other cellular debris. If a subject has a cancer or disease, ctDNA in an extracted sample may be present at a detectable level for diagnosis.

In block 4604, a sequencing library is prepared. During library preparation, unique molecular identifiers (UMI) are added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment. This provides a way to identify sequence reads that came from the same original fragment in downstream analysis.

In block 4606, targeted DNA sequences are enriched from the library. During enrichment, hybridization probes (also referred to herein as “probes”) are used to target, and pull down, nucleic acid fragments informative for the presence or absence of cancer (or disease), cancer status, or a cancer classification (e.g., cancer type or tissue of origin). For a given workflow, the probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10s, 100s, or 1000s of base pairs. In one embodiment, the probes are designed based on a gene panel to analyze particular mutations or target regions of the genome (e.g., of the human or another organism) that are suspected to correspond to certain cancers or other types of diseases. Moreover, the probes may cover overlapping portions of a target region.

FIG. 36 is a graphical representation of the process for obtaining nucleic acid fragment sequences according to one embodiment. FIG. 36 depicts one example of a nucleic acid segment 3600 from the sample. Here, the nucleic acid segment 3600 can be a single-stranded nucleic acid segment, such as a single stranded. In some embodiments, the nucleic acid segment 3600 is a double-stranded cfDNA segment. The illustrated example depicts three regions 3605A, 3605B, and 3605C of the nucleic acid segment that can be targeted by different probes. Specifically, each of the three regions 3605A, 3605B, and 3605C includes an overlapping position on the nucleic acid segment 3600. An example overlapping position is depicted in FIG. 36 as the cytosine (“C”) nucleotide base 3602. The cytosine nucleotide base 3602 is located near a first edge of region 3605A, at the center of region 3605B, and near a second edge of region 3605C.

In some embodiments, one or more (or all) of the probes are designed based on a gene panel to analyze particular mutations or target regions of the genome (e.g., of the human or another organism) that are suspected to correspond to certain cancers or other types of diseases. By using a targeted gene panel rather than sequencing all expressed genes of a genome, also known as “whole exome sequencing,” the method 2400 may be used to increase sequencing depth of the target regions, where depth refers to the count of the number of times a given target sequence within the sample has been sequenced. Increasing sequencing depth reduces required input amounts of the nucleic acid sample.

Hybridization of the nucleic acid sample 3600 using one or more probes results in an understanding of a target sequence 3670. As shown in FIG. 36, the target sequence 3670 is the nucleotide base sequence of the region 3605 that is targeted by a hybridization probe. The target sequence 3670 can also be referred to as a hybridized nucleic acid fragment. For example, target sequence 3670A corresponds to region 3605A targeted by a first hybridization probe, target sequence 3670B corresponds to region 3605B targeted by a second hybridization probe, and target sequence 3670C corresponds to region 3605C targeted by a third hybridization probe. Given that the cytosine nucleotide base 3602 is located at different locations within each region 3605A-C targeted by a hybridization probe, each target sequence 3670 includes a nucleotide base that corresponds to the cytosine nucleotide base 3602 at a particular location on the target sequence 3670.

After a hybridization step, the hybridized nucleic acid fragments are captured and may also be amplified using PCR. For example, the target sequences 3670 can be enriched to obtain enriched sequences 3680 that can be subsequently sequenced. In some embodiments, each enriched sequence 3680 is replicated from a target sequence 3670. Enriched sequences 3680A and 3680C that are amplified from target sequences 3670A and 3670C, respectively, also include the thymine nucleotide base located near the edge of each sequence read 3680A or 3680C. As used hereafter, the mutated nucleotide base (e.g., thymine nucleotide base) in the enriched sequence 3680 that is mutated in relation to the reference allele (e.g., cytosine nucleotide base 3602) is considered as the alternative allele. Additionally, each enriched sequence 3680B amplified from target sequence 3670B includes the cytosine nucleotide base located near or at the center of each enriched sequence 2480B.

In block 4608, nucleic acid fragment sequences are generated from the enriched DNA sequences, e.g., enriched sequences 3680 shown in FIG. 36. Sequencing data may be acquired from the enriched DNA sequences by known means in the art. For example, the method 4600 may include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In some embodiments, the nucleic acid fragment sequences may be aligned to a reference genome using known methods in the art to determine alignment position information. The alignment position information may indicate a beginning position and an end position of a region in the reference genome that corresponds to a beginning nucleotide base and end nucleotide base of a given nucleic acid fragment sequence. Alignment position information may also include nucleic acid fragment sequence length, which can be determined from the beginning position and end position. A region in the reference genome may be associated with a gene or a segment of a gene.

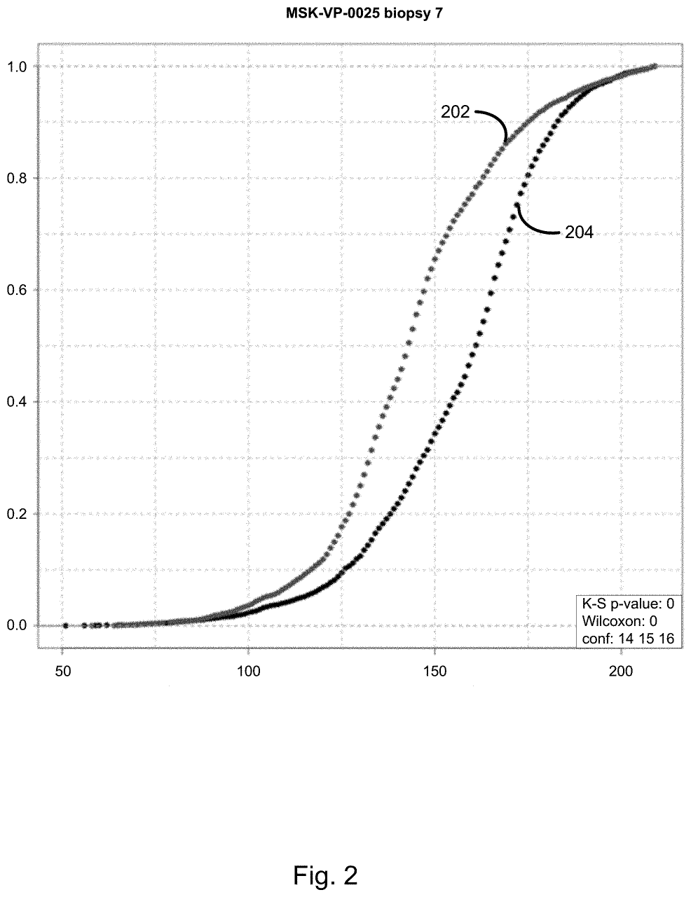

In various embodiments, a sequence read is comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format may be generated and output for further analysis such as described above in conjunction with FIG. 2.

FIGS. 37A-37D are flow diagrams illustrating a method 3700 for segmenting all or a portion of a reference genome for a species of a subject using a measure of the distribution of DNA fragment lengths of cell-free DNA fragments isolated from the blood of the subject which encompass an allele of interest. Method 3700 is performed at a computer system (e.g., computer system 100 in FIG. 1) having one or more processors, and memory storing one or more programs for execution by the one or more processors for segmenting all of a portion of a reference genome for the species of the subject. Some operations in method 3700 are, optionally, combined and/or the order of some operations is, optionally, changed.

In some embodiments, method 3700 is performed at a computer system comprising one or more processors, and memory storing one or more programs for execution by the one or more processors. The method includes obtaining (3704) a dataset comprising a plurality of nucleic acid fragment sequences in electronic form from cell-free DNA in a first biological sample from the subject, where each respective nucleic acid fragment sequence in the plurality of nucleic acid fragment sequences represents all or a portion of a respective cell-free DNA molecule in a population of cell-free DNA molecules in the biological sample, the respective nucleic acid fragment sequence encompassing a corresponding locus in a plurality of loci, wherein each locus in the plurality of loci is represented by at least two different alleles (e.g., a reference allele and a variant allele, where the variant allele is a SNP, insertion, deletion, inversion, etc.) within the population of cell-free DNA molecules.

For example, as described above, it is known that mono- and di-nucleosomes fragmented from the genomes of non-cancerous somatic cells, hematopoietic cells (e.g., white blood cells), and (when the subject has cancer) cancerous cells. Thus, in some embodiments, the cell-free DNA molecules in the sample originate from at least non-cancerous somatic cells and hematopoietic cells (e.g., white blood cells). In some embodiments, sample also includes cell-free DNA molecules originating from cancerous cells. In some embodiments, it is unknown whether the subject has cancer and, thus, whether cell-free DNA originating from cancerous cells in present in the sample prior to analysis. Accordingly, in some embodiments, the subject has not been diagnosed as having cancer (3718). In some embodiments, the subject has already been diagnosed with cancer and, accordingly, it is known that the cell-free DNA originating from cancerous cells is present in the sample prior to analysis. In some embodiments, the subject is a human (3716).

In some embodiments, the obtaining step of the method includes collecting (3702) the plurality of sequencing reads from the cell-free DNA in the biological sample from the subject using a nucleic acid sequencer. However, in other embodiments, method 3700 only includes obtaining the sequencing data from a prior sequencing reaction of cell-free DNA from a biological sample.

Methods for collecting suitable sequencing data for the methods described herein (e.g., method 3700) are described above, and are not reiterated here for reasons of brevity. Regardless of the exact sequencing method used, however, in some embodiments, each respective nucleic acid fragment sequence in the plurality of nucleic acid fragment sequences is obtained by generating complementary sequence reads from both ends of a respective cell-free DNA molecule in the population of cell-free DNA (3706), where the complementary sequence reads are combined to form a respective sequence read, which is collapsed with other respective sequence reads of the same unique nucleic acid fragment to form the respective nucleic acid fragment sequence. For example, in some embodiments, complementary sequence reads are stitched together based on an overlapping region of sequence shared between the complementary sequence reads and/or by matching the sequences from complementary sequence reads to corresponding sequences in a reference genome for the species of the subject.

In some embodiments, the first biological sample is a blood sample (3708), e.g., a whole-blood sample, a blood serum sample, or a blood plasma sample. In some embodiments, the blood sample is a whole blood sample, and prior to generating the plurality of nucleic acid fragment sequences from the whole blood sample, white blood cells are removed from the whole blood sample (3710). In some embodiments, the white blood cells are collected as a second type of sample, e.g., according to a buffy coat extraction method, from which additional sequencing data may or may not be obtained. Methods for buffy coat extraction of white blood cells are known in the art, for example, as described in U.S. Patent Application Serial No. U.S. Provisional Application No. 62/679,347, filed on Jun. 1, 2018, the content of which is incorporated herein by reference in its entirety. In some embodiments, the method further includes obtaining (3712) a second plurality of nucleic acid fragment sequences in electronic form of genomic DNA from the white blood cells removed from the whole blood sample. In some embodiments, the second plurality of nucleic acid fragment sequences is used to identify allele variants arising from clonal hematopoiesis, as opposed to germline allele variants and/or allele variants arising from a cancer in the subject. Likewise, in some embodiments, fragment length distributions obtained for fragments encompassing an allele are used to seed a classification algorithm, e.g., an expectation maximization (EM) algorithm. In some embodiments, the blood sample is a blood serum sample (3714).

In some embodiments, the plurality of loci is selected from a predetermined set of loci that includes less than all loci in the genome of the subject (3720). In some embodiments, nucleic acid fragment sequences of the cell-free DNA molecules in the sample are generated for a predetermined set of loci, e.g., by targeted panel sequencing. In some embodiments, a target panel includes probes targeting dozens or hundreds of markers for detecting a genetic condition (including somatic mutations in cancer). In some embodiments, a marker can be a full-length gene. In some embodiments, a marker can be an allele, including but not limited to point mutations and indels within a gene. Many targeted panels for sequencing alleles of interest, e.g., related to cancer diagnostics, are known to those of skill in the art. Although not reiterated here for reasons of brevity, any of these targeted panels can be used in the methods described herein. In some embodiments, the targeted panel includes loci known to provide diagnostic or prognostic power for cancer diagnostics, e.g., loci at which an allele has been linked to a characteristic of a cancer. In some embodiments, the targeted panel includes alleles that are distributed throughout the genome of the species of the subject, e.g., to provide representation for a large portion of the genome.

In some embodiments, the predetermined set of loci includes at least 100 loci (3722). In some embodiments, the predetermined set of loci includes at least 500 loci (3724). In some embodiments, the predetermined set of loci includes at least 1000 loci (3726). In some embodiments, the predetermined set of loci includes at least 5000 loci (3728). In some embodiments, the predetermined set of loci includes at least 100, 200, 300, 400, 500, 600, 700, 800, 900, 1000, 2000, 3000, 4000, 5000, 6000, 7000, 8000, 9000, 10,000, 15,000, 20,000, 25,000, 50,000, 75,000, 100,000, or more loci. In some embodiments, the predetermined set of loci includes from 100 to 100,000 loci, from 100 to 50,000 loci, from 100 to 25,000 loci, from 100 to 10,000 loci, from 100 to 5000 loci, from 100 to 2000 loci, from 100 to 1000 loci, from 500 to 100,000 loci, from 500 to 50,000 loci, from 500 to 25,000 loci, from 500 to 10,000 loci, from 500 to 5000 loci, from 500 to 2000 loci, from 500 to 1000 loci, from 1000 to 100,000 loci, from 1000 to 50,000 loci, from 1000 to 25,000 loci, from 1000 to 10,000 loci, from 1000 to 5000 loci, or from 1000 to 2000 loci.

In some embodiments, the average coverage rate of nucleic acid fragment sequences of the predetermined set of loci taken from the sample is at least 50× (3730). In some embodiments, the average coverage rate of nucleic acid fragment sequences of the predetermined set of loci taken from the sample is at least 50×, 100×, 200×, 300×, 400×, 500×, 750×, 1000×, 2000×, 3000×, 4000×, 5000×, 6000×, 7000×, 8000×, 9000×, 10,000×, or more. In some embodiments, it is possible to accurately determine a locus at a read depth lower than 50×; for example, when calling a germline allele. In some embodiments, the average coverage rate of nucleic acid fragment sequences of the predetermined set of loci taken from the sample is from 50× to 250×, 100× to 500×, 500× to 5000×, from 500× to 2500×, from 500× to 1000×, from 1000× to 5000×, from 1000× to 2500×, or from 2500× to 5000×.

In some embodiments, all of the cell-free DNA molecules in the sample are sequenced (3732), e.g., by whole genome sequencing, and nucleic acid fragment sequences corresponding to cell-free DNA molecules encompassing the predetermined set of loci are selected for the analysis. As described above, many methods for whole genome sequencing are known to those of skill in the art. In some embodiments, the average coverage rate of nucleic acid fragment sequences across the genome of the subject is at least 20× (3734). In some embodiments, the average coverage rate of nucleic acid fragment sequences across the genome of the subject is at least 10×, 20×, 30×, 40×, 50×, 100×, 200×, 300×, 400×, 500×, 750×, 1000×, or more. In some embodiments, the average coverage rate of nucleic acid fragment sequences of the predetermined set of loci taken from the sample is from 20× to 1000×, from 20× to 500×, from 20× to 100×, from 20× to 50×, from 50× to 1000×, from 50× to 500×, or from 50× to 100×.

In some embodiments, the at least two different alleles of a respective locus include a reference allele and a variant allele. In some embodiments, the at least two different alleles of a respective locus include a variant allele that is a single nucleotide polymorphism relative to a reference allele for the locus (3736). In some embodiments, the preceding claims, wherein the at least two different alleles of a respective locus include a variant allele that is a deletion of twenty-five nucleotides or less, encompassing the respective locus, relative to a reference allele for the locus (3738). In some embodiments, the at least two different alleles of a respective locus include a variant allele that is a single nucleotide deletion relative to a reference allele for the locus (3740). In some embodiments, the at least two different alleles of a respective locus include a variant allele that is an insertion of twenty-five nucleotides or less, encompassing the respective locus, relative to a reference allele for the locus (3742). In some embodiments, the at least two different alleles of a respective locus include a variant allele that is a single nucleotide insertion relative to a reference allele for the locus (3744).

Method 3700 also includes assigning (3746), for each respective allele represented at each locus in the plurality of loci, a size-distribution metric (e.g., a median length, a median shift in length, a measure of central tendency of length across the distribution, a measure of central tendency of shift in length across the distribution, or a statistical distribution) based on a characteristic of the distribution of the fragment lengths of the cell-free DNA molecules in the population of cell-free DNA molecules (e.g., that are represented by a respective nucleic acid fragment sequence in the plurality of nucleic acid fragment sequences) that encompass the allele, thereby obtaining a set of size-distribution metrics. Because the set of size-distribution metrics is smaller than the set of individual nucleic acid fragment sequences, this step compresses the data in order to make the method more computationally efficient, e.g., by allowing the computer to apply an algorithm to the smaller dataset (the set size distribution metrics) rather than the full dataset (the nucleic acid fragment sequences themselves). In one embodiment, the size-distribution metric is a measure of central tendency of length across the distribution (3748). In some embodiments, the measure of central tendency of length across the distribution is an arithmetic mean, weighted mean, midrange, midhinge, trimean, Winsorized mean, median, or mode of the distribution (3750).

Method 3700 also includes assigning (3752), for each respective allele represented at each locus in the plurality of loci, one or both of: (1) a read-depth metric based on a frequency of nucleic acid fragment sequences, in the plurality of nucleic acid fragment sequences, associated with the respective allele (e.g., a frequency of nucleic acid fragment sequences containing the respective allele or a frequency of nucleic acid fragment sequences that correspond to a same portion of a reference genome (e.g., a bin) for the species of the subject as the locus represented by the respective allele, in a plurality of different and non-overlapping portions of the reference genome), thereby obtaining a set of read-depth metrics (e.g., determining read depth for each allele at a loci or region of the genome of interest), and (2) an allele-frequency metric based on (i) a frequency of occurrence of the respective allele of the respective locus across the plurality of nucleic acid fragment sequences and (ii) a frequency of occurrence of a second allele of the respective locus across the plurality of nucleic acid fragment sequences, thereby obtaining a set of allele-frequency metrics (e.g., determining allele ratios for respective alleles at a loci of interest).

Method 3700 also includes using (3754) the set of size-distribution metrics and one or both of the set of (1) read-depth metrics and (2) allele-frequency metrics to segment all or a portion of the reference genome (e.g., to identify regions of the genome having copy number aberrations based on cell-free DNA fragment length distributions and/or one or both of read-depths for alleles in the cell-free DNA and allele-frequencies in the cell-free DNA) for the species of the subject. In some embodiments, both of the set of read-depth metrics and the set of frequency metrics are used to segment all or a portion of the reference genome for the species of the subject (3760). In some embodiments, the set of read-depth metrics, but not frequency metrics, are used to segment all or a portion of the reference genome for the species of the subject (3762). In some embodiments, the set of frequency metrics, but not read-depth metrics, are used to segment all or a portion of the reference genome for the species of the subject (3764).

Methods for identifying copy number aberrations using metrics other than cell-free DNA fragment lengths are known in the art. See, for example, Hodgson G., et al., Nat. Genet., 29:459-64 (2001) (three-component Gaussian mixture model); Autio, R., et al., Bioinformatics 19(13):1714-15 (2003) (k-means clustering and dynamic programming), Fridlyand J., et al., J. Multivar. Anal., 90:132-53 (2004) (Hidden Markov model); Wang et al., Biostatistics, 6(1):45-58 (2005) (hierarchical clustering); Tibshirani R, et al., Biostatistics 9(1):18-29 (2008) (fused lasso logistic regression); and Olshen A B, et al., Biostatistics 5(4):557-72 (2004) (circular binary segmentation), the contents of which are incorporated herein by reference. In some embodiments, a conventional method for identifying copy number aberrations is supplemented by including analysis of cell-free DNA fragment-length distribution. Because fragment-length distribution is orthogonal information relative to conventional information used for identifying copy number aberrations (e.g., allele-frequency and/or allele read-depth), the inclusion of fragment length distribution increases the power of the algorithm used to detect chromosomal copy number aberrations.

In some embodiments, segmenting all or a portion of the reference genome includes rank transforming (3756) each size-distribution metric in the set of size-distribution metrics and one or both of (1) each read-depth metric in the set of read-depth metrics and (2) each frequency metric in the set of frequency metrics. In some embodiments, the segmenting then includes applying (3758) circular binary segmentation to a multivariate distribution statistic generated for each allele represented at each locus in the plurality of loci, wherein the multivariate distribution statistic incorporates the corresponding rank-transformed size-distribution metric and one or both of (1) the corresponding rank-transformed read-depth metric and (2) the corresponding rank-transformed allele-frequency metric, for the allele represented at the locus. For a review of the use of circular binary segmentation, see, Olshen A B, et al., Biostatistics 5(4):557-72 (2004), the content of which is incorporated herein by reference. In some embodiments, the multivariate distribution statistic is Hotelling's T-squared distribution (3766). For a review of Hotelling's T-squared distribution, see Hotelling, H., Ann. Math. Statist. 2(3):360-78 (1931), the content of which is incorporated herein by reference.

It should be understood that the particular order in which the operations in FIGS. 37A-37D have been described is merely an example and is not intended to indicate that the described order is the only order in which the operations could be performed. One of ordinary skill in the art would recognize various ways to reorder the operations described herein. Additionally, it should be noted that details of other processes described herein with respect to other methods described herein (e.g., methods 3800, 3900, 4000, 4100, and 4200) are also applicable in an analogous manner to method 3700 described above with respect to FIGS. 37A-37D. Further, in some embodiments, method 3800 can be used in conjunction with any other method described herein (e.g., methods 3700, 3900, 4000, 4100, and 4200). The operations in the information processing methods described above are, optionally implemented by running one or more functional modules in information processing apparatus such as general purpose processors (e.g., as described above with respect to FIGS. 1A and 1B) or application specific chips.

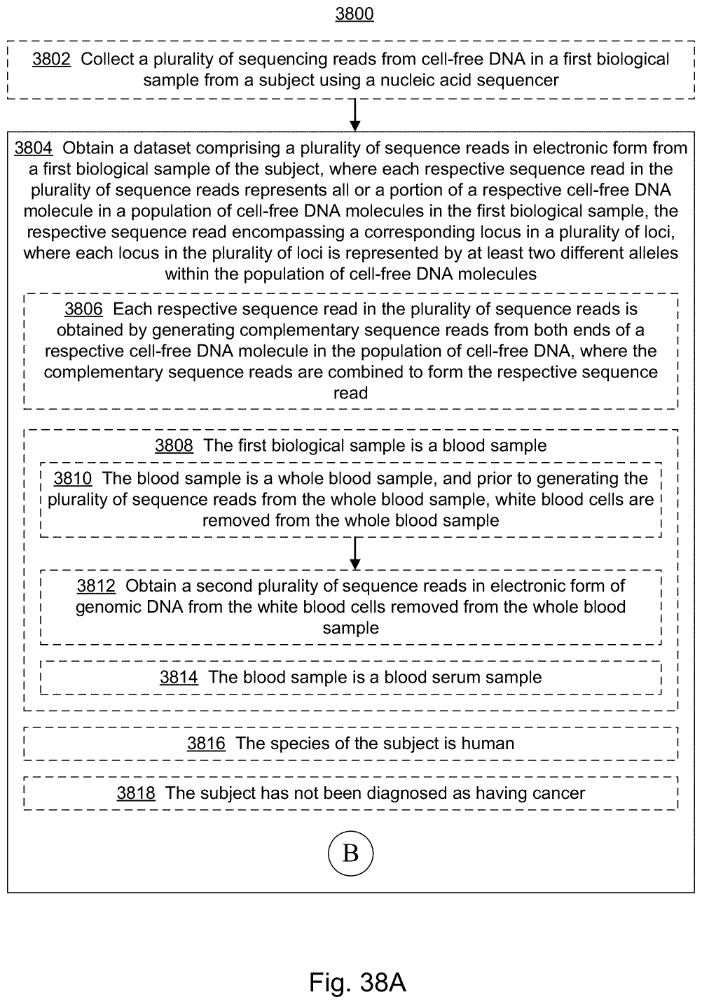

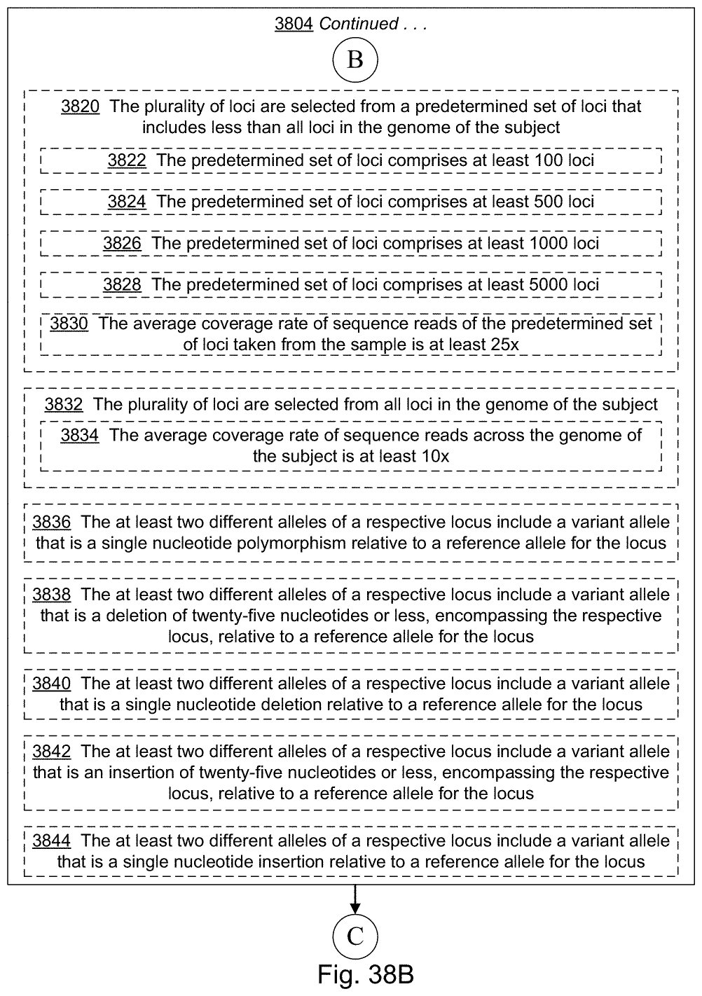

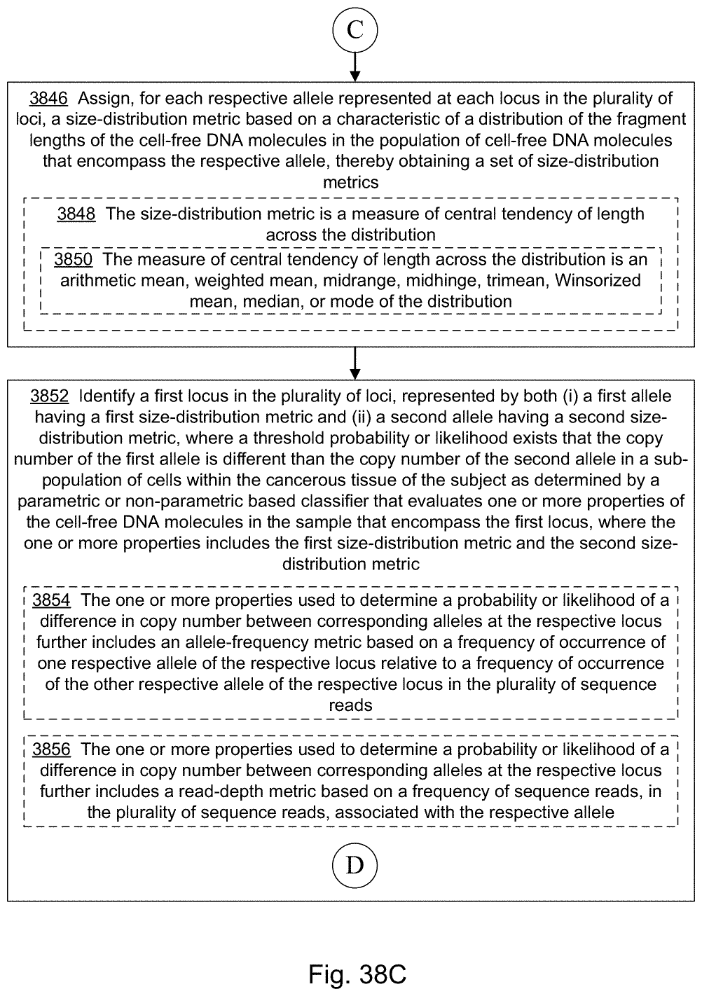

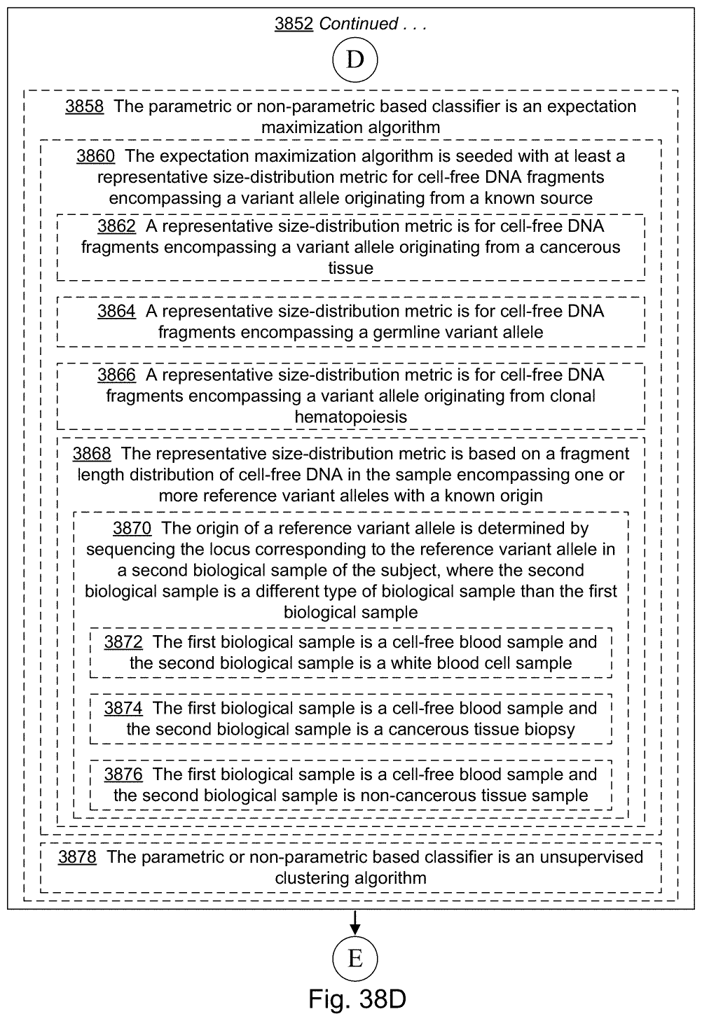

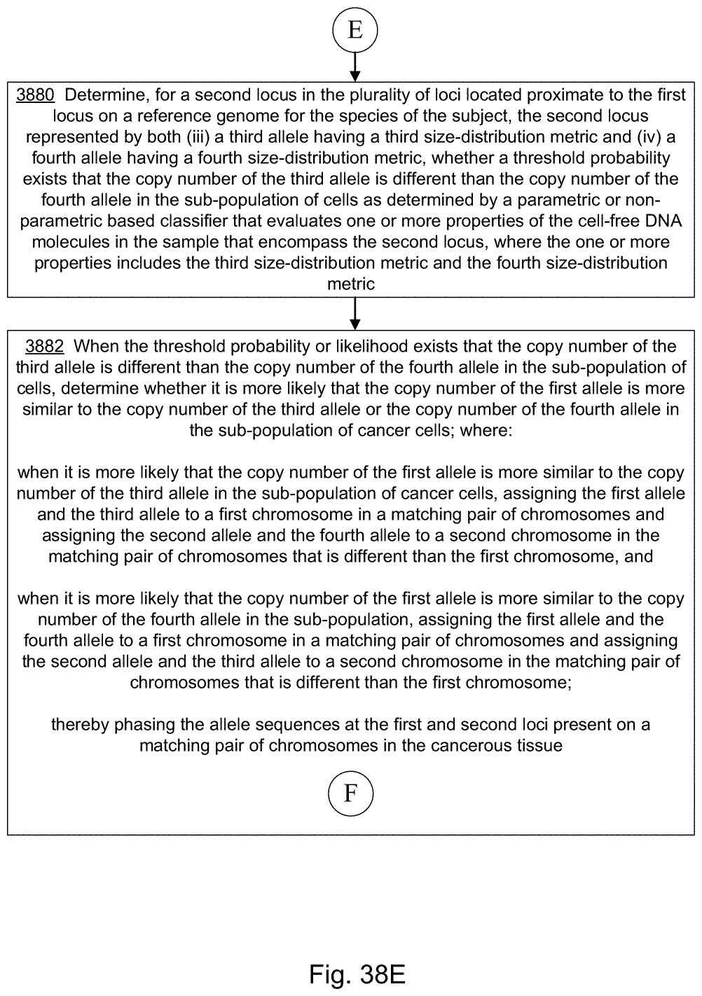

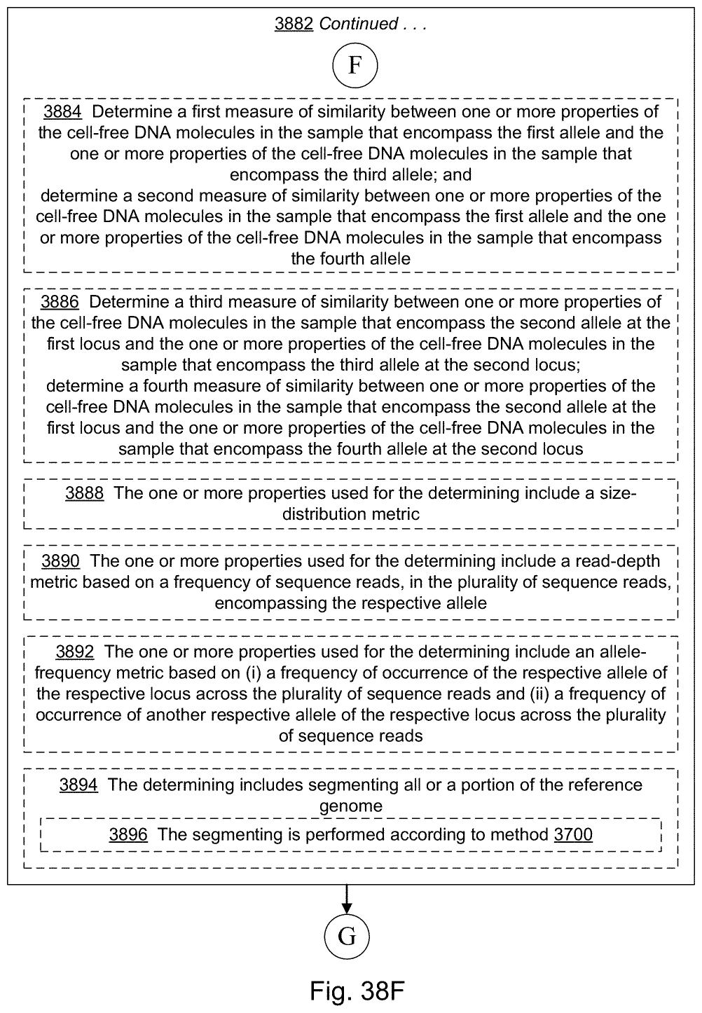

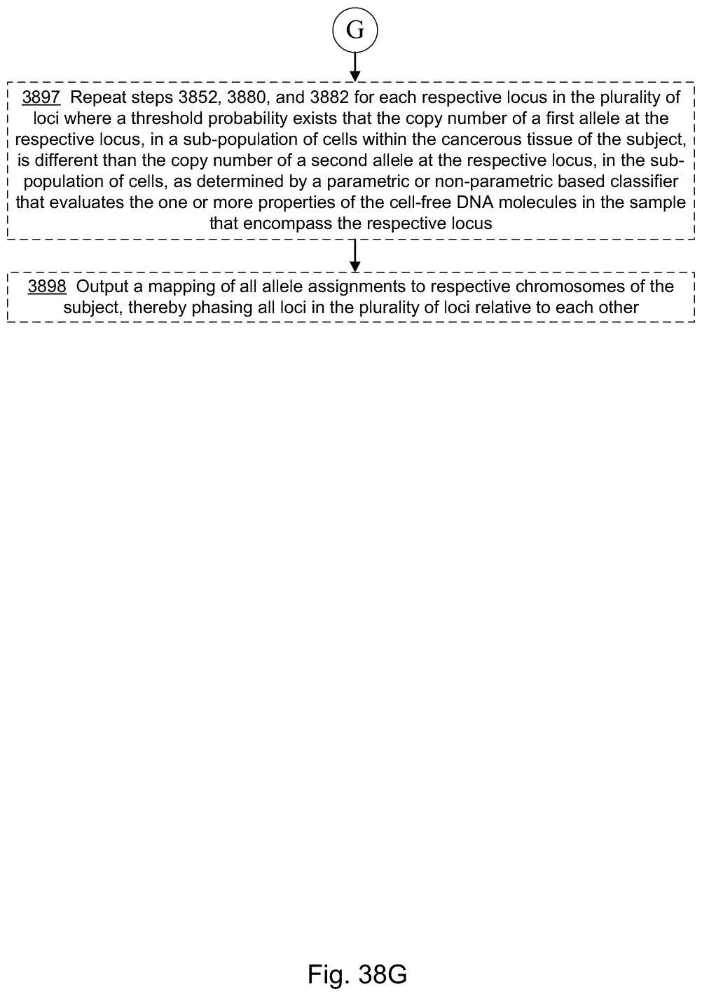

FIGS. 38A-38G are flow diagrams illustrating a method 3800 for phasing alleles present on a matching pair of chromosomes in a cancerous tissue of a subject that is a member of a species using a measure of the distribution of DNA fragment lengths of cell-free DNA fragments isolated from the blood of the subject which encompass an allele of interest. Method 3800 is performed at a computer system (e.g., computer system 100 or 150 in FIG. 1) having one or more processors, and memory storing one or more programs for execution by the one or more processors for phasing alleles present on a matching pair of chromosomes in a cancerous tissue of a subject. Some operations in method 3800 are, optionally, combined and/or the order of some operations is, optionally, changed.

In some embodiments, method 3800 is performed at a computer system comprising one or more processors, and memory storing one or more programs for execution by the one or more processors. The method includes obtaining (3804) a dataset comprising a plurality of nucleic acid fragment sequences in electronic form from a first biological sample of the subject, where each respective nucleic acid fragment sequence in the plurality of nucleic acid fragment sequences represents all or a portion of a respective cell-free DNA molecule in a population of cell-free DNA molecules in the first biological sample, the respective nucleic acid fragment sequence encompassing a corresponding locus in a plurality of loci, where each locus in the plurality of loci is represented by at least two different alleles within the population of cell-free DNA molecules. In some embodiments, the at least two different alleles are two different germline alleles, e.g., two different reference alleles found at the loci of respective maternal and paternal chromosomes within the germline of the subject, or one reference allele and one variant allele found at the loci of respective maternal and paternal chromosomes within the germline of the subject. In some embodiments, the at least two different alleles include a reference or variant allele represented within the germline of the subject and a variant allele arising from a cancerous tissue of the subject, at the respective locus.

For example, as described above, it is known that mono- and di-nucleosomes fragmented from the genomes of non-cancerous somatic cells, hematopoietic cells (e.g., white blood cells), and (when the subject has cancer) cancerous cells. Thus, in some embodiments, the cell-free DNA molecules in the sample originate from at least non-cancerous somatic cells and hematopoietic cells (e.g., white blood cells). In some embodiments, sample also includes cell-free DNA molecules originating from cancerous cells. In some embodiments, it is unknown whether the subject has cancer and, thus, whether cell-free DNA originating from cancerous cells in present is the sample prior to analysis. Accordingly, in some embodiments, the subject has not been diagnosed as having cancer (3818). In some embodiments, the subject has already been diagnosed with cancer and, accordingly, it is known that the cell-free DNA originating from cancerous cells is present in the sample prior to analysis. In some embodiments, the subject is a human (3816).

In some embodiments, the obtaining step of the method includes collecting (3802) the plurality of sequencing reads from the cell-free DNA in the biological sample from the subject using a nucleic acid sequencer. However, in other embodiments, method 3800 only includes obtaining the sequencing data from a prior sequencing reaction of cell-free DNA from a biological sample.

Methods for collecting suitable sequencing data for the methods described herein (e.g., method 3800) are described above, and are not reiterated here for reasons of brevity. Regardless of the exact sequencing method used, however, in some embodiments, each respective nucleic acid fragment sequence in the plurality of nucleic acid fragment sequences is obtained by generating complementary sequence reads from both ends of a respective cell-free DNA molecule in the population of cell-free DNA (3806), where the complementary sequence reads are combined to form a respective sequence read, which is collapsed with other respective sequence reads of the same unique nucleic acid fragment to form the respective nucleic acid fragment sequence. For example, in some embodiments, complementary sequence reads are stitched together based on an overlapping region of sequence shared between the complementary sequence reads and/or by matching the sequences from complementary sequence reads to corresponding sequences in a reference genome for the species of the subject.

In some embodiments, the first biological sample is a blood sample (3808), e.g., a whole-blood sample, a blood serum sample, or a blood plasma sample. In some embodiments, the blood sample is a whole blood sample, and prior to generating the plurality of nucleic acid fragment sequences from the whole blood sample, white blood cells are removed from the whole blood sample (3810). In some embodiments, the white blood cells are collected as a second type of sample, e.g., according to a buffy coat extraction method, from which additional sequencing data may or may not be obtained. In some embodiments, the method further includes obtaining (3812) a second plurality of nucleic acid fragment sequences in electronic form of genomic DNA from the white blood cells removed from the whole blood sample. In some embodiments, the second plurality of nucleic acid fragment sequences is used to identify allele variants arising from clonal hematopoiesis, as opposed to germline allele variants and/or allele variants arising from a cancer in the subject. Likewise, in some embodiments, fragment length distributions obtained for fragments encompassing an allele are used to seed a classification algorithm, e.g., an expectation maximization (EM) algorithm. In some embodiments, the blood sample is a blood serum sample (3814).

In some embodiments, the plurality of loci is selected from a predetermined set of loci that includes less than all loci in the genome of the subject (3820). In some embodiments, nucleic acid fragment sequences of the cell-free DNA molecules in the sample are generated for a predetermined set of loci, e.g., by targeted panel sequencing. As described above, many targeted panels for sequencing alleles of interest, e.g., related to cancer diagnostics, are known to those of skill in the art. Although not reiterated here for reasons of brevity, any of these targeted panels can be used in the methods described herein. In some embodiments, the targeted panel includes loci known to provide diagnostic or prognostic power for cancer diagnostics, e.g., loci at which an allele has been linked to a characteristic of a cancer. In some embodiments, the targeted panel includes alleles that are distributed throughout the genome of the species of the subject, e.g., to provide representation for a large portion of the genome.

In some embodiments, the predetermined set of loci includes at least 100 loci (3822). In some embodiments, the predetermined set of loci includes at least 500 loci (3824). In some embodiments, the predetermined set of loci includes at least 1000 loci (3826). In some embodiments, the predetermined set of loci includes at least 5000 loci (3828). In some embodiments, the predetermined set of loci includes at least 100, 200, 300, 400, 500, 600, 700, 800, 900, 1000, 2000, 3000, 4000, 5000, 6000, 7000, 8000, 9000, 10,000, 15,000, 20,000, 25,000, 50,000, 75,000, 100,000, or more loci. In some embodiments, the predetermined set of loci includes from 100 to 100,000 loci, from 100 to 50,000 loci, from 100 to 25,000 loci, from 100 to 10,000 loci, from 100 to 5000 loci, from 100 to 2000 loci, from 100 to 1000 loci, from 500 to 100,000 loci, from 500 to 50,000 loci, from 500 to 25,000 loci, from 500 to 10,000 loci, from 500 to 5000 loci, from 500 to 2000 loci, from 500 to 1000 loci, from 1000 to 100,000 loci, from 1000 to 50,000 loci, from 1000 to 25,000 loci, from 1000 to 10,000 loci, from 1000 to 5000 loci, or from 1000 to 2000 loci.

In some embodiments, the average coverage rate of nucleic acid fragment sequences of the predetermined set of loci taken from the sample is at least 25× (3830). In some embodiments, the average coverage rate of nucleic acid fragment sequences of the predetermined set of loci taken from the sample is at least 50×, 100×, 200×, 300×, 400×, 500×, 750×, 1000×, 2000×, 3000×, 4000×, 5000×, or more. In some embodiments, the average coverage rate of nucleic acid fragment sequences of the predetermined set of loci taken from the sample is from 25× to 5000×, from 25× to 2500×, from 25× to 1000×, from 25× to 500×, from 25× to 100×, from 100× to 5000×, from 100× to 2500×, from 100× to 1000×, or from 100× to 500×.

In some embodiments, all of the cell-free DNA molecules in the sample are sequenced (3832), e.g., by whole genome sequencing, and nucleic acid fragment sequences corresponding to cell-free DNA molecules encompassing the predetermined set of loci are selected for the analysis. As described above, many methods for whole genome sequencing are known to those of skill in the art. In some embodiments, the average coverage rate of nucleic acid fragment sequences across the genome of the subject is at least 10× (3834). In some embodiments, the average coverage rate of nucleic acid fragment sequences across the genome of the subject is at least 25×, 50×, 100×, 200×, 300×, 400×, 500×, 750×, 1000×, or more. In some embodiments, the average coverage rate of nucleic acid fragment sequences of the predetermined set of loci taken from the sample is from 10× to 1000×, from 10× to 500×, from 10× to 100×, from 10× to 50×, from 50× to 1000×, from 50× to 500×, or from 50× to 100×.

In some embodiments, the at least two different alleles of a respective locus include a reference allele and a variant allele. In some embodiments, the at least two different alleles of a respective locus include a variant allele that is a single nucleotide polymorphism relative to a reference allele for the locus (3836). In some embodiments, the preceding claims, wherein the at least two different alleles of a respective locus include a variant allele that is a deletion of twenty-five nucleotides or less, encompassing the respective locus, relative to a reference allele for the locus (3838). In some embodiments, the at least two different alleles of a respective locus include a variant allele that is a single nucleotide deletion relative to a reference allele for the locus (3840). In some embodiments, the at least two different alleles of a respective locus include a variant allele that is an insertion of twenty-five nucleotides or less, encompassing the respective locus, relative to a reference allele for the locus (3842). In some embodiments, the at least two different alleles of a respective locus include a variant allele that is a single nucleotide insertion relative to a reference allele for the locus (3844).

Method 3800 also includes assigning (3846), for each respective allele represented at each locus in the plurality of loci, a size-distribution metric (e.g., a median length, a median shift in length, a measure of central tendency of length across the distribution, a measure of central tendency of shift in length across the distribution, or a statistical distribution) based on a characteristic of the distribution of the fragment lengths of the cell-free DNA molecules in the population of cell-free DNA molecules (e.g., that are represented by a respective nucleic acid fragment sequence in the plurality of nucleic acid fragment sequences) that encompass the respective allele, thereby obtaining a set of size-distribution metrics. Because the set of size-distribution metrics is smaller than the set of individual nucleic acid fragment sequences, this step compresses the data in order to make the method more computationally efficient, e.g., by allowing the computer to apply an algorithm to the smaller dataset (the set size distribution metrics) rather than the full dataset (the nucleic acid fragment sequences themselves). In one embodiment, the size-distribution metric is a measure of central tendency of length across the distribution (3848). In some embodiments, the measure of central tendency of length across the distribution is an arithmetic mean, weighted mean, midrange, midhinge, trimean, Winsorized mean, median, or mode of the distribution (3850).

Method 3800 also includes identifying (3852) a first locus in the plurality of loci, represented by both (i) a first allele having a first size-distribution metric (e.g., in the set of size-distribution metrics) and (ii) a second allele having a second size-distribution metric (e.g., in the set of size-distribution metrics), where a threshold probability or likelihood exists that the copy number of the first allele is different than the copy number of the second allele in a subpopulation of cells within the cancerous tissue of the subject as determined by a parametric or non-parametric based classifier that evaluates one or more properties of the cell-free DNA molecules in the sample that encompass the first locus. The one or more properties includes the first size-distribution metric and the second size-distribution metric. E.g., the first locus is identified, at least in part, by detecting a characteristic shift in the fragment length shift of cell free DNA molecules encompassing one allele at the locus relative to the fragment length of cell free DNA molecules encompassing the other allele at the locus, representing a likelihood that one of the alleles was lost in at least a first clonal population of cancers cells within the subject.

In some embodiments, the one or more properties used to determine a probability or likelihood of a difference in copy number between corresponding alleles at the respective locus further includes an allele-frequency metric based on a frequency of occurrence of one respective allele of the respective locus (e.g., the first allele at the first locus and/or the third allele at the second locus) relative to a frequency of occurrence of the other respective allele of the respective locus (e.g., the second allele at the first locus and/or the fourth allele at the second locus) in the plurality of nucleic acid fragment sequences (3854).

In some embodiments, the one or more properties used to determine a probability or likelihood of a difference in copy number between corresponding alleles at the respective locus further includes a read-depth metric based on a frequency of nucleic acid fragment sequences, in the plurality of nucleic acid fragment sequences, associated with the respective allele (3856). E.g., a frequency of nucleic acid fragment sequences containing the respective allele or a frequency of nucleic acid fragment sequences that correspond to a same portion of a reference genome (e.g., a bin) for the species of the subject as the locus represented by the respective allele, in a plurality of different and non-overlapping portions of the reference genome.

In some embodiments, the parametric or non-parametric based classifier is an expectation maximization algorithm (3858). In some embodiments, the expectation maximization algorithm is seeded with at least a representative size-distribution or size distribution metric for cell-free DNA fragments encompassing a variant allele originating from a known source (3860). In some embodiments, a representative size-distribution metric is for cell-free DNA fragments encompassing a variant allele originating from a cancerous tissue (3862). In some embodiments, a representative size-distribution metric is for cell-free DNA fragments encompassing a germline variant allele (3864). In some embodiments, a representative size-distribution metric is for cell-free DNA fragments encompassing a variant allele originating from clonal hematopoiesis (3866). In some embodiments, the representative size-distribution metric is based on a fragment length distribution of cell-free DNA in the sample encompassing one or more reference variant alleles with a known origin (3868).

In some embodiments, the origin of a reference variant allele is determined by sequencing the locus corresponding to the reference variant allele in a second biological sample of the subject, where the second biological sample is a different type of biological sample than the first biological sample (3870). In some embodiments, the first biological sample is a cell-free blood sample and the second biological sample is a white blood cell sample (3872). For instance, in some embodiments, a blood sample containing at least blood serum and white blood cells is collected from the subject, the white blood cells are removed from the sample (e.g., via buffy coat extraction), and loci of interest are sequenced in both the cell-free portion and the white blood cell portion of the original sample (e.g., which were separated from each other). Accordingly, variant alleles sequenced in the cell-free portion of the sample, which do not originate from the germline of the subject and which match variant alleles sequenced in the white blood cell sample can be positively identified as originating from clonal hematopoiesis, and can be used to seed the expectation maximization algorithm. In some embodiments, the first biological sample is a cell-free blood sample and the second biological sample is a cancerous tissue biopsy (3874). For instance, in some embodiments, a blood sample and a tumor biopsy are collected from the subject, and loci of interest are sequenced from both samples. Accordingly, variant alleles sequenced in the cell-free portion of the sample, which do not originate from the germline of the subject and which match variant alleles sequenced in the tumor biopsy can be positively identified as originating from cancerous tissue in the subject, and can be used to seed the expectation maximization algorithm. In some embodiments, the first biological sample is a cell-free blood sample and the second biological sample is non-cancerous tissue sample (3876). For instance, in some embodiments, a blood sample and a non-cancerous tissue sample are collected from the subject, and loci of interest are sequenced from both samples. Accordingly, variant alleles sequenced in the cell-free portion of the sample, which match variant alleles sequenced in the non-cancerous tissue sample can be positively identified as originating from the germline of the subject, and can be used to seed the expectation maximization algorithm.

In some embodiments, the parametric or non-parametric based classifier is an unsupervised clustering algorithm (3878). For example, as illustrated in FIG. 11, when the allele frequency of a germline variant allele in cell-free DNA is plotted as a function of the mean shift in fragment-length of cell-free DNA fragments encompassing the variant allele, relative to the mean fragment-length of cell-free DNA fragments encompassing the corresponding reference allele, the alleles appear to cluster into five distinct groups, likely corresponding to loci at which cancer cells have lost a chromosomal copy of the variant allele (1102), loci at which cancer cells have gained a copy of the reference allele (1104), loci at which cancer cells have not gained or lost a copy of either allele (1106), loci at which cancer cells have gained a copy of the variant allele (1108), and loci at which cancer cells have lost a copy of the reference allele (1110). Accordingly, in some embodiments, a clustering algorithm (e.g., supervised or unsupervised) is used to identify chromosomal copy number aberrations based on identification of the alleles and loci in each cluster. Thus, alleles that are located near each other on the same chromosome, and which are clustered into the same group, are likely phased together on either the maternal chromosome or the paternal chromosome in the subject.

Method 3800 also includes determining (3880), for a second locus in the plurality of loci located proximate to the first locus on a reference genome for the species of the subject, the second locus represented by both (iii) a third allele having a third size-distribution metric (e.g., in the set of size-distribution metrics) and (iv) a fourth allele having a fourth size-distribution metric (e.g., in the set of size-distribution metrics), whether a threshold probability exists that the copy number of the third allele is different than the copy number of the fourth allele in the sub-population of cells as determined by a parametric or non-parametric based classifier that evaluates one or more properties of the cell-free DNA molecules in the sample that encompass the second locus. The one or more properties includes the third size-distribution metric and the fourth size-distribution metric. E.g., determining whether there is a likelihood that one of the alleles at the second locus was also lost in at least a first clonal population of cancers cells within the subject is done, at least in part, by detecting a characteristic shift in the fragment length shift of cell free DNA molecules encompassing one allele at the second locus relative to the fragment length of cell free DNA molecules encompassing the other allele at the second locus.

When the threshold probability or likelihood exists that the copy number of the third allele is different than the copy number of the fourth allele in the sub-population of cells, method 3800 includes determining (3882) whether it is more likely that the copy number of the first allele is more similar to the copy number of the third allele or the copy number of the fourth allele in the sub-population of cancer cells (e.g., by determining which of the third size-distribution metric and the fourth size-distribution metric most closely matches the first size-distribution metric, e.g., by comparing the first size-distribution metric to the third size-distribution metric and further comparing the first size-distribution metric to the fourth size-distribution metric). When it is more likely that the copy number of the first allele is more similar to the copy number of the third allele in the subpopulation of cancer cells, method 3800 includes assigning the first allele and the third allele to a first chromosome in a matching pair of chromosomes and assigning the second allele and the fourth allele to a second chromosome in the matching pair of chromosomes that is different than the first chromosome. When it is more likely that the copy number of the first allele is more similar to the copy number of the fourth allele in the sub-population, method 3800 includes assigning the first allele and the fourth allele to a first chromosome in a matching pair of chromosomes and assigning the second allele and the third allele to a second chromosome in the matching pair of chromosomes that is different than the first chromosome. Accordingly, the allele sequences at the first and second loci present on a matching pair of chromosomes in the cancerous tissue are phased relative to each other.

In some embodiments, determining (3882) whether it is more likely that the copy number of the first allele is more similar to the copy number of the third allele or the copy number of the fourth allele in the sub-population of cancer cells includes determining (3884) a first measure of similarity between one or more properties of the cell-free DNA molecules in the sample that encompass the first allele and the one or more properties of the cell-free DNA molecules in the sample that encompass the third allele, and determining a second measure of similarity between one or more properties of the cell-free DNA molecules in the sample that encompass the first allele and the one or more properties of the cell-free DNA molecules in the sample that encompass the fourth allele, e.g., and determining which of the measures of similarity is greater.

In some embodiments, determining (3882) whether it is more likely that the copy number of the first allele is more similar to the copy number of the third allele or the copy number of the fourth allele in the sub-population of cancer cells includes determining (3886) a third measure of similarity between one or more properties of the cell-free DNA molecules in the sample that encompass the second allele at the first locus and the one or more properties of the cell-free DNA molecules in the sample that encompass the third allele at the second locus, and determining a fourth measure of similarity between one or more properties of the cell-free DNA molecules in the sample that encompass the second allele at the first locus and the one or more properties of the cell-free DNA molecules in the sample that encompass the fourth allele at the second locus, e.g., and determining which of the measures of similarity is greater.

In some embodiments, the one or more properties used for the determining (3882) include a size-distribution metric (3888), e.g., a median length, a median shift in length, a measure of central tendency of length across the distribution, a measure of central tendency of shift in length across the distribution, or a statistical distribution. In some embodiments, the one or more properties used for the determining (3882) include a read-depth metric based on a frequency of nucleic acid fragment sequences, in the plurality of nucleic acid fragment sequences, encompassing the respective allele (3890). In some embodiments, the one or more properties used for the determining (3882) include an allele-frequency metric based on (i) a frequency of occurrence of the respective allele of the respective locus across the plurality of nucleic acid fragment sequences and (ii) a frequency of occurrence of another respective allele of the respective locus across the plurality of nucleic acid fragment sequences (3892).

In some embodiments, the determining (3882) includes segmenting all or a portion of the reference genome (3894). In some embodiments, the segmenting is performed according to method 3700 (3896).

In some embodiments, method 3800 includes repeating (3897) steps 3852, 3880, and 3882 for respective loci (e.g., all or some of the loci) in the plurality of loci where a threshold probability exists that the copy number of a first allele at the respective locus, in a sub-population of cells within the cancerous tissue of the subject, is different than the copy number of a second allele at the respective locus, in the sub-population of cells, as determined by a parametric or non-parametric based classifier that evaluates the one or more properties of the cell-free DNA molecules in the sample that encompass the respective locus.

In some embodiments, method 3800 includes outputting (3898) (e.g., writing to a file) a mapping of all allele assignments to respective chromosomes of the subject, thereby phasing all loci in the plurality of loci relative to each other. In some embodiments, this output is useful for a precision medicine approach for treating a disorder (e.g., cancer) in the subject.

It should be understood that the particular order in which the operations in FIGS. 38A-38G have been described is merely an example and is not intended to indicate that the described order is the only order in which the operations could be performed. One of ordinary skill in the art would recognize various ways to reorder the operations described herein. Additionally, it should be noted that details of other processes described herein with respect to other methods described herein (e.g., methods 3700, 3900, 4000, 4100, and 4200) are also applicable in an analogous manner to method 3800 described above with respect to FIGS. 38A-38G. Further, in some embodiments, method 3800 can be used in conjunction with any other method described herein (e.g., methods 3700, 3900, 4000, 4100, and 4200). The operations in the information processing methods described above are, optionally implemented by running one or more functional modules in information processing apparatus such as general purpose processors (e.g., as described above with respect to FIGS. 1A and 1B) or application specific chips.

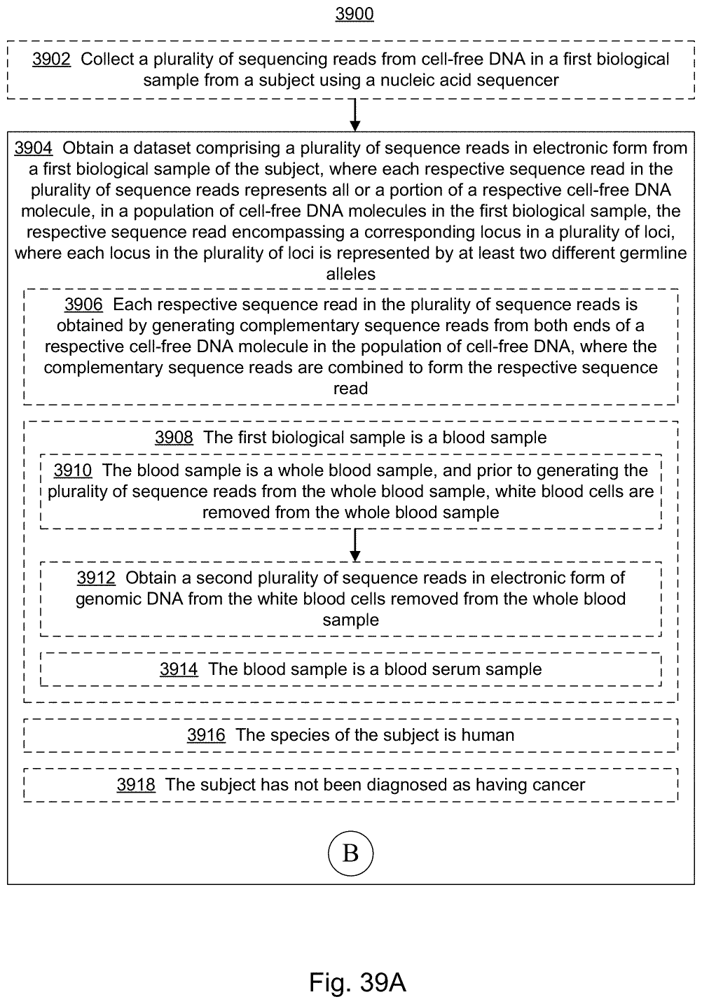

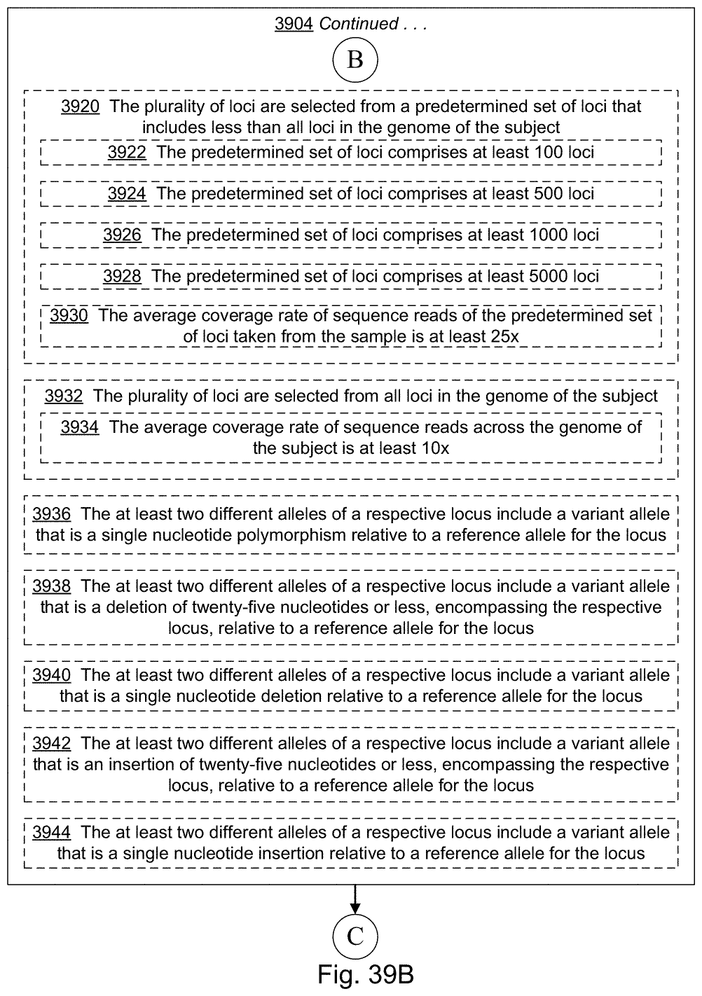

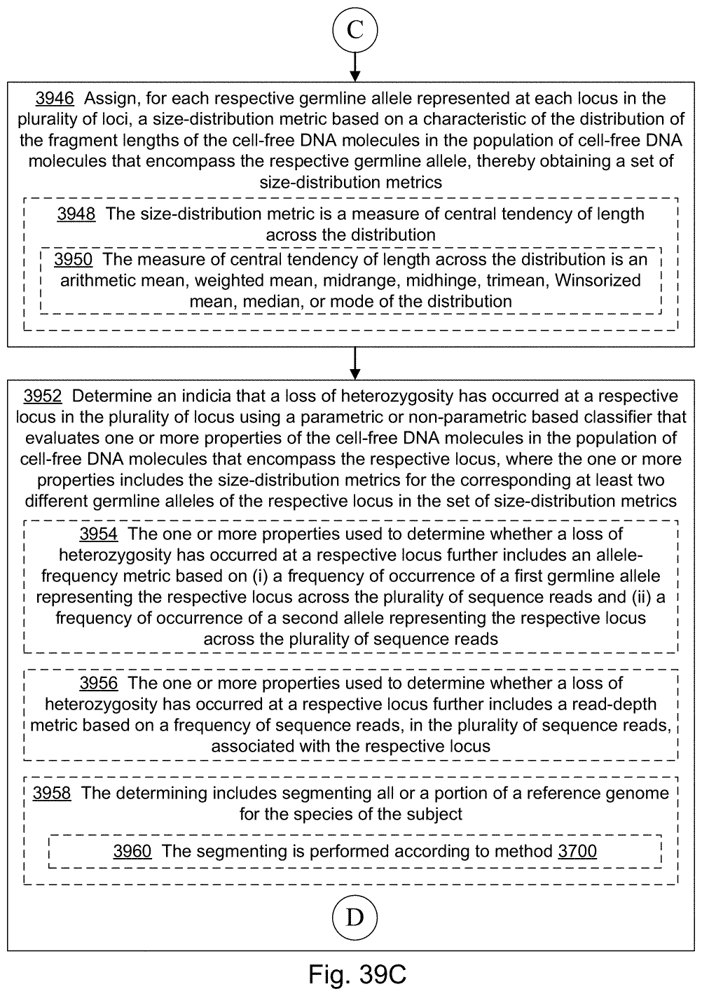

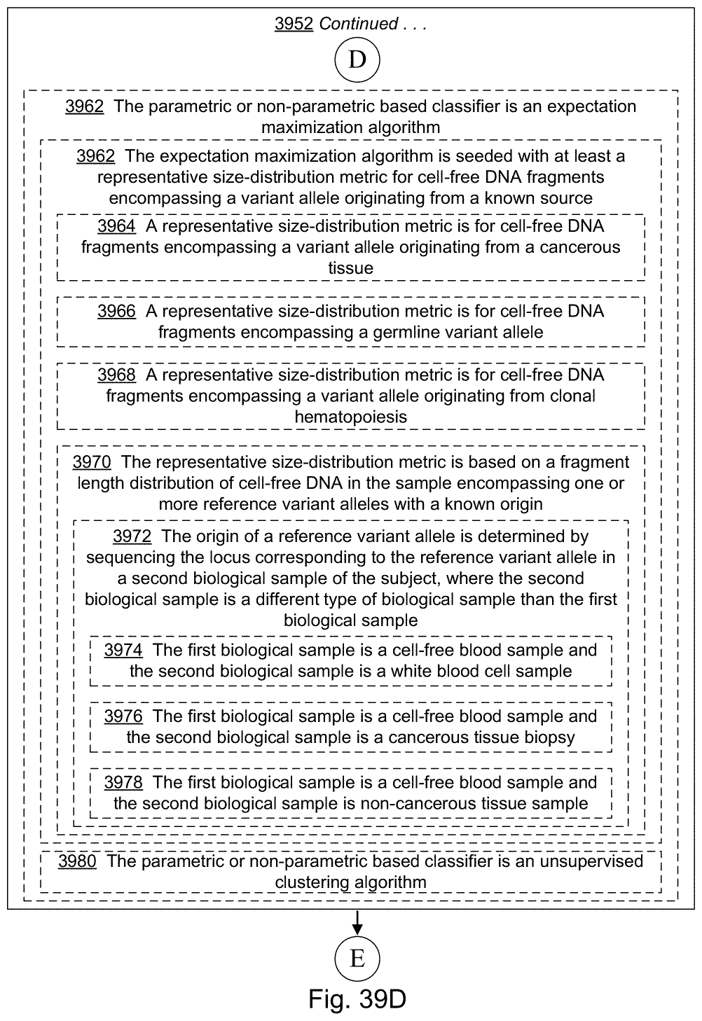

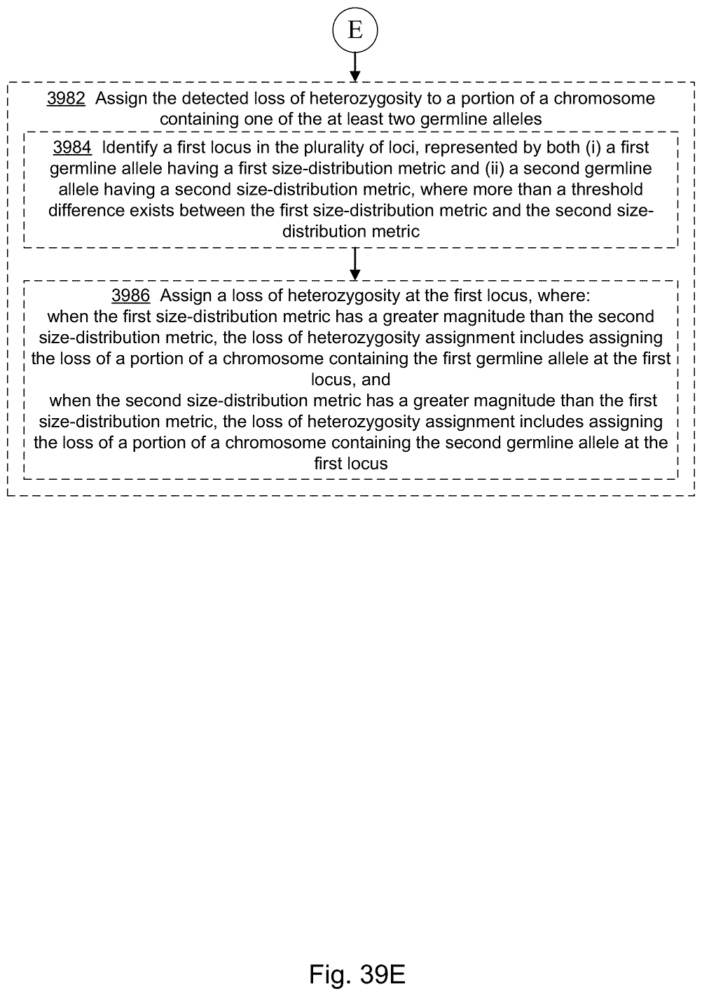

FIGS. 39A-38E are flow diagrams illustrating a method 3900 for detecting a loss in heterozygosity at a genomic locus in a cancerous tissue of a subject using a measure of the distribution of DNA fragment lengths of cell-free DNA fragments isolated from the blood of the subject which encompass an allele of interest. Method 3900 is performed at a computer system (e.g., computer system 100 or 150 in FIG. 1) having one or more processors, and memory storing one or more programs for execution by the one or more processors for phasing alleles present on a matching pair of chromosomes in a cancerous tissue of a subject. Some operations in method 3900 are, optionally, combined and/or the order of some operations is, optionally, changed.

In some embodiments, method 3900 is performed at a computer system comprising one or more processors, and memory storing one or more programs for execution by the one or more processors. The method includes obtaining (3904) a dataset comprising a plurality of nucleic acid fragment sequences in electronic form from a first biological sample of the subject, where each respective nucleic acid fragment sequence in the plurality of nucleic acid fragment sequences represents all or a portion of a respective cell-free DNA molecule in a population of cell-free DNA molecules in the first biological sample, the respective nucleic acid fragment sequence encompassing a corresponding locus in a plurality of loci, wherein each locus in the plurality of loci is represented by at least two different germline alleles within the population of cell-free DNA molecules, e.g., two different reference alleles found at the loci of respective maternal and paternal chromosomes within the germline of the subject, or one reference allele and one variant allele found at the loci of respective maternal and paternal chromosomes within the germline of the subject.

For example, as described above, it is known that mono- and di-nucleosomes fragmented from the genomes of non-cancerous somatic cells, hematopoietic cells (e.g., white blood cells), and (when the subject has cancer) cancerous cells. Thus, in some embodiments, the cell-free DNA molecules in the sample originate from at least non-cancerous somatic cells and hematopoietic cells (e.g., white blood cells). In some embodiments, sample also includes cell-free DNA molecules originating from cancerous cells. In some embodiments, it is unknown whether the subject has cancer and, thus, whether cell-free DNA originating from cancerous cells in present in the sample prior to analysis. Accordingly, in some embodiments, the subject has not been diagnosed as having cancer (3918). In some embodiments, the subject has already been diagnosed with cancer and, accordingly, it is known that the cell-free DNA originating from cancerous cells is present in the sample prior to analysis. In some embodiments, the subject is a human (3916).

In some embodiments, the obtaining step of the method includes collecting (3902) the plurality of sequencing reads from the cell-free DNA in the biological sample from the subject using a nucleic acid sequencer. However, in other embodiments, method 3900 only includes obtaining the sequencing data from a prior sequencing reaction of cell-free DNA from a biological sample.

Methods for collecting suitable sequencing data for the methods described herein (e.g., method 3900) are described above, and are not reiterated here for reasons of brevity. Regardless of the exact sequencing method used, however, in some embodiments, each respective nucleic acid fragment sequence in the plurality of nucleic acid fragment sequences is obtained by generating complementary sequence reads from both ends of a respective cell-free DNA molecule in the population of cell-free DNA (3906), where the complementary sequence reads are combined to form a respective sequence read, which is collapsed with other respective sequence reads of the same unique nucleic acid fragment to form the respective nucleic acid fragment sequence. For example, in some embodiments, complementary sequence reads are stitched together based on an overlapping region of sequence shared between the complementary sequence reads and/or by matching the sequences from complementary sequence reads to corresponding sequences in a reference genome for the species of the subject.

In some embodiments, the first biological sample is a blood sample (3908), e.g., a whole-blood sample, a blood serum sample, or a blood plasma sample. In some embodiments, the blood sample is a whole blood sample, and prior to generating the plurality of nucleic acid fragment sequences from the whole blood sample, white blood cells are removed from the whole blood sample (3910). In some embodiments, the white blood cells are collected as a second type of sample, e.g., according to a buffy coat extraction method, from which additional sequencing data may or may not be obtained. In some embodiments, the method further includes obtaining (3912) a second plurality of nucleic acid fragment sequences in electronic form of genomic DNA from the white blood cells removed from the whole blood sample. In some embodiments, the second plurality of nucleic acid fragment sequences is used to identify allele variants arising from clonal hematopoiesis, as opposed to germline allele variants and/or allele variants arising from a cancer in the subject. Likewise, in some embodiments, fragment length distributions obtained for fragments encompassing an allele are used to seed a classification algorithm, e.g., an expectation maximization (EM) algorithm. In some embodiments, the blood sample is a blood serum sample (3914).

In some embodiments, the plurality of loci are selected from a predetermined set of loci that includes less than all loci in the genome of the subject (3920). In some embodiments, nucleic acid fragment sequences of the cell-free DNA molecules in the sample are generated for a predetermined set of loci, e.g., by targeted panel sequencing. As described above, many targeted panels for sequencing alleles of interest, e.g., related to cancer diagnostics, are known to those of skill in the art. Although not reiterated here for reasons of brevity, any of these targeted panels can be used in the methods described herein. In some embodiments, the targeted panel includes loci known to provide diagnostic or prognostic power for cancer diagnostics, e.g., loci at which an allele has been linked to a characteristic of a cancer. In some embodiments, the targeted panel includes alleles that are distributed throughout the genome of the species of the subject, e.g., to provide representation for a large portion of the genome.

In some embodiments, the predetermined set of loci includes at least 100 loci (3922). In some embodiments, the predetermined set of loci includes at least 500 loci (3924). In some embodiments, the predetermined set of loci includes at least 1000 loci (3926). In some embodiments, the predetermined set of loci includes at least 5000 loci (3928). In some embodiments, the predetermined set of loci includes at least 100, 200, 300, 400, 500, 600, 700, 800, 900, 1000, 2000, 3000, 4000, 5000, 6000, 7000, 8000, 9000, 10,000, 15,000, 20,000, 25,000, 50,000, 75,000, 100,000, or more loci. In some embodiments, the predetermined set of loci includes from 100 to 100,000 loci, from 100 to 50,000 loci, from 100 to 25,000 loci, from 100 to 10,000 loci, from 100 to 5000 loci, from 100 to 2000 loci, from 100 to 1000 loci, from 500 to 100,000 loci, from 500 to 50,000 loci, from 500 to 25,000 loci, from 500 to 10,000 loci, from 500 to 5000 loci, from 500 to 2000 loci, from 500 to 1000 loci, from 1000 to 100,000 loci, from 1000 to 50,000 loci, from 1000 to 25,000 loci, from 1000 to 10,000 loci, from 1000 to 5000 loci, or from 1000 to 2000 loci.

In some embodiments, the average coverage rate of nucleic acid fragment sequences of the predetermined set of loci taken from the sample is at least 25× (3930). In some embodiments, the average coverage rate of nucleic acid fragment sequences of the predetermined set of loci taken from the sample is at least 50×, 100×, 200×, 300×, 400×, 500×, 750×, 1000×, 2000×, 3000×, 4000×, 5000×, or more. In some embodiments, the average coverage rate of nucleic acid fragment sequences of the predetermined set of loci taken from the sample is from 25× to 5000×, from 25× to 2500×, from 25× to 1000×, from 25× to 500×, from 25× to 100×, from 100× to 5000×, from 100× to 2500×, from 100× to 1000×, or from 100× to 500×.

In some embodiments, all of the cell-free DNA molecules in the sample are sequenced (3932), e.g., by whole genome sequencing, and nucleic acid fragment sequences corresponding to cell-free DNA molecules encompassing the predetermined set of loci are selected for the analysis. As described above, many methods for whole genome sequencing are known to those of skill in the art. In some embodiments, the average coverage rate of nucleic acid fragment sequences across the genome of the subject is at least 10× (3934). In some embodiments, the average coverage rate of nucleic acid fragment sequences across the genome of the subject is at least 25×, 50×, 100×, 200×, 300×, 400×, 500×, 750×, 1000×, or more. In some embodiments, the average coverage rate of nucleic acid fragment sequences of the predetermined set of loci taken from the sample is from 10× to 1000×, from 10× to 500×, from 10× to 100×, from 10× to 50×, from 50× to 1000×, from 50× to 500×, or from 50× to 100×.

In some embodiments, the at least two different alleles of a respective locus include a reference allele and a variant allele. In some embodiments, the at least two different alleles of a respective locus include a variant allele that is a single nucleotide polymorphism relative to a reference allele for the locus (3936). In some embodiments, the preceding claims, wherein the at least two different alleles of a respective locus include a variant allele that is a deletion of twenty-five nucleotides or less, encompassing the respective locus, relative to a reference allele for the locus (3938). In some embodiments, the at least two different alleles of a respective locus include a variant allele that is a single nucleotide deletion relative to a reference allele for the locus (3940). In some embodiments, the at least two different alleles of a respective locus include a variant allele that is an insertion of twenty-five nucleotides or less, encompassing the respective locus, relative to a reference allele for the locus (3942). In some embodiments, the at least two different alleles of a respective locus include a variant allele that is a single nucleotide insertion relative to a reference allele for the locus (3944).

Method 3900 also includes assigning (3946), for each respective germline allele represented at each locus in the plurality of loci, a size-distribution metric (e.g., a median length, a median shift in length, a measure of central tendency of length across the distribution, a measure of central tendency of shift in length across the distribution, or a statistical distribution) based on a characteristic of the distribution of the fragment lengths of the cell-free DNA molecules in the population of cell-free DNA molecules (e.g., that are represented by a respective nucleic acid fragment sequence in the plurality of nucleic acid fragment sequences) that encompass the respective germline allele, thereby obtaining a set of size-distribution metrics. Because the set of size-distribution metrics is smaller than the set of individual nucleic acid fragment sequences, this step compresses the data in order to make the method more computationally efficient, e.g., by allowing the computer to apply an algorithm to the smaller dataset (the set size distribution metrics) rather than the full dataset (the nucleic acid fragment sequences themselves). In one embodiment, the size-distribution metric is a measure of central tendency of length across the distribution (3948). In some embodiments, the measure of central tendency of length across the distribution is an arithmetic mean, weighted mean, midrange, midhinge, trimean, Winsorized mean, median, or mode of the distribution (3950).

Method 3900 also includes determining (3952) an indicia that a loss of heterozygosity has occurred at a respective locus in the plurality of locus using a parametric or non-parametric based classifier that evaluates one or more properties of the cell-free DNA molecules in the population of cell-free DNA molecules (e.g., that are represented by a respective nucleic acid fragment sequence in the plurality of nucleic acid fragment sequences) that encompass the respective locus, where the one or more properties includes the size-distribution metrics for the corresponding at least two different germline alleles of the respective locus in the set of size-distribution metrics. E.g., the loss of heterozygosity is identified for an allele, at least in part, by detecting a characteristic shift in the fragment length shift of cell free DNA molecules encompassing the allele at a locus relative to the fragment length of cell free DNA molecules encompassing another allele at the locus, representing a likelihood that the allele was lost in at least a first clonal population of cancers cells within the subject.

In some embodiments, the one or more properties used to determine whether a loss of heterozygosity has occurred at a respective locus further includes an allele-frequency metric based on (i) a frequency of occurrence of a first germline allele representing the respective locus across the plurality of nucleic acid fragment sequences and (ii) a frequency of occurrence of a second allele representing the respective locus across the plurality of nucleic acid fragment sequences (3954).

In some embodiments, the one or more properties used to determine whether a loss of heterozygosity has occurred at a respective locus further includes (3956) a read-depth metric based on a frequency of nucleic acid fragment sequences, in the plurality of nucleic acid fragment sequences, associated with the respective locus, e.g., a frequency of nucleic acid fragment sequences containing the respective locus or a frequency of nucleic acid fragment sequences that correspond to a same portion of a reference genome (e.g., a bin) for the species of the subject as the respective locus, in a plurality of different and non-overlapping portions of the reference genome.

In some embodiments, the determining (3952) includes segmenting all or a portion of the reference genome (3958). In some embodiments, the segmenting is performed according to method 3700 (3960).

In some embodiments, the parametric or non-parametric based classifier is an expectation maximization algorithm (3962). In some embodiments, the expectation maximization algorithm is seeded with at least a representative size-distribution or size distribution metric for cell-free DNA fragments encompassing a variant allele originating from a known source (3962). In some embodiments, a representative size-distribution metric is for cell-free DNA fragments encompassing a variant allele originating from a cancerous tissue (3964). In some embodiments, a representative size-distribution metric is for cell-free DNA fragments encompassing a germline variant allele (3966). In some embodiments, a representative size-distribution metric is for cell-free DNA fragments encompassing a variant allele originating from clonal hematopoiesis (3968). In some embodiments, the representative size-distribution metric is based on a fragment length distribution of cell-free DNA in the sample encompassing one or more reference variant alleles with a known origin (3970).

In some embodiments, the origin of a reference variant allele is determined by sequencing the locus corresponding to the reference variant allele in a second biological sample of the subject, where the second biological sample is a different type of biological sample than the first biological sample (3972). In some embodiments, the first biological sample is a cell-free blood sample and the second biological sample is a white blood cell sample (3974). For instance, in some embodiments, a blood sample containing at least blood serum and white blood cells is collected from the subject, the white blood cells are removed from the sample (e.g., via buffy coat extraction), and loci of interest are sequenced in both the cell-free portion and the white blood cell portion of the original sample (e.g., which were separated from each other). Accordingly, variant alleles sequenced in the cell-free portion of the sample, which do not originate from the germline of the subject and which match variant alleles sequenced in the white blood cell sample can be positively identified as originating from clonal hematopoiesis, and can be used to seed the expectation maximization algorithm. In some embodiments, the first biological sample is a cell-free blood sample and the second biological sample is a cancerous tissue biopsy (3976). For instance, in some embodiments, a blood sample and a tumor biopsy are collected from the subject, and loci of interest are sequenced from both samples. Accordingly, variant alleles sequenced in the cell-free portion of the sample, which do not originate from the germline of the subject and which match variant alleles sequenced in the tumor biopsy can be positively identified as originating from cancerous tissue in the subject, and can be used to seed the expectation maximization algorithm. In some embodiments, the first biological sample is a cell-free blood sample and the second biological sample is non-cancerous tissue sample (3978). For instance, in some embodiments, a blood sample and a non-cancerous tissue sample are collected from the subject, and loci of interest are sequenced from both samples. Accordingly, variant alleles sequenced in the cell-free portion of the sample, which match variant alleles sequenced in the non-cancerous tissue sample can be positively identified as originating from the germline of the subject, and can be used to seed the expectation maximization algorithm.

In some embodiments, the parametric or non-parametric based classifier is an unsupervised clustering algorithm (3980). For example, as illustrated in FIG. 11, when the allele frequency of a germline variant allele in cell-free DNA is plotted as a function of the mean shift in fragment-length of cell-free DNA fragments encompassing the variant allele, relative to the mean fragment-length of cell-free DNA fragments encompassing the corresponding reference allele, the alleles appear to cluster into five distinct groups, likely corresponding to loci at which cancer cells have lost a chromosomal copy of the variant allele (1102), loci at which cancer cells have gained a copy of the reference allele (1104), loci at which cancer cells have not gained or lost a copy of either allele (1106), loci at which cancer cells have gained a copy of the variant allele (1108), and loci at which cancer cells have lost a copy of the reference allele (1110). Accordingly, in some embodiments, a clustering algorithm (e.g., supervised or unsupervised) is used to identify chromosomal copy number aberrations based on identification of the alleles and loci in each cluster. Thus, loci that are clustered into a group representative of a loss of either the germline variant allele (1102) or the reference allele (1110) indicate instances where the cancer has lost heterozygosity.

In some embodiments, method 3900 includes assigning (3982) the detected loss of heterozygosity to a portion of a chromosome containing one of the at least two germline alleles. In some embodiments, the assigning includes identifying (3984) a first locus in the plurality of loci, represented by both (i) a first germline allele having a first size-distribution metric (in the set of size-distribution metrics) and (ii) a second germline allele having a second size-distribution metric (in the set of size-distribution metrics), wherein more than a threshold difference exists between the first size-distribution metric and the second size-distribution metric. In some embodiments, the method then includes assigning (3986) a loss of heterozygosity at the first locus, where: when the first size-distribution metric has a greater magnitude than the second size-distribution metric (e.g., where comparison of the first size-distribution metric and the second size-distribution metric indicates that, on average, nucleic acids encompassing the first allele are longer than nucleic acids encompassing the second allele in the population of cell-free nucleic acids), the loss of heterozygosity assignment includes assigning the loss of a portion of a chromosome containing the first germline allele at the first locus, and when the second size-distribution metric has a greater magnitude than the first size-distribution metric (e.g., where comparison of the first size-distribution metric and the second size-distribution metric indicates that, on average, nucleic acids encompassing the second allele are longer than nucleic acids encompassing the first allele in the population of cell-free nucleic acids), the loss of heterozygosity assignment includes assigning the loss of a portion of a chromosome containing the second germline allele at the first locus.

It should be understood that the particular order in which the operations in FIGS. 39A-39E have been described is merely an example and is not intended to indicate that the described order is the only order in which the operations could be performed. One of ordinary skill in the art would recognize various ways to reorder the operations described herein. Additionally, it should be noted that details of other processes described herein with respect to other methods described herein (e.g., methods 3700, 3800, 4000, 4100, and 4200) are also applicable in an analogous manner to method 3900 described above with respect to FIGS. 39A-39E. Further, in some embodiments, method 3900 can be used in conjunction with any other method described herein (e.g., methods 3700, 3800, 4000, 4100, and 4200). The operations in the information processing methods described above are, optionally implemented by running one or more functional modules in information processing apparatus such as general purpose processors (e.g., as described above with respect to FIGS. 1A and 1B) or application specific chips.

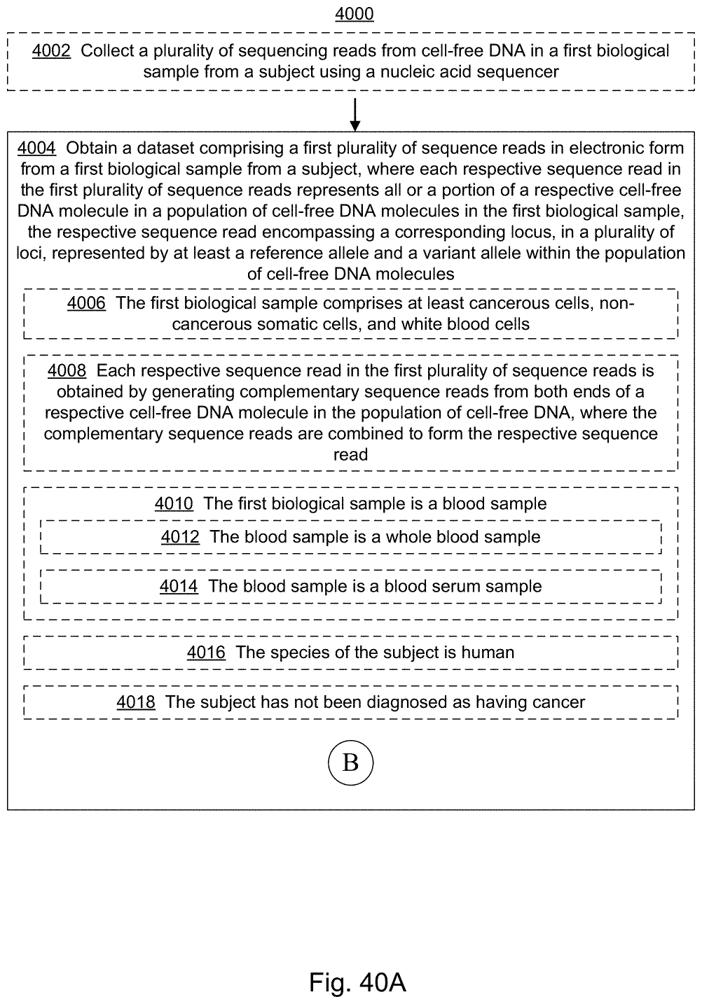

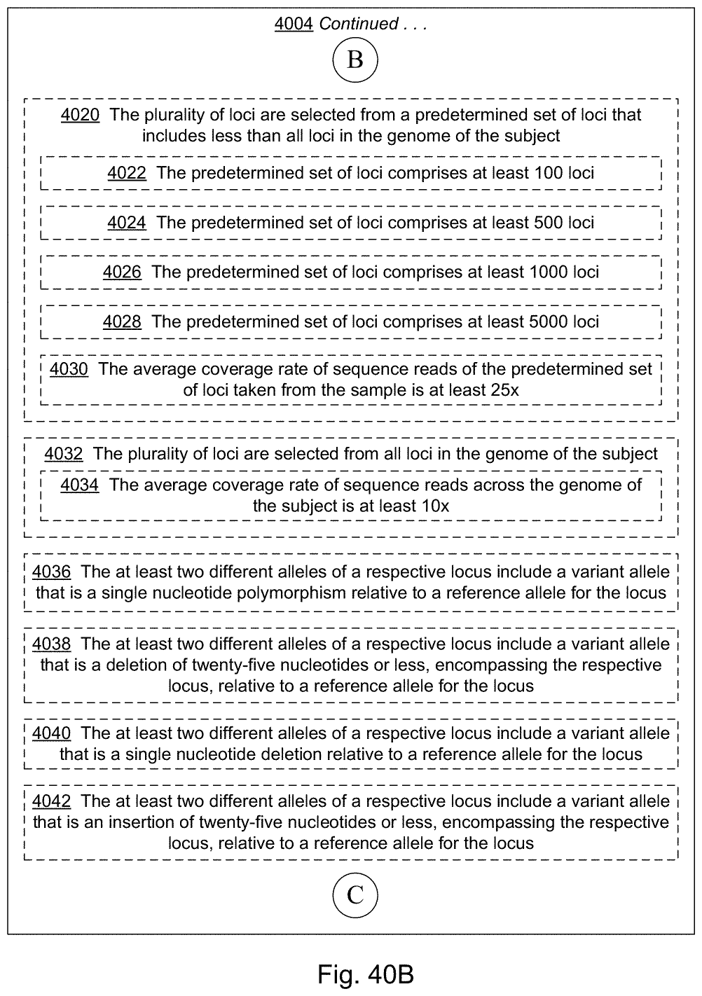

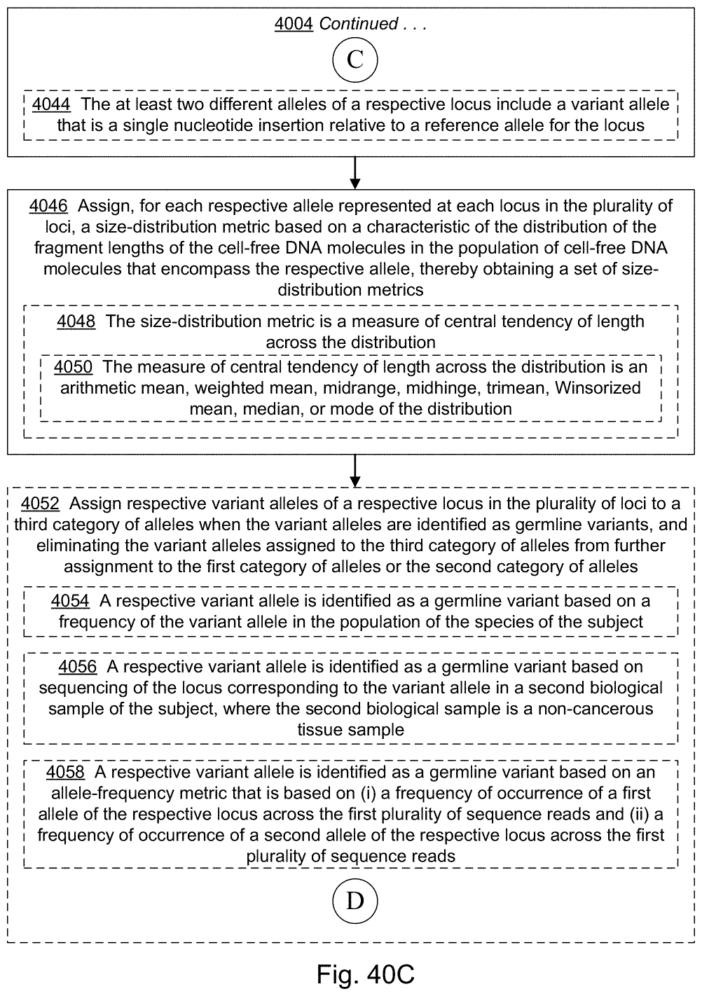

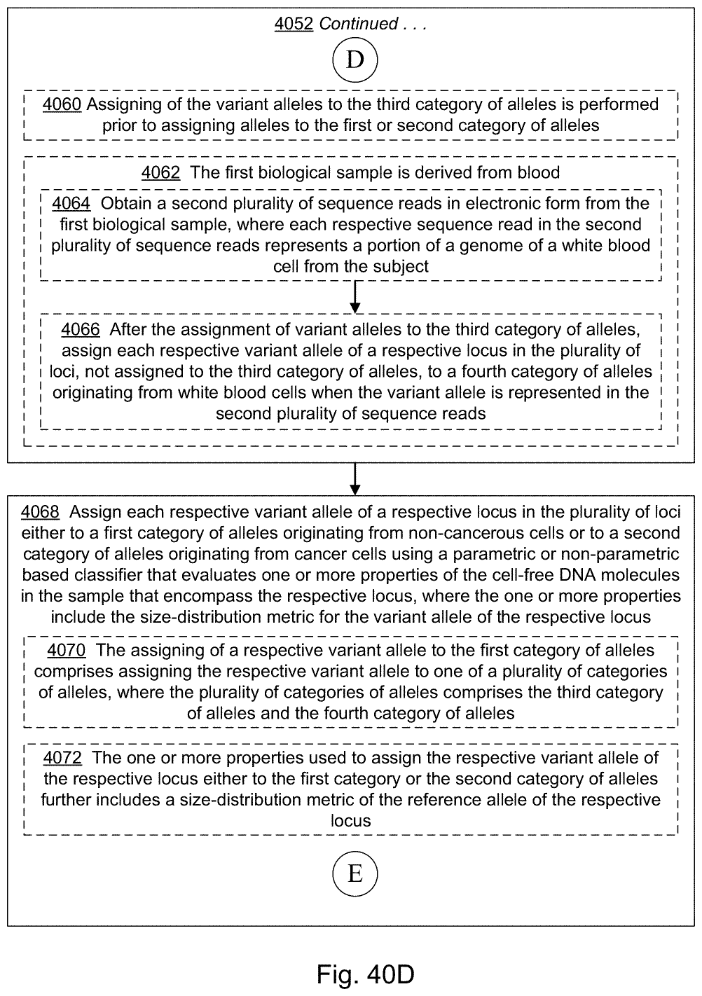

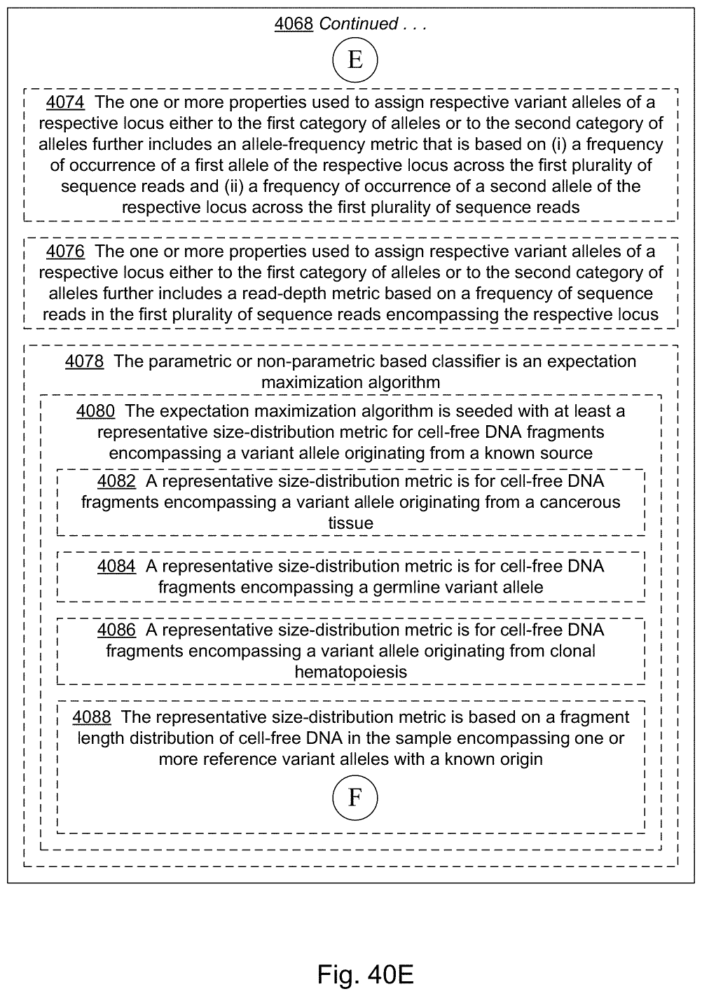

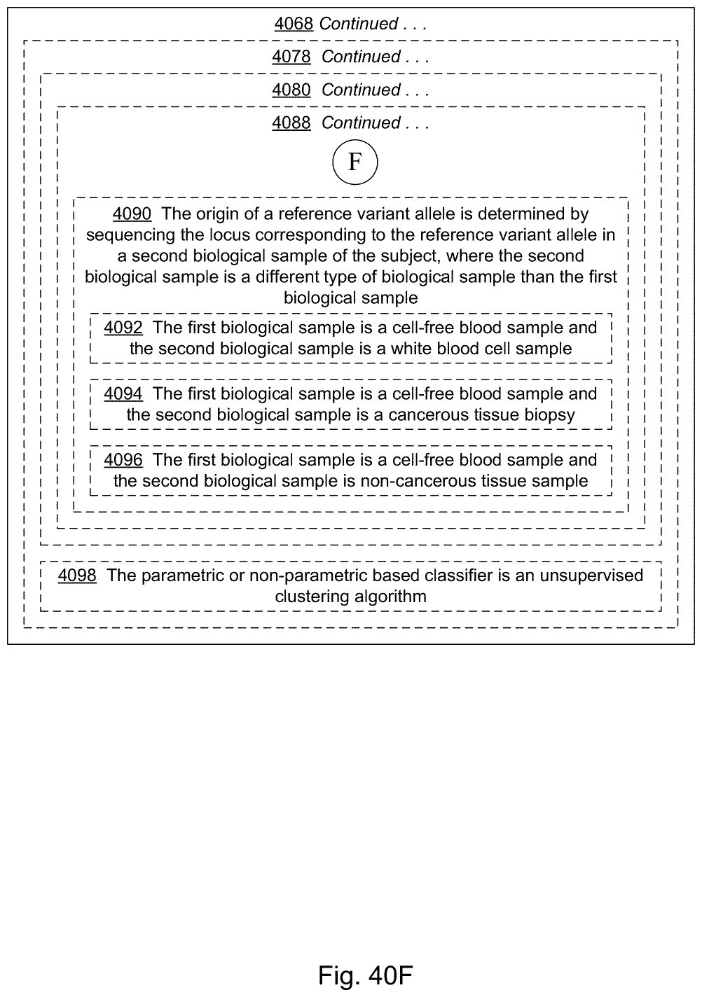

FIGS. 40A-40E are flow diagrams illustrating a method 4000 for determining the cellular origin of variant alleles present in a biological sample using a measure of the distribution of DNA fragment lengths of cell-free DNA fragments isolated from the blood of the subject which encompass an allele of interest. Method 4000 is performed at a computer system (e.g., computer system 100 or 150 in FIG. 1) having one or more processors, and memory storing one or more programs for execution by the one or more processors for phasing alleles present on a matching pair of chromosomes in a cancerous tissue of a subject. Some operations in method 4000 are, optionally, combined and/or the order of some operations is, optionally, changed.

In some embodiments, method 4000 is performed at a computer system comprising one or more processors, and memory storing one or more programs for execution by the one or more processors. The method includes obtaining (4004) a dataset comprising a plurality of nucleic acid fragment sequences in electronic form from a first biological sample of the subject, where each respective nucleic acid fragment sequence in the plurality of nucleic acid fragment sequences represents all or a portion of a respective cell-free DNA molecule in a population of cell-free DNA molecules in the first biological sample, the respective nucleic acid fragment sequence encompassing a corresponding locus in a plurality of loci, represented by at least a reference allele and a variant allele within the population of cell-free DNA molecules.

For example, as described above, it is known that mono- and di-nucleosomes fragmented from the genomes of non-cancerous somatic cells, hematopoietic cells (e.g., white blood cells), and (when the subject has cancer) cancerous cells. Thus, in some embodiments, the cell-free DNA molecules in the sample originate from at least non-cancerous somatic cells and hematopoietic cells (e.g., white blood cells). In some embodiments, sample also includes cell-free DNA molecules originating from cancerous cells. Accordingly, in some embodiments, the first biological sample includes cell-free DNA originating from at least cancerous cells, non-cancerous somatic cells, and white blood cells.

In some embodiments, it is unknown whether the subject has cancer and, thus, whether cell-free DNA originating from cancerous cells in present in the sample prior to analysis. Accordingly, in some embodiments, the subject has not been diagnosed as having cancer (4018). In some embodiments, the subject has already been diagnosed with cancer and, accordingly, it is known that the cell-free DNA originating from cancerous cells is present in the sample prior to analysis. In some embodiments, the subject is a human (4016).

In some embodiments, the obtaining step of the method includes collecting (4002) the plurality of sequencing reads from the cell-free DNA in the biological sample from the subject using a nucleic acid sequencer. However, in other embodiments, method 4000 only includes obtaining the sequencing data from a prior sequencing reaction of cell-free DNA from a biological sample.

Methods for collecting suitable sequencing data for the methods described herein (e.g., method 4000) are described above, and are not reiterated here for reasons of brevity. Regardless of the exact sequencing method used, however, in some embodiments, each respective nucleic acid fragment sequence in the plurality of nucleic acid fragment sequences is obtained by generating complementary sequence reads from both ends of a respective cell-free DNA molecule in the population of cell-free DNA (4006), where the complementary sequence reads are combined to form a respective sequence read, which is collapsed with other respective sequence reads of the same unique nucleic acid fragment to form the respective nucleic acid fragment sequence. For example, in some embodiments, complementary sequence reads are stitched together based on an overlapping region of sequence shared between the complementary sequence reads and/or by matching the sequences from complementary sequence reads to corresponding sequences in a reference genome for the species of the subject.

In some embodiments, the first biological sample is a blood sample (4010), e.g., a whole-blood sample, a blood serum sample, or a blood plasma sample. In some embodiments, the blood sample is a whole blood sample, and prior to generating the plurality of nucleic acid fragment sequences from the whole blood sample, white blood cells are removed from the whole blood sample. In some embodiments, the white blood cells are collected as a second type of sample, e.g., according to a buffy coat extraction method, from which additional sequencing data may or may not be obtained. In some embodiments, the method further includes obtaining a second plurality of nucleic acid fragment sequences in electronic form of genomic DNA from the white blood cells removed from the whole blood sample. In some embodiments, the second plurality of nucleic acid fragment sequences is used to identify allele variants arising from clonal hematopoiesis, as opposed to germline allele variants and/or allele variants arising from a cancer in the subject. Likewise, in some embodiments, fragment length distributions obtained for fragments encompassing an allele are used to seed a classification algorithm, e.g., an expectation maximization (EM) algorithm. In some embodiments, the blood sample is a blood serum sample (4014).

In some embodiments, the plurality of loci are selected from a predetermined set of loci that includes less than all loci in the genome of the subject (4020). In some embodiments, nucleic acid fragment sequences of the cell-free DNA molecules in the sample are generated for a predetermined set of loci, e.g., by targeted panel sequencing. As described above, many targeted panels for sequencing alleles of interest, e.g., related to cancer diagnostics, are known to those of skill in the art. Although not reiterated here for reasons of brevity, any of these targeted panels can be used in the methods described herein. In some embodiments, the targeted panel includes loci known to provide diagnostic or prognostic power for cancer diagnostics, e.g., loci at which an allele has been linked to a characteristic of a cancer. In some embodiments, the targeted panel includes alleles that are distributed throughout the genome of the species of the subject, e.g., to provide representation for a large portion of the genome.

In some embodiments, the predetermined set of loci includes at least 100 loci (4022). In some embodiments, the predetermined set of loci includes at least 500 loci (4024). In some embodiments, the predetermined set of loci includes at least 1000 loci (4026). In some embodiments, the predetermined set of loci includes at least 5000 loci (4028). In some embodiments, the predetermined set of loci includes at least 100, 200, 300, 400, 500, 600, 700, 800, 900, 1000, 2000, 3000, 4000, 5000, 6000, 7000, 8000, 9000, 10,000, 15,000, 20,000, 25,000, 50,000, 75,000, 100,000, or more loci. In some embodiments, the predetermined set of loci includes from 100 to 100,000 loci, from 100 to 50,000 loci, from 100 to 25,000 loci, from 100 to 10,000 loci, from 100 to 5000 loci, from 100 to 2000 loci, from 100 to 1000 loci, from 500 to 100,000 loci, from 500 to 50,000 loci, from 500 to 25,000 loci, from 500 to 10,000 loci, from 500 to 5000 loci, from 500 to 2000 loci, from 500 to 1000 loci, from 1000 to 100,000 loci, from 1000 to 50,000 loci, from 1000 to 25,000 loci, from 1000 to 10,000 loci, from 1000 to 5000 loci, or from 1000 to 2000 loci.

In some embodiments, the average coverage rate of nucleic acid fragment sequences of the predetermined set of loci taken from the sample is at least 25× (4030). In some embodiments, the average coverage rate of nucleic acid fragment sequences of the predetermined set of loci taken from the sample is at least 50×, 100×, 200×, 300×, 400×, 500×, 750×, 1000×, 2000×, 3000×, 4000×, 5000×, or more. In some embodiments, the average coverage rate of nucleic acid fragment sequences of the predetermined set of loci taken from the sample is from 25× to 5000×, from 25× to 2500×, from 25× to 1000×, from 25× to 500×, from 25× to 100×, from 100× to 5000×, from 100× to 2500×, from 100× to 1000×, or from 100× to 500×.

In some embodiments, all of the cell-free DNA molecules in the sample are sequenced (4032), e.g., by whole genome sequencing, and nucleic acid fragment sequences corresponding to cell-free DNA molecules encompassing the predetermined set of loci are selected for the analysis. As described above, many methods for whole genome sequencing are known to those of skill in the art. In some embodiments, the average coverage rate of nucleic acid fragment sequences across the genome of the subject is at least 10× (4034). In some embodiments, the average coverage rate of nucleic acid fragment sequences across the genome of the subject is at least 25×, 50×, 100×, 200×, 300×, 400×, 500×, 750×, 1000×, or more. In some embodiments, the average coverage rate of nucleic acid fragment sequences of the predetermined set of loci taken from the sample is from 10× to 1000×, from 10× to 500×, from 10× to 100×, from 10× to 50×, from 50× to 1000×, from 50× to 500×, or from 50× to 100×.

In some embodiments, the at least two different alleles of a respective locus include a reference allele and a variant allele. In some embodiments, the at least two different alleles of a respective locus include a variant allele that is a single nucleotide polymorphism relative to a reference allele for the locus (4036). In some embodiments, the preceding claims, wherein the at least two different alleles of a respective locus include a variant allele that is a deletion of twenty-five nucleotides or less, encompassing the respective locus, relative to a reference allele for the locus (4038). In some embodiments, the at least two different alleles of a respective locus include a variant allele that is a single nucleotide deletion relative to a reference allele for the locus (4040). In some embodiments, the at least two different alleles of a respective locus include a variant allele that is an insertion of twenty-five nucleotides or less, encompassing the respective locus, relative to a reference allele for the locus (4042). In some embodiments, the at least two different alleles of a respective locus include a variant allele that is a single nucleotide insertion relative to a reference allele for the locus (4044).

Method 4000 also includes assigning (4046), for each respective allele represented at each locus in the plurality of loci, a size-distribution metric (e.g., a median length, a median shift in length, a measure of central tendency of length across the distribution, a measure of central tendency of shift in length across the distribution, or a statistical distribution) based on a characteristic of the distribution of the fragment lengths of the cell-free DNA molecules in the population of cell-free DNA molecules (e.g., that are represented by a respective nucleic acid fragment sequence in the plurality of nucleic acid fragment sequences) that encompass the respective allele, thereby obtaining a set of size-distribution metrics. Because the set of size-distribution metrics is smaller than the set of individual nucleic acid fragment sequences, this step compresses the data in order to make the method more computationally efficient, e.g., by allowing the computer to apply an algorithm to the smaller dataset (the set size distribution metrics) rather than the full dataset (the nucleic acid fragment sequences themselves). In one embodiment, the size-distribution metric is a measure of central tendency of length across the distribution (4048). In some embodiments, the measure of central tendency of length across the distribution is an arithmetic mean, weighted mean, midrange, midhinge, trimean, Winsorized mean, median, or mode of the distribution (4050).

Method 4000 also includes assigning (4068) each respective variant allele of a respective locus in the plurality of loci either to a first category of alleles originating from non-cancerous cells (e.g., where the first category includes germline tissue or hematopoietic cells, e.g., white blood cells where the variant allele has arisen from clonal hematopoiesis) or to a second category of alleles originating from cancer cells using a parametric or non-parametric based classifier that evaluates one or more properties of the cell-free DNA molecules in the sample that encompass the respective locus, where the one or more properties include the size-distribution metric for the variant allele of the respective locus. In some embodiments, the one or more properties used to assign the respective variant allele of the respective locus either to the first category or the second category of alleles further includes a size-distribution metric of the reference allele of the respective locus (4072).

In some embodiments, the one or more properties used to assign respective variant alleles of a respective locus either to the first category of alleles or to the second category of alleles further includes an allele-frequency metric that is based on (i) a frequency of occurrence of a first allele of the respective locus across the first plurality of nucleic acid fragment sequences and (ii) a frequency of occurrence of a second allele of the respective locus across the first plurality of nucleic acid fragment sequences (4074).

In some embodiments, the one or more properties used to assign respective variant alleles of a respective locus either to the first category of alleles or to the second category of alleles further includes a read-depth metric based on a frequency of nucleic acid fragment sequences in the first plurality of nucleic acid fragment sequences encompassing the respective locus, e.g., a frequency of nucleic acid fragment sequences containing the respective locus or a frequency of nucleic acid fragment sequences that correspond to a same portion of a reference genome (e.g., a bin) for the species of the subject as the respective locus, in a plurality of different and non-overlapping portions of the reference genome.

In some embodiments, the assigning (4068) of a respective variant allele to the first category of alleles includes assigning (4070) the respective variant allele to one of a plurality of categories of alleles, wherein the plurality of categories of alleles includes a third category of alleles originating from a germline cell and a fourth category of alleles originating from a hematopoietic cell, e.g., a white blood cell. That is, rather than just classifying the allele as arising from a cancerous origin or non-cancerous origin, the method classifies the allele as arising from a cancerous origin or from one of two or more non-cancerous origins (e.g., somatic germline cells or white blood cells).

In some embodiments, a respective variant allele is identified as a germline variant based on a frequency of the variant allele in the population of the species of the subject (4054). That is, except in cases where a very high tumor burden exists, the majority of the cell-free DNA found in the blood will be derived either from somatic cells or from hematopoietic cells. Thus, allele variants arising from a cancerous tissue will be far less prevalent in the blood than germline alleles, since only a small fraction of the cell-free DNA is from cancer cells. Similarly, since mutagenesis via clonal hematopoiesis affects only a clonal subpopulation of all hematopoietic cells, the majority of cell-free DNA from hematopoietic cells in the blood includes a germline sequence. Thus, allele variants arising via clonal hematopoiesis will be far less prevalent in the blood than germline alleles. Accordingly, only germline variant alleles will be found at a prevalence approaching 50% of all cell-free DNA encompassing the locus in the blood. Thus, in some embodiments, a respective variant allele is identified as a germline variant when the prevalence of the allele, relative to all sequenced alleles at the respective locus, is at a level of least a threshold percentage, e.g., at least 25%, 30%, 35%, 40%, 45%, or more, e.g., depending upon the variability and depth of sequencing. In some embodiments, allele population frequencies available in compiled databases can be used, e.g., alone or in combination with other information, as a predictive model for determining whether a variant allele originated from a particular source, e.g., germline, clonal hematopoiesis, or cancerous cells.

In some embodiments, a respective variant allele is identified as a germline variant based on sequencing of the locus corresponding to the variant allele in a second biological sample of the subject, wherein the second biological sample is a non-cancerous tissue sample (4056). For example, in some embodiments, a blood sample and a non-cancerous tissue sample are collected from the subject, and loci of interest are sequenced from both samples. Accordingly, variant alleles sequenced in the cell-free portion of the sample, which match variant alleles sequenced in the non-cancerous tissue sample can be positively identified as originating from the germline of the subject. Similarly, in some embodiments, loci of interest are sequenced from both a cell-free blood sample and a sample of white blood cells, and variant alleles sequenced in the white blood cell sample that have a prevalence approaching 50%, indicating that they are derived from the germline rather than from clonal hematopoiesis, can be identified with a high likelihood of originating from the germline of the subject.

In some embodiments, a respective variant allele is identified as a germline variant based on an allele-frequency metric that is based on (i) a frequency of occurrence of a first allele of the respective locus across the first plurality of nucleic acid fragment sequences and (ii) a frequency of occurrence of a second allele of the respective locus across the first plurality of nucleic acid fragment sequences (4058). For example, assigning, for each respective locus in the plurality of loci, an allele-frequency metric based on (i) a frequency of occurrence of a first allele of the respective locus across the first plurality of nucleic acid fragment sequences and (ii) a frequency of occurrence of a second allele of the respective locus across the first plurality of nucleic acid fragment sequences, thereby obtaining a set of allele-frequency metrics; and assigning each respective variant allele of a respective locus in the plurality of loci to a first category of alleles originating from the germline of the subject when the respective locus has an allele-frequency metric that is within a threshold amount of a value representing an equal representation of reference and variant alleles at the respective locus across the first plurality of nucleic acid fragment sequences.

In some embodiments, the assigning of the variant alleles to the third category of alleles (e.g., identifying a variant allele as a germline allele) is performed (4060) prior to the assigning (4068), e.g., prior to determining whether the variant allele arises from a cancerous origin. In some embodiments, the first biological sample is derived from blood (4062), and the method further includes obtaining (4064) a second plurality of nucleic acid fragment sequences in electronic form from the first biological sample, wherein each respective nucleic acid fragment sequence in the second plurality of nucleic acid fragment sequences represents a portion of a genome of a white blood cell from the subject. In some embodiments, after the assignment of variant alleles to the third category of alleles, the method includes assigning (4066) each respective variant allele of a respective locus in the plurality of loci, not assigned to the third category of alleles, to a fourth category of alleles originating from white blood cells (e.g., where the variant allele has arisen from clonal hematopoiesis) when the variant allele is represented in the second plurality of nucleic acid fragment sequences.

In some embodiments, the parametric or non-parametric based classifier is an expectation maximization algorithm (4078). In some embodiments, the expectation maximization algorithm is seeded with at least a representative size-distribution or size distribution metric for cell-free DNA fragments encompassing a variant allele originating from a known source (4080). In some embodiments, a representative size-distribution metric is for cell-free DNA fragments encompassing a variant allele originating from a cancerous tissue (4082). In some embodiments, a representative size-distribution metric is for cell-free DNA fragments encompassing a germline variant allele (4084). In some embodiments, a representative size-distribution metric is for cell-free DNA fragments encompassing a variant allele originating from clonal hematopoiesis (4086). In some embodiments, the representative size-distribution metric is based on a fragment length distribution of cell-free DNA in the sample encompassing one or more reference variant alleles with a known origin (4088).

In some embodiments, the origin of a reference variant allele is determined by sequencing the locus corresponding to the reference variant allele in a second biological sample of the subject, where the second biological sample is a different type of biological sample than the first biological sample (4090). In some embodiments, the first biological sample is a cell-free blood sample and the second biological sample is a white blood cell sample (4092). For instance, in some embodiments, a blood sample containing at least blood serum and white blood cells is collected from the subject, the white blood cells are removed from the sample (e.g., via buffy coat extraction), and loci of interest are sequenced in both the cell-free portion and the white blood cell portion of the original sample (e.g., which were separated from each other). Accordingly, variant alleles sequenced in the cell-free portion of the sample, which do not originate from the germline of the subject and which match variant alleles sequenced in the white blood cell sample can be positively identified as originating from clonal hematopoiesis, and can be used to seed the expectation maximization algorithm. In some embodiments, the first biological sample is a cell-free blood sample and the second biological sample is a cancerous tissue biopsy (4094). For instance, in some embodiments, a blood sample and a tumor biopsy are collected from the subject, and loci of interest are sequenced from both samples. Accordingly, variant alleles sequenced in the cell-free portion of the sample, which do not originate from the germline of the subject and which match variant alleles sequenced in the tumor biopsy can be positively identified as originating from cancerous tissue in the subject, and can be used to seed the expectation maximization algorithm. In some embodiments, the first biological sample is a cell-free blood sample and the second biological sample is non-cancerous tissue sample (4096). For instance, in some embodiments, a blood sample and a non-cancerous tissue sample are collected from the subject, and loci of interest are sequenced from both samples. Accordingly, variant alleles sequenced in the cell-free portion of the sample, which match variant alleles sequenced in the non-cancerous tissue sample can be positively identified as originating from the germline of the subject, and can be used to seed the expectation maximization algorithm. In some embodiments, the parametric or non-parametric based classifier is an unsupervised clustering algorithm (4098).

It should be understood that the particular order in which the operations in FIGS. 40A-40F have been described is merely an example and is not intended to indicate that the described order is the only order in which the operations could be performed. One of ordinary skill in the art would recognize various ways to reorder the operations described herein. Additionally, it should be noted that details of other processes described herein with respect to other methods described herein (e.g., methods 3700, 3800, 3900, 4100, and 4200) are also applicable in an analogous manner to method 3900 described above with respect to FIGS. 40A-40F. Further, in some embodiments, method 4000 can be used in conjunction with any other method described herein (e.g., methods 3700, 3800, 3900, 4100, and 4200). The operations in the information processing methods described above are, optionally implemented by running one or more functional modules in information processing apparatus such as general purpose processors (e.g., as described above with respect to FIGS. 1A and 1B) or application specific chips.

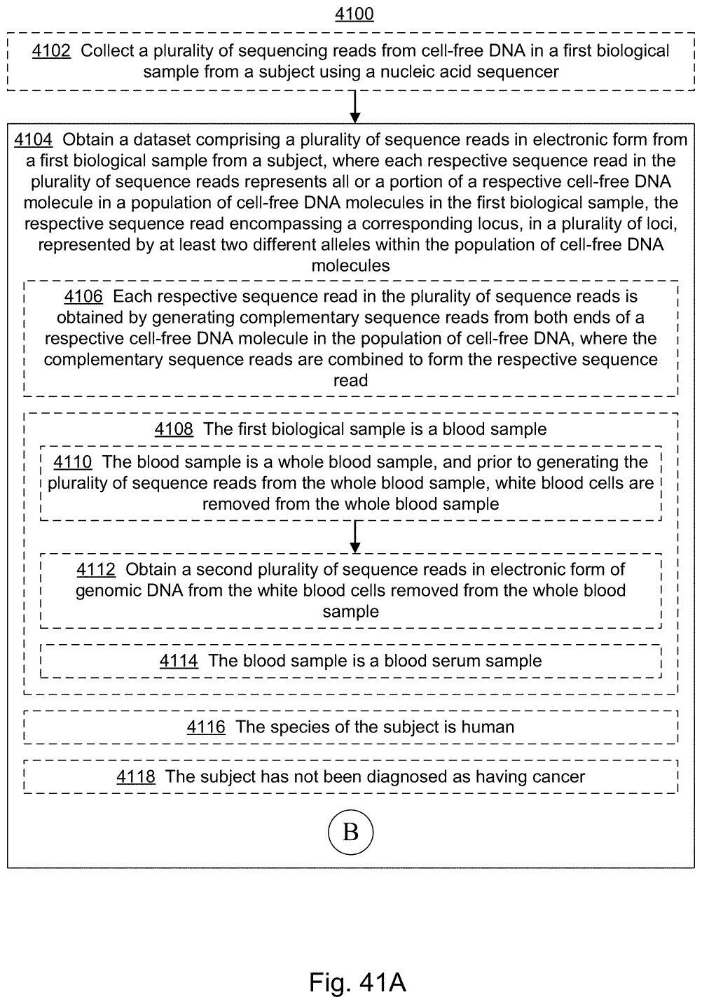

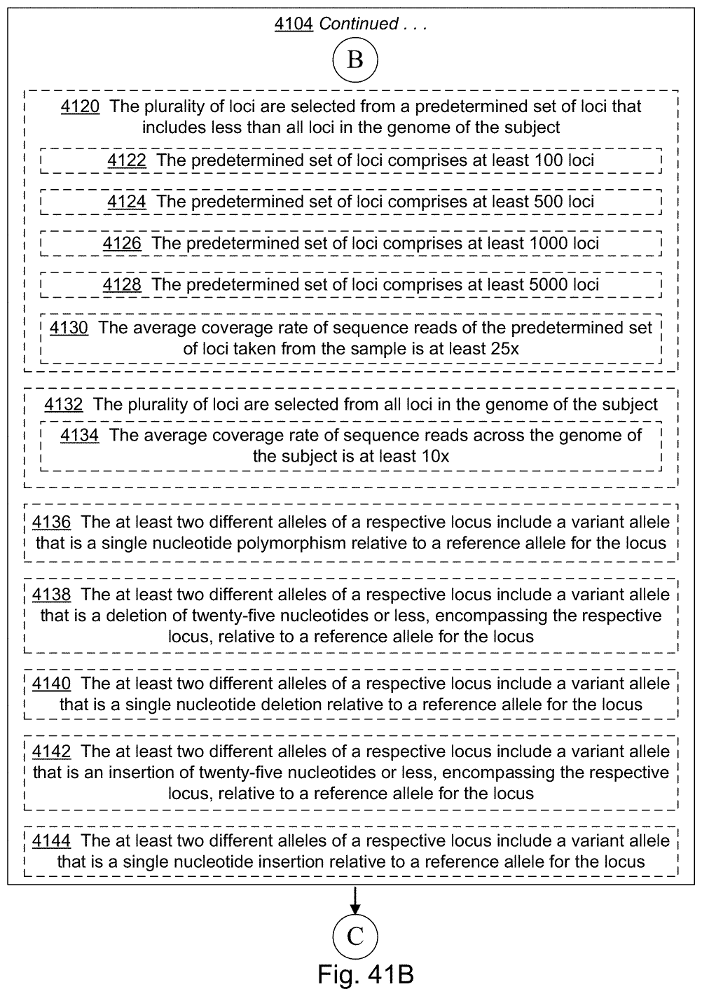

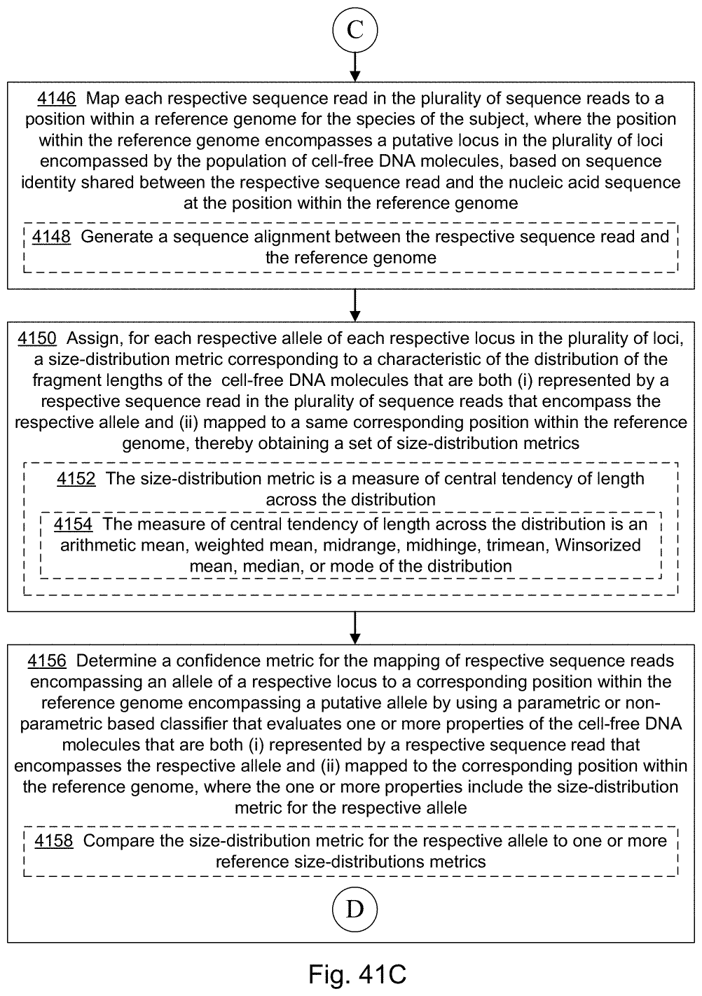

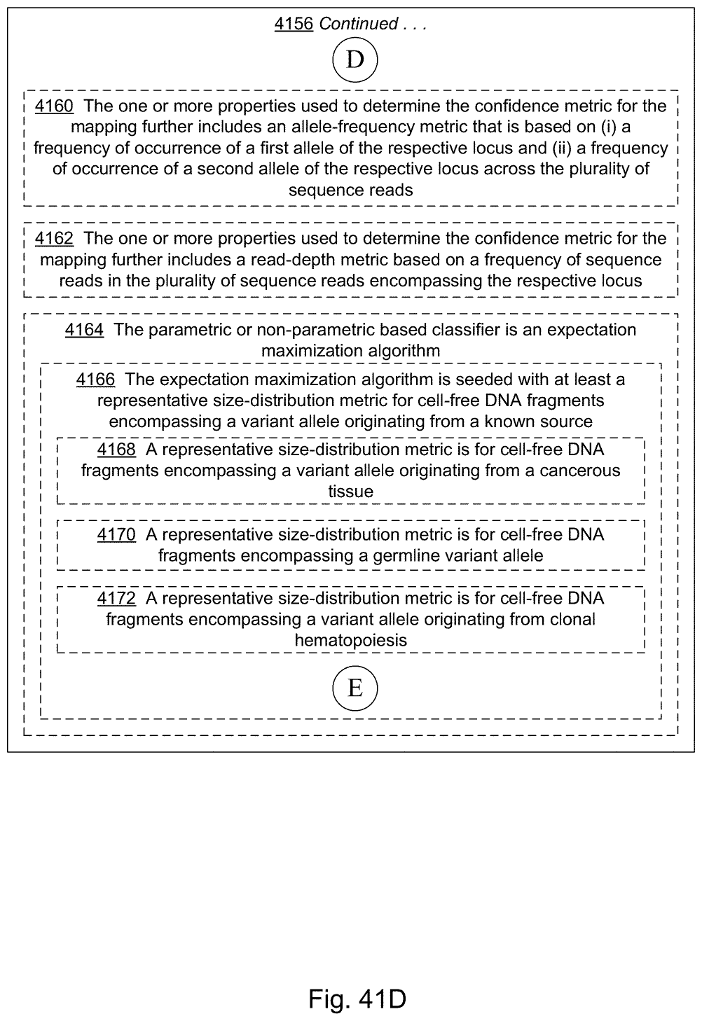

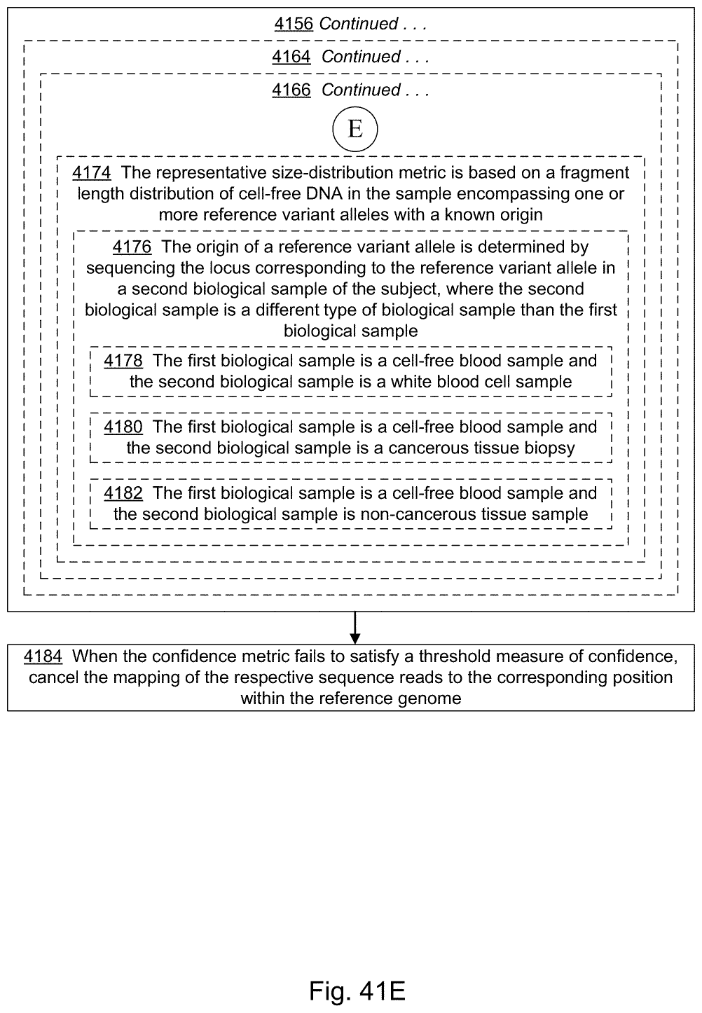

FIGS. 41A-41E are flow diagrams illustrating a method 4100 for identifying and canceling an incorrect mapping of a nucleic acid fragment sequence to a position within a reference genome using a measure of the distribution of DNA fragment lengths of cell-free DNA fragments isolated from the blood of a subject which encompass an allele of interest. Method 4100 is performed at a computer system (e.g., computer system 100 or 150 in FIG. 1) having one or more processors, and memory storing one or more programs for execution by the one or more processors for phasing alleles present on a matching pair of chromosomes in a cancerous tissue of a subject. Some operations in method 4100 are, optionally, combined and/or the order of some operations is, optionally, changed.

In some embodiments, method 4100 is performed at a computer system comprising one or more processors, and memory storing one or more programs for execution by the one or more processors. The method includes obtaining (4104) a dataset comprising a plurality of nucleic acid fragment sequences in electronic form from a first biological sample of the subject, where each respective nucleic acid fragment sequence in the plurality of nucleic acid fragment sequences represents all or a portion of a respective cell-free DNA molecule in a population of cell-free DNA molecules in the first biological sample, the respective nucleic acid fragment sequence encompassing a corresponding locus in a plurality of loci, where each locus in the plurality of loci is represented by at least two different alleles within the population of cell-free DNA molecules. In some embodiments, the at least two different alleles are two different germline alleles, e.g., two different reference alleles found at the loci of respective maternal and paternal chromosomes within the germline of the subject, or one reference allele and one variant allele found at the loci of respective maternal and paternal chromosomes within the germline of the subject. In some embodiments, the at least two different alleles include a reference or variant allele represented within the germline of the subject and a variant allele arising from a cancerous tissue of the subject, at the respective locus.

For example, as described above, it is known that mono- and di-nucleosomes fragmented from the genomes of non-cancerous somatic cells, hematopoietic cells (e.g., white blood cells), and (when the subject has cancer) cancerous cells. Thus, in some embodiments, the cell-free DNA molecules in the sample originate from at least non-cancerous somatic cells and hematopoietic cells (e.g., white blood cells). In some embodiments, sample also includes cell-free DNA molecules originating from cancerous cells. Accordingly, in some embodiments, the first biological sample includes cell-free DNA originating from at least cancerous cells, non-cancerous somatic cells, and white blood cells.

In some embodiments, it is unknown whether the subject has cancer and, thus, whether cell-free DNA originating from cancerous cells in present in the sample prior to analysis. Accordingly, in some embodiments, the subject has not been diagnosed as having cancer (4118). In some embodiments, the subject has already been diagnosed with cancer and, accordingly, it is known that the cell-free DNA originating from cancerous cells is present in the sample prior to analysis. In some embodiments, the subject is a human (4116).

In some embodiments, the obtaining step of the method includes collecting (4102) the plurality of sequencing reads from the cell-free DNA in the biological sample from the subject using a nucleic acid sequencer. However, in other embodiments, method 4100 only includes obtaining the sequencing data from a prior sequencing reaction of cell-free DNA from a biological sample.

Methods for collecting suitable sequencing data for the methods described herein (e.g., method 4100) are described above, and are not reiterated here for reasons of brevity. Regardless of the exact sequencing method used, however, in some embodiments, each respective nucleic acid fragment sequence in the plurality of nucleic acid fragment sequences is obtained by generating complementary sequence reads from both ends of a respective cell-free DNA molecule in the population of cell-free DNA (4106), where the complementary sequence reads are combined to form a respective sequence read, which is collapsed with other respective sequence reads of the same unique nucleic acid fragment to form the respective nucleic acid fragment sequence. For example, in some embodiments, complementary sequence reads are stitched together based on an overlapping region of sequence shared between the complementary sequence reads and/or by matching the sequences from complementary sequence reads to corresponding sequences in a reference genome for the species of the subject.

In some embodiments, the first biological sample is a blood sample (4108), e.g., a whole-blood sample, a blood serum sample, or a blood plasma sample. In some embodiments, the blood sample is a whole blood sample, and prior to generating the plurality of nucleic acid fragment sequences from the whole blood sample, white blood cells are removed from the whole blood sample (4110). In some embodiments, the white blood cells are collected as a second type of sample, e.g., according to a buffy coat extraction method, from which additional sequencing data may or may not be obtained. In some embodiments, the method further includes obtaining a second plurality of nucleic acid fragment sequences in electronic form of genomic DNA from the white blood cells removed from the whole blood sample (4112). In some embodiments, the second plurality of nucleic acid fragment sequences is used to identify allele variants arising from clonal hematopoiesis, as opposed to germline allele variants and/or allele variants arising from a cancer in the subject. Likewise, in some embodiments, fragment length distributions obtained for fragments encompassing an allele are used to seed a classification algorithm, e.g., an expectation maximization (EM) algorithm. In some embodiments, the blood sample is a blood serum sample (4114).

In some embodiments, the plurality of loci is selected from a predetermined set of loci that includes less than all loci in the genome of the subject (4120). In some embodiments, nucleic acid fragment sequences of the cell-free DNA molecules in the sample are generated for a predetermined set of loci, e.g., by targeted panel sequencing. As described above, many targeted panels for sequencing alleles of interest, e.g., related to cancer diagnostics, are known to those of skill in the art. Although not reiterated here for reasons of brevity, any of these targeted panels can be used in the methods described herein. In some embodiments, the targeted panel includes loci known to provide diagnostic or prognostic power for cancer diagnostics, e.g., loci at which an allele has been linked to a characteristic of a cancer. In some embodiments, the targeted panel includes alleles that are distributed throughout the genome of the species of the subject, e.g., to provide representation for a large portion of the genome.

In some embodiments, the predetermined set of loci includes at least 100 loci (4122). In some embodiments, the predetermined set of loci includes at least 500 loci (4124). In some embodiments, the predetermined set of loci includes at least 1000 loci (4126). In some embodiments, the predetermined set of loci includes at least 5000 loci (4128). In some embodiments, the predetermined set of loci includes at least 100, 200, 300, 400, 500, 600, 700, 800, 900, 1000, 2000, 3000, 4000, 5000, 6000, 7000, 8000, 9000, 10,000, 15,000, 20,000, 25,000, 50,000, 75,000, 100,000, or more loci. In some embodiments, the predetermined set of loci includes from 100 to 100,000 loci, from 100 to 50,000 loci, from 100 to 25,000 loci, from 100 to 10,000 loci, from 100 to 5000 loci, from 100 to 2000 loci, from 100 to 1000 loci, from 500 to 100,000 loci, from 500 to 50,000 loci, from 500 to 25,000 loci, from 500 to 10,000 loci, from 500 to 5000 loci, from 500 to 2000 loci, from 500 to 1000 loci, from 1000 to 100,000 loci, from 1000 to 50,000 loci, from 1000 to 25,000 loci, from 1000 to 10,000 loci, from 1000 to 5000 loci, or from 1000 to 2000 loci.

In some embodiments, the average coverage rate of nucleic acid fragment sequences of the predetermined set of loci taken from the sample is at least 25× (4130). In some embodiments, the average coverage rate of nucleic acid fragment sequences of the predetermined set of loci taken from the sample is at least 50×, 100×, 200×, 300×, 400×, 500×, 750×, 1000×, 2000×, 3000×, 4000×, 5000×, or more. In some embodiments, the average coverage rate of nucleic acid fragment sequences of the predetermined set of loci taken from the sample is from 25× to 5000×, from 25× to 2500×, from 25× to 1000×, from 25× to 500×, from 25× to 100×, from 100× to 5000×, from 100× to 2500×, from 100× to 1000×, or from 100× to 500×.

In some embodiments, all of the cell-free DNA molecules in the sample are sequenced (4132), e.g., by whole genome sequencing, and nucleic acid fragment sequences corresponding to cell-free DNA molecules encompassing the predetermined set of loci are selected for the analysis. As described above, many methods for whole genome sequencing are known to those of skill in the art. In some embodiments, the average coverage rate of nucleic acid fragment sequences across the genome of the subject is at least 10× (4134). In some embodiments, the average coverage rate of nucleic acid fragment sequences across the genome of the subject is at least 25×, 50×, 100×, 200×, 300×, 400×, 500×, 750×, 1000×, or more. In some embodiments, the average coverage rate of nucleic acid fragment sequences of the predetermined set of loci taken from the sample is from 10× to 1000×, from 10× to 500×, from 10× to 100×, from 10× to 50×, from 50× to 1000×, from 50× to 500×, or from 50× to 100×.

In some embodiments, the at least two different alleles of a respective locus include a reference allele and a variant allele. In some embodiments, the at least two different alleles of a respective locus include a variant allele that is a single nucleotide polymorphism relative to a reference allele for the locus (4136). In some embodiments, the preceding claims, wherein the at least two different alleles of a respective locus include a variant allele that is a deletion of twenty-five nucleotides or less, encompassing the respective locus, relative to a reference allele for the locus (4138). In some embodiments, the at least two different alleles of a respective locus include a variant allele that is a single nucleotide deletion relative to a reference allele for the locus (4140). In some embodiments, the at least two different alleles of a respective locus include a variant allele that is an insertion of twenty-five nucleotides or less, encompassing the respective locus, relative to a reference allele for the locus (4142). In some embodiments, the at least two different alleles of a respective locus include a variant allele that is a single nucleotide insertion relative to a reference allele for the locus (4144).

Method 4100 also includes mapping (4146) each respective nucleic acid fragment sequence in the plurality of nucleic acid fragment sequences to a position within a reference genome for the species of the subject, wherein the position within the reference genome encompasses a putative locus in the plurality of loci encompassed by the population of cell-free DNA molecules, based on sequence identity shared between the respective nucleic acid fragment sequence and the nucleic acid sequence at the position within the reference genome. In some embodiments, the mapping includes generating (4148) a sequence alignment between the respective sequence and the reference genome.

Method 4100 also includes assigning (4150) for each respective allele of each respective locus in the plurality of loci, a size-distribution metric (e.g., a median length, a median shift in length, a measure of central tendency of length across the distribution, a measure of central tendency of shift in length across the distribution, or a statistical distribution) corresponding to a characteristic of the distribution of the fragment lengths of the cell-free DNA molecules that are both (i) represented by a respective nucleic acid fragment sequence in the plurality of nucleic acid fragment sequences that encompass the respective allele and (ii) mapped to a same corresponding position within the reference genome, thereby obtaining a set of size-distribution metrics. Because the set of size-distribution metrics is smaller than the set of individual nucleic acid fragment sequences, this step compresses the data in order to make the method more computationally efficient, e.g., by allowing the computer to apply an algorithm to the smaller dataset (the set size distribution metrics) rather than the full dataset (the nucleic acid fragment sequences themselves). In one embodiment, the size-distribution metric is a measure of central tendency of length across the distribution (4152). In some embodiments, the measure of central tendency of length across the distribution is an arithmetic mean, weighted mean, midrange, midhinge, trimean, Winsorized mean, median, or mode of the distribution (4154).

Method 4100 also includes determining (4158) a confidence metric for the mapping of respective nucleic acid fragment sequences encompassing an allele of a respective locus to a corresponding position within the reference genome encompassing a putative allele by using a parametric or non-parametric based classifier that evaluates one or more properties of the cell-free DNA molecules that are both (i) represented by a respective nucleic acid fragment sequence that encompasses the respective allele and (ii) mapped to the corresponding position within the reference genome, wherein the one or more properties include the size-distribution metric for the respective allele. In some embodiments, the determining (4158) includes comparing (4160) the size-distribution metric for the respective allele to one or more reference size-distributions metrics (e.g., a model size distribution metric for a nucleosomal-derived cell-free DNA, e.g., sequenced from a sample from a subject with or without cancer, or a size distribution metric from cell-free DNA's sequenced within the sample that encompass another allele, e.g., which is known to be correctly mapped to the reference genome for the species of the subject).

In some embodiments, the one or more properties used to determine the confidence metric for the mapping further includes an allele-frequency metric based on (i) a frequency of occurrence of a first germline allele representing the respective locus across the plurality of nucleic acid fragment sequences and (ii) a frequency of occurrence of a second allele representing the respective locus across the plurality of nucleic acid fragment sequences (4160).

In some embodiments, the one or more properties used to determine the confidence metric for the mapping further includes (4162) a read-depth metric based on a frequency of nucleic acid fragment sequences, in the plurality of nucleic acid fragment sequences, associated with the respective locus, e.g., a frequency of nucleic acid fragment sequences containing the respective locus or a frequency of nucleic acid fragment sequences that correspond to a same portion of a reference genome (e.g., a bin) for the species of the subject as the respective locus, in a plurality of different and non-overlapping portions of the reference genome.

In some embodiments, the parametric or non-parametric based classifier is an expectation maximization algorithm (4164). In some embodiments, the expectation maximization algorithm is seeded with at least a representative size-distribution or size distribution metric for cell-free DNA fragments encompassing a variant allele originating from a known source (4166). In some embodiments, a representative size-distribution metric is for cell-free DNA fragments encompassing a variant allele originating from a cancerous tissue (4168). In some embodiments, a representative size-distribution metric is for cell-free DNA fragments encompassing a germline variant allele (4170). In some embodiments, a representative size-distribution metric is for cell-free DNA fragments encompassing a variant allele originating from clonal hematopoiesis (4172). In some embodiments, the representative size-distribution metric is based on a fragment length distribution of cell-free DNA in the sample encompassing one or more reference variant alleles with a known origin (4174).

In some embodiments, the origin of a reference variant allele is determined by sequencing the locus corresponding to the reference variant allele in a second biological sample of the subject, where the second biological sample is a different type of biological sample than the first biological sample (4176). In some embodiments, the first biological sample is a cell-free blood sample and the second biological sample is a white blood cell sample (4178). For instance, in some embodiments, a blood sample containing at least blood serum and white blood cells is collected from the subject, the white blood cells are removed from the sample (e.g., via buffy coat extraction), and loci of interest are sequenced in both the cell-free portion and the white blood cell portion of the original sample (e.g., which were separated from each other). Accordingly, variant alleles sequenced in the cell-free portion of the sample, which do not originate from the germline of the subject and which match variant alleles sequenced in the white blood cell sample can be positively identified as originating from clonal hematopoiesis, and can be used to seed the expectation maximization algorithm. In some embodiments, the first biological sample is a cell-free blood sample and the second biological sample is a cancerous tissue biopsy (4180). For instance, in some embodiments, a blood sample and a tumor biopsy are collected from the subject, and loci of interest are sequenced from both samples. Accordingly, variant alleles sequenced in the cell-free portion of the sample, which do not originate from the germline of the subject and which match variant alleles sequenced in the tumor biopsy can be positively identified as originating from cancerous tissue in the subject, and can be used to seed the expectation maximization algorithm. In some embodiments, the first biological sample is a cell-free blood sample and the second biological sample is non-cancerous tissue sample (4182). For instance, in some embodiments, a blood sample and a non-cancerous tissue sample are collected from the subject, and loci of interest are sequenced from both samples. Accordingly, variant alleles sequenced in the cell-free portion of the sample, which match variant alleles sequenced in the non-cancerous tissue sample can be positively identified as originating from the germline of the subject, and can be used to seed the expectation maximization algorithm.

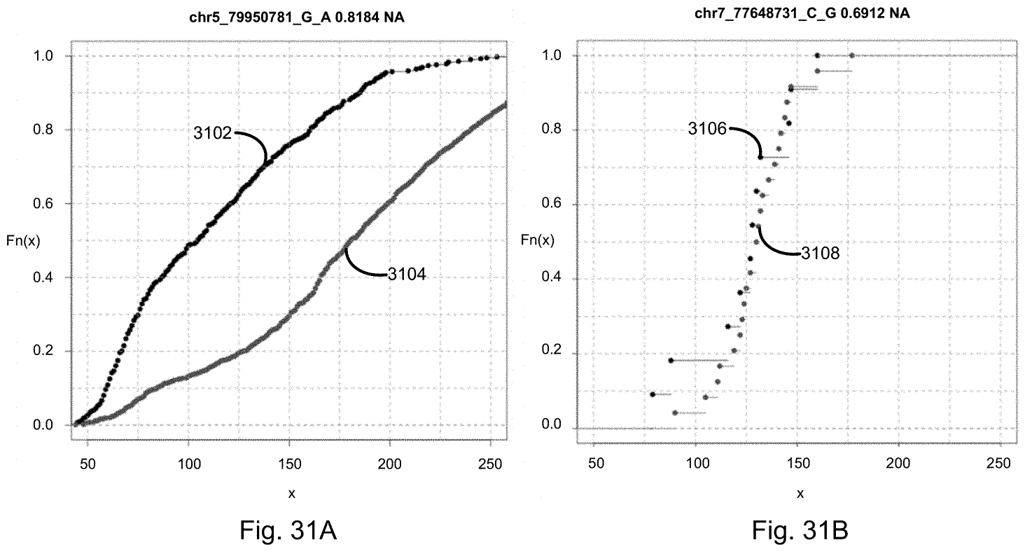

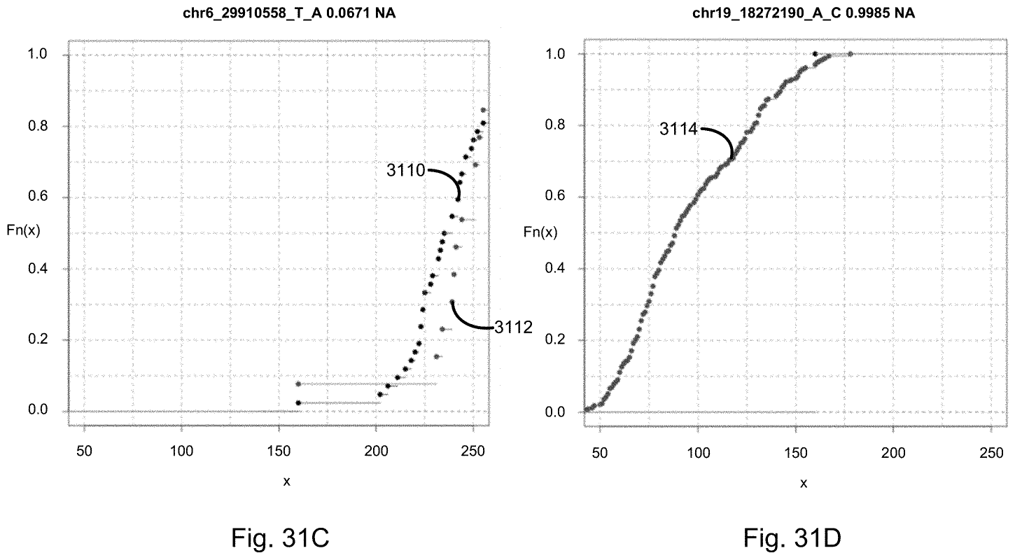

When the confidence metric fails to satisfy a threshold measure of confidence (e.g., is below a predetermined threshold), the method includes canceling (4182) the mapping of the respective nucleic acid fragment sequences to the corresponding position within the reference genome. For instance, as described in Example 12, several cell-free DNA fragment length distributions have been identified that indicate that the fragment sequences have been mapped to an incorrect location in the reference genome. For example, FIGS. 30A-30C illustrate three distributions which appear to show a significant shift shorter of the fragment lengths. However, these fragments were mis-mapped to the reference genome because the segment of the subject's genome from which these fragments arose was not part of the reference genome. This was a result of a hereditary region in the subject family, that is not present in most human genomes. Thus, significantly larger fragment lengths shifts can indicate mis-mappings. Similarly, FIGS. 31A-31D show other fragment length distributions which indicate that the fragments were mis-matched, rather than indicating an associated biological feature that is relevant to cancer.

It should be understood that the particular order in which the operations in FIGS. 41A-41E have been described is merely an example and is not intended to indicate that the described order is the only order in which the operations could be performed. One of ordinary skill in the art would recognize various ways to reorder the operations described herein. Additionally, it should be noted that details of other processes described herein with respect to other methods described herein (e.g., methods 3700, 3800, 3900, 4000, and 4200) are also applicable in an analogous manner to method 4100 described above with respect to FIGS. 41A-41E. Further, in some embodiments, method 4100 can be used in conjunction with any other method described herein (e.g., methods 3700, 3800, 3900, 4000, and 4200). The operations in the information processing methods described above are, optionally implemented by running one or more functional modules in information processing apparatus such as general purpose processors (e.g., as described above with respect to FIGS. 1A and 1B) or application specific chips.

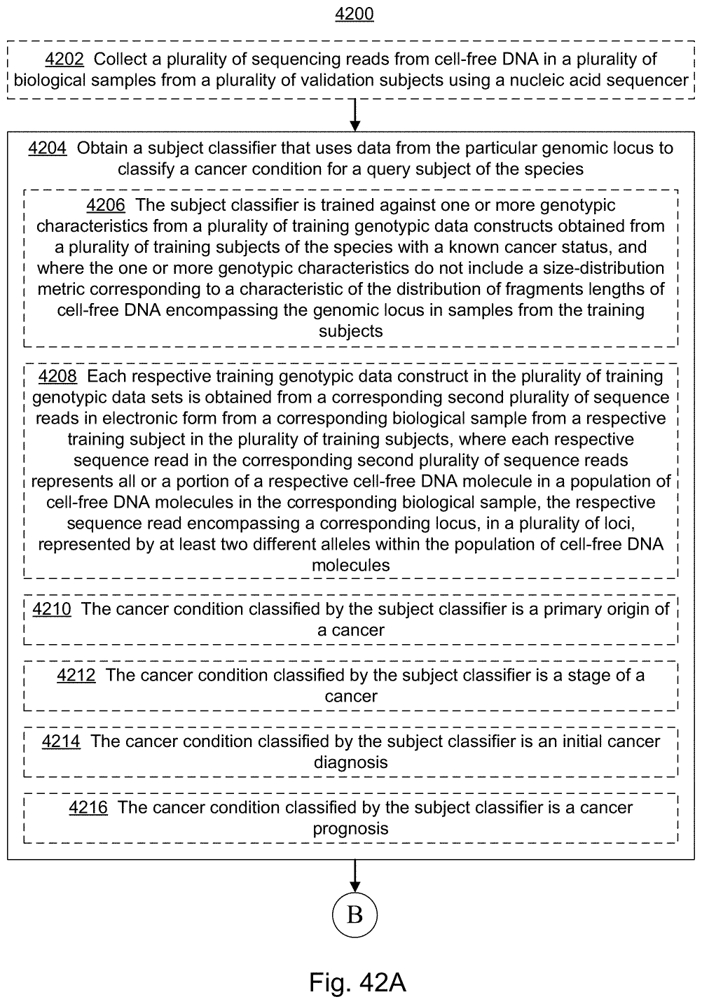

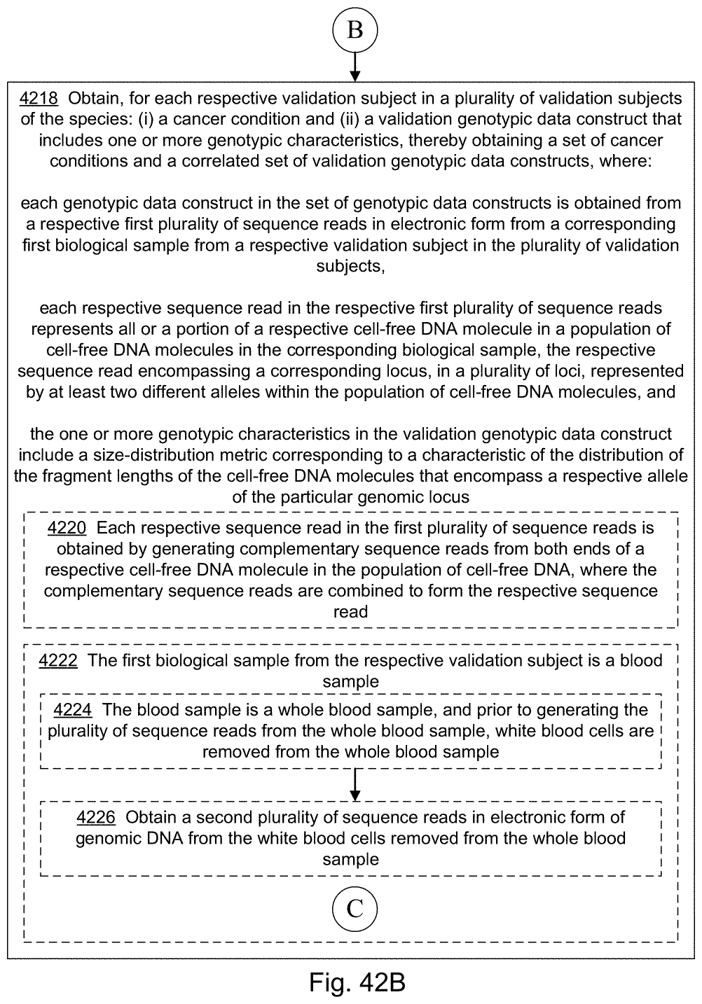

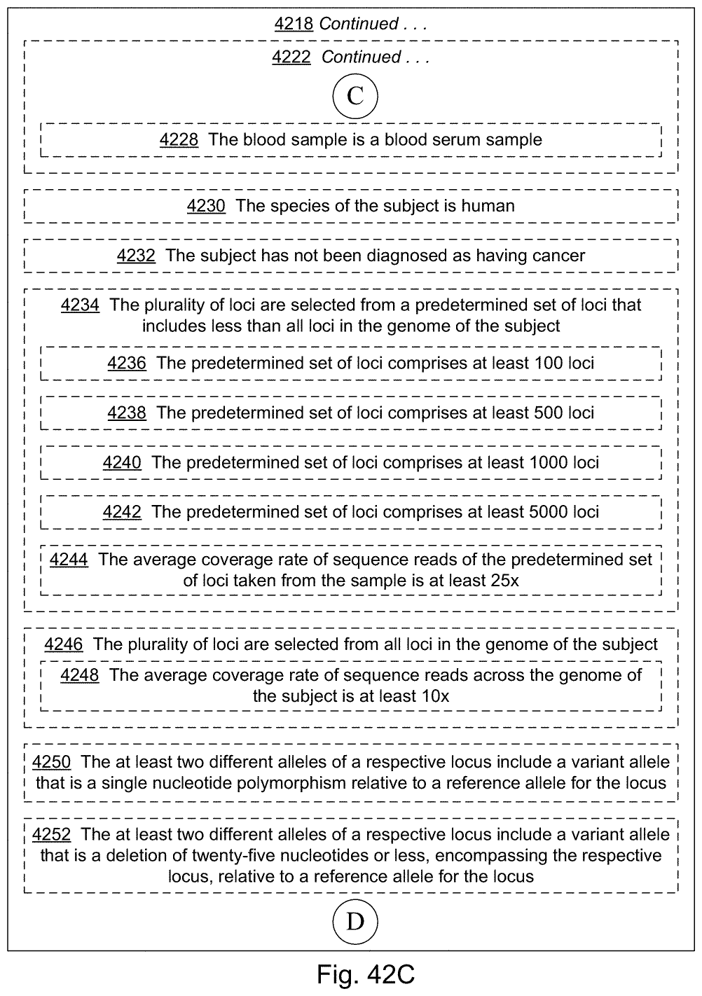

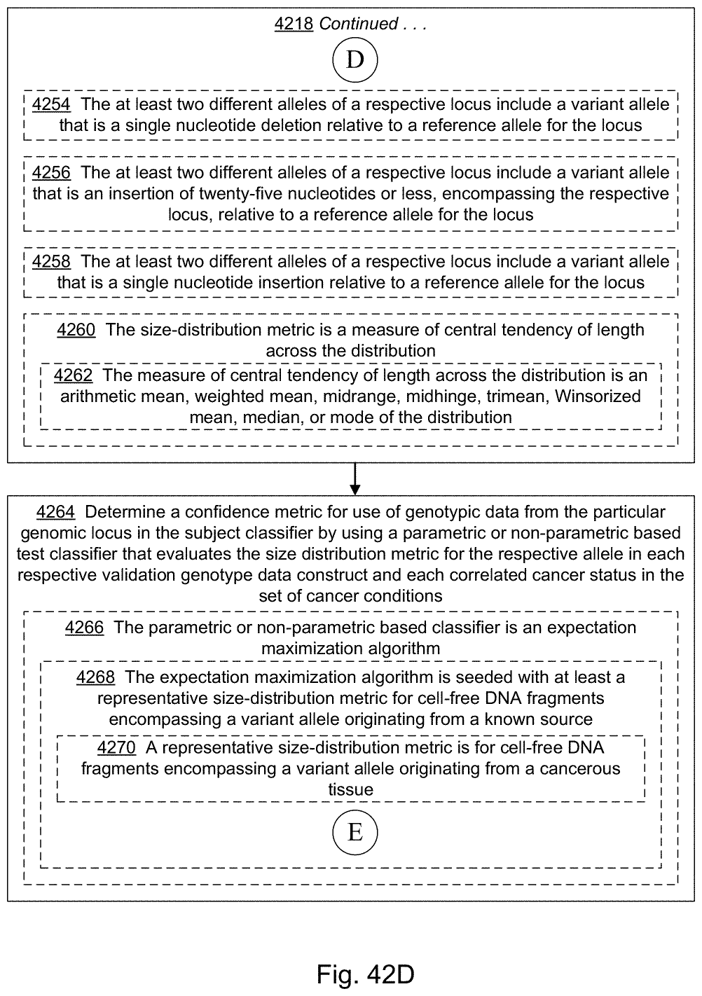

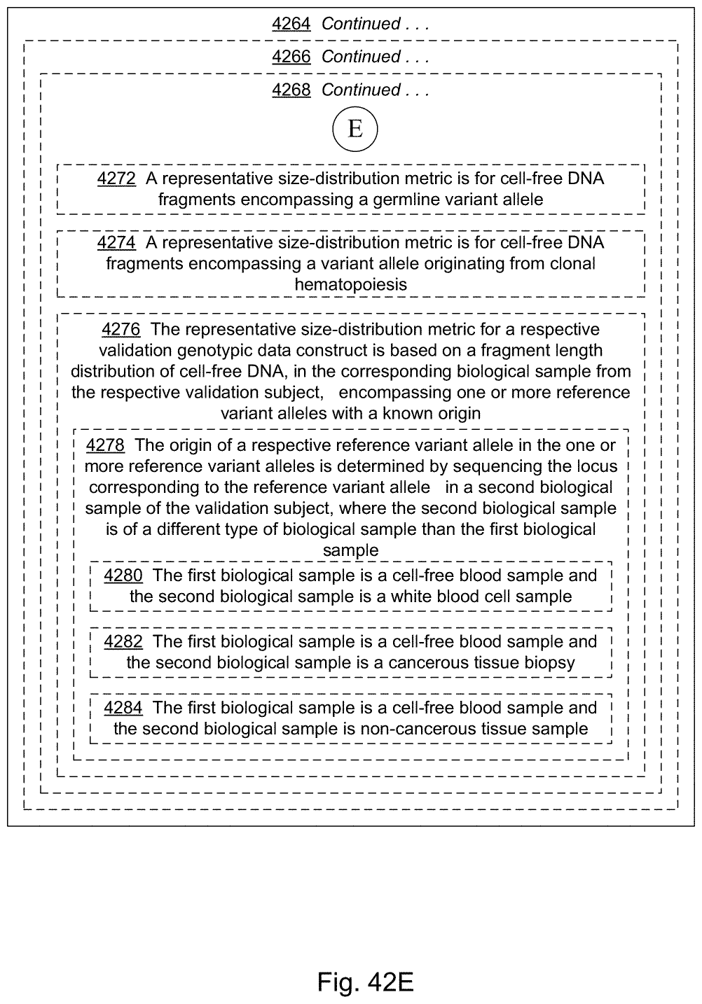

FIGS. 42A-42E are flow diagrams illustrating a method 4200 for validating the use of genotypic data from a particular genomic locus in a subject classifier for classifying a cancer condition for a species using a measure of the distribution of DNA fragment lengths of cell-free DNA fragments isolated from the blood of the subject which encompass an allele of interest. Method 4200 is performed at a computer system (e.g., computer system 100 or 150 in FIG. 1) having one or more processors, and memory storing one or more programs for execution by the one or more processors for phasing alleles present on a matching pair of chromosomes in a cancerous tissue of a subject. Some operations in method 4200 are, optionally, combined and/or the order of some operations is, optionally, changed.

In some embodiments, method 4200 is performed at a computer system comprising one or more processors, and memory storing one or more programs for execution by the one or more processors. The method includes obtaining (4204) a subject classifier that uses data from the particular genomic locus to classify the cancer condition for a query subject of the species (e.g., that was trained against one or more genotypic characteristics from a plurality of training genotypic data constructs obtained for a plurality of training subjects of the species with a known cancer status).

In some embodiments, the subject classifier is trained against one or more genotypic characteristics from a plurality of training genotypic data constructs obtained from a plurality of training subjects of the species with a known cancer status, and wherein the one or more genotypic characteristics do not include a size-distribution metric corresponding to a characteristic of the distribution of fragments lengths of cell-free DNA encompassing the genomic locus in samples from the training subjects (4206). That is, in some embodiments, because the classifier is not trained using data on the distribution of fragment lengths of cell-free DNA, this type of data can be used as an orthogonal source of data to evaluate the fitness of the trained classifier, since this type of data is not related to other types of data used to build cancer classifiers. For example, in some embodiments, the classifier is trained against one or more types of gene expression data (e.g., mRNA abundance assayed by microarray, qPCR, hybridization, mass spectroscopy or microRNA abundance assayed using a similar technique), proteomic data (e.g., protein expression data assayed by microarray, immunoassay, mass spectroscopy, etc.), genomic data (e.g., variant allele analysis, copy number analysis, read depth analysis, allelic ratio analysis, etc.), and/or epigenetic data (e.g., methylation analysis, histone modification analysis, etc.).

In some embodiments, each respective training genotypic data construct in the plurality of training genotypic data sets is obtained from a corresponding training (e.g., second) plurality of nucleic acid fragment sequences in electronic form from a corresponding biological sample from a respective training subject in the plurality of training subjects, where each respective nucleic acid fragment sequence in the corresponding training (e.g., second) plurality of nucleic acid fragment sequences represents all or a portion of a respective cell-free DNA molecule in a population of cell-free DNA molecules in the corresponding biological sample, the respective nucleic acid fragment sequence encompassing a corresponding locus, in a plurality of loci, represented by at least two different alleles (e.g., a reference allele sequence and a variant allele sequence, where the allele is a SNP, insertion, deletion, inversion, etc.) within the population of cell-free DNA molecules (e.g., originating from at least cancerous cells, non-cancerous somatic cells, and white blood cells).

The subject classifier may provide any type of diagnostic or prognostic evaluation of the cancer condition of a subject. For instance, in some embodiments, the cancer condition classified by the subject classifier is a primary origin of a cancer (4210). In some embodiments, the cancer condition classified by the subject classifier is a stage of a cancer (4212). In some embodiments, the cancer condition classified by the subject classifier is an initial cancer diagnosis (4214). In some embodiments, the cancer condition classified by the subject classifier is a cancer prognosis (4216), e.g., a prognosis as to growth or spread of the cancer, a life expectancy, an expected response to a therapy, etc. Many classifiers for providing diagnostic or prognostic information about a cancer conditions are known in the art.

In some embodiments, the subject classifier provides diagnostic and/or prognostic information for one or more cancers selected from a breast cancer, a lung cancer, a prostate cancer, a colorectal cancer, a renal cancer, a uterine cancer, a pancreatic cancer, an esophageal cancer, a lymphoma, a head/neck cancer, an ovarian cancer, a hepatobiliary cancer, a melanoma, a cervical cancer, a multiple myeloma, a leukemia, a thyroid cancer, a bladder cancer, a gastric cancer, or a combination thereof.

Method 4200 includes obtaining (4218) for each respective validation subject in a plurality of validation subjects of the species: (i) a cancer condition and (ii) a validation genotypic data construct that includes one or more genotypic characteristics, thereby obtaining a set of cancer conditions and a correlated set of validation genotypic data constructs. Each genotypic data construct in the set of genotypic data constructs is obtained from a respective validation (e.g., first) plurality of nucleic acid fragment sequences in electronic form from a corresponding validation (e.g., first) biological sample from a respective validation subject in the plurality of validation subjects. Each respective nucleic acid fragment sequence in the respective validation (e.g., first) plurality of nucleic acid fragment sequences represents all or a portion of a respective cell-free DNA molecule in a population of cell-free DNA molecules in the corresponding biological sample, the respective nucleic acid fragment sequence encompassing a corresponding locus, in a plurality of loci, represented by at least two different alleles within the population of cell-free DNA molecules. In some embodiments, the at least two different alleles are two different germline alleles, e.g., two different reference alleles found at the loci of respective maternal and paternal chromosomes within the germline of the subject, or one reference allele and one variant allele found at the loci of respective maternal and paternal chromosomes within the germline of the subject. In some embodiments, the at least two different alleles include a reference or variant allele represented within the germline of the subject and a variant allele arising from a cancerous tissue of the subject, at the respective locus. The one or more genotypic characteristics in the validation genotypic data construct include a size-distribution metric corresponding to a characteristic of the distribution of the fragment lengths of the cell-free DNA molecules that encompass a respective allele of the particular genomic locus. Because a set of size-distribution metrics is smaller than the set of individual nucleic acid fragment sequences, use of the size-distribution metrics, rather than the full data set, compresses the data in order to make the method more computationally efficient, e.g., by allowing the computer to apply an algorithm to the smaller dataset (the set size distribution metrics) rather than the full dataset (the nucleic acid fragment sequences themselves). In one embodiment, the size-distribution metric is a measure of central tendency of length across the distribution (4260). In some embodiments, the measure of central tendency of length across the distribution is an arithmetic mean, weighted mean, midrange, midhinge, trimean, Winsorized mean, median, or mode of the distribution (4262).

For example, as described above, it is known that mono- and di-nucleosomes fragmented from the genomes of non-cancerous somatic cells, hematopoietic cells (e.g., white blood cells), and (when the subject has cancer) cancerous cells. Thus, in some embodiments, the cell-free DNA molecules in a respective validation sample originate from at least non-cancerous somatic cells and hematopoietic cells (e.g., white blood cells). In some embodiments, the validation sample also includes cell-free DNA molecules originating from cancerous cells. In some embodiments, the validation subject has already been diagnosed with cancer (4232) and, accordingly, it is known that the cell-free DNA originating from cancerous cells is present in the sample prior to analysis. In some embodiments, the validation subject is a human (4234).

In some embodiments, the obtaining step of the method includes collecting (4202) a plurality of sequencing reads from cell-free DNA in a plurality of validation biological samples from a plurality of validation subjects using a nucleic acid sequencer. However, in other embodiments, method 4200 only includes obtaining the sequencing data from prior sequencing reactions of cell-free DNA from the plurality of validation biological samples.

Methods for collecting suitable sequencing data for the methods described herein (e.g., method 4200) are described above, and are not reiterated here for reasons of brevity. Regardless of the exact sequencing method used, however, in some embodiments, each respective nucleic acid fragment sequence in the plurality of nucleic acid fragment sequences is obtained by generating complementary sequence reads from both ends of a respective cell-free DNA molecule in the population of cell-free DNA (4220), where the complementary sequence reads are combined to form a respective sequence read, which is collapsed with other respective sequence reads of the same unique nucleic acid fragment to form the respective nucleic acid fragment sequence. For example, in some embodiments, complementary sequence reads are stitched together based on an overlapping region of sequence shared between the complementary sequence reads and/or by matching the sequences from complementary sequence reads to corresponding sequences in a reference genome for the species of the subject.

In some embodiments, the first biological sample from a respective validation subject is a blood sample (4222), e.g., a whole-blood sample, a blood serum sample, or a blood plasma sample. In some embodiments, the blood sample is a whole blood sample, and prior to generating the plurality of nucleic acid fragment sequences from the whole blood sample, white blood cells are removed from the whole blood sample (4224). In some embodiments, the white blood cells are collected as a second type of sample, e.g., according to a buffy coat extraction method, from which additional sequencing data may or may not be obtained. In some embodiments, the method further includes obtaining (4226) a third plurality of nucleic acid fragment sequences in electronic form of genomic DNA from the white blood cells removed from the validation whole blood sample. In some embodiments, the third plurality of nucleic acid fragment sequences is used to identify allele variants arising from clonal hematopoiesis, as opposed to germline allele variants and/or allele variants arising from a cancer in the subject. Likewise, in some embodiments, fragment length distributions obtained for fragments encompassing an allele are used to seed a classification algorithm, e.g., an expectation maximization (EM) algorithm. In some embodiments, the blood sample is a blood serum sample (4228).

In some embodiments, the plurality of loci are selected from a predetermined set of loci that includes less than all loci in the genome of the subject (4234). In some embodiments, nucleic acid fragment sequences of the cell-free DNA molecules in the sample are generated for a predetermined set of loci, e.g., by targeted panel sequencing. As described above, many targeted panels for sequencing alleles of interest, e.g., related to cancer diagnostics, are known to those of skill in the art. Although not reiterated here for reasons of brevity, any of these targeted panels can be used in the methods described herein. In some embodiments, the targeted panel includes loci known to provide diagnostic or prognostic power for cancer diagnostics, e.g., loci at which an allele has been linked to a characteristic of a cancer. In some embodiments, the targeted panel includes alleles that are distributed throughout the genome of the species of the subject, e.g., to provide representation for a large portion of the genome.

In some embodiments, the predetermined set of loci includes at least 100 loci (4236). In some embodiments, the predetermined set of loci includes at least 500 loci (4238). In some embodiments, the predetermined set of loci includes at least 1000 loci (4240). In some embodiments, the predetermined set of loci includes at least 5000 loci (4242). In some embodiments, the predetermined set of loci includes at least 100, 200, 300, 400, 500, 600, 700, 800, 900, 1000, 2000, 3000, 4000, 5000, 6000, 7000, 8000, 9000, 10,000, 15,000, 20,000, 25,000, 50,000, 75,000, 100,000, or more loci. In some embodiments, the predetermined set of loci includes from 100 to 100,000 loci, from 100 to 50,000 loci, from 100 to 25,000 loci, from 100 to 10,000 loci, from 100 to 5000 loci, from 100 to 2000 loci, from 100 to 1000 loci, from 500 to 100,000 loci, from 500 to 50,000 loci, from 500 to 25,000 loci, from 500 to 10,000 loci, from 500 to 5000 loci, from 500 to 2000 loci, from 500 to 1000 loci, from 1000 to 100,000 loci, from 1000 to 50,000 loci, from 1000 to 25,000 loci, from 1000 to 10,000 loci, from 1000 to 5000 loci, or from 1000 to 2000 loci.

In some embodiments, the average coverage rate of nucleic acid fragment sequences of the predetermined set of loci taken from the sample is at least 25× (4244). In some embodiments, the average coverage rate of nucleic acid fragment sequences of the predetermined set of loci taken from the sample is at least 50×, 100×, 200×, 300×, 400×, 500×, 750×, 1000×, 2000×, 3000×, 4000×, 5000×, or more. In some embodiments, the average coverage rate of nucleic acid fragment sequences of the predetermined set of loci taken from the sample is from 25× to 5000×, from 25× to 2500×, from 25× to 1000×, from 25× to 500×, from 25× to 100×, from 100× to 5000×, from 100× to 2500×, from 100× to 1000×, or from 100× to 500×.

In some embodiments, plurality of loci are selected from all loci in the genome of the subject (4246), e.g., all of the cell-free DNA molecules in the sample are sequenced, e.g., by whole genome sequencing, and nucleic acid fragment sequences corresponding to cell-free DNA molecules encompassing the predetermined set of loci are selected for the analysis. As described above, many methods for whole genome sequencing are known to those of skill in the art. In some embodiments, the average coverage rate of nucleic acid fragment sequences across the genome of the subject is at least 10× (4248). In some embodiments, the average coverage rate of nucleic acid fragment sequences across the genome of the subject is at least 25×, 50×, 100×, 200×, 300×, 400×, 500×, 750×, 1000×, or more. In some embodiments, the average coverage rate of nucleic acid fragment sequences of the predetermined set of loci taken from the sample is from 10× to 1000×, from 10× to 500×, from 10× to 100×, from 10× to 50×, from 50× to 1000×, from 50× to 500×, or from 50× to 100×.

In some embodiments, the at least two different alleles of a respective locus include a reference allele and a variant allele. In some embodiments, the at least two different alleles of a respective locus include a variant allele that is a single nucleotide polymorphism relative to a reference allele for the locus (4250). In some embodiments, the preceding claims, wherein the at least two different alleles of a respective locus include a variant allele that is a deletion of twenty-five nucleotides or less, encompassing the respective locus, relative to a reference allele for the locus (4252). In some embodiments, the at least two different alleles of a respective locus include a variant allele that is a single nucleotide deletion relative to a reference allele for the locus (4254). In some embodiments, the at least two different alleles of a respective locus include a variant allele that is an insertion of twenty-five nucleotides or less, encompassing the respective locus, relative to a reference allele for the locus (4256). In some embodiments, the at least two different alleles of a respective locus include a variant allele that is a single nucleotide insertion relative to a reference allele for the locus (4258).

Method 4200 also includes determining (4264) a confidence metric for use of genotypic data from the particular genomic locus in the subject classifier by using a parametric or non-parametric based test classifier that evaluates the size distribution metric for the respective allele in each respective validation genotype data construct and each correlated cancer status in the set of cancer conditions.

In some embodiments, the parametric or non-parametric based classifier is an expectation maximization algorithm (4266). In some embodiments, the expectation maximization algorithm is seeded with at least a representative size-distribution or size distribution metric for cell-free DNA fragments encompassing a variant allele originating from a known source (4268). In some embodiments, a representative size-distribution metric is for cell-free DNA fragments encompassing a variant allele originating from a cancerous tissue (4270). In some embodiments, a representative size-distribution metric is for cell-free DNA fragments encompassing a germline variant allele (4272). In some embodiments, a representative size-distribution metric is for cell-free DNA fragments encompassing a variant allele originating from clonal hematopoiesis (4274). In some embodiments, the representative size-distribution metric is based on a fragment length distribution of cell-free DNA in the sample encompassing one or more reference variant alleles with a known origin (4276).

In some embodiments, the origin of a reference variant allele is determined by sequencing the locus corresponding to the reference variant allele in a second biological sample from the validation subject, where the second biological sample is a different type of biological sample than the first biological sample (4278). In some embodiments, the first biological sample is a cell-free blood sample and the second biological sample is a white blood cell sample (4280). For instance, in some embodiments, a blood sample containing at least blood serum and white blood cells is collected from the validation subject, the white blood cells are removed from the sample (e.g., via buffy coat extraction), and loci of interest are sequenced in both the cell-free portion and the white blood cell portion of the original sample (e.g., which were separated from each other). Accordingly, variant alleles sequenced in the cell-free portion of the sample, which do not originate from the germline of the validation subject and which match variant alleles sequenced in the white blood cell sample can be positively identified as originating from clonal hematopoiesis, and can be used to seed the expectation maximization algorithm. In some embodiments, the first validation biological sample is a cell-free blood sample and the second validation biological sample is a cancerous tissue biopsy (4282). For instance, in some embodiments, a blood sample and a tumor biopsy are collected from the validation subject, and loci of interest are sequenced from both samples. Accordingly, variant alleles sequenced in the cell-free portion of the sample, which do not originate from the germline of the validation subject and which match variant alleles sequenced in the tumor biopsy can be positively identified as originating from cancerous tissue in the validation subject, and can be used to seed the expectation maximization algorithm. In some embodiments, the first biological sample is a cell-free blood sample and the second biological sample is non-cancerous tissue sample (4284). For instance, in some embodiments, a blood sample and a non-cancerous tissue sample are collected from the validation subject, and loci of interest are sequenced from both samples. Accordingly, variant alleles sequenced in the cell-free portion of the validation sample, which match variant alleles sequenced in the non-cancerous validation tissue sample can be positively identified as originating from the germline of the validation subject, and can be used to seed the expectation maximization algorithm.

### EXAMPLES

The data used in the analyses presented in Examples 1-13 below was collected in conjunction with Memorial Sloan Kettering Cancer Center (MSKCC). Briefly, cell-free DNA was isolated from blood samples collected from approximately 250 cancer subjects, about 50 subjects confirmed to have each of the following cancers: metastatic breast cancer, metastatic lung cancer, metastatic prostate cancer, early breast cancer, and early lung cancer. Blood samples from 50 subjects not having cancer were used as controls in the analyses. A custom DNA capture panel was used to sequence the isolated cell-free DNA fragments containing over 500 loci of interest.

For most of the blood samples, white blood cells were isolated using a buffy coat separation method. Genomic preparations from the white blood cells were then sequenced to provide a matching nucleic acid fragment sequences of the loci of interest, e.g., for positive assignment of sequence variants arising from clonal hematopoiesis. For many of the subjects, matching tissue biopsies and/or samples of non-cancerous tissue (e.g., collected via buccal swab or saliva sample) were also collected and sequenced to provide matching nucleic acid fragment sequences of the loci of interest, e.g., for positive assignment of sequence variants arising from cancerous tissue or from within the germline.

### Example 1—Identification of Tumor-Matched Single Nucleotide Variants

The distribution of cell-free DNA fragment lengths was investigated to determine whether it could be used to determine, and thereby assign, the origin of a cancer-derived variant allele. The basic model is that cell-free DNA fragments containing a reference allele are a mixture of tumor-derived and non-tumor derived DNA fragments, however, since cancer normally has one mutated chromosome at a given allele, cell-free DNA fragments containing a variant allele that originated from the cancerous tissue are a pure population that is derived only from cancer cells. Thus, if there is any difference in the length of DNA fragments that originate from cancers, as compared to the length of DNA fragments that originate from non-cancerous cells, the difference would manifest itself as a difference in the distribution of fragment-lengths of fragments containing a reference allele as compared to the distribution of fragment-lengths of fragments containing a variant allele originating from a cancerous tissue.

Targeted, capture-based DNA sequencing of cell-free DNA in one blood sample from a subject confirmed to have metastatic prostate cancer were generated and mapped to a reference genome using the Pecan alignment program (Patent, B., et al., Genome Res., 18(11):1814-28 (2008), the content of which is incorporated by reference herein, in its entirety, for all purposes). Single nucleotide variants (SNVs) detected at the loci of interest were identified in the sequencing data. Genomic DNA in biopsy tissue obtained from the subject was also sequenced, and SNVs detected in the biopsy tissue were matched to SNVs detected in the cell-free DNA obtained from the blood sample, allowing positive identification of seven SNVs originating from cancerous tissue.

Because the cell-free DNA fragments are derived from mono-nucleosome and di-nucleosome constructs in the blood, the data was then filtered to include only nucleic acid fragment sequences having a length of 210 nucleotides or less. This was done to reduce the contribution of fragments derived from di-nucleosome fragments. Briefly, mono-nucleosome derived cell-free DNA fragments have a normal distribution peak around 160 nucleotides, while di-nucleosome derived cell-free DNA fragments peak have a normal distribution centered around 300 nucleotides. However, because of readout of the sequencing sensor is censored at 288 nucleotides, the peak of the distribution of fragment lengths from di-nucleosome derived fragments is not represented in the raw data.

Further, limiting the data to substantially fragment lengths derived from mono-nucleosomal constructs facilitates easier manual evaluation of fragment length shifts. However, for sequencing methodologies that sequence from both ends of the fragment molecule, it is possible to estimate the length of DNA fragments that are longer than the sensor readout by matching the ends of complementary fragments to a reference genome and determining the distance between the ends of the two sequence reads. Moreover, computational analysis of mixture of mono-nucleosomal and di-nucleosomal derived DNA fragments can be completed just as readily as analysis of data only corresponding to mono-nucleosomal derived DNA fragments.

The lengths of the cell-free DNA fragments, filtered to 210 nucleotides or less, containing the loci that correspond to the SNVs identified as originating from cancerous tissue were then cumulatively plotted as either containing a variant allele (i.e., the biopsy matched SNV) (202) or containing a reference allele (204), as illustrated in FIG. 2. As can be seen from FIG. 2, on average, the length of cell-free DNA fragments containing a variant allele, which is known to originate from a cancer cell, are shorter on median than cell-free DNA fragments originating from a normal distribution of cell-free DNA fragments which are a mixture of fragments originating from normal somatic cells, cancer cells, and white blood cells, as represented by nucleic acid fragment sequences containing a reference allele (204) at the locus. Thus, this experiment suggests that variant alleles arising from a cancerous tissue can be identified as originating from a cancerous tissue by identifying a shift shorter in the fragment length distribution of cell-free DNA molecules containing the variant allele, relative to the normal fragment length distribution of cell-free DNA molecules originating from a mixture of normal non-cancerous cells, cancer cells, and white blood cells.

### Example 2—Identification of Blood-Matched Clonal Hematopoiesis Variants

The distribution of cell-free DNA fragment lengths was investigated to determine whether it could be used to determine, and thereby assign, the origin of a variant allele originating from clonal hematopoiesis. The basic model is that cell-free DNA fragments containing a reference allele are a mixture of tumor-derived and non-tumor derived DNA fragments, however, since mutation arising from clonal hematopoiesis will result in a variant allele that is not present in the germline cells or the cancerous tissue, cell-free DNA fragments containing a variant allele that originated from clonal hematopoiesis are a pure population that is derived only from white blood cells. Thus, if there is a difference in the length of DNA fragments that originate from white blood cells, as compared to the length of DNA fragments that originate from non-cancerous germline and/or cancer cells, the difference would manifest itself as a difference in the distribution of fragment-lengths of fragments containing a reference allele as compared to the distribution of fragment-lengths of fragments containing a variant allele originating from a clonal hematopoiesis.

Targeted, capture-based DNA sequencing of cell-free DNA in a blood sample from a subject confirmed to have metastatic prostate cancer were generated and mapped to a reference genome using the Pecan alignment program. Single nucleotide variants (SNVs) detected at the loci of interest were identified in the sequencing data. Genomic DNA in white blood cells obtained from the subject was also sequenced, and SNVs detected in the white blood cells were matched to SNVs detected in the cell-free DNA obtained from the blood sample, allowing positive identification of thirteen SNVs originating from clonal hematopoiesis.

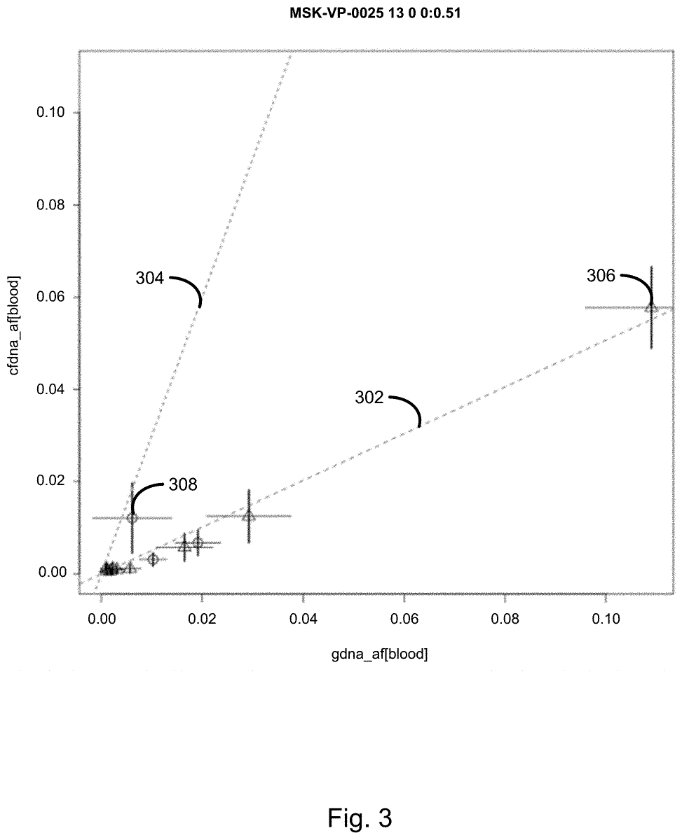

The allele-frequency of the thirteen blood-matched SNVs in the cell-free DNA sample was plotted against the allele-frequency of the thirteen blood-matched SNVs in the white blood cell sample, as illustrated in FIG. 3.

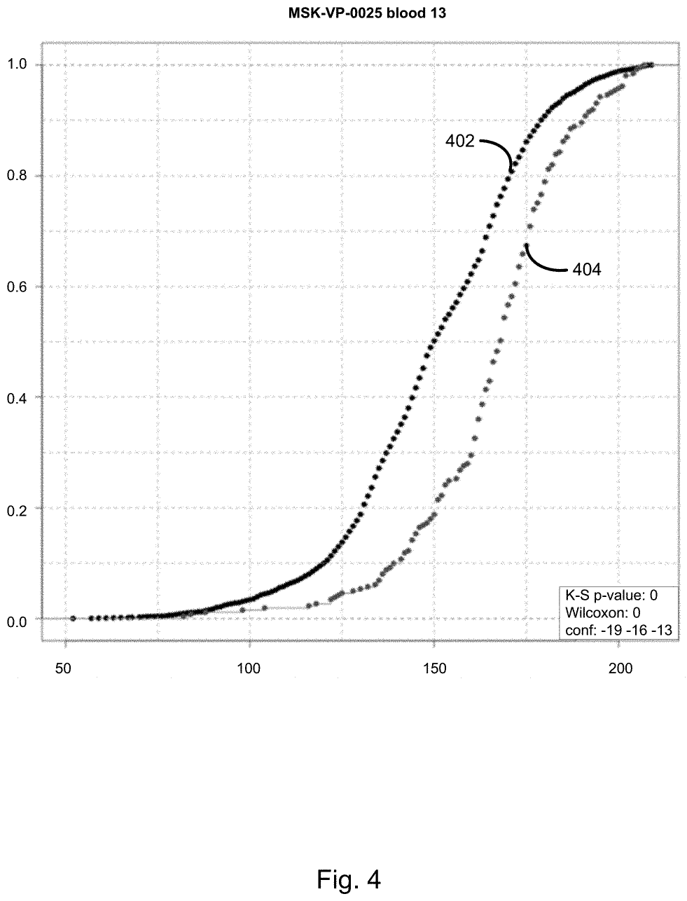

The lengths of the cell-free DNA fragments, filtered to 210 nucleotides or less (as discussed in Example 1), containing the loci that correspond to the SNVs identified as originating from clonal hematopoiesis were then cumulatively plotted as either containing a variant allele (i.e., a white blood cell matched SNV) (404) or containing a reference allele (402), as illustrated in FIG. 4. As can be seen from FIG. 4, on average, the length of cell-free DNA fragments containing a variant allele, which is known to originate from clonal hematopoiesis (404), are longer on median than cell-free DNA fragments originating from a normal distribution of cell-free DNA fragments which are a mixture of fragments originating from normal somatic cells, cancer cells, and white blood cells, as represented by nucleic acid fragment sequences containing a reference allele (402) at the locus. Thus, this experiment suggests that variant alleles arising from clonal hematopoiesis can be identified as originating from clonal hematopoiesis by identifying a shift longer in the fragment length distribution of cell-free DNA molecules containing the variant allele, relative to the normal fragment length distribution of cell-free DNA molecules originating from a mixture of normal non-cancerous cells, cancer cells, and white blood cells.

### Example 3—Fragment-Length Evaluation of Germline-Derived Variant Alleles

The distribution of fragment lengths of cell-free DNA fragment encompassing germline-derived variant alleles from a cancer patient was investigated to determine whether any information about the patient's cancer could be determined. Because germline alleles should be represented equally in a tumor, it could be expected that the distribution of fragment lengths of cell-free DNA—which is derived from a mixture of germline cells, white blood cells, and cancer cells in a patient with cancer—should be the same for reference allele as for the variant allele. On average, this hypothesis was borne out by the data.

Targeted, capture-based DNA sequencing of cell-free DNA in a blood sample from a subject confirmed to have metastatic prostate cancer were generated and mapped to a reference genome using the Pecan alignment program. Single nucleotide variants (SNVs) detected at the loci of interest were identified in the sequencing data. Genomic DNA obtained from a non-cancerous sample obtained from the subject was also sequenced, and SNVs detected in the normal (“germline”) genome were matched to SNVs detected in the cell-free DNA obtained from the blood sample, allowing positive identification of 785 SNVs originating from the germline of the patient.

The lengths of the cell-free DNA fragments, filtered to 210 nucleotides or less (as discussed in Example 1), containing the loci that correspond to the SNVs identified as originating from the germline of the subject were then cumulatively plotted as either containing a variant allele (i.e., a germline matched SNV) (504) or containing a reference allele (502), as illustrated in FIG. 5. As can be seen from FIG. 5, on average, the distribution of lengths of cell-free DNA fragments containing a germline allele is the same regardless of whether the DNA fragment contains a reference (502) or variant (504) allele, as expected by the model.

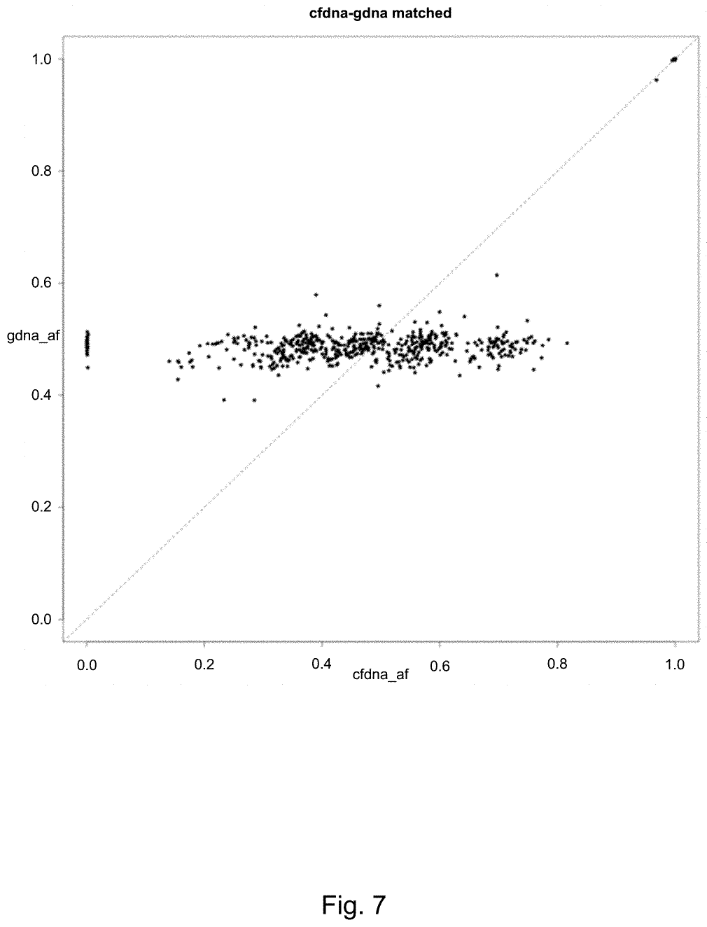

However, when the allele frequencies of individual germline alleles are plotted, a very different pattern is revealed for the allele frequency of germline alleles in cell-free DNA than the allele frequency of germline alleles in white blood cells. Briefly, as shown in FIG. 6, the allele frequency of germline alleles at different positions along the genome in white blood cells is roughly 50:50 for all germline alleles (602; open circles). Copy number aberrations in cancer cells can also been seen by plotting the allele frequency of the germline alleles in cell-free DNA against the allele frequency of the same allele in white blood cells, as shown in FIG. 7.

However, the allele frequency of germline alleles in cell-free DNA is highly variable (604; closed circles), depending upon the position of the allele along the genome. Further, it appears that the magnitude of the shift in allele frequency away from 50:50 (e.g., the distance between an axis representing a 50:50 distribution of alleles and the allele frequency plotted for any particular allele) is dependent upon which chromosome the allele resides. For example, as shown in FIG. 6, the allele frequency of germline alleles, as measured in cell-free DNA, residing on chromosome 10 is tightly clustered around 50:50. By contrast, the allele frequency of germline alleles, as measured in cell-free DNA, residing on chromosome 7 is skewed, either upwards or downwards, by 20-25% away from the 50:50 distribution. Similarly, the allele frequency of germline alleles, as measured in cell-free DNA, residing on chromosome 10 is also skewed away from the 50:50 distribution, but only by about 10%.

The allele-frequency skew away from a theoretical 50:50 distribution is explained by copy number aberrations in cancerous cells, i.e., the loss and/or gain of individual chromosomes or regions of chromosomes in cancerous cells. Because the genomes of individual cancer cells vary, even within a single tumor, the percentage of cancer cells that contain a copy number aberration with respect to any one chromosome is variable. This suggests that when a higher percentage of cancer cells lose or gain a chromosome, the shift in the allele frequency of alleles located on that chromosome, as measured in cell-free DNA, will become more pronounced and can be visualized by plotting the allele-frequencies as a function of position within the genome, as shown in FIG. 6. This experiment, thus, suggests that information about relative chromosome copy number aberrations in the population of cancer cells in a patient can be derived from determining the allele frequency of germline alleles along the various chromosomes. For example, the data presented in FIG. 6 indicates that a higher number of cancer cells in this particular patient have lost or gained one copy of chromosome 7 than the number of cancer cells in the patient that have lost or gained chromosome 9. Moreover, this data suggests that very few of the cancer cells in this patient have lost or gained a copy of chromosome 10, because the allele ratio of germline alleles along chromosome 10 is approximately 50:50.

It was next determined whether cell-free DNA fragments encompassing loci that displayed shifts in allele-frequency away from a 50:50 distribution also demonstrate variations in fragment length. Briefly, the lengths of cell-free DNA fragments, filtered to 210 nucleotides or less, containing individual loci that correspond to two of the SNVs identified as originating from the germline (T116382034A located on chromosome 7 and A12011772G located on chromosome 12), and found to have allele frequency shifts of approximately the same magnitude in opposite directions (allele frequencies of 0.6905 and 0.3058, respectively) were plotted as either containing a variant allele (i.e., the germline matched SNV) (802 and 904) or containing a reference allele (804 and 902), as illustrated in FIGS. 8 and 9. As can be seen from these figures, shifts in the distribution of fragment lengths occur in fragments containing either the reference allele or the variant allele. However, unlike the case with cancer-matched and white blood cell-matched SNVs, the fragment-length shift demonstrated with germline-matched SNVs cannot be predicted based on which set of fragments contain the variant allele.

For instance, cell-free DNA fragments containing the variant allele at position 116382034 on chromosome 7 have a fragment-length distribution (802) that is shifted smaller relative to cell-free DNA fragments containing the reference allele at position 116382034 on chromosome 7 (804). In contrast, cell-free DNA fragments containing the reference allele at position 12011772 on chromosome 12 have a fragment-length distribution (902) that is shifted smaller relative to cell-free DNA fragments containing the variant allele at position 12011772 on chromosome 12 (904).

The shifts in fragment-length distribution may be explained here, not by the origin of the variant allele, but instead by losses of heterozygosity within cancer cells in the patient. In one model, when cancer cells, which were shown to generate cell-free DNA fragments having shorter lengths, lose heterozygosity at a particular locus (e.g., by loss of a chromosome or portion of a chromosome that includes the locus), the cell-free DNA fragments in the subject containing the allele that was lost in the cancer cells includes cell-free DNA fragments from non-cancerous germline cells and white blood cells, but not cancer cells. In contrast the cell-free DNA fragments in the subject containing the allele that was not lost in the cancer cells includes cell-free DNA fragments from non-cancerous germline cells, white blood cells, and cancer cells. Thus, the distribution of fragment-lengths of cell-free fragments containing the allele that was not lost in the cancer cells is shifted shorter, relative to the distribution of fragment-lengths of cell free fragments containing the allele that was lost in the cancer cells, because of the contribution of shorter fragments originating from the cancer cells. Thus, this experiment suggests that loss of heterozygosity at a particular locus in a cancer can be identified by detecting a shift in the lengths of cell-free DNA encompassing one germline allele at the locus relative to the lengths of cell-free DNA encompassing the other germline allele at the locus. Further, the experiment suggests that the identity of the germline allele that was lost in the cancer can be identified by detecting an apparent shift shorter in the fragment lengths of cell-free DNA encompassing the other germline allele at the locus.

Similarly, in a non-mutually exclusive model, when cancer cells gain a copy of a particular locus (e.g., by gaining a chromosome or duplication of a portion of a chromosome), a higher proportion of cell-free DNA fragments in the subject will encompass the allele that was gained than the proportion of cell-free DNA fragments that encompass the other germline allele represented at the locus (e.g., the allele that was not gained in the cancer cells). Thus, the distribution of fragment-lengths of cell-free fragments containing the allele that was gained in the cancer cells is shifted shorter, relative to the distribution of fragment-lengths of cell free fragments containing the allele that was not gained in the cancer cells, because of the higher contribution of shorter fragments originating from the cancer cells. Thus, this experiment suggests that gain of a particular locus in a cancer can be identified by detecting a shift in the lengths of cell-free DNA fragments encompassing one germline allele at the locus relative to the lengths of cell-free DNA fragments encompassing the other germline allele at the locus. Further, the experiment suggests that the identity of the germline allele that is gained in the cancer can be identified by detecting an apparent shift shorter in the fragment lengths of cell-free DNA fragments encompassing the allele.

Further evidence that shifts in fragments lengths correlate with shifts in allele-frequency, due to chromosomal number aberrations (e.g., gains and losses) is seen when mean fragments lengths of the reference and variant germline alleles are plotted as a function of their position in the genome, as shown in FIG. 10, where mean fragment length of fragments encompassing the reference germline allele are shown as closed, black circles and mean fragment length of fragments encompassing the variant germline allele are shown as open, red circles. As can be seen in FIG. 10, the pattern of fragment-length shift across the genome appears to match the pattern of allele-frequency shift, as shown in FIG. 6. For example, significant shifts in fragment lengths are shown for loci located on chromosome 7 in FIG. 10, like the significant shifts in allele-frequency shown for loci located on chromosome 7 in FIG. 6. Similarly, no significant shift in fragment lengths are shown for loci located on chromosome 10 in FIG. 10, like no significant shifts in allele-frequency were seen for loci located on chromosome 10 in FIG. 6.

This is also shown in FIG. 11, where shifts in the allele-frequency of the reference allele at loci identified to include a germline variant are plotted as a function of the mean shift in the lengths of cell-free DNA fragments encompassing the variant allele, relative to the mean lengths of cell-free DNA fragments encompassing the reference allele. The data appear to show five distinct clusters of loci, which represent loci at which cancer cells have lost a chromosomal copy of the reference allele (1102), loci at which cancer cells have gained a copy of the variant allele (1104), loci at which cancer cells have not gained or lost a copy of either allele, or alternatively have gained or lost of copy of both alleles (1106), loci at which cancer cells have gained a copy of the reference allele (1108), and loci at which cancer cells have lost a copy of the variant allele (1110).

Further, the fragment-length shift information can be used to determine which alleles are present together on the same chromosome in the cancer based on which fragment-length distributions are similar to each other. That is, the alleles present at nearby loci on each chromosome can be phased together by determining whether the fragment length distribution for either the reference allele or germline variant allele at a first locus is more similar to the fragment-length distribution of the reference allele or the germline allele at the second locus, because alleles that are genetically linked should be lost or gained together when a chromosomal aberration event occurs, e.g., when a chromosome or part of a chromosome is lost or gained in the cancer. As proof of this, the allele ratio, which is defined in FIG. 6 as the frequency of the reference allele divided by the frequency of the variant allele, is defined in FIG. 12 as the frequency of the allele corresponding to the cell-free DNA fragments encompassing the corresponding loci that have the shorter distribution of fragment-lengths (regardless of whether it is the reference allele or the germline variant allele) divided by the frequency of the allele corresponding to the cell-free DNA fragments encompassing the corresponding loci that have the longer distribution of fragment lengths. As is seen in FIG. 12, this definition results in a phasing of the alleles onto shared chromosomes, such that all of the allele-ratios are at or shifted above a 50:50 distribution, indicating the alleles with similar fragment-length distributions in cell-free DNA fragments are on the same chromosome. In FIG. 12, the allele frequency of germline alleles at different positions along the genome in white blood cells is roughly 50:50 for all germline alleles (1202; open circles). However, the allele frequency of germline alleles in cell-free DNA is highly variable (1204; closed circles), depending upon the position of the allele along the genome.

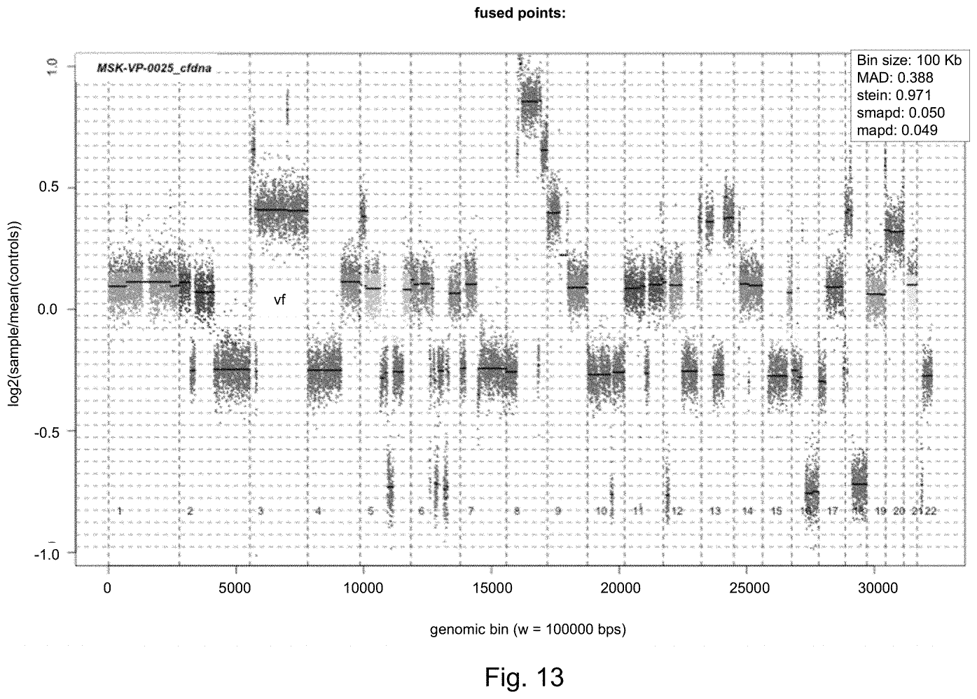

A genetic map, showing the relative density of read counts across the chromosomes indicative of their copy number, of the cancer genome of the subject used in this example is shown in FIG. 13.

### Example 4—Classification of Novel Somatic Variants

Targeted, capture-based DNA sequencing of cell-free DNA in a blood sample from a subject confirmed to have metastatic prostate cancer were generated and mapped to a reference genome, as described above. 807 single nucleotide variants (SNVs) detected at the loci of interest were identified in the sequencing data. These loci were also sequenced in genomic DNA from (i) a tumor biopsy (e.g., cancer cells) from the subject, (ii) white blood cells from the subject, and (iii) a non-cancerous tissue sample from the subject. The origin of the 807 SNVs identified in the cell-free DNA were then matched to the three tissue types, allowing identification of the origins of each of the variants, as described in Examples 1-3. Of the variant alleles, seven were identified as originating from cancer cells, 13 were identified as originating from clonal hematopoiesis (e.g., from white blood cells), and 785 were identified as originating from the germline. Two SNVs, however, were not matched to any of these sources. These two SNVs were used as a test case to determine whether their origin could be determined based on the fragment distribution of cell-free DNA encompassing the corresponding loci.

Briefly, when the lengths of cell-free DNA fragments encompassing loci associated with SNVs matched to a cancerous origin were cumulatively plotted as containing a variant allele (1402) or containing a reference allele (1404), the distribution of lengths matched the expected model, where cell-free DNA fragments encompassing the variant allele (1402) had smaller lengths on average than cell-free DNA fragments encompassing the reference allele (1404), as shown in FIG. 14A. Similarly, when the lengths of cell-free DNA fragments encompassing loci associated with SNVs matched to white blood cells were cumulatively plotted as containing a variant allele (1408) or containing a reference allele (1406), the distribution of lengths matched the expected model, where cell-free DNA fragments encompassing the variant allele (1408) had greater lengths on average than cell-free DNA fragments encompassing the reference allele (1406), as shown in FIG. 14B. Likewise, when the lengths of cell-free DNA fragments encompassing loci associated with SNVs matched to the germline were cumulatively plotted as containing a variant allele (1412) or containing a reference allele (1410), the distribution of lengths matched the expected model, where cell-free DNA fragments encompassing the variant allele (1412) had similar lengths on average to cell-free DNA fragments encompassing the reference allele (1410), as shown in FIG. 14C. When the lengths of cell-free DNA fragments encompassing the two loci associated with SNVs with an unidentified origin were cumulatively plotted as containing a variant allele (1414) or containing a reference allele (1416), it could be seen that the distribution of lengths of the cell-free DNA fragments encompassing the variant alleles (1414) was shifted shorter than the distribution of lengths of the cell-free DNA fragments encompassing the reference alleles (1416), as shown in FIG. 14D. This result is consistent with a hypothesis that the unidentified variants arose from cancer cells, because the shift in fragment lengths appears to be consistent with the model behavior expected of variant alleles arising from a cancer cell.

In order to validate the hypothesis that the two unmatched variants did arise from cancer cells, a mixture model was trained against the fragment length distribution of cell-free DNA encompassing the seven loci corresponding to the variant alleles that were positively matched to a cancer origin, as shown in FIG. 15, which include cell-free DNA fragments encompassing the variant allele (1502) and cell-free DNA fragments encompassing the reference allele (1504). An expectation maximization algorithm was then used to test the mixture model against the populations of cell-free DNA encompassing each of the 807 loci at which a single nucleotide variant was identified.

As shown in FIG. 16, the EM algorithm assigned a high level of responsibility to each of the seven loci corresponding to the biopsy-matched variants, as expected, indicating that these variant alleles originated from cancer cells. Consistently, the EM algorithm assigned a low level of responsibility to each of the 13 loci corresponding to the white-blood cell-matched variants, as expected, indicating that these variants did not originate from cancer cells. The EM algorithm provided a wide range of responsibilities for the 785 loci corresponding to germline-matched variants because, as demonstrated in Example 3, copy number variance of loci represented by a germline variant affect the fragment length distribution of cell-free DNA fragments encompassing these loci. Finally, the EM algorithm assigned a high level of responsibility to both of the loci corresponding to the unmatched variants, indicating that these variant alleles originated from cancer cells.

### Example 5—Classification of Novel Somatic Variants in a Subject with a Low Tumor Burden

Targeted, capture-based DNA sequencing of cell-free DNA in a blood sample from a subject confirmed to have metastatic cancer, but having a low tumor burden, were generated and mapped to a reference genome, as described above. 752 single nucleotide variants (SNVs) detected at the loci of interest were identified in the sequencing data. These loci were also sequenced in genomic DNA from (i) a tumor biopsy (e.g., cancer cells) from the subject, (ii) white blood cells from the subject, and (iii) a non-cancerous tissue sample from the subject. The origin of the 752 SNVs identified in the cell-free DNA were then matched to the three tissue types, allowing identification of the origins of each of the variants, as described in Examples 1-3. Of the variant alleles, seven were identified as originating from cancer cells, 10 were identified as originating from clonal hematopoiesis (e.g., from white blood cells), and 720 were identified as originating from the germline. 15 SNVs, however, were not matched to any of these sources. An expectation maximization algorithm was then used to determine whether these 15 unmatched variants originated from cancer cells, as described above.

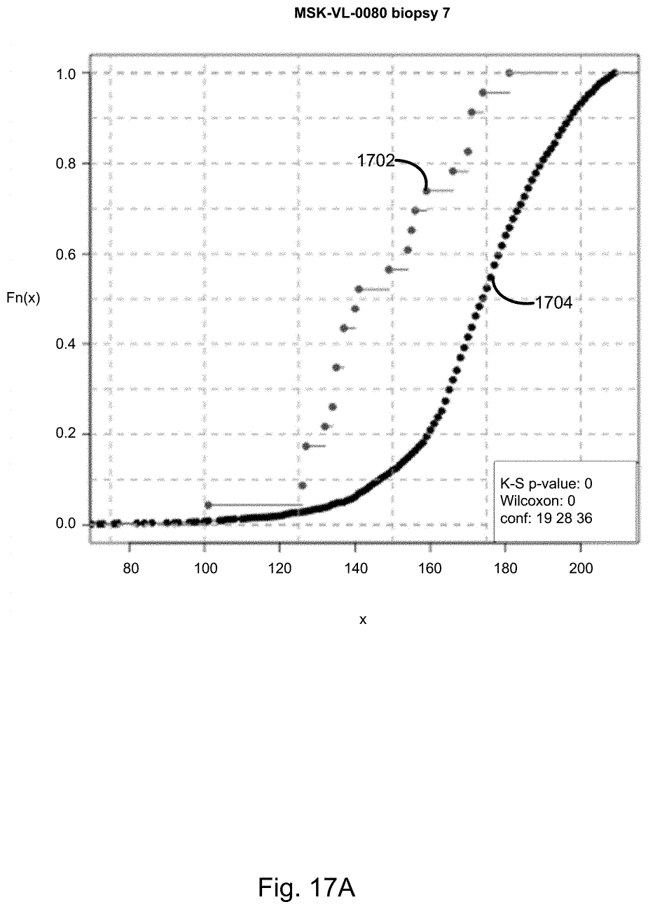

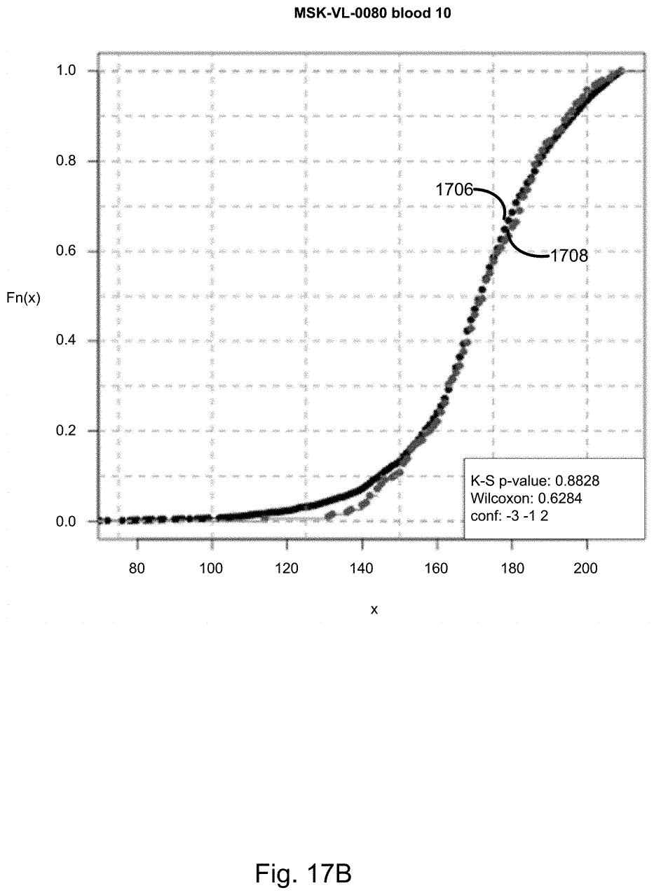

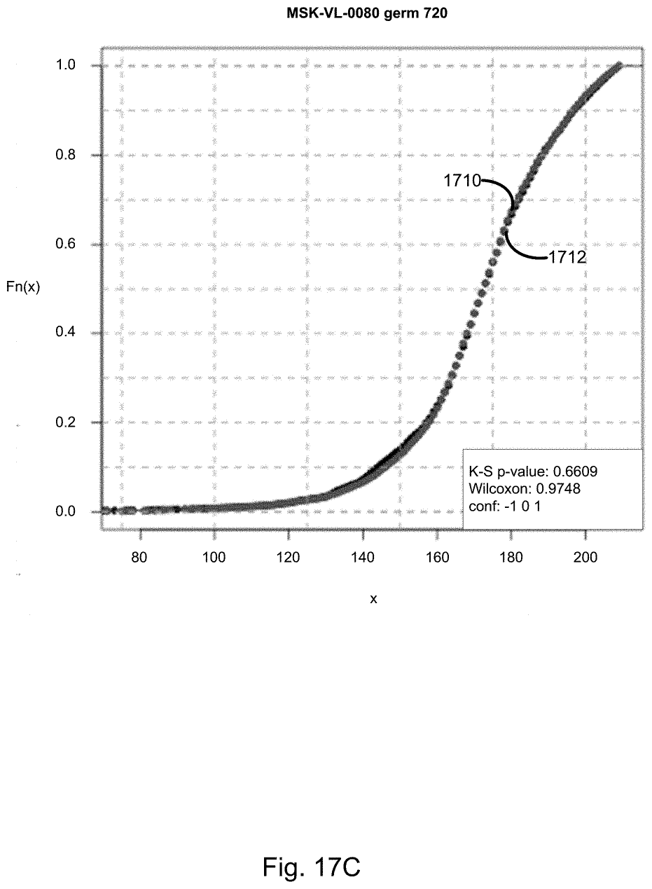

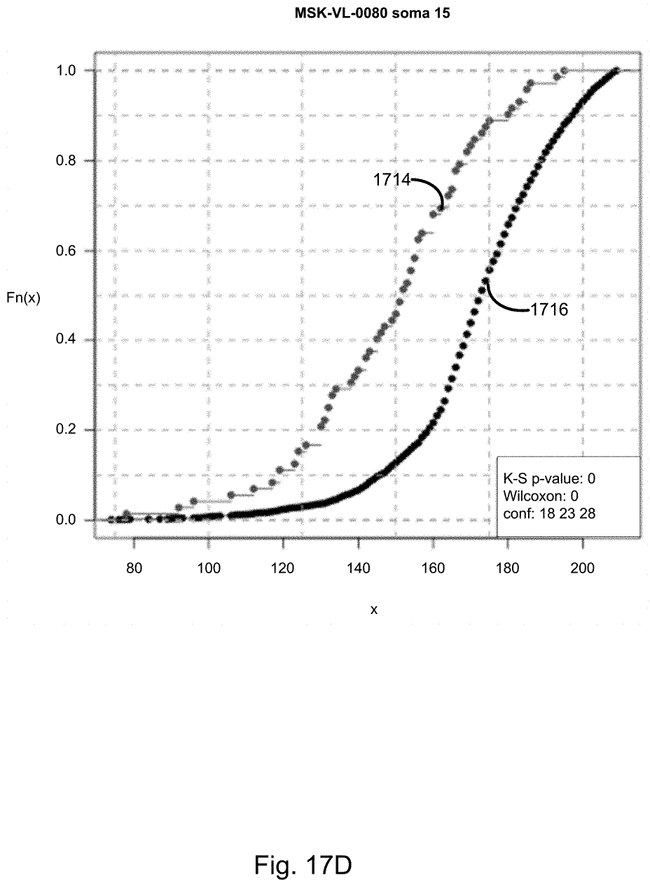

Briefly, when the lengths of cell-free DNA fragments encompassing loci associated with SNVs matched to a cancerous origin were cumulatively plotted as containing a variant allele (1702) or containing a reference allele (1704), the distribution of lengths matched the expected model, where cell-free DNA fragments encompassing the variant allele (1702) had smaller lengths on average than cell-free DNA fragments encompassing the reference allele (1704), as shown in FIG. 17A. However, when the lengths of cell-free DNA fragments encompassing loci associated with SNVs matched to white blood cells were cumulatively plotted as containing a variant allele (1708) or containing a reference allele (1706), the distribution of lengths for DNA fragments were approximately the same for both populations, as shown in FIG. 17B. This can be explained by the low tumor burden in the subject, resulting in only a small contribution of cell-free DNA fragments from cancer cells. As such, any considerable shift that would be caused by the shorter DNA fragments originating from cancer cells is diluted out by the DNA fragments originating from the germline cells and the white blood cells, which are in great excess. When the lengths of cell-free DNA fragments encompassing loci associated with SNVs matched to the germline were cumulatively plotted as containing a variant allele (1710) or containing a reference allele (1712), the distribution of lengths matched the expected model, where cell-free DNA fragments encompassing the variant allele (1710) had similar lengths on average to cell-free DNA fragments encompassing the reference allele (1712), as shown in FIG. 17C. When the lengths of cell-free DNA fragments encompassing the 15 loci associated with SNVs with an unidentified origin were cumulatively plotted as containing a variant allele (1714) or containing a reference allele (1716), it could be seen that the distribution of lengths of the cell-free DNA fragments encompassing the variant alleles (1714) was shifted shorter than the distribution of lengths of the cell-free DNA fragments encompassing the reference alleles (1716), as shown in FIG. 17D. This result is consistent with a hypothesis that the unidentified variants arose from cancer cells, because the shift in fragment lengths appears to be consistent with the model behavior expected of variant alleles arising from a cancer cell.

In order to validate the hypothesis that the fifteen unmatched variants did arise from cancer cells, a mixture model was trained against the fragment length distribution of cell-free DNA encompassing the seven loci corresponding to the variant alleles that were positively matched to a cancer origin (distributions not shown). An expectation maximization algorithm was then used to test the mixture model against the populations of cell-free DNA encompassing each of the 752 loci at which a single nucleotide variant was identified.

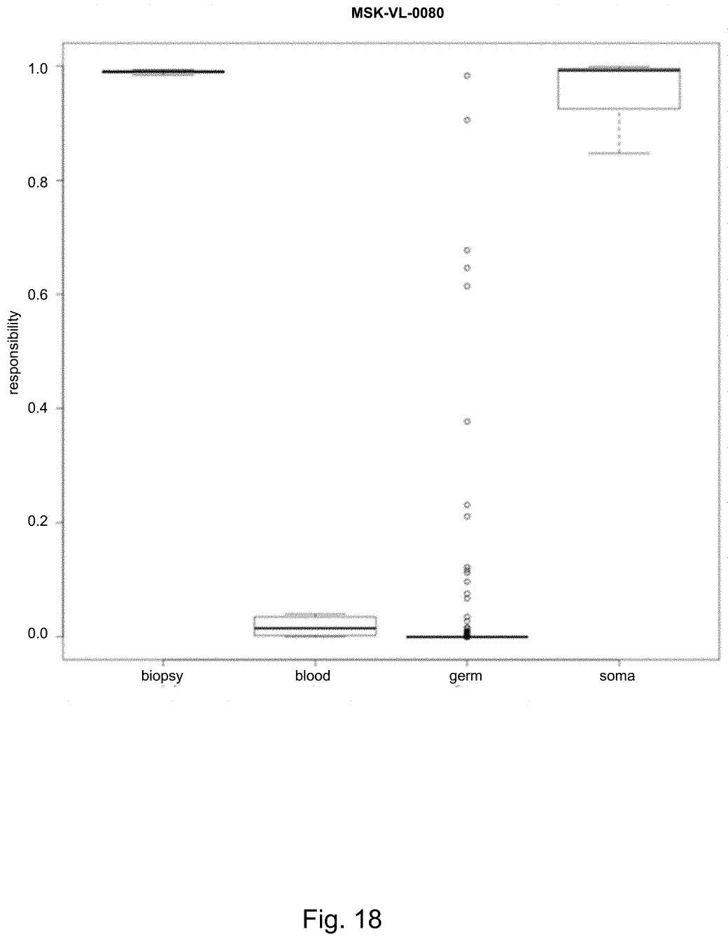

As shown in FIG. 18, the EM algorithm assigned a high level of responsibility to each of the seven loci corresponding to the biopsy-matched variants, as expected, indicating that these variant alleles originated from cancer cells. Consistently, the EM algorithm assigned a low level of responsibility to each of the 10 loci corresponding to the white-blood cell-matched variants, as expected, indicating that these variants did not originate from cancer cells. The EM algorithm provided a range of responsibilities for the 720 loci corresponding to germline-matched variants. However, unlike in Example 4, only eight of the 720 loci were assigned responsibilities above 20%. This can be explained by the low tumor burden in the patient, which dilutes out the size effect caused by the chromosomal copy number aberrations. Finally, the EM algorithm assigned a high level of responsibility to all 15 of the loci corresponding to the unmatched variants, indicating that these variant alleles originated from cancer cells.

### Example 6—Classification of Novel Somatic Variants

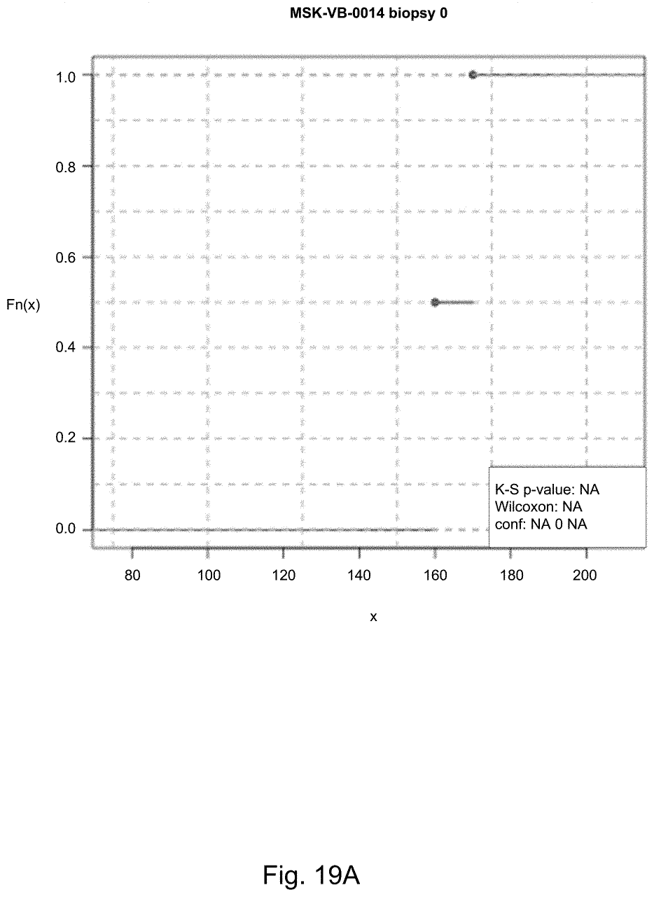

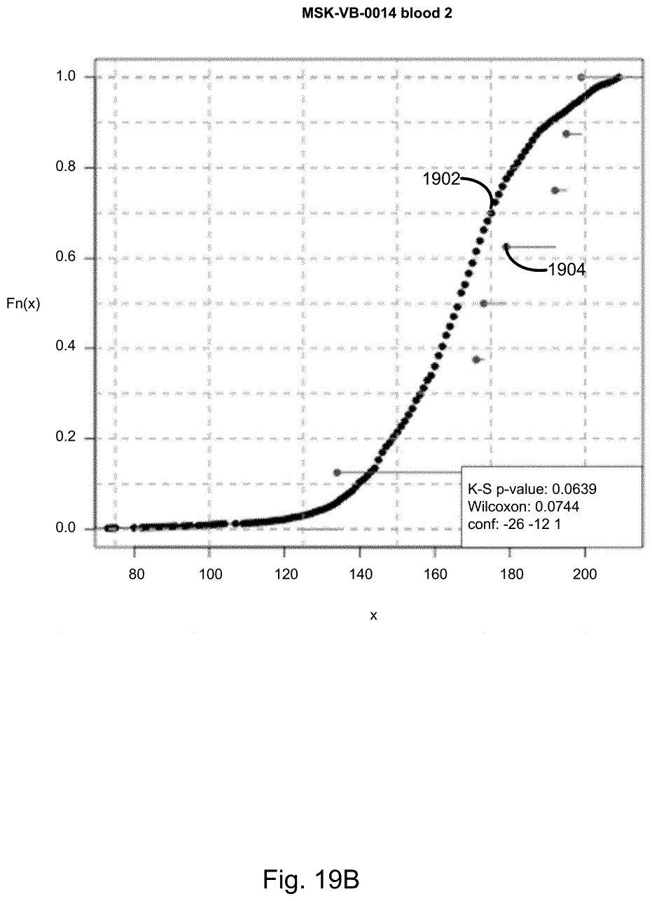

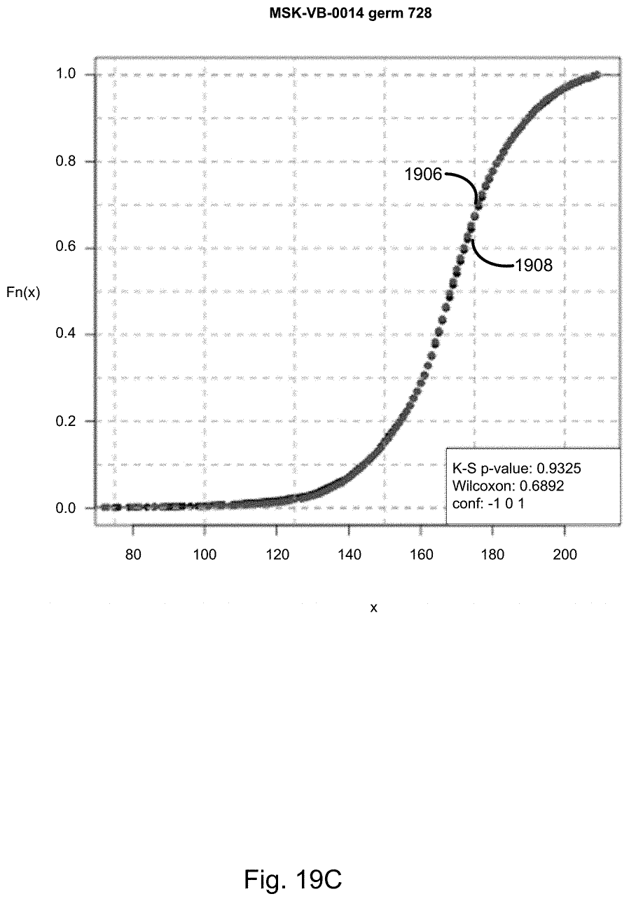

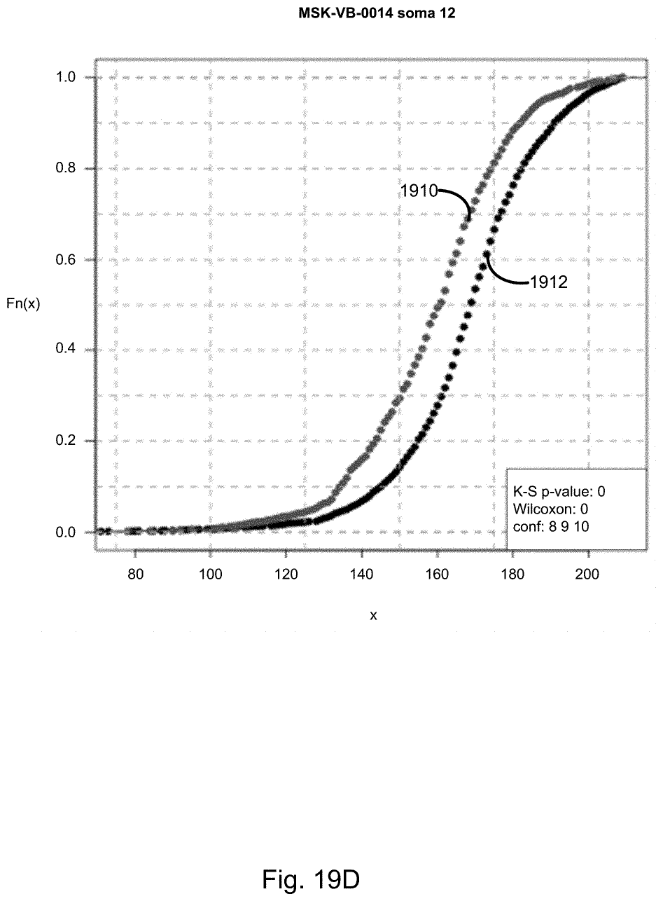

Targeted, capture-based DNA sequencing of cell-free DNA in a blood sample from a subject confirmed to have metastatic cancer were generated and mapped to a reference genome, as described above. 742 single nucleotide variants (SNVs) detected at the loci of interest were identified in the sequencing data. These loci were also sequenced in genomic DNA from (i) a tumor biopsy (e.g., cancer cells) from the subject, (ii) white blood cells from the subject, and (iii) a non-cancerous tissue sample from the subject. The origin of the 742 SNVs identified in the cell-free DNA were then matched to the three tissue types, allowing identification of the origins of each of the variants, as described in Examples 1-3. Of the variant alleles, none were identified as originating from cancer cells (FIG. 19A), 2 were identified as originating from clonal hematopoiesis (e.g., from white blood cells), and 728 were identified as originating from the germline. 12 SNVs, however, were not matched to any of these sources.

When the lengths of cell-free DNA fragments encompassing loci associated with SNVs matched to white blood cells were cumulatively plotted as containing a variant allele (1904) or containing a reference allele (1902), the distribution of lengths matched the expected model, where cell-free DNA fragments encompassing the variant allele (1904) had greater lengths on average than cell-free DNA fragments encompassing the reference allele (1902), as shown in FIG. 19B. Likewise, when the lengths of cell-free DNA fragments encompassing loci associated with SNVs matched to the germline were cumulatively plotted as containing a variant allele (1906) or containing a reference allele (1904), the distribution of lengths matched the expected model, where cell-free DNA fragments encompassing the variant allele (1908) had similar lengths on average to cell-free DNA fragments encompassing the reference allele (1906), as shown in FIG. 19C. When the lengths of cell-free DNA fragments encompassing the 12 loci associated with SNVs with an unidentified origin were cumulatively plotted as containing a variant allele (1910) or containing a reference allele (1912), it could be seen that the distribution of lengths of the cell-free DNA fragments encompassing the variant alleles (1910) was shifted shorter than the distribution of lengths of the cell-free DNA fragments encompassing the reference alleles (1912), as shown in FIG. 14D. This result is consistent with a hypothesis that the unidentified variants arose from cancer cells, because the shift in fragment lengths appears to be consistent with the model behavior expected of variant alleles arising from a cancer cell.

### Example 7—Classification of Novel Somatic Variants

Targeted, capture-based DNA sequencing of cell-free DNA in a blood sample from a subject confirmed to have metastatic cancer were generated and mapped to a reference genome, as described above. 1010 single nucleotide variants (SNVs) detected at the loci of interest were identified in the sequencing data. These loci were also sequenced in genomic DNA from (i) a tumor biopsy (e.g., cancer cells) from the subject, (ii) white blood cells from the subject, and (iii) a non-cancerous tissue sample from the subject. The origin of the 1010 SNVs identified in the cell-free DNA were then matched to the three tissue types, allowing identification of the origins of each of the variants, as described in Examples 1-3. Of the variant alleles, seven were identified as originating from cancer cells, 18 were identified as originating from clonal hematopoiesis (e.g., from white blood cells), and 967 were identified as originating from the germline. 18 SNVs, however, were not matched to any of these sources. An expectation maximization algorithm was then used to determine whether these 15 unmatched variants originated from cancer cells, as described above.

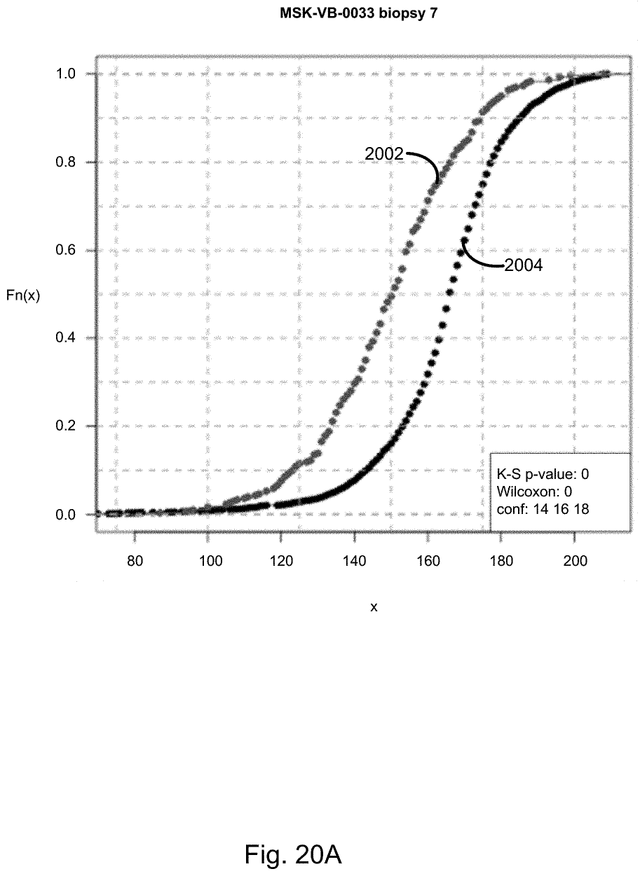

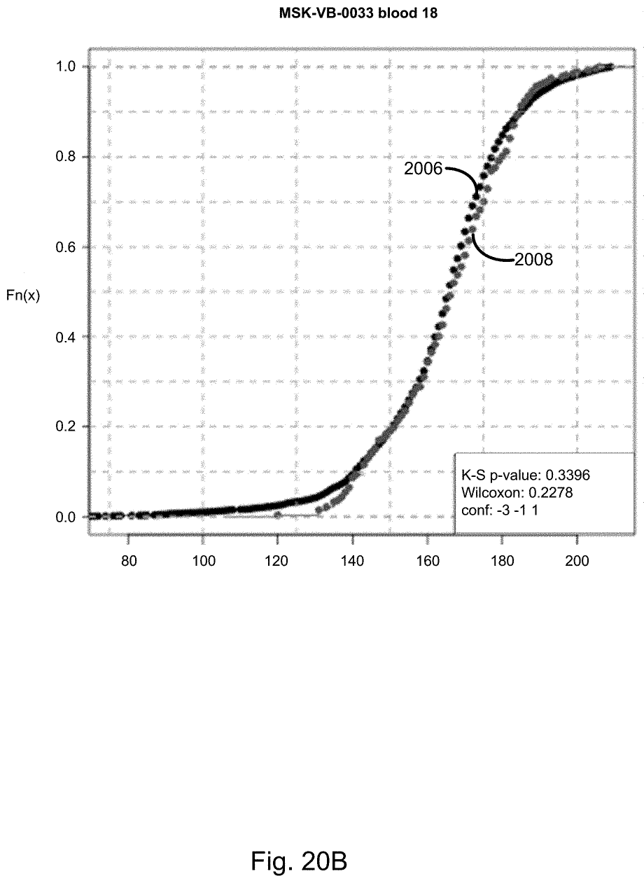

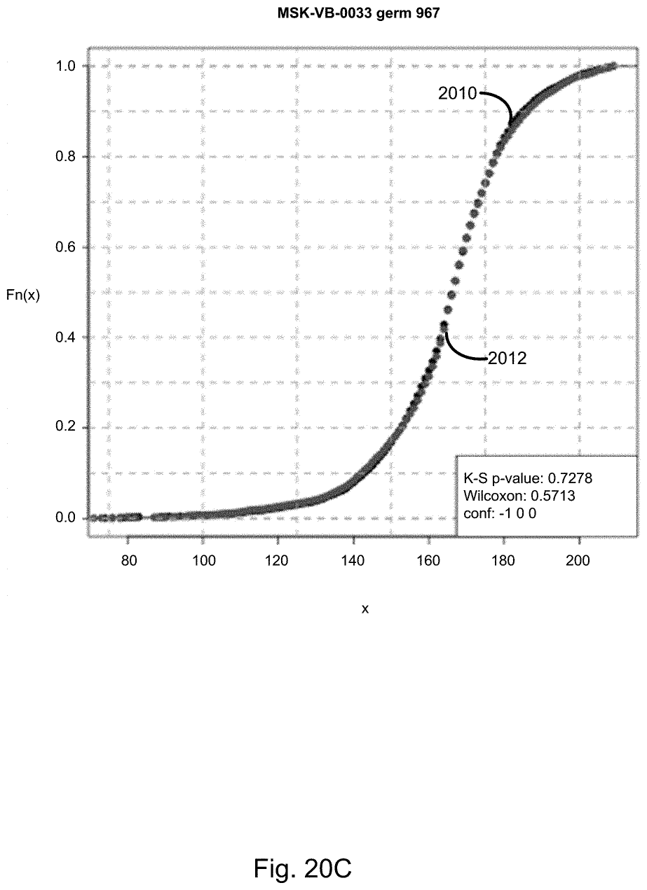

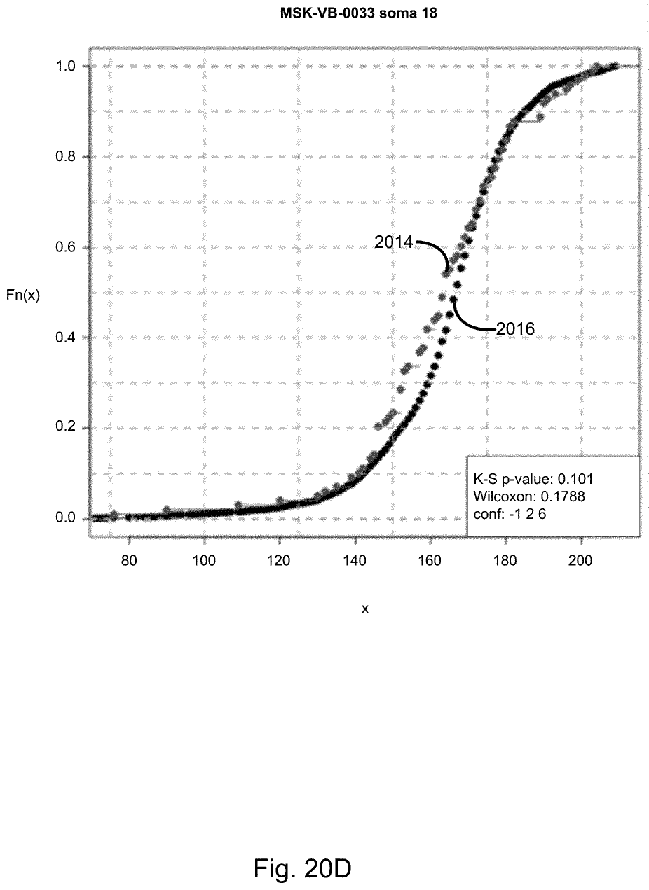

Briefly, when the lengths of cell-free DNA fragments encompassing loci associated with SNVs matched to a cancerous origin were cumulatively plotted as containing a variant allele (2002) or containing a reference allele (2004), the distribution of lengths matched the expected model, where cell-free DNA fragments encompassing the variant allele (2002) had smaller lengths on average than cell-free DNA fragments encompassing the reference allele (2004), as shown in FIG. 20A. However, when the lengths of cell-free DNA fragments encompassing loci associated with SNVs matched to white blood cells were cumulatively plotted as containing a variant allele (2008) or containing a reference allele (2006), the distribution of lengths for DNA fragments were approximately the same for both populations, as shown in FIG. 20B. This can be explained by the low tumor burden in the subject, resulting in only a small contribution of cell-free DNA fragments from cancer cells. As such, any considerable shift that would be caused by the shorter DNA fragments originating from cancer cells is diluted out by the DNA fragments originating from the germline cells and the white blood cells, which are in great excess. When the lengths of cell-free DNA fragments encompassing loci associated with SNVs matched to the germline were cumulatively plotted as containing a variant allele (2012) or containing a reference allele (2010), the distribution of lengths matched the expected model, where cell-free DNA fragments encompassing the variant allele (2012) had similar lengths on average to cell-free DNA fragments encompassing the reference allele (2010), as shown in FIG. 20C. When the lengths of cell-free DNA fragments encompassing the 18 loci associated with SNVs with an unidentified origin were cumulatively plotted as containing a variant allele (2014) or containing a reference allele (2016), the distribution of lengths for DNA fragments were approximately the same for both populations, as shown in FIG. 20D. This result suggests that the unidentified variants did not arise from cancer cells, because a characteristic shift smaller is not seen for the cell-free DNA encompassing the variant alleles, cumulatively.

In order to validate the hypothesis that the 18 unmatched variants did not arise from cancer cells, a mixture model was trained against the fragment length distribution of cell-free DNA encompassing the seven loci corresponding to the variant alleles that were positively matched to a cancer origin (distributions not shown). An expectation maximization algorithm was then used to test the mixture model against the populations of cell-free DNA encompassing each of the 1010 loci at which a single nucleotide variant was identified.

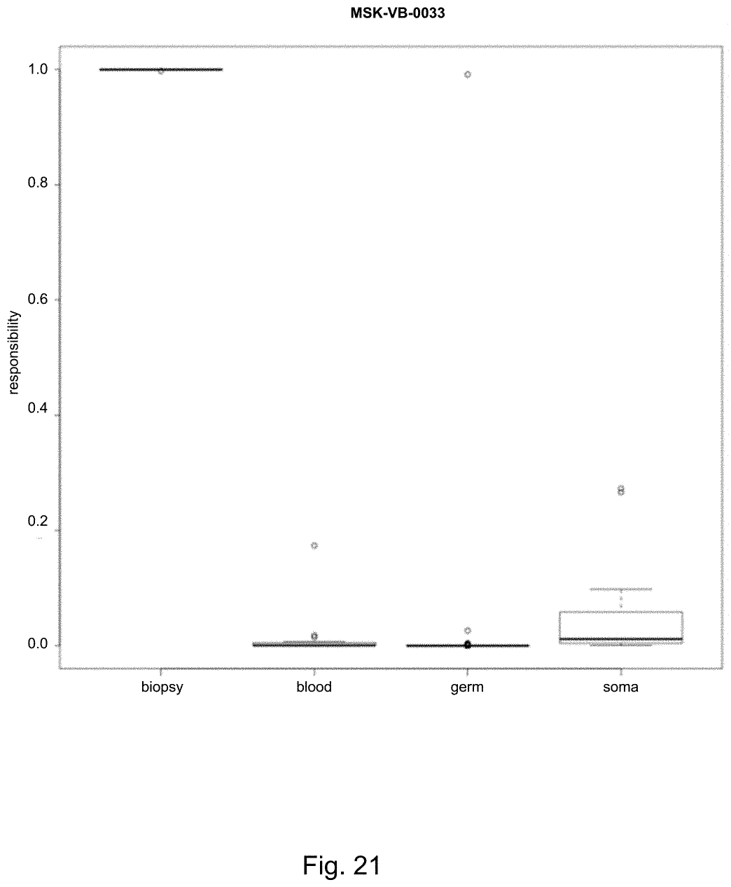

As shown in FIG. 21, the EM algorithm assigned a high level of responsibility to each of the seven loci corresponding to the biopsy-matched variants, as expected, indicating that these variant alleles originated from cancer cells. Consistently, the EM algorithm assigned a low level of responsibility to each of the 18 loci corresponding to the white-blood cell-matched variants, as expected, indicating that these variants did not originate from cancer cells. The EM algorithm assigned a low level of responsibility to all but one of the 967 loci corresponding to germline-matched variants. This can be explained by the low tumor burden in the patient, which dilutes out the size effect caused by the chromosomal copy number aberrations. Finally, the EM algorithm assigned a low level of responsibility to all 18 of the loci corresponding to the unmatched variants, indicating that these variant alleles did not originate from cancer cells.

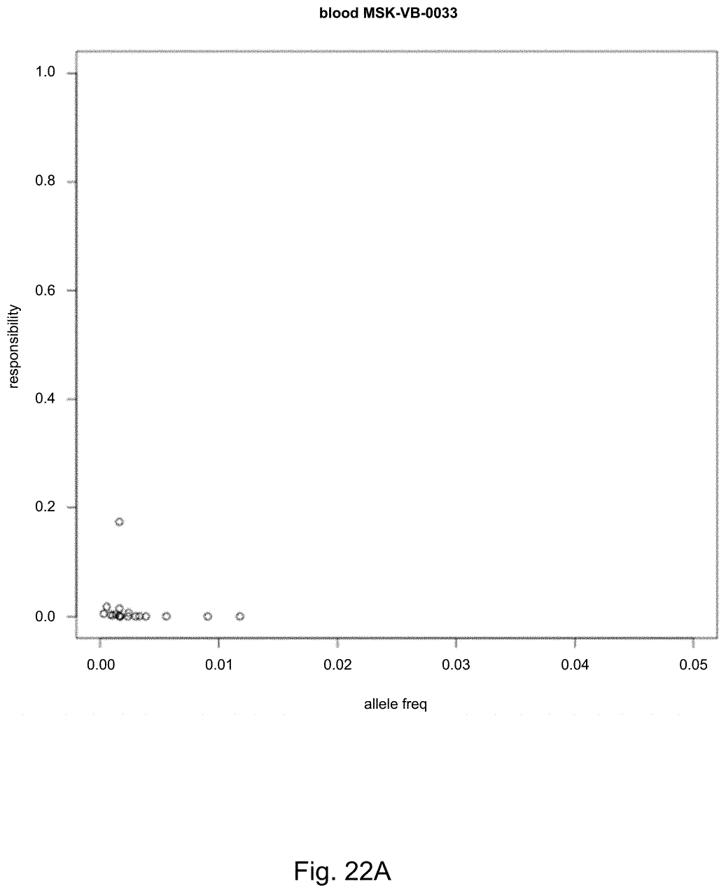

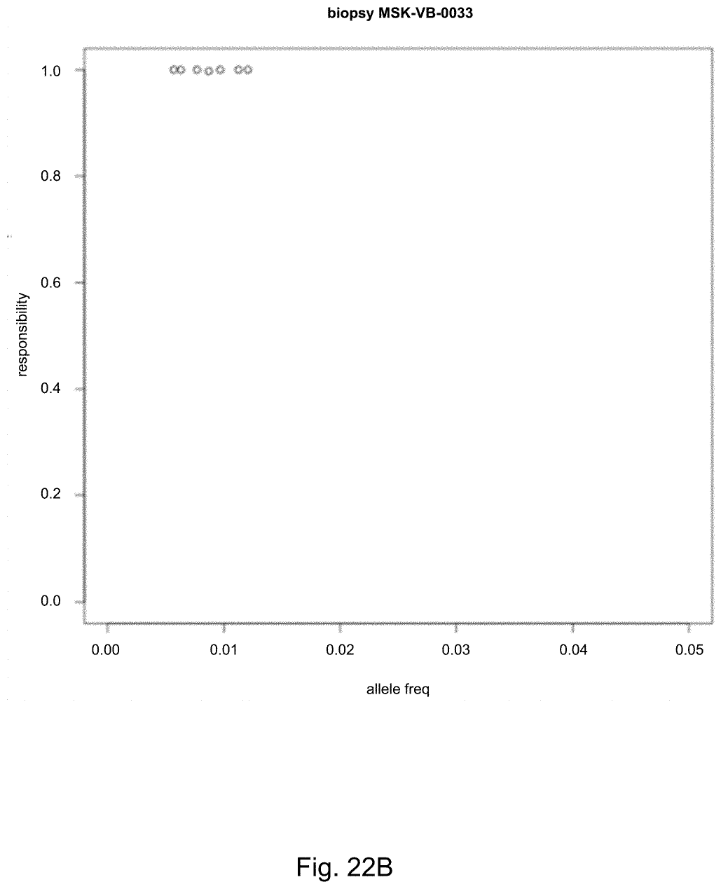

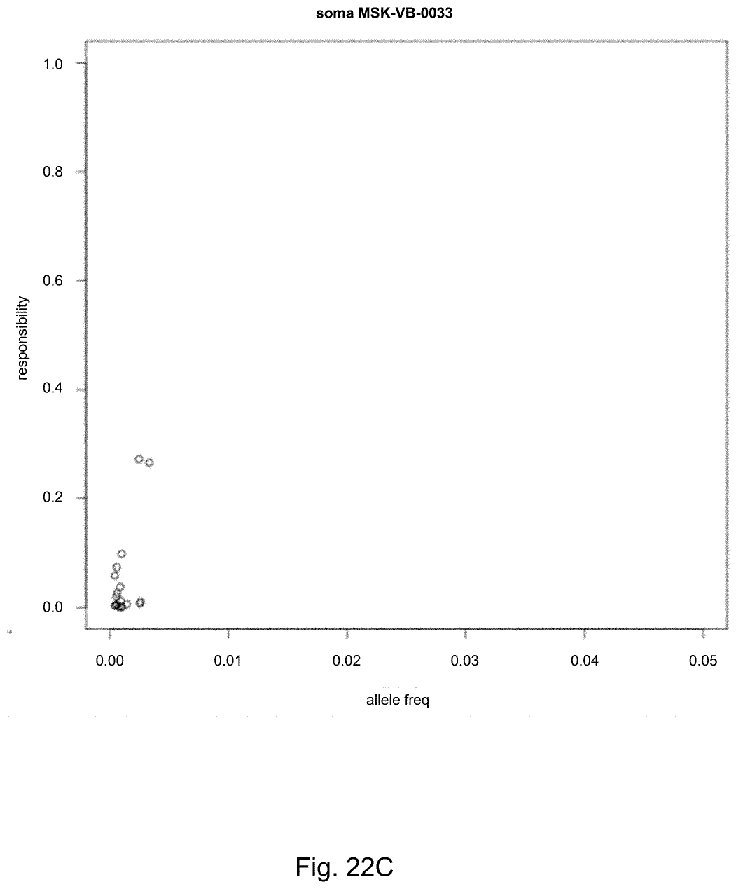

FIG. 22 illustrates the output of the EM algorithm for each individual loci, plotted as a function of allele frequency for the variant allele. As shown in FIG. 22A, the EM algorithm assigned a low level of responsibility to each of the 18 loci corresponding to the white-blood cell-matched variants. As shown in FIG. 22B, the EM algorithm assigned a high level of responsibility to each of the seven loci corresponding to the biopsy-matched variants. Similarly, the EM algorithm assigned a low level of responsibility to all 18 of the loci corresponding to the unmatched variants, as shown in FIG. 22C. Because the EM results for each of the unassigned variants appear to be similar to the EM results for the white-blood cell-matched variant alleles, it suggests the unmatched variants originate from clonal hematopoiesis, rather than from cancer cells.

### Example 8—Classification of Novel Somatic Variants

Targeted, capture-based DNA sequencing of cell-free DNA in a blood sample from a subject confirmed to have early lung cancer, were generated and mapped to a reference genome, as described above. 806 single nucleotide variants (SNVs) detected at the loci of interest were identified in the sequencing data. These loci were also sequenced in genomic DNA from (i) a tumor biopsy (e.g., cancer cells) from the subject, (ii) white blood cells from the subject, and (iii) a non-cancerous tissue sample from the subject. The origin of the 806 SNVs identified in the cell-free DNA were then matched to the three tissue types, allowing identification of the origins of each of the variants, as described in Examples 1-3. Of the variant alleles, five were identified as originating from cancer cells, 26 were identified as originating from clonal hematopoiesis (e.g., from white blood cells), and 745 were identified as originating from the germline. 30 SNVs, however, were not matched to any of these sources. An expectation maximization algorithm was then used to determine whether these 30 unmatched variants originated from cancer cells, as described above.

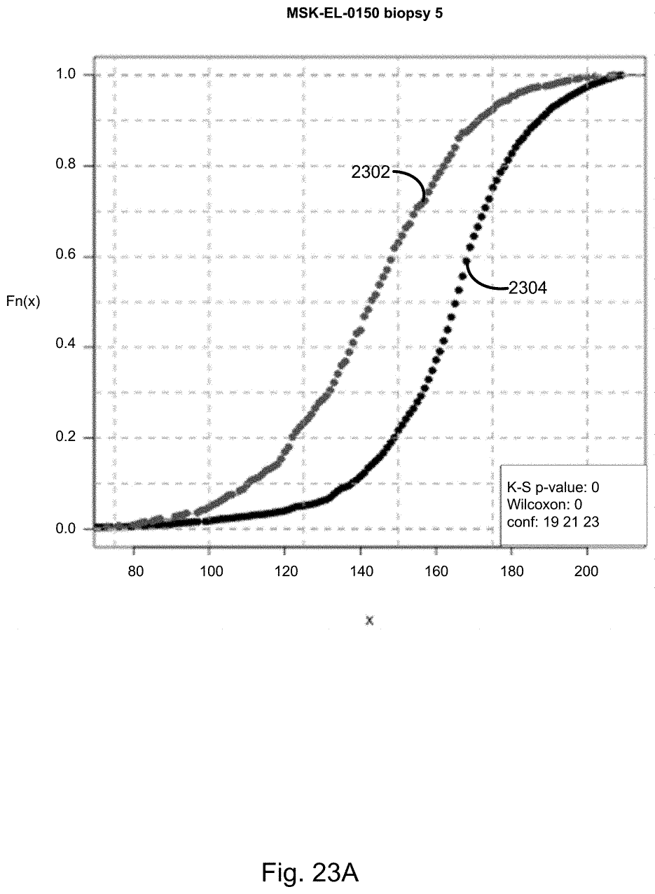

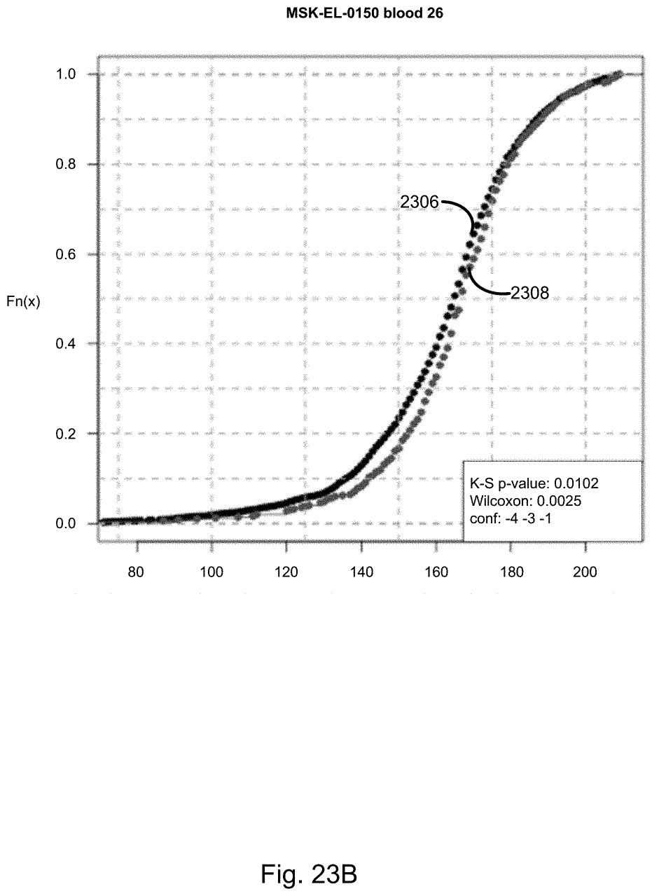

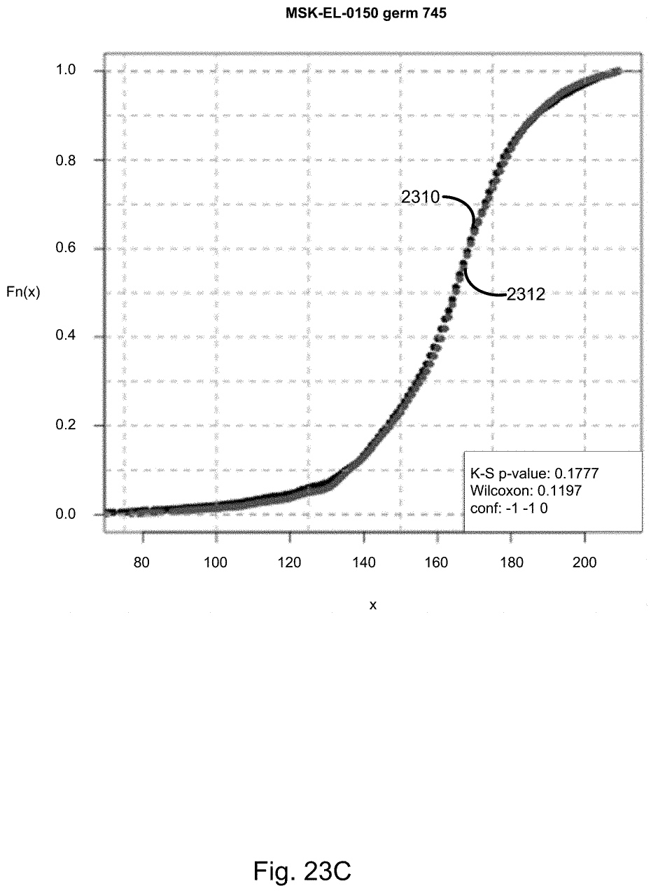

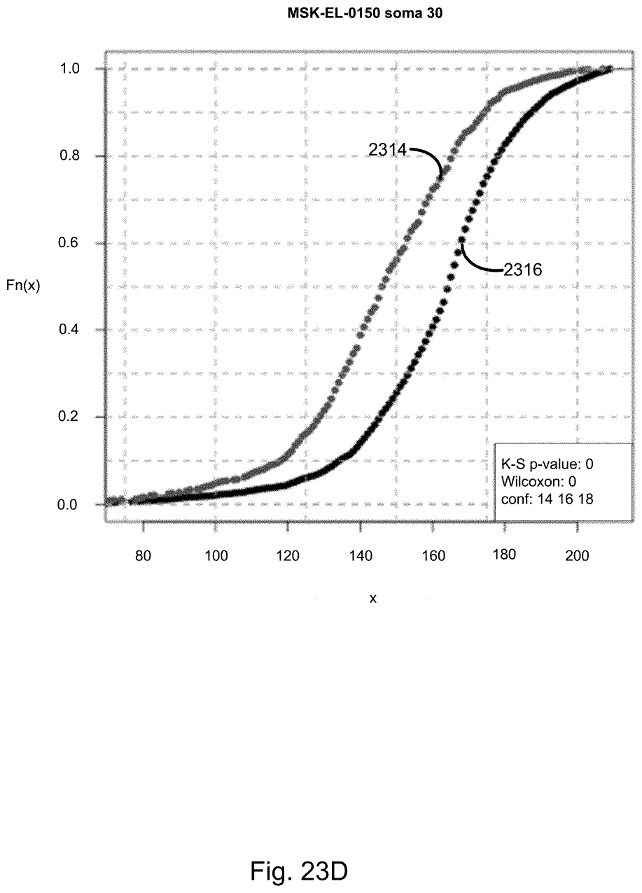

Briefly, when the lengths of cell-free DNA fragments encompassing loci associated with SNVs matched to a cancerous origin were cumulatively plotted as containing a variant allele (2302) or containing a reference allele (2304), the distribution of lengths matched the expected model, where cell-free DNA fragments encompassing the variant allele (2302) had smaller lengths on average than cell-free DNA fragments encompassing the reference allele (2304), as shown in FIG. 23A. When the lengths of cell-free DNA fragments encompassing loci associated with SNVs matched to white blood cells were cumulatively plotted as containing a variant allele (2308) or containing a reference allele (2306), the distribution of lengths matched the expected model, where cell-free DNA fragments encompassing the variant allele (2304) had greater lengths on average than cell-free DNA fragments encompassing the reference allele (2302), as shown in FIG. 23B. When the lengths of cell-free DNA fragments encompassing loci associated with SNVs matched to the germline were cumulatively plotted as containing a variant allele (2312) or containing a reference allele (2310), the distribution of lengths matched the expected model, where cell-free DNA fragments encompassing the variant allele (2312) had similar lengths on average to cell-free DNA fragments encompassing the reference allele (2310), as shown in FIG. 23C. When the lengths of cell-free DNA fragments encompassing the 30 loci associated with SNVs with an unidentified origin were cumulatively plotted as containing a variant allele (2314) or containing a reference allele (2316), it could be seen that the distribution of lengths of the cell-free DNA fragments encompassing the variant alleles (2314) was shifted shorter than the distribution of lengths of the cell-free DNA fragments encompassing the reference alleles (2316), as shown in FIG. 23D. This result is consistent with a hypothesis that the unidentified variants arose from cancer cells, because the shift in fragment lengths appears to be consistent with the model behavior expected of variant alleles arising from a cancer cell.

In order to validate the hypothesis that the 30 unmatched variants did arise from cancer cells, a mixture model was trained against the fragment length distribution of cell-free DNA encompassing the five loci corresponding to the variant alleles that were positively matched to a cancer origin (distributions not shown). An expectation maximization algorithm was then used to test the mixture model against the populations of cell-free DNA encompassing each of the 806 loci at which a single nucleotide variant was identified.

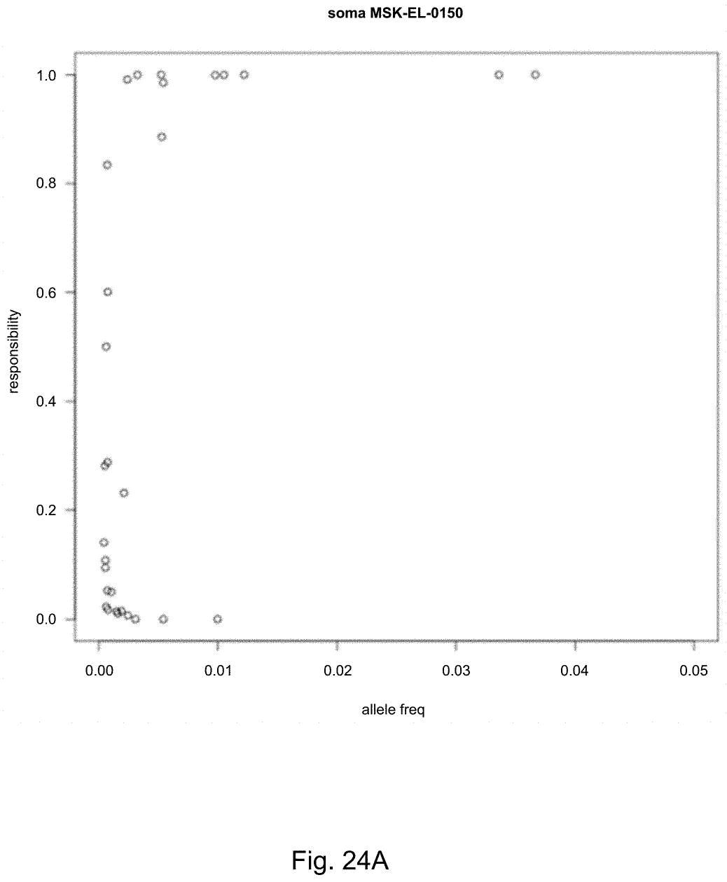

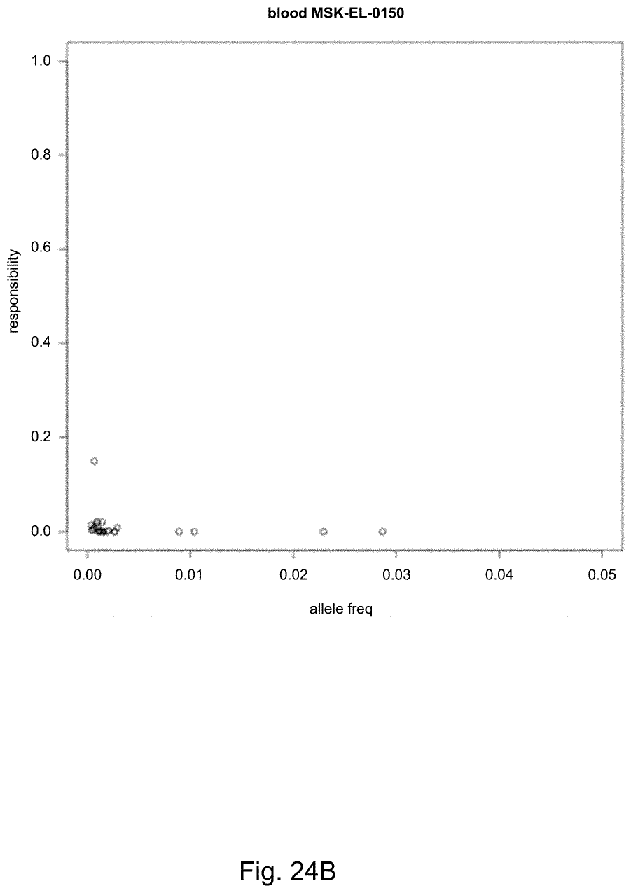

As shown in FIG. 24A, the EM algorithm assigned a mixture of responsibilities to the 30 loci corresponding to the unmatched variant alleles, suggesting that some, but not all, of the unmatched variants arose from cancer cells. However, the EM algorithm assigned a high responsibility to the high-frequency variants of the unmatched variants. In contrast, the EM algorithm assigned a low level of responsibility to each of the 26 loci corresponding to the white-blood cell-matched variants, indicating that these variants did not originate from cancer cells, as shown in FIG. 24B.

### Example 9—Classification of Novel Somatic Variants

Targeted, capture-based DNA sequencing of cell-free DNA in a blood sample from a subject confirmed to have early lung cancer, were generated and mapped to a reference genome, as described above. 841 single nucleotide variants (SNVs) detected at the loci of interest were identified in the sequencing data. These loci were also sequenced in genomic DNA from (i) a tumor biopsy (e.g., cancer cells) from the subject, (ii) white blood cells from the subject, and (iii) a non-cancerous tissue sample from the subject. The origin of the 814 SNVs identified in the cell-free DNA were then matched to the three tissue types, allowing identification of the origins of each of the variants, as described in Examples 1-3. Of the variant alleles, 15 were identified as originating from cancer cells, 9 were identified as originating from clonal hematopoiesis (e.g., from white blood cells), and 790 were identified as originating from the germline. 27 SNVs, however, were not matched to any of these sources. An expectation maximization algorithm was then used to determine whether these 27 unmatched variants originated from cancer cells, as described above.

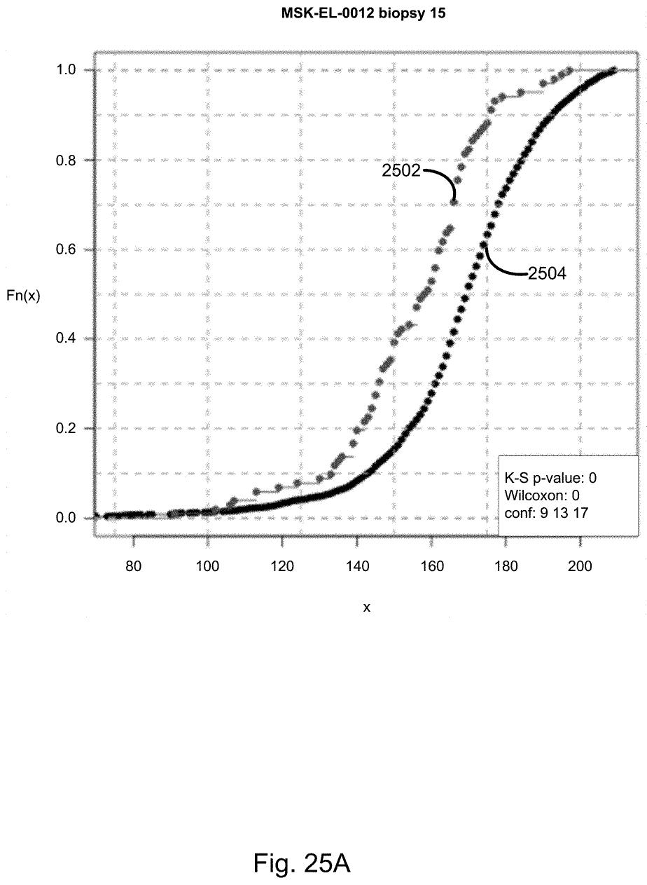

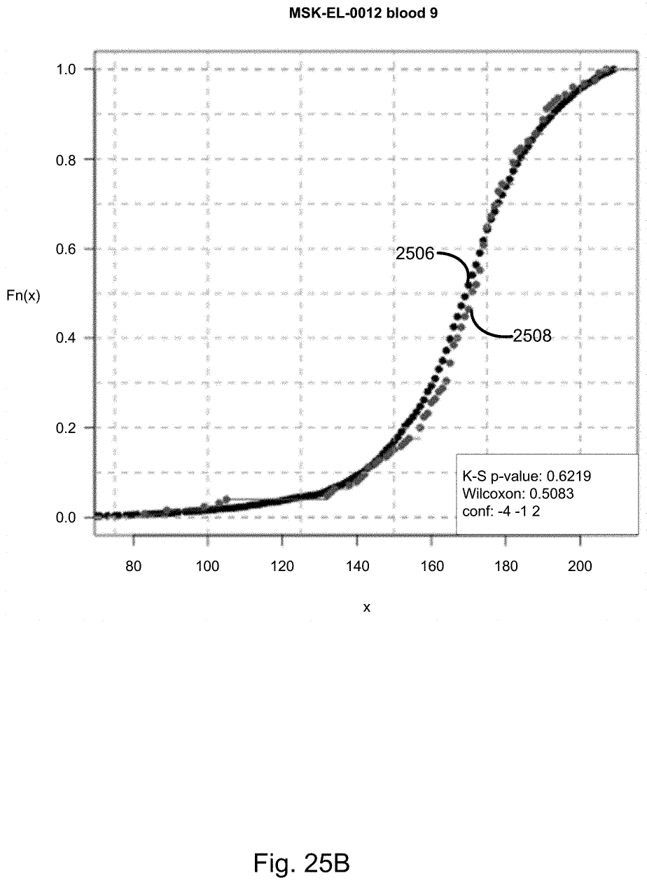

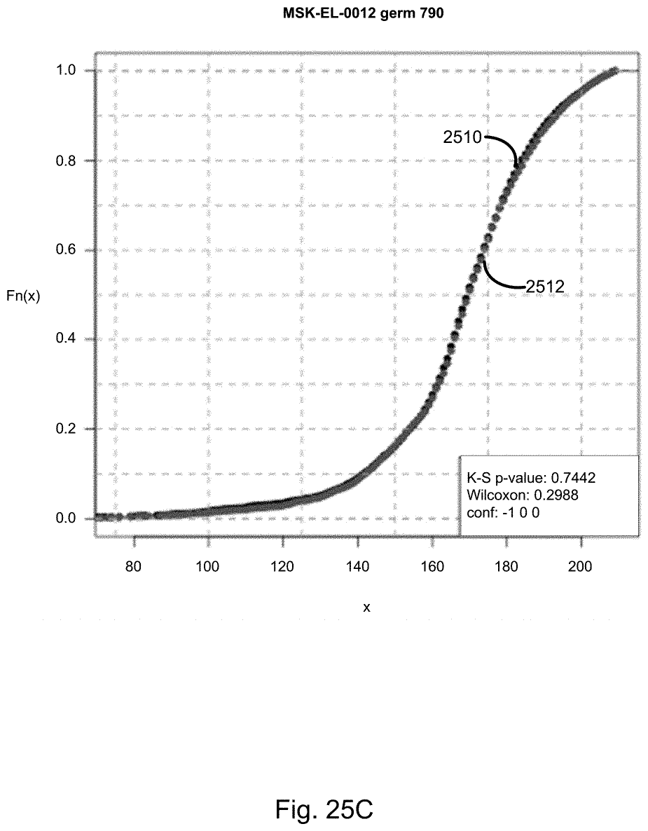

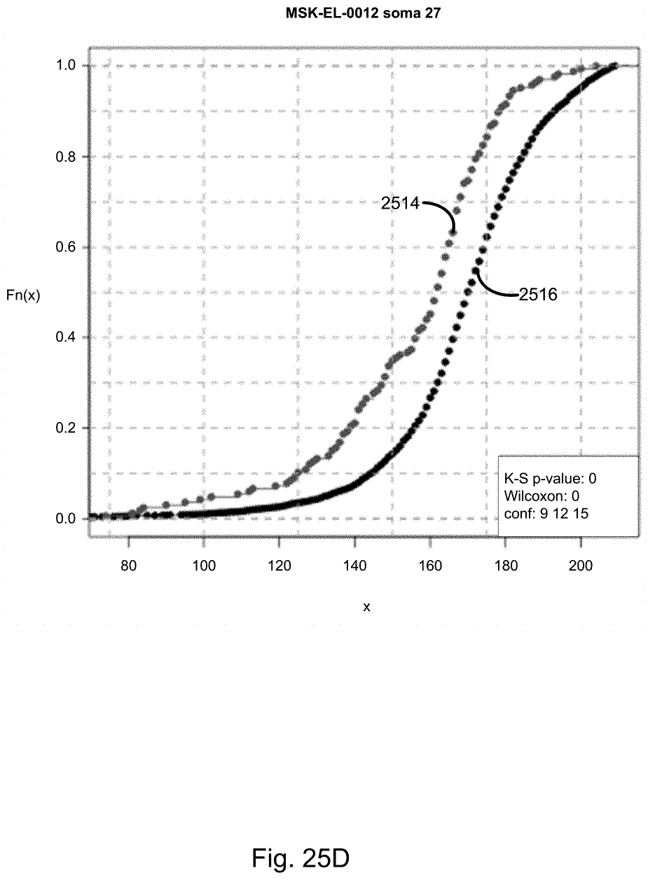

Briefly, when the lengths of cell-free DNA fragments encompassing loci associated with SNVs matched to a cancerous origin were cumulatively plotted as containing a variant allele (2502) or containing a reference allele (2504), the distribution of lengths matched the expected model, where cell-free DNA fragments encompassing the variant allele (2502) had smaller lengths on average than cell-free DNA fragments encompassing the reference allele (2504), as shown in FIG. 25A. However, when the lengths of cell-free DNA fragments encompassing loci associated with SNVs matched to white blood cells were cumulatively plotted as containing a variant allele (2508) or containing a reference allele (2506), the distribution of lengths for DNA fragments were approximately the same for both populations, as shown in FIG. 25B. This can be explained by the low tumor burden in the subject, resulting in only a small contribution of cell-free DNA fragments from cancer cells. When the lengths of cell-free DNA fragments encompassing loci associated with SNVs matched to the germline were cumulatively plotted as containing a variant allele (2512) or containing a reference allele (2510), the distribution of lengths matched the expected model, where cell-free DNA fragments encompassing the variant allele (2512) had similar lengths on average to cell-free DNA fragments encompassing the reference allele (2510), as shown in FIG. 25C. When the lengths of cell-free DNA fragments encompassing the 27 loci associated with SNVs with an unidentified origin were cumulatively plotted as containing a variant allele (2514) or containing a reference allele (2516), it could be seen that the distribution of lengths of the cell-free DNA fragments encompassing the variant alleles (2514) was shifted shorter than the distribution of lengths of the cell-free DNA fragments encompassing the reference alleles (2516), as shown in FIG. 23D. This result is consistent with a hypothesis that the unidentified variants arose from cancer cells, because the shift in fragment lengths appears to be consistent with the model behavior expected of variant alleles arising from a cancer cell.

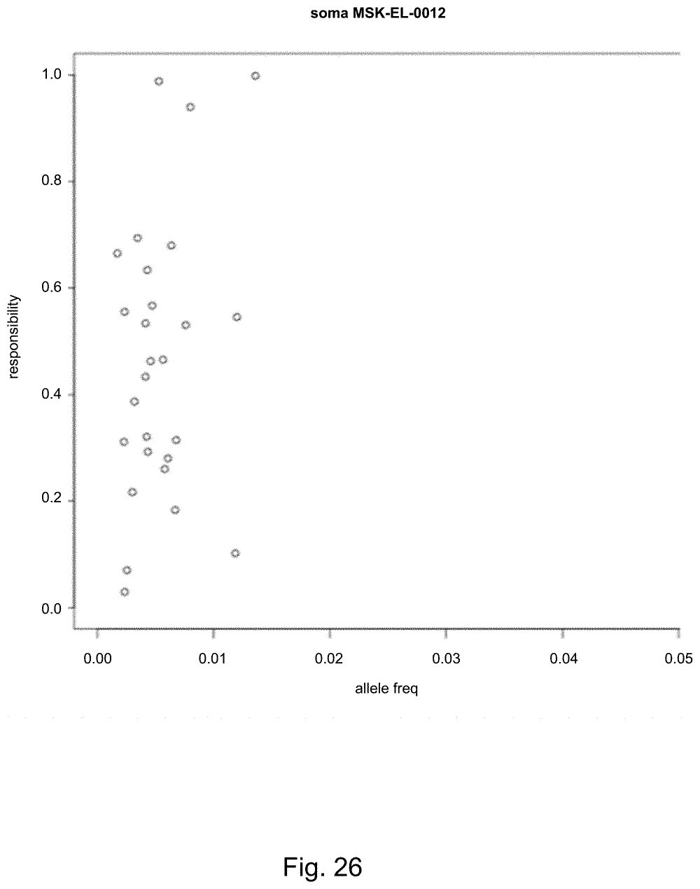

In order to test the hypothesis that the 27 unmatched variants did arise from cancer cells, a mixture model was trained against the fragment length distribution of cell-free DNA encompassing the 15 loci corresponding to the variant alleles that were positively matched to a cancer origin (distributions not shown). An expectation maximization algorithm was then used to test the mixture model against the populations of cell-free DNA encompassing each of the 27 loci at an unassigned variant was identified. In fact, despite that when plotted in aggregate there was a significant shift shorter in the fragment-length distribution of the cell-free DNA fragments encompassing the unmatched variant alleles (as shown in FIG. 25D), the EM algorithm assigned a high responsibility to only three of the 27 corresponding loci (as shown in FIG. 26).

### Example 10—Analysis of Cell-free DNA Fragments from a Subject Without Cancer

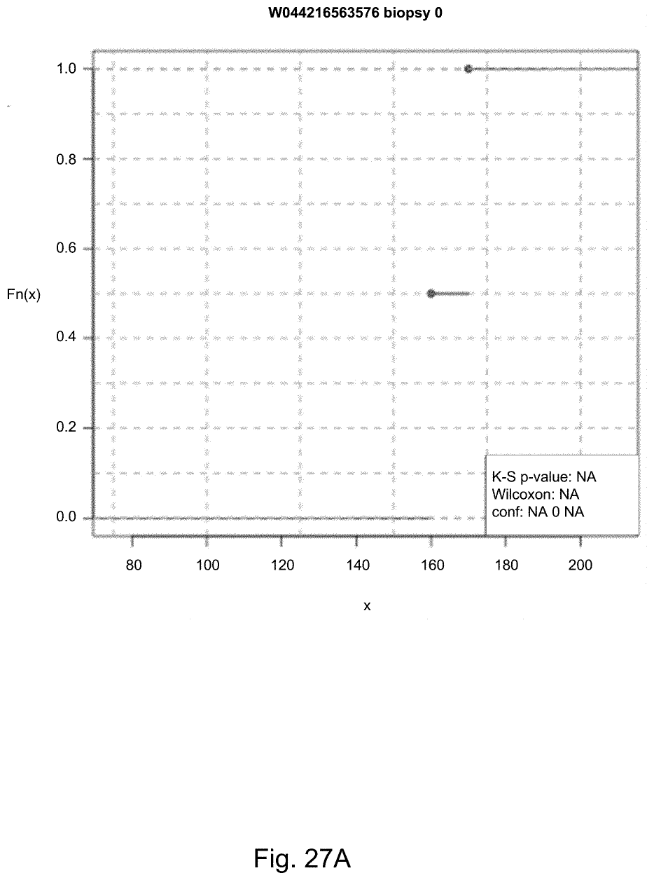

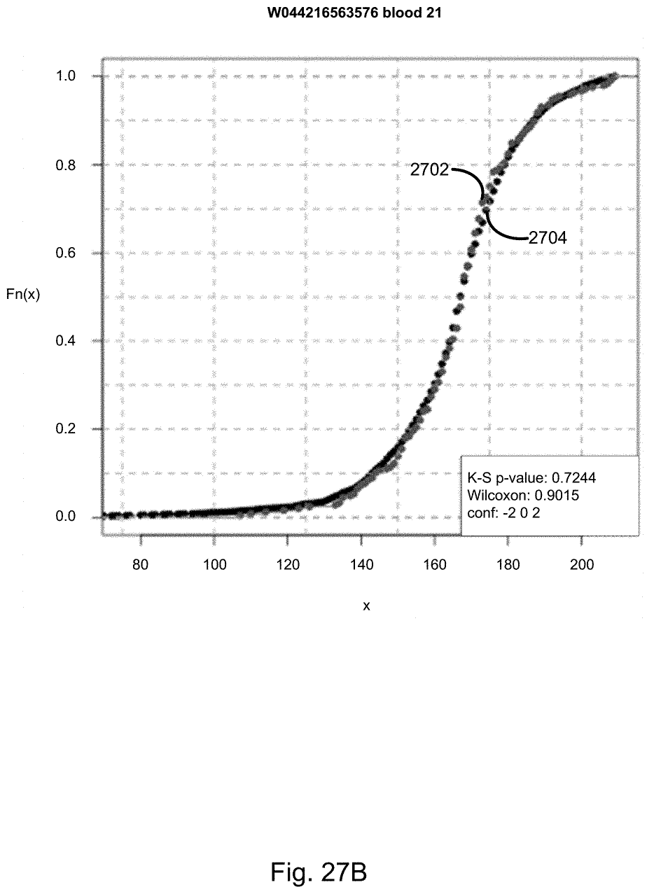

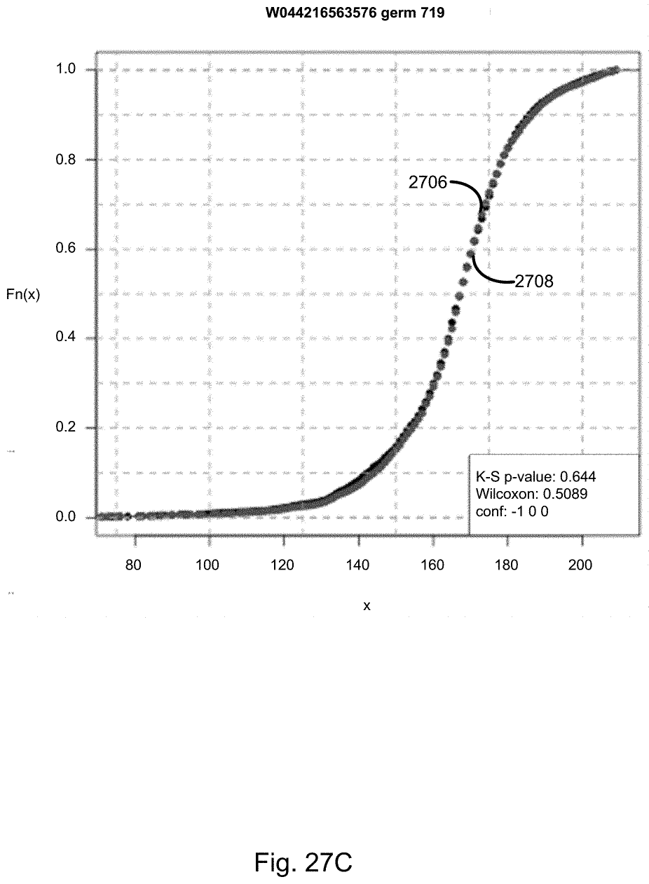

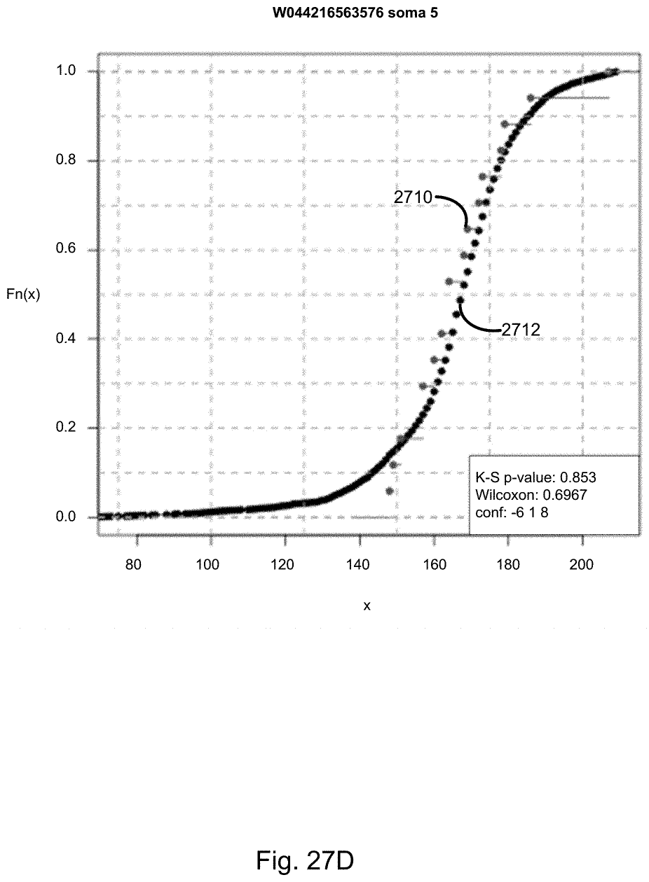

In order to further validate that the cell-free DNA fragment shift phenomenon observed is relevant to cancer biology, cell-free DNA fragments from a subject who does not have cancer were evaluated. Briefly, targeted, capture-based DNA sequencing of cell-free DNA in a blood sample from a subject confirmed not to have cancer, were generated and mapped to a reference genome, as described above. 745 single nucleotide variants (SNVs) detected at the loci of interest were identified in the sequencing data. These loci were also sequenced in genomic DNA from (i) white blood cells from the subject and (ii) a non-cancerous tissue sample from the subject. The origin of the 745 SNVs identified in the cell-free DNA were then matched to the tissue types, allowing identification of the origins of each of the variants, as described in Examples 1-3. Of the variant alleles, none were identified as originating from cancer cells (as illustrated in FIG. 27A because the subject did not have cancer, 21 were identified as originating from clonal hematopoiesis (e.g., from white blood cells), and 719 were identified as originating from the germline. 5 SNVs, however, were not matched to any of these sources.

When the lengths of cell-free DNA fragments encompassing loci associated with SNVs matched to white blood cells were cumulatively plotted as containing a variant allele (2702) or containing a reference allele (2704), the distribution of lengths for DNA fragments were approximately the same for both populations, as shown in FIG. 27B. This is consistent with the model, in which cell-free DNA fragments encompassing a white-blood cell-matched variant allele have a distribution of fragment lengths that is shifted longer, relative to the distribution of fragments lengths for the corresponding reference allele at the same locus, due to the presence of the reference allele, but not the variant allele, in cancer cells. Therefore, when the reference allele is not represented in cancer cells—such as here where the subject doesn't have cancer—no shift in the distribution of fragment lengths of cell-free DNA encompassing variant alleles matched to white blood cells is expected. When the lengths of cell-free DNA fragments encompassing loci associated with SNVs matched to the germline were cumulatively plotted as containing a variant allele (2706) or containing a reference allele (2708), the distribution of lengths matched the expected model, where cell-free DNA fragments encompassing the variant allele (2706) had similar lengths on average to cell-free DNA fragments encompassing the reference allele (2708), as shown in FIG. 27C. When the lengths of cell-free DNA fragments encompassing the 5 loci associated with SNVs with an unidentified origin were cumulatively plotted as containing a variant allele (2710) or containing a reference allele (2712), the variant alleles (2710) had similar lengths on average to cell-free DNA fragments encompassing the reference alleles (2712), as shown in FIG. 27D, consistent with a model for a subject who does not have cancer.

### Example 11—Classification of Novel Somatic Variants in a Hypermutation Subject with a High Tumor Burden

Targeted, capture-based DNA sequencing of cell-free DNA in a blood sample from a subject confirmed to have a hypermutation metastatic cancer, having a high tumor burden of approximately 80%, were generated and mapped to a reference genome, as described above. 2333 single nucleotide variants (SNVs) detected at the loci of interest were identified in the sequencing data. These loci were also sequenced in genomic DNA from (i) a tumor biopsy (e.g., cancer cells) from the subject, (ii) white blood cells from the subject, and (iii) a non-cancerous tissue sample from the subject. The origin of the 2333 SNVs identified in the cell-free DNA were then matched to the three tissue types, allowing identification of the origins of each of the variants, as described in Examples 1-3. Of the variant alleles, 16 were identified as originating from cancer cells, 6 were identified as originating from clonal hematopoiesis (e.g., from white blood cells), and 782 were identified as originating from the germline. 1529 SNVs, however, were not matched to any of these sources. An expectation maximization algorithm was then used to attempt to determine whether these 1529 unmatched variants originated from cancer cells, as described above.

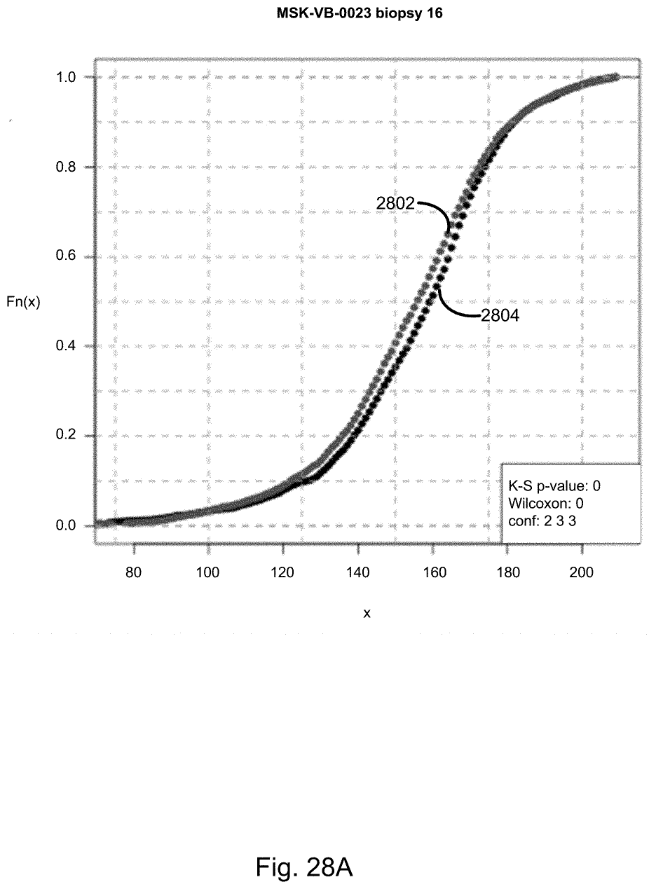

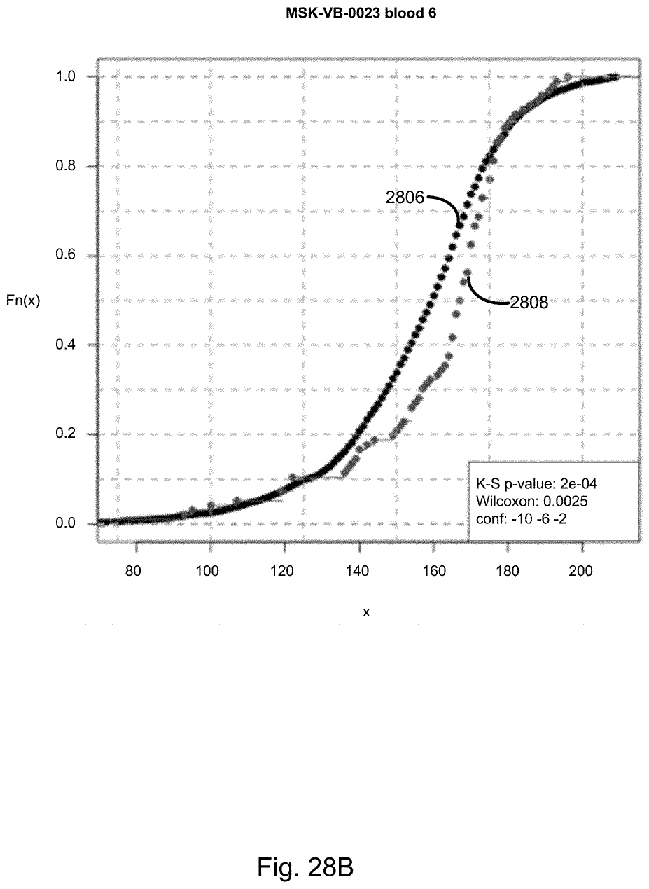

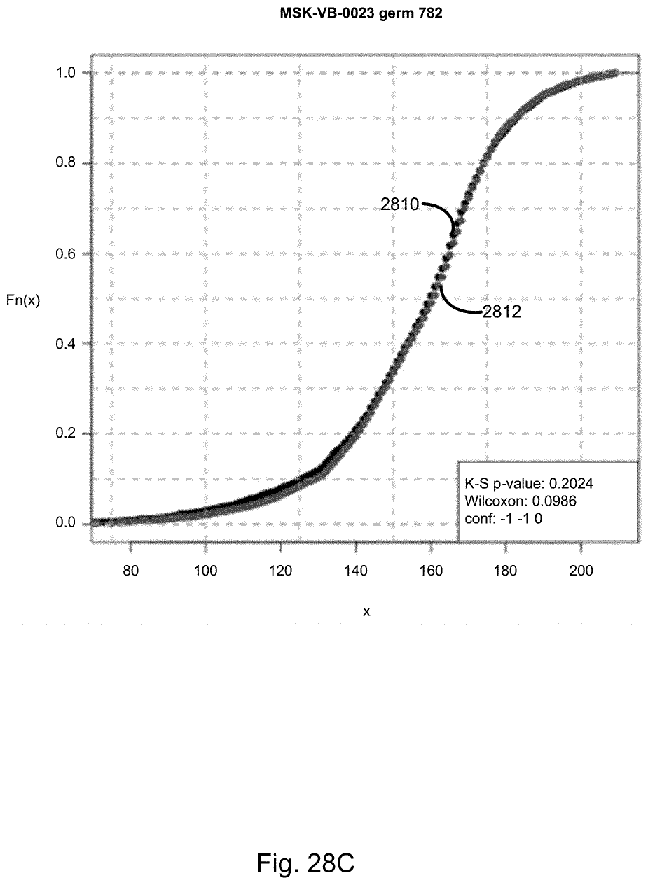

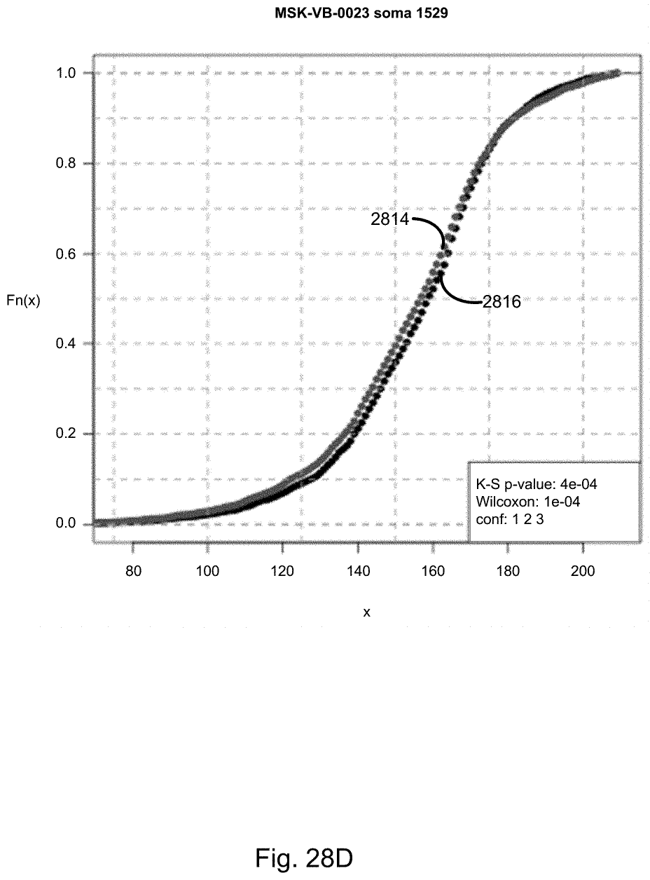

Briefly, when the lengths of cell-free DNA fragments encompassing loci associated with SNVs matched to a cancerous origin were cumulatively plotted as containing a variant allele (2802) or containing a reference allele (2804), only a small shift in the distribution of fragment lengths of cell-free DNA fragments encompassing cancer-matched variants, relative to cell-free DNA fragments encompassing the reference allele, was observed. This is due to the extremely high tumor burden in the subject, which causes a majority of the cell-free DNA fragments in the blood to be from cancer cells. Because cell-free DNA fragments from non-cancerous cells and white blood cells are under-represented into the sample, the distribution of fragment lengths of cell-free DNA encompassing the reference allele is also shift shorter since most of these fragments originate from cancer cells. However, when the lengths of cell-free DNA fragments encompassing loci associated with SNVs matched to white blood cells were cumulatively plotted as containing a variant allele (2808) or containing a reference allele (2806), the distribution of lengths matched the expected model, where cell-free DNA fragments encompassing the variant allele (2808) had greater lengths on average than cell-free DNA fragments encompassing the reference allele (2806), as shown in FIG. 28B, since the cancer cells do not contain the white blood cell-matched variants. When the lengths of cell-free DNA fragments encompassing loci associated with SNVs matched to the germline were cumulatively plotted as containing a variant allele (2812) or containing a reference allele (2810), the distribution of lengths matched the expected model, where cell-free DNA fragments encompassing the variant allele (2812) had similar lengths on average to cell-free DNA fragments encompassing the reference allele (2810), as shown in FIG. 28C. When the lengths of cell-free DNA fragments encompassing the 1529 loci associated with SNVs with an unidentified origin were cumulatively plotted as containing a variant allele (2814) or containing a reference allele (2816), only a slight shift shorter in the fragment-length distribution of the of cell-free DNA fragments encompassing the variant alleles (2814), relative to the distribution of lengths of cell-free DNA fragments encompassing the reference allele (2816) was observed, see FIG. 28D. This pattern would be consistent with the presence of a large number of variants arising from cancer cells, but not matched to a biopsy sample, in a sample where the majority of cell-free DNA is being generated from cancer cells. In hypermutation types of cancer, each sub-clonal population of cancerous cells would be expected to have a different set of novel variant alleles, such that the sequencing of one clonal population of cancer cells from the subject would not identify most of the cancer variants found in cell-free DNA, which is derived from a mixture of all the clonal cancer populations.

To test the hypothesis that the 1529 unmatched variants did arise from cancer cells, a mixture model was trained against the fragment length distribution of cell-free DNA encompassing the 16 loci corresponding to the variant alleles that were positively matched to a cancer origin (distributions not shown). An expectation maximization algorithm was then used to test the mixture model against the populations of cell-free DNA encompassing each of the 2333 loci at which a single nucleotide variant was identified.

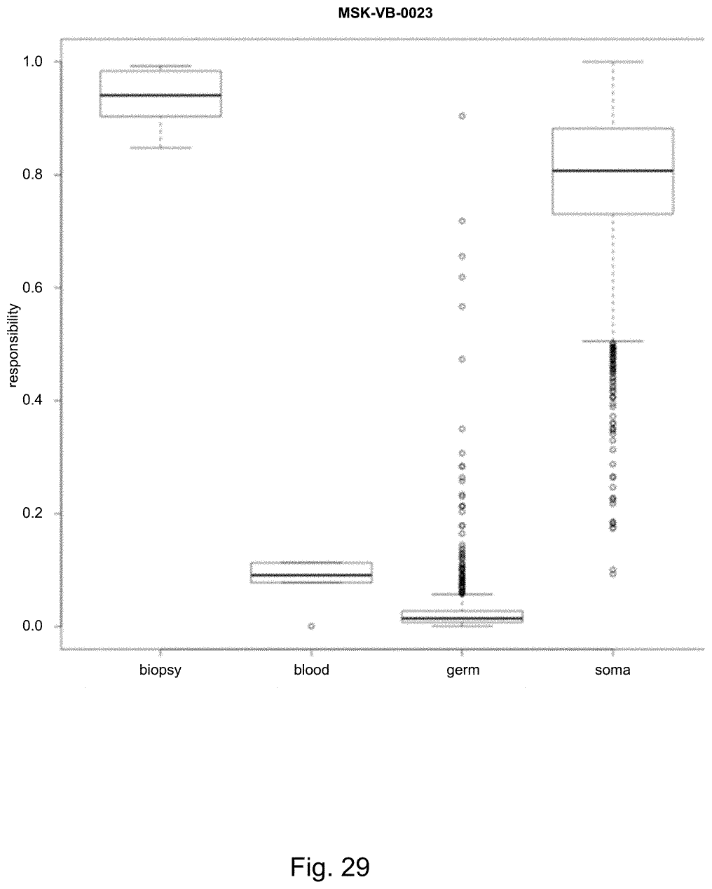

As shown in FIG. 29, the EM algorithm assigned a high level of responsibility to each of the 16 loci corresponding to the biopsy-matched variants, as expected, indicating that these variant alleles originated from cancer cells. Consistently, the EM algorithm assigned a low level of responsibility to each of the six loci corresponding to the white-blood cell-matched variants, as expected, indicating that these variants did not originate from cancer cells. The EM algorithm provided a range of responsibilities for the 782 loci corresponding to germline-matched variants. This can be explained by the combination of chromosomal copy number aberrations in the cancer cells and the extremely high tumor burden in the subject, resulting in a majority of cell-free DNA fragments encompassing germline variant and reference alleles originating from the cancer cells. Likewise, the EM algorithm assigned a range of responsibilities to the 1529 loci corresponding to the unmatched variants, suggesting that additional analysis is needed to definitively assign origins for these variant alleles. This, again, is explained by the extremely high tumor burden in the subject.

### Example 12—Detection of Mis-Mapping Assignments

Targeted, capture-based DNA sequencing of cell-free DNA in a blood sample from a cancer subject were generated and mapped to a reference genome, as described above. Analysis of the fragment-length distribution of three apparent single nucleotide variants at positions 236649, 236653, and 236678 on chromosome 5 showed very pronounced fragment shifts shorter, relative to the fragment-length distribution of cell-free DNA fragments encompassing the corresponding reference alleles. In fact, as shown in FIGS. 30A, 30B, and 30C, the majority of the fragments encompassing the putative variant alleles have fragment lengths (3002, 3006, and 3010, respectively) that are less than 100 nucleotides. This is in contrast to the cell-free DNA fragments encompassing the corresponding reference alleles, which have fragments lengths (3004, 3008, and 3012, respectively), showing a normal distribution centered between 160 and 170 nucleotides.

There were two observations that suggested that the mappings of these sequence variants was incorrect. First, it was unusual that the DNA fragment-length shifts were much larger than seen previously for other variants, and the complete absence of longer DNA fragments. Second, it was unusual to have three variant alleles located so closely together, all within 30 nucleotides of each other. In fact, when the alignments were inspected by hand, it was determined that longer reads containing the three putative variants mapped elsewhere in the genome. But, but there was evidence that the longer reads were also mis-mapped at the other position. Rather, the DNA fragments containing these putative variants actually map to positions in the subject's genome that are not represented in the human reference genome used.

This experiment suggests that mis-mappings can be identified based on the detection of fragment-length distribution anomalies, as shown in FIG. 30. That is, where a fragment length distribution for an allele (e.g., a variant allele) does not match a known distribution pattern (e.g., accounting for the source of the variant, the tumor burden of the subject, etc.), a hypothesis can be made that the fragments have been mis-aligned to the reference genome. Likewise, mis-mappings can be identified based on the detection of an unusually high density of variant alleles in a region of the genome.

Other examples of fragment-length distributions that do not appear to be related to cancer biology, and likely indicate the mis-alignment of cell-free DNA fragment sequences to the reference genome, are shown in FIGS. 31A-31D, where the fragment length distribution of cell-free DNA fragments encompassing apparent variant alleles (3104, 3108, 3112, and 3114, respectively) and/or the fragment length distribution of cell-free DNA fragments encompassing corresponding reference alleles (3102, 3106, 3110, and not detected, respectively) do fit an expected distribution profile.

### Example 13—Validation of Trained Models Using Fragment Length Distribution

Fragment length distributions were used as part of a feedback loop to determine whether or not variant calling filters were operating correctly to leave relevant biology intact. On average, as shown above, allele variants arising from cancer should result in cell-free DNA fragments with length distributions that are shifted shorter than cell-free DNA fragments encompassing the corresponding reference allele.

First, the lengths of fragments encompassing loci corresponding to identified variant alleles in the TP53 gene were evaluated in the context of two variant calling algorithms, Q60 and PASS, to determine whether the algorithms are correctly identifying variant alleles in the TP53 gene that are relevant to cancer biology. Briefly, as shown in FIG. 32, 72 variant allele loci in the TP53 gene, identified in cell-free DNA isolated from cancer patients, were applied to the Q60 noise model variant allele identification filter. As shown in the figure, the lengths of fragments encompassing a reference allele at a location associated with an identified variant allele (NORMALQ60) were longer, on average, then the lengths of fragments encompassing a variant allele passing the Q60 filter, e.g., identified as variants that are relevant to the biology of the patient's cancer. This shift in median fragment length is indicative of fragments that originated from cancerous cells, suggesting that the variants passing the Q60 filter are enriched for variants that are relevant to the biology of the cancer. Examples of variant noise filters are described, for example, in U.S. Provisional Application No. 62/679,347, filed on Jun. 1, 2018, the content of which is expressly incorporated by reference, in its entirety, for all purposes, and particularly for its description of models for variant calling and quality control.

Also as shown in FIG. 32, 99 variant allele loci in the TP53 gene, identified in cell-free DNA isolated from cancer patients, were applied to the Q60 bioinformatics variant allele identification filter. As shown in the figure, the lengths of fragments encompassing a reference allele at a location associated with an identified variant allele (NORMAL) were the same size, on average, as the lengths of fragments encompassing a variant allele passing the PASS filter, e.g., identified as variants that are relevant to the biology of the patient's cancer. The lack of a shift in median fragment length of the PASS fragments, relative to the NORMAL fragments, indicates that the variants identified by the PASS filter are either noise or not relevant to the biology of the cancer.

Finally, as also shown in FIG. 32, 16 variant allele loci in the TP53 gene, identified in cell-free DNA isolated from cancer patients with a hypermutator phenotype and a high tumor burden, were applied to the Q60 noise model variant allele identification filter. As shown in the figure, the Q60 filter is still able to enrich for variant alleles relevant to the biology of the cancer, even though the average length of fragments encompassing a reference allele are partially shifted due to the influence fragments containing the reference alleles from cancerous cells. Specifically, the lengths of fragments encompassing a reference allele at a location associated with an identified variant allele (HN60) were still longer, on average, than the lengths of fragments encompassing a variant allele passing the Q60 filter (HQ60), e.g., identified as variants that are relevant to the biology of the patient's cancer, although the distribution of lengths of fragments encompassing reference alleles and variant alleles overlaps almost entirely.

Taken together, these results provide diagnostic evidence that the Q60 noise modeling filtering technique is enriching for variant alleles in the TP53 gene that originate from the cancer of the patient. These results also provide diagnostic evidence that the PASS bioinformatics filtering technique is not enriching for variant alleles in the TP53 gene that originate from the cancer of the patient.

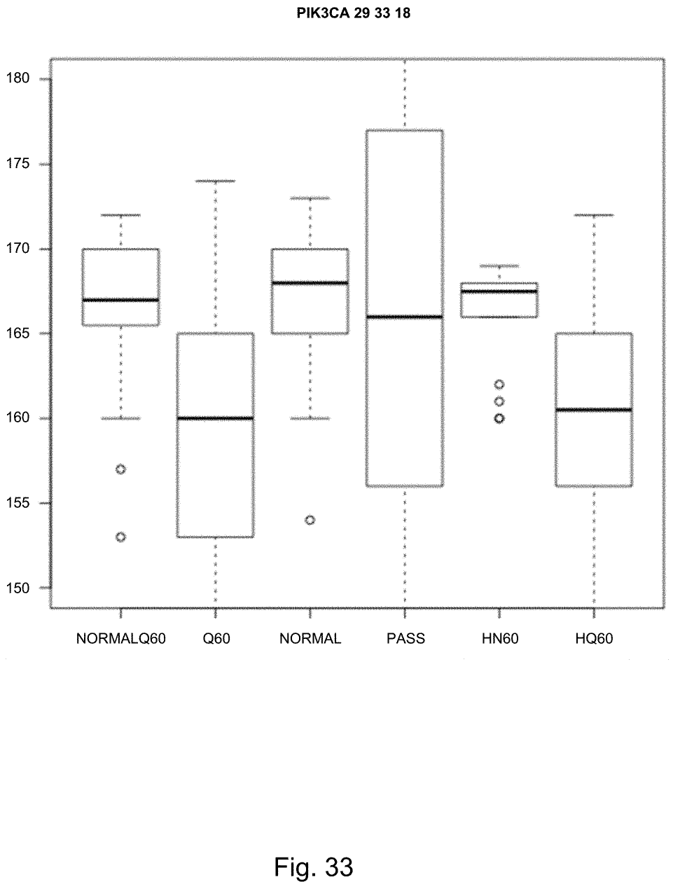

Next, the lengths of fragments encompassing loci corresponding to identified variant alleles in the PIK3CA gene were evaluated in the context of two variant calling algorithms, Q60 and PASS, to determine whether the algorithms are correctly identifying variant alleles in the PIK3CA gene that are relevant to cancer biology. As shown in FIG. 33, and similar to the results for the TP53 gene, the 29 PIK3CA variant alleles identified as informative by the Q60 noise filter display, on average, a fragment length shift characteristic of fragments derived from cancerous cells, while the 33 PIK3CA variant alleles identified as informative by the PASS bioinformatics filter display only a very modest shift in average length. Likewise, the 18 PIK3CA variant alleles identified from patients with hypermutator phenotypes having high tumor burdens also appear to be correctly classified by the Q60 noise model filter.

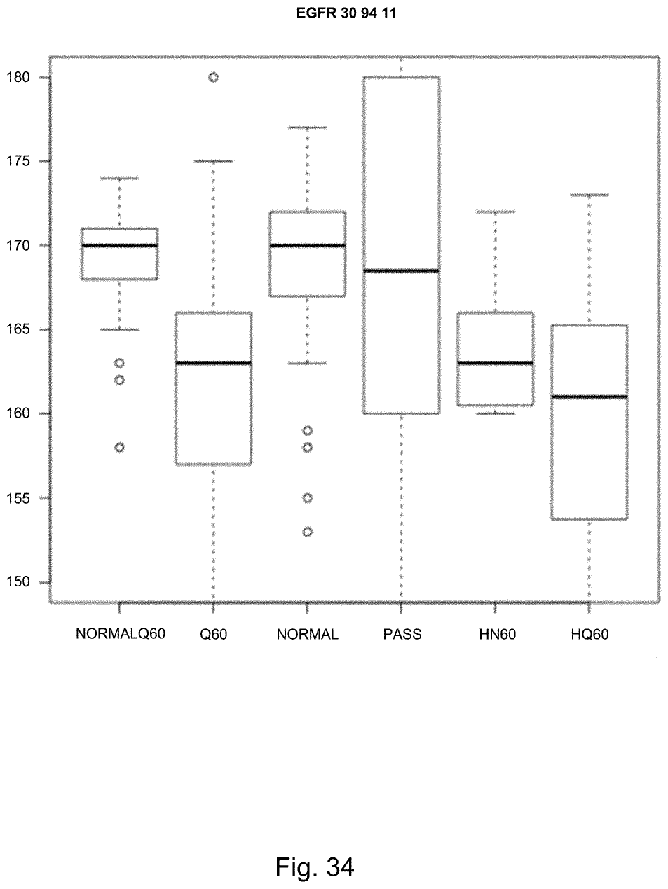

Next, the lengths of fragments encompassing loci corresponding to identified variant alleles in the EGFR gene were evaluated in the context of two variant calling algorithms, Q60 and PASS, to determine whether the algorithms are correctly identifying variant alleles in the EGFR gene that are relevant to cancer biology. As shown in FIG. 34, and similar to the results for the TP53 gene, the 30 EGFR variant alleles identified as informative by the Q60 noise filter display, on average, a fragment length shift characteristic of fragments derived from cancerous cells, while the 94 EGFR variant alleles identified as informative by the PASS bioinformatics filter display only a very modest shift in average length. Likewise, the 11 EGFR variant alleles identified from patients with hypermutator phenotypes having high tumor burdens also appear to be correctly classified by the Q60 noise model filter, although the shift is significantly less pronounced.

Finally, the lengths of fragments encompassing loci corresponding to identified variant alleles in the TET2 gene were evaluated in the context of two variant calling algorithms, Q60 and PASS, to determine whether the algorithms are correctly identifying variant alleles in the TET2 gene that are relevant to cancer biology. As shown in FIG. 35, and unlike for the TP53, PIK3CA, and EGFR variant alleles, neither the 16 TET2 variant alleles identified as informative by the Q60 filter not the 92 TET2 variant alleles identified as informative by the PASS filter display the fragment length shift characteristic of cancer cell-derived fragments, suggesting that both filters are selecting too many of the TET2 variants. This result is explained, in part, by the biology of the TET2 gene, which is associated with high rates of mutation during clonal hematopoiesis. Accordingly, many of the TET2 variants found in cell-free DNA should be arising from white blood cells, rather than from cancer cells.

### Example 14—Classification of Novel Somatic Variants

Targeted, capture-based DNA sequencing of cell-free DNA in a blood sample from a subject confirmed to cancer were generated and mapped to a reference genome, as described above. A total of 947 single nucleotide variants (SNVs) detected at the loci of interest were identified in the sequencing data. These loci were also sequenced in genomic DNA from (i) a tumor biopsy (e.g., cancer cells) from the subject, (ii) white blood cells from the subject, and (iii) a non-cancerous tissue sample from the subject. The origin of the 947 SNVs identified in the cell-free DNA were then matched to the three tissue types, allowing identification of the origins of each of the variants, as described in Examples 1-3. Of the variant alleles, nine were identified as originating from cancer cells, 14 were identified as originating from clonal hematopoiesis (e.g., from white blood cells), and 909 were identified as originating from the germline. 15 SNVs, however, were not matched to any of these sources.

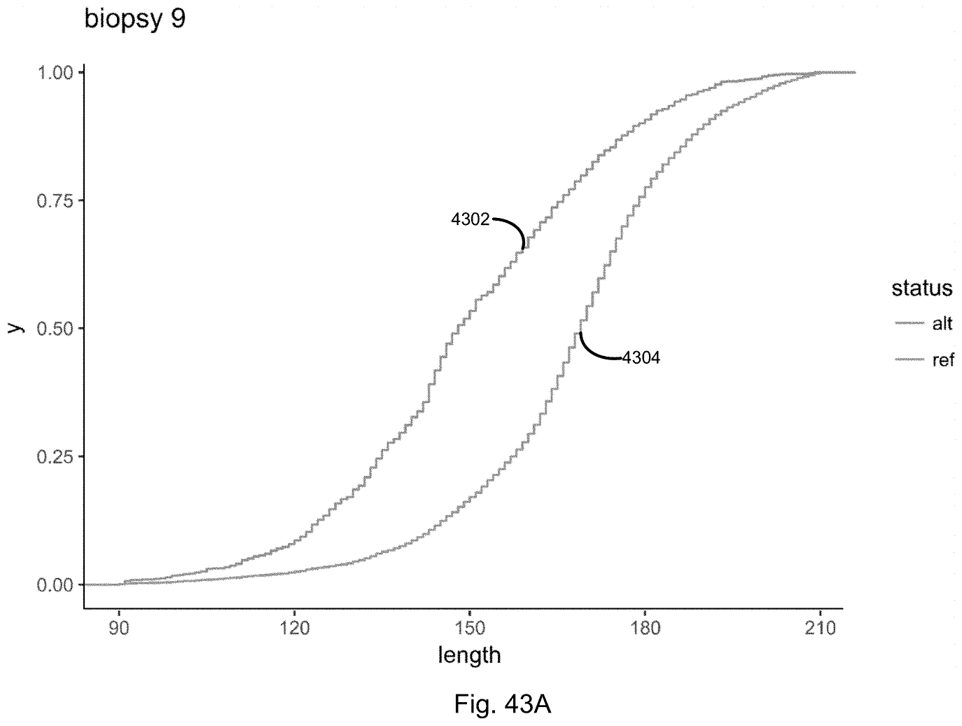

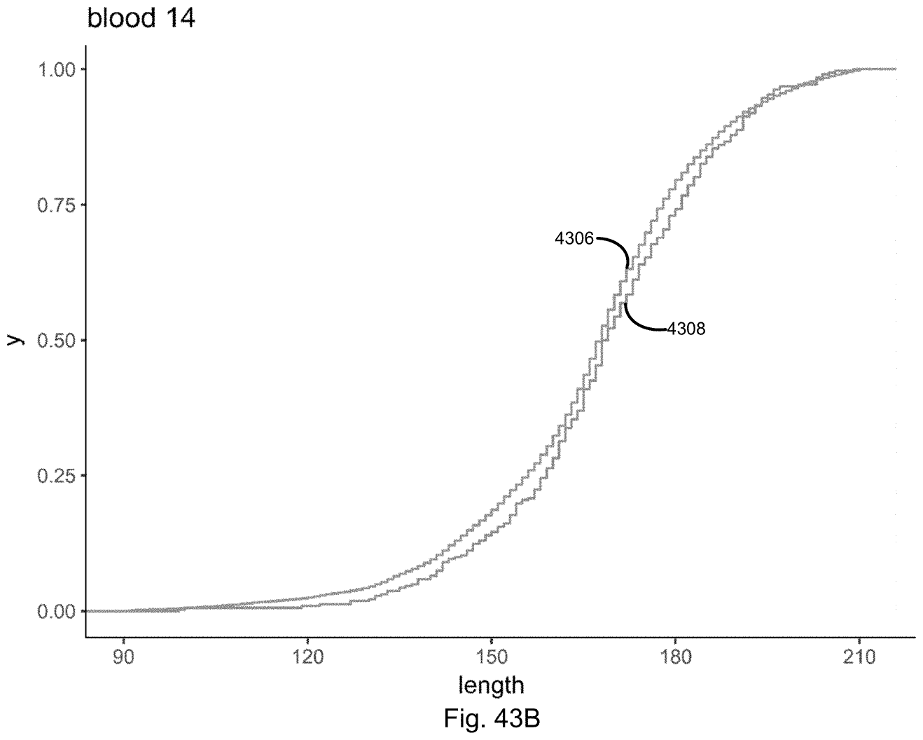

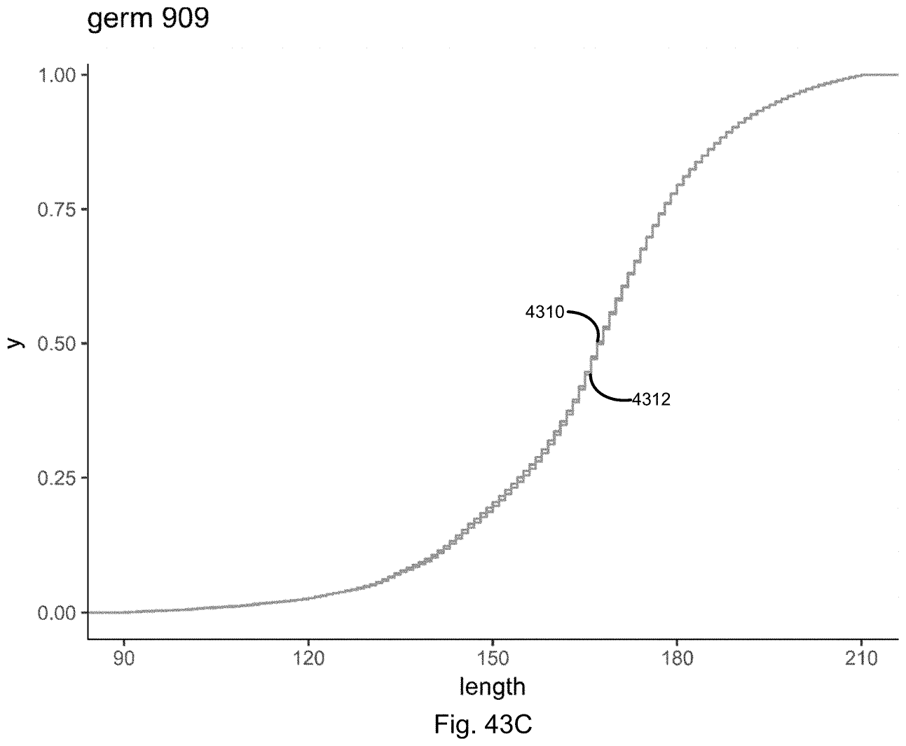

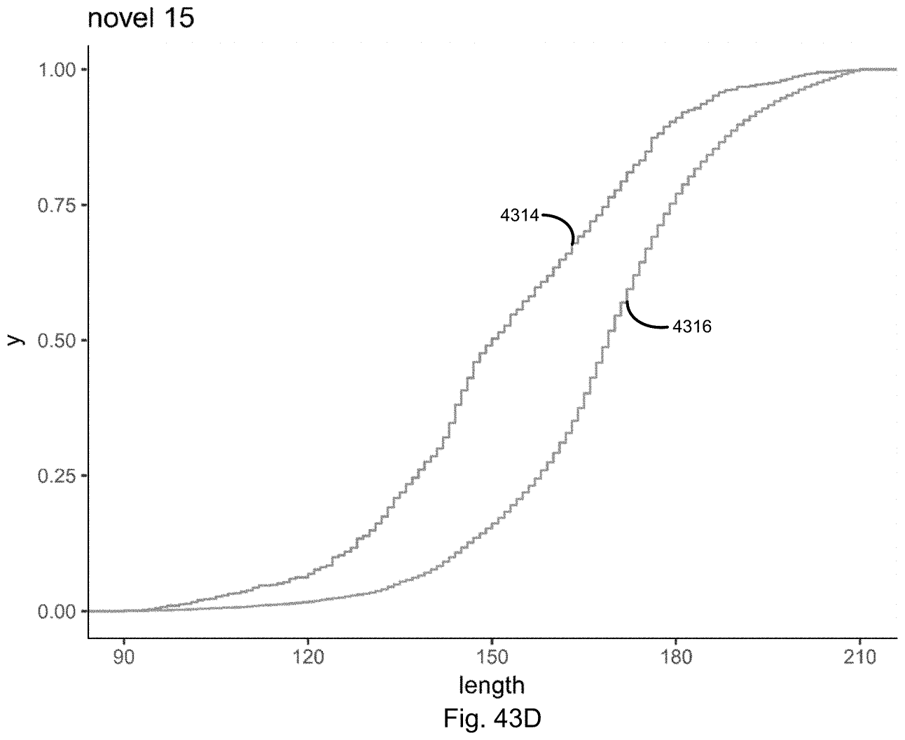

Briefly, when the lengths of cell-free DNA fragments encompassing loci associated with SNVs matched to a cancerous origin were cumulatively plotted as containing a variant allele (4302) or containing a reference allele (4304), the distribution of lengths matched the expected model, where cell-free DNA fragments encompassing the variant allele (4302) had smaller lengths on average than cell-free DNA fragments encompassing the reference allele (4304), as shown in FIG. 43A. When the lengths of cell-free DNA fragments encompassing loci associated with SNVs matched to white blood cells were cumulatively plotted as containing a variant allele (4308) or containing a reference allele (4306), the distribution of lengths matched the expected model, where cell-free DNA fragments encompassing the variant allele (4308) had greater lengths on average than cell-free DNA fragments encompassing the reference allele (4306), as shown in FIG. 43B. Likewise, when the lengths of cell-free DNA fragments encompassing loci associated with SNVs matched to the germline were cumulatively plotted as containing a variant allele (4310) or containing a reference allele (4312), the distribution of lengths matched the expected model, where cell-free DNA fragments encompassing the variant allele (4310) had similar lengths on average to cell-free DNA fragments encompassing the reference allele (4312), as shown in FIG. 43C. When the lengths of cell-free DNA fragments encompassing the 15 loci associated with SNVs with an unidentified origin were cumulatively plotted as containing a variant allele (4314) or containing a reference allele (4316), it could be seen that the distribution of lengths of the cell-free DNA fragments encompassing the variant alleles (4314) was shifted shorter than the distribution of lengths of the cell-free DNA fragments encompassing the reference alleles (4316), as shown in FIG. 43D. This result is consistent with a hypothesis that the unidentified variants arose from cancer cells, because the shift in fragment lengths appears to be consistent with the model behavior expected of variant alleles arising from a cancer cell.

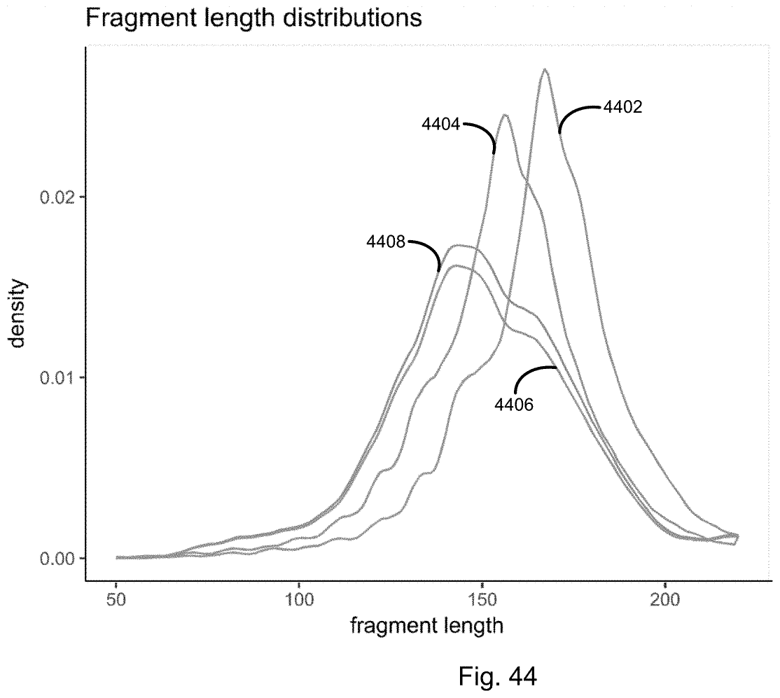

Shown in FIG. 44 is a plot of the underlying fragment length distributions for a global background length distribution obtained from the germline variants (4402), a shifted distribution of fragment lengths based on a typical shift (e.g., seen in cell-free DNA fragments from cancer cells) of about 11 bases (4404), the observed distribution from the alternate alleles in biopsy matched fragments (4406), and a blend of the two distributions, for use when few alternate alleles are available (4408), which can be used to train the EM algorithm.

In order to test the hypothesis that the 15 unmatched variants did arise from cancer cells, a mixture model can be used in conjunction with an expectation maximization (EM) algorithm to determine, for each unidentified allele, a confidence that the allele originated from cancerous or non-cancerous cells. A likelihood can be fit that variants come from the differing length distributions using an EM algorithm. In this algorithm, a latent probability that variants within a class come from the normal length distribution or a shifted distribution is fitted. The shifted distribution either from a shift of the reference distribution, or from a blend of the observed alternate alleles that are biopsy matched and a shift of the reference distribution can be used. In this case, simulating the event where the biopsy matched variants are unknown, the responsibility is fit using the generic shifted distribution, so the biopsy matched variants can be seen to classify effectively as well as the novel somatic variants.

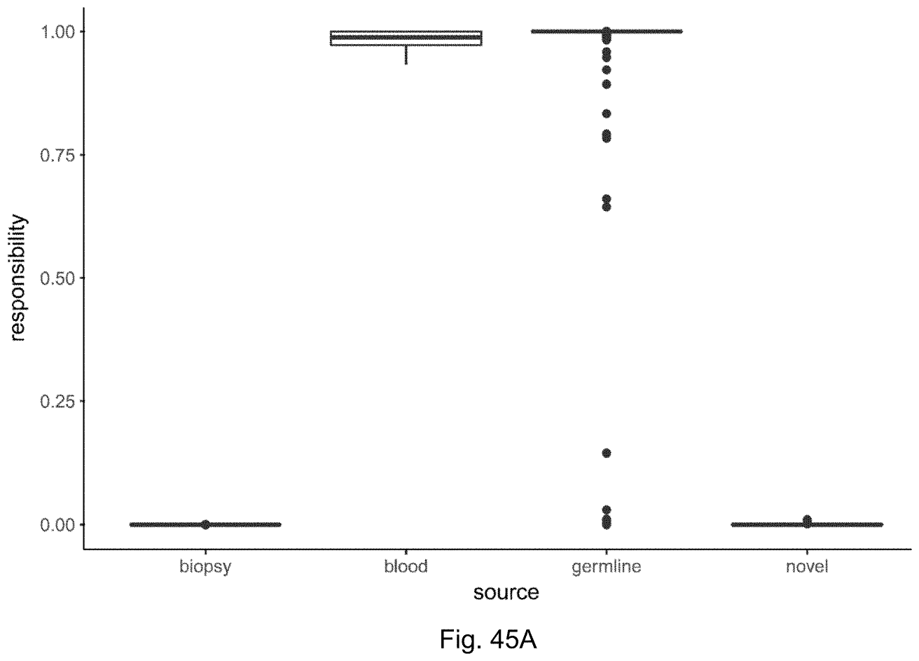

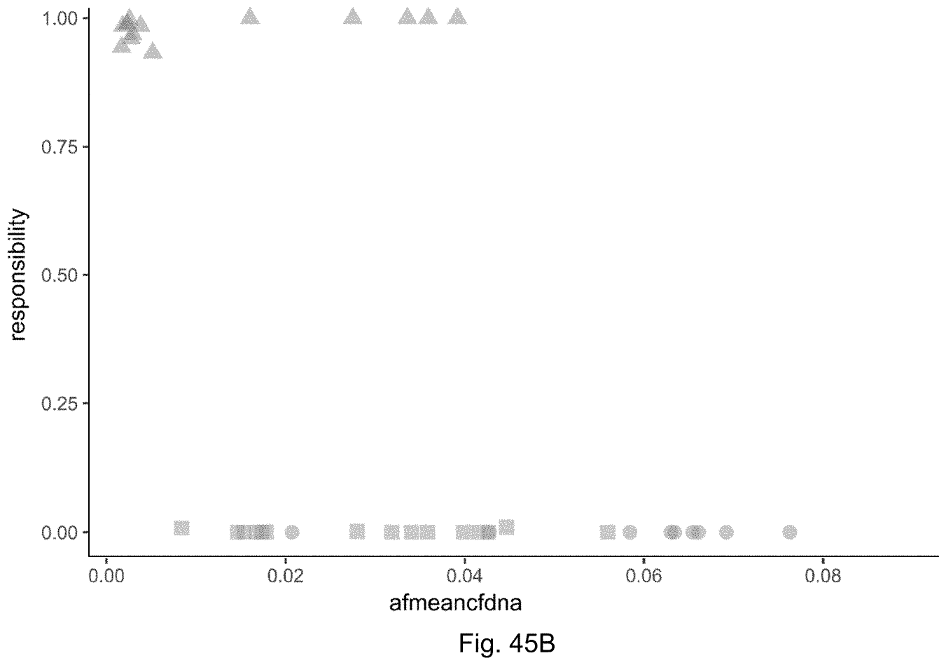

The results of the EM analysis are shown in FIG. 45A, where the responsibility computed from the EM procedure is plotted for each group of variant alleles; that is, the mixture model output of the probability that a variant belongs to the non-cancer related variant distribution. The results can also be visualized by plotting the responsibility as a function of allele frequency for individual alleles, as shown in FIG. 45B. As shown in these figures, the EM algorithm assigned a low level of responsibility to each of the 15 loci corresponding to the biopsy-matched variants, indicating that these variant alleles did not originate from a non-cancerous origin, thus suggesting that they originated from a cancerous origin. As can be seen, the biopsy matched variants were also assigned low responsibility, as expected for variant alleles known to originate from cancer cells. Conversely, the EM algorithm assigned a high responsibility to all 14 loci associated with white blood cell-matched variants, indicating these variants arose from a non-cancerous origin. Similarly, the majority of the 909 loci associated with germline variant alleles were assigned a high responsibility, indicating their origin from a non-cancerous origin. The few loci that were not assigned a high responsibility can likely be explained by the presence of copy number aberrations in the cancer genome of the subject.

### Example 15—Cell-Free DNA (cfDNA) Fragment Length Patterns of Tumor- and Blood-Derived Variants in Participants with and without Cancer

This analysis leverages data from the Circulating Cell-free Genome Atlas study (NCT02889978), a prospective, multi-center, longitudinal observational study designed to develop a single blood test for multiple types of cancer across stages, to examine cfDNA variant fragment lengths across >10 tumor types and to describe the nature of the associated cfDNA variants.

Briefly, plasma samples (N=1406) were evaluated from participants with cancer (n=845) and without cancer (n=561); the breakdown of cancer types is depicted in Table 1.

TABLE 1
Sample breakdown

| Group | N |
|---|---|
| Non-cancer | 561 |
| Lung | 118 |
| Breast | 339 |
| Prostate | 69 |
| Colorectal | 45 |
| Uterine | 27 |
| Pancreas | 26 |
| Renal | 26 |
| Esophageal | 24 |
| Lymphoma | 22 |
| Head/Neck | 19 |
| Ovarian | 17 |
| Remaining* | 113 |

*Cancers with ≤15 samples each.

cfDNA and genomic DNA from white blood cells (WBCs) were subjected to a high-intensity targeted sequencing panel (507 genes, 60000×) with error-correction. 533 of the samples also had matched tumor biopsy tissue that were subjected to whole-genome sequencing (30×). Somatic single-nucleotide variants (SNVs) that passed noise filters were identified and classified using the sequencing results into one of four categories: (i) tumor biopsy-matched (TBM; present in cfDNA and biopsy), (ii) WBC-matched (WM; present in cfDNA and WBC), (iii) non-matched (NM; low probability [P<0.01] of being WBC-derived), and (iv) ambiguous (AMB; unidentifiable source).

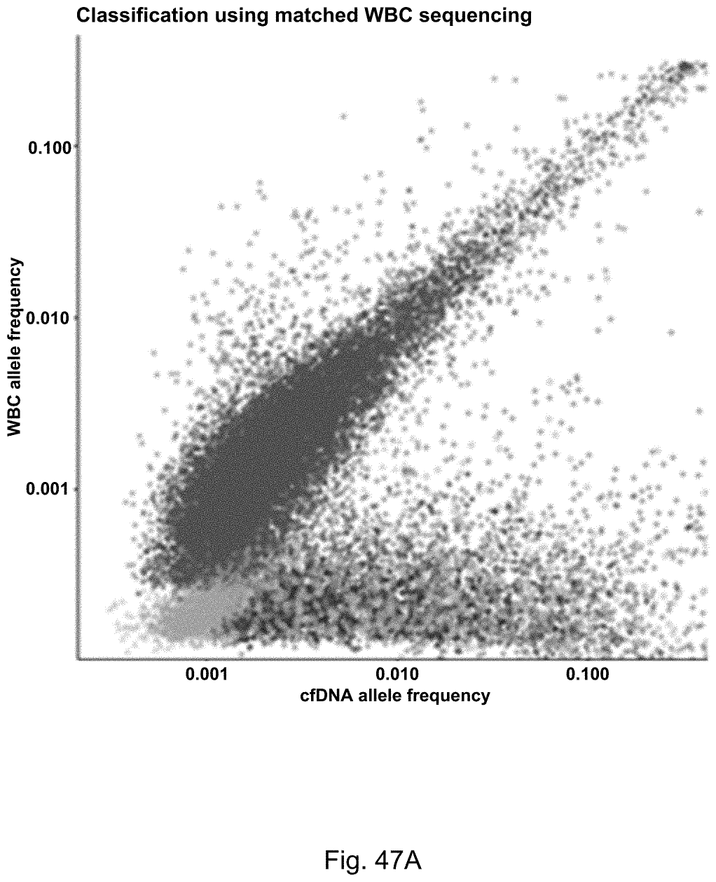

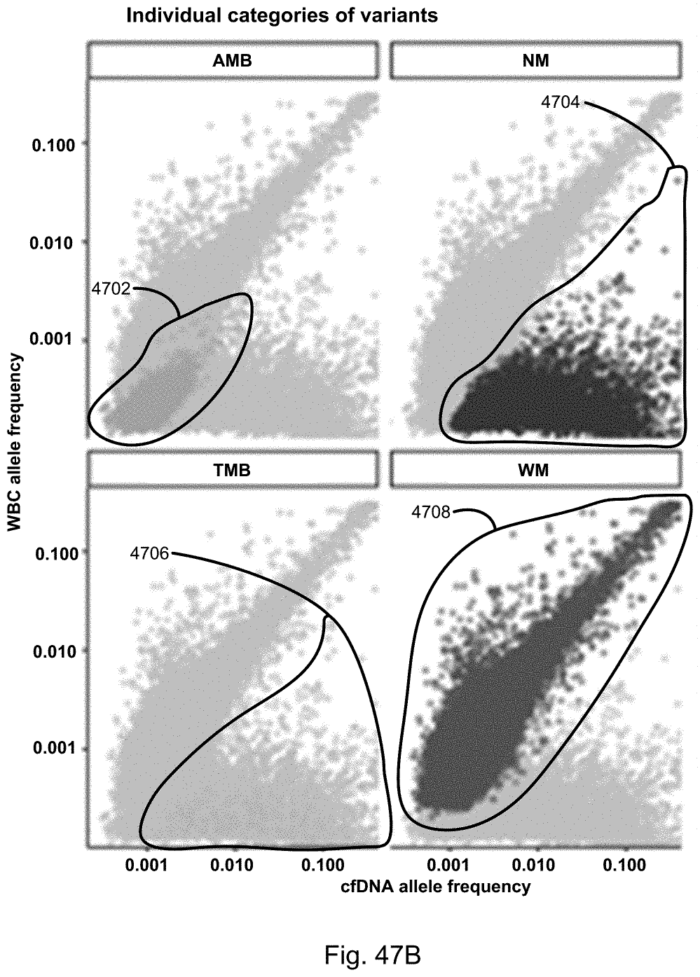

Classification of each of the variant alleles as either cancer or non-cancer derived was accomplished using a joint model between the observed cfDNA alternate allele count given depth and WBC alternate allele count given depth, as illustrated in FIGS. 47A and 47B. Treating both as joint observations from a pair of unknown true frequencies, the likelihood was estimated that the frequency in cfDNA was sufficiently larger than the frequency in WBC that the cfDNA was likely derived from a different source. The joint calling procedure combines a uniform prior on frequency with the observed counts for reference and alternate alleles to compute a posterior mean for the unknown true frequency conditional on the observed values. This posterior mean is always positive, and is used for plotting in the rest of this Example.

Biopsy-matched (TBM) variants were matched to variants detected in tissue samples by simple presence or absence at a location in the genome. “Ambiguous” (AMB) was assigned if the cfDNA frequency could not be determined to be above the WBS frequency with >99% probability, and no alternate alleles were found in the WBC. In this case, there was neither positive evidence for a WBC source, nor could the variant be excluded with sufficient confidence to be accurate.

Statistical Modeling of Source Prediction Based on Fragment Lengths

In all samples, fragment lengths of molecules containing reference and alternate alleles for SNVs were recorded. A statistical model based on fragment lengths was built to predict the likelihood that an SNV belonged to a WBC-like source, without using the WBC sequencing results. This statistical model was constructed as a mixture model: within each individual, a variant was either from a tumor-derived source or a blood-derived source. Under the assumption that the variant is from a given source, the fragment lengths of molecules supporting that variant are each assigned a likelihood from that source distribution based on the density. Aggregating the likelihood over all fragments for a variant, we can compare the total likelihood for the observed data coming from one source to the likelihood that the variant comes from another source to estimate the likelihood that a variant derives from one source or the other. A latent variable representing the overall mixture probability within a sample (i.e., the probability that a randomly selected variant comes from a given source) was constructed as part of the model, and individual variant cluster memberships (responsibilities) were computed by means of an Expectation Maximization algorithm run until convergence.

Likelihoods of fragments of a given length from a given distribution were obtained from an estimated density of fragment lengths for each case. To establish a density for reference alleles, an Epanechnikov kernel was applied to the distribution of reference fragment lengths across samples to estimate density. For alternate alleles, a transformation of this density matching the observed typical distribution of alternate allele lengths in biopsy-matched variants was generated: this avoided overfitting by restricting the degrees of freedom available in the density.

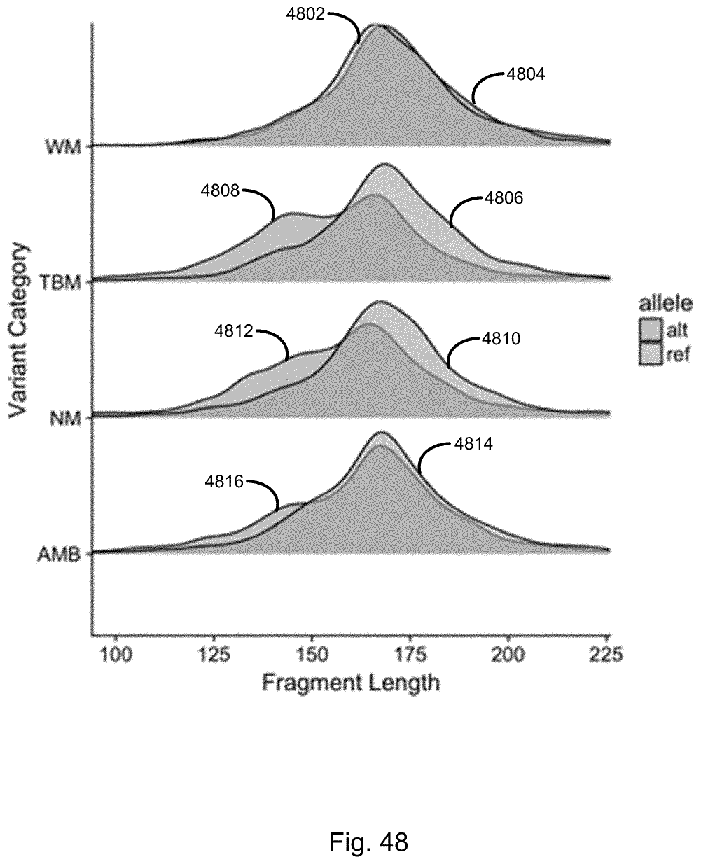

FIG. 48 depicts the four observed size distributions of the plasma DNA fragments. Using the definitive classification derived from matched WBC and tumor tissue, the distribution of fragment lengths was plotted for each category. WBC matched variants had fragment lengths for both reference and alternate alleles, whereas tumor biopsy matched (TBM) variants showed an excess of shorter fragment lengths. Variants not matched to tumor biopsies showed the same shift, suggesting that they are also tumor derived. Variants with ambiguous assignment showed intermediate behavior, and thus were likely a mixture of types. Specifically, tumor biopsy-matched variants (variant allele=4808; reference allele=4806) demonstrated the expected tumor-like shift to the left in the fragment length distribution (Jiang et al., 2015, Proc Natl Acad Sci U.S.A. 112(11), E1317-25; Underhill et al., 2016, PLoS Genet., 12(7):e1006162). Interestingly, non-matched variants showed the same fragment length shift (variant allele=4812; reference allele=4810), suggesting that they are likely not noise, but rather may be variants related to the cancer that were not present in the particular biopsy sample (Gerlinger et al., 2012, N Engl J Med. 366(10), pp. 883-92). As expected, WBC-matched variants (variant allele=4804; reference allele=4802) showed minimal shift in fragment length distribution. Variants that could not be called (AMB; variant allele=4816; reference allele=4814) demonstrated intermediate fragment lengths.

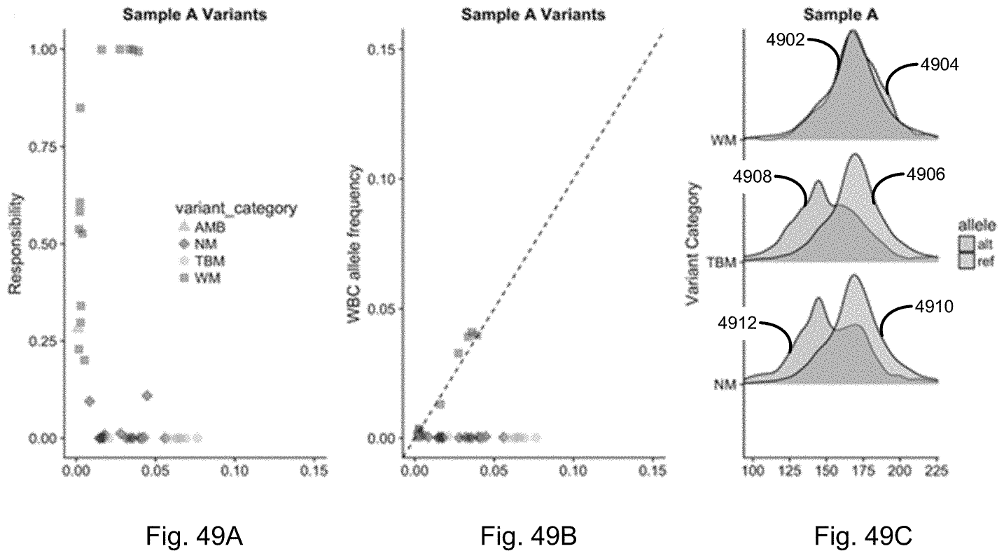

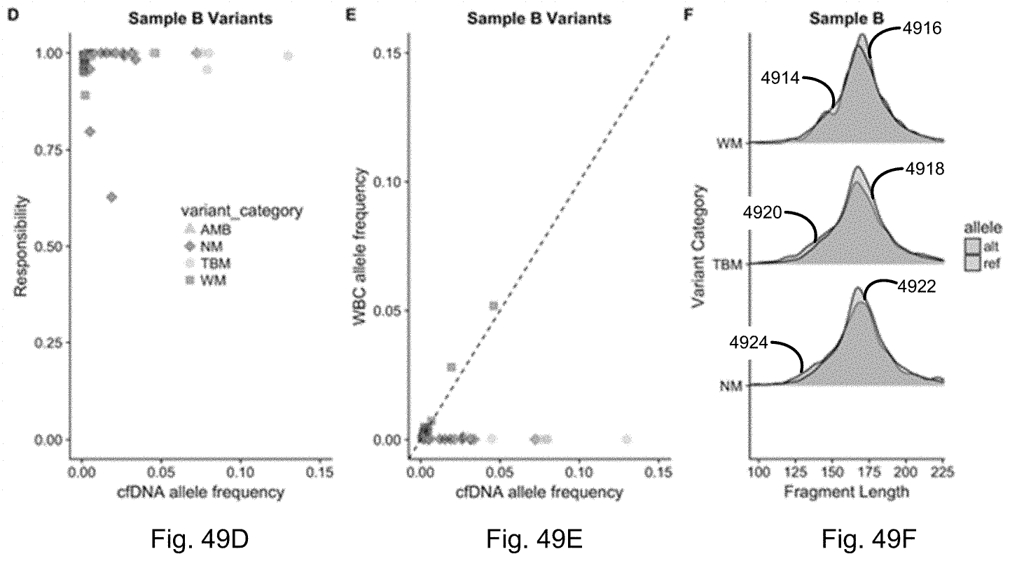

An illustration of the operation of the model is shown in FIG. 49: each variant for a single subject was plotted showing the frequency, responsibility (source probability) for coming from the WBC-matched population of variants. Individual variants of higher frequencies showed clear classification into categories, whereas lower frequency variants had intermediate responsibilities from the model. The participant shown in FIGS. 49A-49C (metastatic esophageal cancer, age 61) shows the expected fragment length shift (FIG. 49C). By contrast, in another individual (FIG. 49D-49F; age 55, metastatic lung cancer) large differences in fragment length were not present (FIG. 49F), limiting the ability to classify variants by means of fragment length within this individual.

Specifically, examples of classification within individual samples are shown in FIGS. 49A-49F. FIG. 49A shows variants classified by fragment length into likely WM (responsibility near 1) and likely tumor derived (NM and TBM), responsibility near 0. Variants with very few alternate alleles were difficult to classify with certainty using fragment length; variants difficult to classify by fragment length were mostly resolved by matched WBC sequencing. FIG. 49B shows variants showing WBC frequency matching. FIG. 49C shows fragment length distributions by allele showing that within Sample A the distributions were very different by category. FIG. 49D shows variants classified by fragment length into likely WM and likely tumor-derived. Note that within Sample B this yielded poor classification performance. FIG. 49E shows variants showing WBC frequency matching. FIG. 49F shows fragment length distributions by allele showing that within Sample B the distributions were not very different even for tumor biopsy-matched variants.

A total of 21,604 SNVs were identified in the cancer and non-cancer samples: 4% were TBM, 68% WM, 19% NM, and 8% AMB (Table 2); the number of samples (non-mutually exclusive) that contributed to each category was 152, 1338, 499, and 761, respectively.

TABLE 2
Variant characteristics

| | | | | | Reference | Alternate |
|---|---|---|---|---|---|---|
| SNV | No. | | No. Samples | | Allele | Allele |
| Category, | SNV | | with SNV | | Length, | Length, |
| Sample | Identified, | | (Total | | Median | Median |
| Type | n (%) | | Samples) | | (SD) | (SD) |
| Tumor- | 811 | (4) | 152 | (1406) | | |
| matched | | | | | | |
| Cancer | 811 | | 152 | (561) | 167 (16.3) | 156 (22.2) |
| Non-cancer | N/A | | N/A | | N/A | N/A |
| WBC- | 14,788 | (68) | 1338 | (1406) | | |
| matched | | | | | | |
| Cancer | 9244 | | 805 | (561) | 168 (16.3) | 169 (14.8) |
| Non-cancer | 5544 | | 533 | (845) | 169 (14.8) | 169 (14.8) |
| Non-matched | 4197 | (19) | 499 | (1406) | | |
| Cancer | 4071 | | 400 | (561) | 167 (17.8) | 158 (20.8) |
| Non-cancer | 126 | | 99 | (845) | 169 (16.3) | 167 (17.8) |
| Ambiguous | 1808 | (8) | 761 | (1406) | | |

| | | | | | |
|---|---|---|---|---|---|
| Cancer | 1,322 | 497 | (561) | 166 (17.8) | 164 (19.3) |
| Non-cancer | 486 | 264 | (845) | 168 (14.8) | 169 (14.8) |

Across SNV categories, the median (SD) length of fragments containing the reference allele was 167 (16.3). In samples derived from cancer participants, the median (SD) fragment lengths of alternate alleles were 156 (22.2; TBM), 169 (14.8; WM), 158 (20.8; NM), and 164 (19.3; AMB), respectively (Table 2). AMB and WM median SNV fragment lengths were similar to that of the reference allele, suggesting that fragment length shifts were minimal in SNVs derived from CH. Fragment lengths of TBM and NM SNVs were similar. Further, most NM SNVs came from cfDNA samples in the cancer cohort, suggesting that NM SNVs may be tumor-derived. Most SNVs occurred in the WM category, which was expected in a population with a median (SD) age of 61 (12.2) due to age-related CH (Genovese et al., 2014; Coombs et al, 2017; Jaiswal et al, 2014).

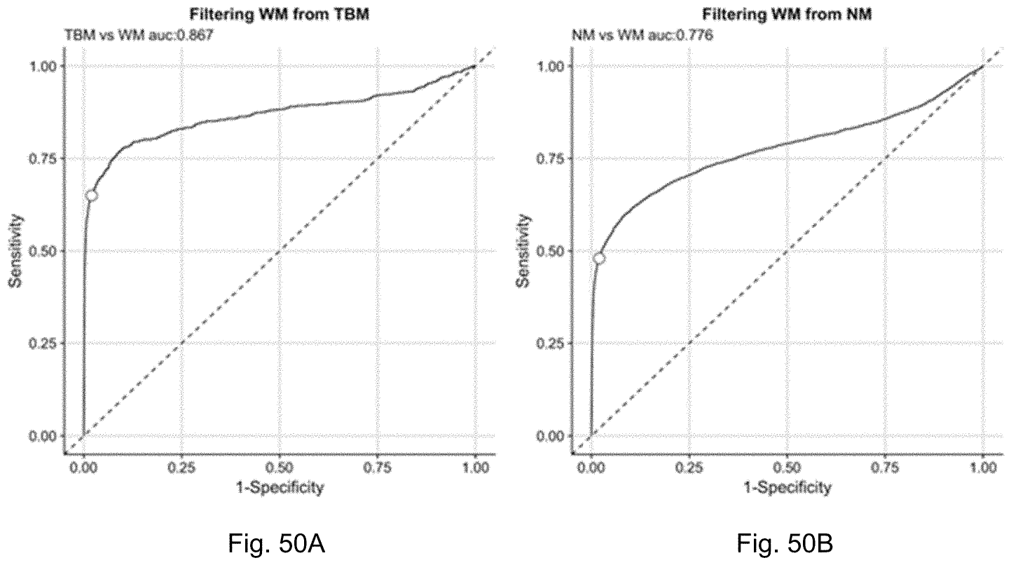

The prediction model distinguished TBM from WM SNVs with an AUC of 0.87. However, at a specificity of 98% (to match filtering based on WBC sequencing), false-negative rates were 35% (TBM; FIG. 50A) and 52% (NM; FIG. 50B). Without white blood cell sequencing, WBC-matched variants are intermixed with other variants passing the noise filter. As shown in FIG. 50A, using fragment length information, it is possible to partially classify WM variants from biopsy matched variants, however at high specificity, many biopsy matched variants are also lost. Similarly, as shown in FIG. 50B, the variants not matched in WBC and not matched to tumor can be partially classified by fragment length, but many are lost at high specificity.

In conclusion, characterizing the sources of cfDNA variants using high-depth, error-corrected sequencing (per-site error rate of <0.001) identified WBC-derived variants with low probability of error. By contrast, because most fragment length distributions from varied sources overlapped, fragment length alone did not strongly distinguish tumor-derived from WBC-derived variants. Therefore, to detect non-metastatic tumors, the lowest possible frequency of mutations needs to be analyzed reliably to find the lowest ctDNA fraction cancer individuals against this background. Together, these data suggest that source prediction based on fragment length alone is less robust than source assignment using individual-matched WBC sequencing, highlighting the importance of accounting for CH-derived SNVs when using targeted cfDNA-based approaches for cancer detection.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in any combination of FIGS. 1A, 1B, and/or as described in FIGS. 37, 38, 39, 40, 41, and 42. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A method of segmenting all or a portion of a reference genome for a species of a subject, the method comprising:
at a computer system comprising one or more processors, and memory storing one or more programs for execution by the one or more processors:
(A) obtaining a dataset comprising a plurality of nucleic acid fragment sequences in electronic form from cell-free DNA in a first biological fluid sample from the subject, wherein each respective nucleic acid fragment sequence in the plurality of nucleic acid fragment sequences represents all or a portion of a respective cell-free DNA molecule in a population of cell-free DNA molecules in the first biological fluid sample, the respective nucleic acid fragment sequence encompassing a corresponding locus in a plurality of loci, wherein each locus in the plurality of loci is represented by at least two different alleles within the population of cell-free DNA molecules;
(B) assigning, for each respective allele represented at each locus in the plurality of loci, a size-distribution metric based on a characteristic of the distribution of the fragment lengths of the cell-free DNA molecules in the population of cell-free DNA molecules that encompass the allele, thereby obtaining a set of size-distribution metrics;
(C) assigning, for each respective allele represented at each locus in the plurality of loci, one or both of:
(1) a read-depth metric based on a frequency of nucleic acid fragment sequences, in the plurality of nucleic acid fragment sequences, associated with the respective allele, thereby obtaining a set of read-depth metrics associated with the plurality of loci, and
(2) an allele-frequency metric based on (i) a frequency of occurrence of the respective allele of the respective locus across the plurality of nucleic acid fragment sequences and (ii) a frequency of occurrence of a second allele of the respective locus across the plurality of nucleic acid fragment sequences, thereby obtaining a set of allele-frequency metrics associated with the plurality of loci;
(D) using the set of size-distribution metrics and one or both of the set of (1) read-depth metrics and (2) allele-frequency metrics to segment all or a portion of the reference genome for the species of the subject.

2-13. (canceled)

14. A method of phasing alleles present on a matching pair of chromosomes in a cancerous tissue of a subject that is a member of a species, the method comprising:
at computer system having one or more processors, and memory storing one or more programs for execution by the one or more processors:
(A) obtaining a dataset comprising a plurality of nucleic acid fragment sequences in electronic form from a first biological fluid sample of the subject, wherein each respective nucleic acid fragment sequence in the plurality of nucleic acid fragment sequences represents all or a portion of a respective cell-free DNA molecule in a population of cell-free DNA molecules in the first biological fluid sample, the respective nucleic acid fragment sequence encompassing a corresponding locus in a plurality of loci, wherein each locus in the plurality of loci is represented by at least two different alleles within the population of cell-free DNA molecules;
(B) compressing the dataset by assigning, for each respective allele represented at each locus in the plurality of loci, a size-distribution metric based on a characteristic of a distribution of the fragment lengths of the cell-free DNA molecules in the population of cell-free DNA molecules that encompass the respective allele, thereby obtaining a set of size-distribution metrics;
(C) identifying a first locus in the plurality of loci, represented by both (i) a first allele having a first size-distribution metric and (ii) a second allele having a second size-distribution metric, wherein a threshold probability or likelihood exists that the copy number of the first allele is different than the copy number of the second allele in a subpopulation of cells within the cancerous tissue of the subject as determined by a parametric or non-parametric based classifier that evaluates one or more properties of the cell-free DNA molecules in the sample that encompass the first locus, wherein the one or more properties includes the first size-distribution metric and the second size-distribution metric;
(D) determining, for a second locus in the plurality of loci located proximate to the first locus on a reference genome for the species of the subject, the second locus represented by both (iii) a third allele having a third size-distribution metric and (iv) a fourth allele having a fourth size-distribution metric, whether a threshold probability exists that the copy number of the third allele is different than the copy number of the fourth allele in the subpopulation of cells as determined by a parametric or non-parametric based classifier that evaluates one or more properties of the cell-free DNA molecules in the sample that encompass the second locus, wherein the one or more properties includes the third size-distribution metric and the fourth size-distribution metric; and
(E) when the threshold probability or likelihood exists that the copy number of the third allele is different than the copy number of the fourth allele in the subpopulation of cells, determining whether it is more likely that the copy number of the first allele is more similar to the copy number of the third allele or the copy number of the fourth allele in the sub-population of cancer cells; wherein:
when it is more likely that the copy number of the first allele is more similar to the copy number of the third allele in the subpopulation of cancer cells, assigning the first allele and the third allele to a first chromosome in a matching pair of chromosomes and assigning the second allele and the fourth allele to a second chromosome in the matching pair of chromosomes that is different than the first chromosome, and
when it is more likely that the copy number of the first allele is more similar to the copy number of the fourth allele in the subpopulation, assigning the first allele and the fourth allele to a first chromosome in a matching pair of chromosomes and assigning the second allele and the third allele to a second chromosome in the matching pair of chromosomes that is different than the first chromosome;
thereby phasing the allele sequences at the first and second loci present on a matching pair of chromosomes in the cancerous tissue.

15-44. (canceled)

45. A method of detecting a loss in heterozygosity at a genomic locus in a cancerous tissue of a subject, the method comprising:
at a computer system having one or more processors, and memory storing one or more programs for execution by the one or more processors:
(A) obtaining a dataset comprising a plurality of nucleic acid fragment sequences in electronic form from a first biological fluid sample of the subject, wherein each respective nucleic acid fragment sequence in the plurality of nucleic acid fragment sequences represents all or a portion of a respective cell-free DNA molecule, in a population of cell-free DNA molecules in the first biological fluid sample, the respective nucleic acid fragment sequence encompassing a corresponding locus in a plurality of loci, wherein each locus in the plurality of loci is represented by at least two different germline alleles;
(B) compressing the dataset by assigning, for each respective germline allele represented at each locus in the plurality of loci, a size-distribution metric based on a characteristic of the distribution of the fragment lengths of the cell-free DNA molecules in the population of cell-free DNA molecules that encompass the respective germline allele, thereby obtaining a set of size-distribution metrics; and
(C) determining an indicia that a loss of heterozygosity has occurred at a respective locus in the plurality of locus using a parametric or non-parametric based classifier that evaluates one or more properties of the cell-free DNA molecules in the population of cell-free DNA molecules that encompass the respective locus, wherein the one or more properties includes the size-distribution metrics for the corresponding at least two different germline alleles of the respective locus in the set of size-distribution metrics.

46-70. (canceled)

71. A method of determining the cellular origin of variant alleles present in a biological fluid sample, the method comprising:
at computer system having one or more processors, and memory storing one or more programs for execution by the one or more processors:
(A) obtaining a dataset comprising a first plurality of nucleic acid fragment sequences in electronic form from a first biological fluid sample from a subject, wherein each respective nucleic acid fragment sequence in the first plurality of nucleic acid fragment sequences represents all or a portion of a respective cell-free DNA molecule in a population of cell-free DNA molecules in the first biological fluid sample, the respective nucleic acid fragment sequence encompassing a corresponding locus, in a plurality of loci, represented by at least a reference allele and a variant allele within the population of cell-free DNA molecules;
(B) compressing the dataset by assigning, for each respective allele represented at each locus in the plurality of loci, a size-distribution metric based on a characteristic of the distribution of the fragment lengths of the cell-free DNA molecules in the population of cell-free DNA molecules that encompass the respective allele, thereby obtaining a set of size-distribution metrics; and
(C) assigning each respective variant allele of a respective locus in the plurality of loci either to a first category of alleles originating from non-cancerous cells or to a second category of alleles originating from cancer cells using a parametric or non-parametric based classifier that evaluates one or more properties of the cell-free DNA molecules in the sample that encompass the respective locus, wherein the one or more properties include the size-distribution metric for the variant allele of the respective locus.

72-101. (canceled)

102. A method of identifying and canceling an incorrect mapping of a nucleic acid fragment sequence to a position within a reference genome, the method comprising:
at computer system having one or more processors, and memory storing one or more programs for execution by the one or more processors:
(A) obtaining a dataset comprising a plurality of nucleic acid fragment sequences in electronic form from a first biological fluid sample from a subject, wherein each respective nucleic acid fragment sequence in the plurality of nucleic acid fragment sequences represents all or a portion of a respective cell-free DNA molecule in a population of cell-free DNA molecules in the first biological fluid sample, the respective nucleic acid fragment sequence encompassing a corresponding locus, in a plurality of loci, represented by at least two different alleles within the population of cell-free DNA molecules;
(B) mapping each respective nucleic acid fragment sequence in the plurality of nucleic acid fragment sequences to a position within a reference genome for the species of the subject, wherein the position within the reference genome encompasses a putative locus in the plurality of loci encompassed by the population of cell-free DNA molecules, based on sequence identity shared between the respective nucleic acid fragment sequence and the nucleic acid sequence at the position within the reference genome;
(C) compressing the dataset by assigning, for each respective allele of each respective locus in the plurality of loci, a size-distribution metric corresponding to a characteristic of the distribution of the fragment lengths of the cell-free DNA molecules that are both (i) represented by a respective nucleic acid fragment sequence in the plurality of nucleic acid fragment sequences that encompass the respective allele and (ii) mapped to a same corresponding position within the reference genome, thereby obtaining a set of size-distribution metrics;
(D) determining a confidence metric for the mapping of respective nucleic acid fragment sequences encompassing an allele of a respective locus to a corresponding position within the reference genome encompassing a putative allele by using a parametric or non-parametric based classifier that evaluates one or more properties of the cell-free DNA molecules that are both (i) represented by a respective nucleic acid fragment sequence that encompasses the respective allele and (ii) mapped to the corresponding position within the reference genome, wherein the one or more properties include the size-distribution metric for the respective allele; and
(E) when the confidence metric fails to satisfy a threshold measure of confidence, canceling the mapping of the respective nucleic acid fragment sequences to the corresponding position within the reference genome.

103-126. (canceled)

127. A method of validating the use of genotypic data from a particular genomic locus in a subject classifier for classifying a cancer condition for a species, the method comprising:
at computer system having one or more processors, and memory storing one or more programs for execution by the one or more processors:
(A) obtaining a subject classifier that uses data from the particular genomic locus to classify the cancer condition for a query subject of the species;
(B) obtaining, for each respective validation subject in a plurality of validation subjects of the species: (i) a cancer condition and (ii) a validation genotypic data construct that includes one or more genotypic characteristics, thereby obtaining a set of cancer conditions and a correlated set of validation genotypic data constructs, wherein:
each genotypic data construct in the set of genotypic data constructs is obtained from a respective first plurality of nucleic acid fragment sequences in electronic form from a corresponding first biological fluid sample from a respective validation subject in the plurality of validation subjects,
each respective nucleic acid fragment sequence in the respective first plurality of nucleic acid fragment sequences represents all or a portion of a respective cell-free DNA molecule in a population of cell-free DNA molecules in the corresponding biological fluid sample, the respective nucleic acid fragment sequence encompassing a corresponding locus, in a plurality of loci, represented by at least two different alleles within the population of cell-free DNA molecules, and
the one or more genotypic characteristics in the validation genotypic data construct include a size-distribution metric corresponding to a characteristic of the distribution of the fragment lengths of the cell-free DNA molecules that encompass a respective allele of the particular genomic locus; and
(C) determining a confidence metric for use of genotypic data from the particular genomic locus in the subject classifier by using a parametric or non-parametric based test classifier that evaluates the size distribution metric for the respective allele in each respective validation genotype data construct and each correlated cancer status in the set of cancer conditions.

128-153. (canceled)

154. The method of claim 71, wherein the subject has not been diagnosed as having cancer.

155. (canceled)

156. The method of claim 71, wherein the plurality of loci is selected from a predetermined set of loci that includes less than all loci in the genome of the subject.

157-160. (canceled)

161. The method of claim 156, wherein the average coverage rate of nucleic acid fragment sequences of the predetermined set of loci taken from the sample is at least 500×.

162. (canceled)

163. The method of claim 71, wherein the plurality of loci is selected from all loci in the genome of the subject.

164. The method of claim 163, wherein an average coverage rate of nucleic acid fragment sequences across the genome of the subject is at least 20×.

165. (canceled)

166. The method of claim 71, wherein the at least two different alleles of a respective locus include a variant allele that is a single nucleotide polymorphism relative to a reference allele for the locus.

167. The method of claim 71, wherein the at least two different alleles of a respective locus include a variant allele that is a deletion of twenty-five nucleotides or less, encompassing the respective locus, relative to a reference allele for the locus.

168. The method of claim 71, wherein the at least two different alleles of a respective locus include a variant allele that is a single nucleotide deletion relative to a reference allele for the locus.

169. The method of claim 71, wherein the at least two different alleles of a respective locus include a variant allele that is an insertion of twenty-five nucleotides or less, encompassing the respective locus, relative to a reference allele for the locus.

170. The method of claim 71, wherein the at least two different alleles of a respective locus include a variant allele that is a single nucleotide insertion relative to a reference allele for the locus.

171. The method of claim 71, wherein the size-distribution metric is a measure of central tendency of length across the distribution.

172. The method of claim 171, wherein the measure of central tendency of length across the distribution is an arithmetic mean, weighted mean, midrange, midhinge, trimean, Winsorized mean, median, or mode of the distribution.

173. An electronic device, comprising:
one or more processors;
memory; and
one or more programs, wherein the one or more programs are stored in the memory and configured to be executed by the one or more processors, the one or more programs including instructions for performing the method of claim 71.

174. A computer readable storage medium storing one or more programs, the one or more programs comprising instructions, which when executed by an electronic device with one or more processors and a memory cause the device to perform the method of claim 71.
